(19) United States
(12) Patent Application Publication
(10) Pub. No.: US 2005/0032094 A1
(43) Pub. Date: Feb. 10, 2005

(54) Regulatory DNA sequences of the human catalytic telomerase sub-unit gene, diagnostic and therapeutic use thereof

(75) Inventors: Gustav Hagen, Leverkusen (DE); Maresa Wick, Koln (DE); Dmitry Zubov, Koln (DE)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Pharmaceuticals Corporation, West Haven, CT (US)

(21) Appl. No.: 10/840,455

(22) Filed: May 6, 2004

Related U.S. Application Data
(63) Continuation of application No. 09/582,246, filed on Sep. 21, 2000, now abandoned.

(30) Foreign Application Priority Data
Dec. 24, 1997 (DE) 197 57 984.1

(51) Int. Cl. C12Q 1/68; C07H 21/04; C12N 9/22; C12N 9/64

(52) U.S. Cl. 435/6; 435/69.1; 435/199; 435/226; 435/320.1; 435/325; 536/23.2

(57) ABSTRACT

This invention relates to regulatory DNA sequences, comprising promoter sequences and intron sequences, for the gene for the human catalytic telomerase subunit. In addition, this invention relates to the use of these DNA sequences for pharmaceutical, diagnostic and therapeutic purposes, especially in the treatment of cancer and ageing.

### STRUCTURE AND FUNCTION OF THE CHROMOSOME ENDS

The genetic material of eukaryotic cells is distributed on linear chromosomes. The ends of hereditary units are termed telomeres, derived from the Greek words telos (end) and meros (part, segment). Most telomeres consist of repeats of short sequences which are mainly composed of thymine and guanine (Zakian, 1995). In all the vertebrates which have so far been investigated, the telomeres consist of the sequence TTAGGG (Meyne et al., 1989).

The telomeres have a variety of important functions. They prevent the fusion of chromosomes (McClintock, 1941) and thus the formation of dicentric hereditary units. Such chromosomes having two centromeres can lead to the development of cancer due to loss of heterozygosis or duplication, or loss of genes.

In addition, telomeres serve the purpose of distinguishing intact hereditary units from damaged hereditary units. Thus, yeast cells ceased their cell division when they contained a chromosome without a telomere (Sandell and Zakian, 1993).

Telomeres fulfil another important task in association with the replication of eukaryotic cell DNA. In contrast to the circular genomes of prokaryotes, the linear chromosomes of eukaryotes cannot be completely replicated by the DNA polymerase complex. RNA primers are required to initiate DNA replication. After elimination of the RNA primers, extension of the Okazaki fragments and subsequent ligation, the newly synthesized DNA strand lacks the 5′ end since the RNA primer cannot be replaced by DNA at that point. Without special protective mechanisms, the chromosomes would therefore shrink with each cell division (“end-replication problem”; Harley et al., 1990). The non-coding telomere sequences presumably constitute a buffer-zone for preventing the loss of genes (Sandell and Zakian, 1993).

In addition to this, telomeres also play an import role in regulating cell ageing (Olovnikov, 1973). Human somatic cells exhibit a limited capacity for replication in culture; after a certain period of time, they become senescent. In this state, the cells no longer divide even after having been stimulated with growth factors; however, they do not die and remain metabolically active (Goldstein, 1990). Various observations support the hypothesis that a cell determines how many more times it can divide on the basis of the length of its telomeres (Allsopp et al., 1992).

In summary, the telomeres consequently possess key functions in the ageing of cells, and in stabilizing the genetic material and preventing cancer.

The Enzyme Telomerase Synthesizes the Telomeres

As described above, organisms which possess linear chromosomes can only replicate their genome incompletely in the absence of a special protective mechanism. Most eukaryotes use a special enzyme, i.e. telomerase, for regenerating the telomere sequences. Telomerase is expressed constitutively in the single-cell organisms which have so far been investigated. On the other hand, telomerase activity has only been measured in humans in germ cells and tumour cells, whereas neighbouring somatic tissue did not contain any telomerase (Kim et al., 1994).

Telomerase can also be designated functionally as terminal telomere transferase, which is located in the cell nucleus as a multiprotein complex. While the RNA moiety of human telomerase has been known for a relatively long, period of time (Feng et al., 1995), the catalytic subunit of this enzyme group was recently identified in a variety of organisms (Lingner et al., 1997; cf. our application PCT EP/98/03468 which is likewise pending). These catalytic subunits of telomerase are strikingly homologous both among themselves and in relation to all previously known reverse transcriptases.

WO 98/14592 also describes nucleic acid and amino acid sequences of the catalytic telomerase subunit.

Activation of Telomerase in Human Tumours

It was originally only possible to demonstrate telomerase activity in humans in germ line cells and not in normal somatic cells (Hastie el al., 1990; Kim et al., 1994). Following the development of a more sensitive detection method (Kim et al., 1994), a low telomerase activity was also detected in hematopoietic cells (Broccoli el al., 1995; Counter et al., 1995; Hiyama et al., 1995). It is true, however, that these cells nevertheless exhibited a reduction in the telomeres (Vaziri et al., 1994; Counter et al., 1995). It has still not been resolved whether the quantity of enzyme in these cells is not sufficient for compensating the telomere loss or whether the telomerase activity which is measured stems from a subpopulation, e.g. incompletely differentiated CD34⁺38⁺ precursor cells (Hiyama et al., 1995). In order to resolve this, it would be necessary to detect telomerase activity in a single cell.

Interestingly, however, significant telomerase activity was detected in a large number of the tumour tissues which had thus far been tested (1734/2031, 85%; Shay, 1997), whereas no activity was found in normal somatic tissue (1/196, <1%, Shay, 1997). In addition various investigations have shown that the telomeres still shrank in senescent cells which were transformed with viral oncoproteins and it was only possible to detect telomerase in the subpopulation which survived the growth crisis (Counter et al., 1992). The telomeres were also stable in these immortalized cells. (Counter et al., 1992). Similar findings from investigations in mice (Blasco et al., 1996) support the assumption that reactivation of the telomerase is a late event in tumorigenesis.

Based on these results, a “telomerase hypothesis” was developed which links the loss of telomere sequences and cell ageing with telomerase activity and the development of cancer. In long-lived species such as humans, the shrinking of the telomeres can be regarded as being a mechanism for suppressing tumours. Differentiated cells which do not contain any telomerase cease their cell division at a particular telomere length. If such a cell mutates, it can only form a tumour it the cell can extend its telomeres. Otherwise, the cell would continue to lose telomere sequences until its chromosomes became unstable and it was finally destroyed. Telomerase reactivation is presumably the main mechanism used by tumour cells to stabilize their telomeres.

It follows from these observations and considerations that it should be possible to treat tumours by inhibiting the telomerase. Conventional cancer therapies using cytostatic agents or short-wave radiation damage all the dividing cells in the body in addition to the tumour cells. However, since only germ line cells, apart from tumour cells, contain significant telomerase activity, telomerase inhibitors would attack the tumour cells more specifically and consequently elicit fewer undesirable side effects. Telomerase activity has been detected in all the tumour tissues which have so far been tested, which means that these therapeutic agents could be employed against all types of cancer. The effect of telomerase inhibitors would then set in when the telomeres of the cells had shortened to such an extent that the genome became unstable. Since tumour cells usually possess telomeres which are shorter than those of normal somatic cells, cancer cells would be the first to be eliminated by the telomerase inhibitors. By contrast, cells possessing long telomeres, such as the germ cells, would only be damaged at a much later date. Telomerase inhibitors consequently represent a potential way forward in the treatment of cancer.

It becomes possible to obtain unambiguous answers to the question of the nature and points of attack of physiological telomerase inhibitors once the manner in which expression of the telomerase gene is regulated has also been identified.

Regulation of Gene Expression in Eukaryotes

There are a large number of points in eukaryotic gene expression, i.e. the cellular flow of information from the DNA to the protein by way of the RNA, at which regulatory mechanisms can exert an effect. Examples of individual control steps are gene amplification the recombination of gene loci, chromatin structure, DNA methylation, transcription, post-transcriptional modifications of mRNA, mRNA transport, translation and post-translational modifications of proteins. Studies which have been carried out to date indicate that control at the level of transcription initiation is of the greatest importance (Latchman, 1991).

A region which is responsible for regulating transcription, and which is designated the promoter region, is located directly upstream of the transcription start of a gene which is transcribed by RNA polymerase II. Comparison of the nucleotide sequences of promoter regions from a large number of known genes shows that particular sequence motifs occur regularly in this region. These elements include, inter alia, the TATA box, the CCAAT box and the GC box, which elements are recognized by specific proteins. The TATA box, which is located about 30 nucleotides upstream of the transcription start, is, for example, recognized by the TFIID subunit TBP (“TATA box-binding protein”), whereas particular GC-rich sequence segments are specifically bound by the transcription factor Sp1 (“specificity protein 1”).

The promoter can be functionally subdivided into a regulatory segment and a constitutive segment (Latchman, 1991). The constitutive control region comprises the so-called core promoter switch enables transcription to be initiated correctly. This promoter contains the sequence elements which are described as UPE's (upstream promoter elements) which are necessary for efficient transcription. The regulatory control segments, which can be interlaced with the UPE's, possess sequence elements which can be involved in the signal-dependent regulation of transcription by hormones, growth factors, etc. They impart tissue-specific or cell-specific promoter properties.

DNA segments which are able to exert an influence on gene expression over relatively large distances are a characteristic feature of eukaryotic genes. These elements can be located upstream or downstream of a transcription unit, or within the unit, and can perform their function independently of their orientation. These sequence segments may reinforce (enhancers) or attenuate (silencers) promoter activity. In a similar way to the promoter regions, enhancers and silencers also accommodate several binding sites for transcription factors.

The invention relates to the DNA sequences from the 5′-flanking region of the gene for the catalytically active human telomerase subunit and intron sequences for this gene.

The invention particularly relates to the 5′-flanking regulatory DNA sequence which contains the promoter DNA sequence for the gene for the human catalytic telomerase subunit, as depicted in FIG. 10 (SEQ ID NO 3).

The invention furthermore relates to part regions of the 5′-flanking regulatory DNA sequence, as depicted in FIG. 4 (SEQ ID NO 1), which has a regulatory effect.

Intron sequences for the gene for the human catalytic telomerase subunit, in particular those sequences which have a regulatory effect, are also part of the subject-matter of the present invention. The intron sequences according to the invention are described in detail in the context of Example 5 (cf. SEQ ID NO 4, 5, 6, 7, 8, 9, 10, 11, 12, 13, 14, 15, 16, 17, 18, 19 and 20).

The invention furthermore relates to a recombinant construct which comprises the DNA sequences according to the invention, in particular the 5′-flanking DNA sequence of the gene for the human catalytic telomerase subunit, or part regions thereof.

Preference is given to recombinant constructs which, in addition to the DNA sequences according to the invention, in particular the 5′-flanking DNA sequence of the gene for the human catalytic telomerase subunit, or part regions thereof, also contain one or more additional DNA sequences which encode polypeptides or proteins.

According to a particularly preferred embodiment, these additional DNA sequences encode antineoplastic proteins.

Particular preference is given to those antineoplastic proteins which inhibit angiogenesis directly or indirectly. Examples of these proteins are:

Plasminogen activator inhibitor (PAI-1), PAI-2, PAI-3, angiostatin, endostatin, platelet factor 4, TIMP-1, TIMP-2, TIMP-3 and leukaemia inhibitory factor (LIF).

Antineoplastic proteins which have a direct or indirect cytostatic effect on tumours are likewise particularly preferred. These proteins include, in particular: perforin, granzyme, IL-2, IL-4,- IL-12, interferons, such as IFN-α, IFN-β and IFN-γ, TNF, TNF-α, TNF-β, oncostatin M; tumour suppressor genes, such as p53, retinoblastoma.

Particular preference is furthermore given to antineoplastic proteins which, where appropriate in addition to their antineoplastic effect, stimulate inflammations and thereby contribute to the elimination of tumour cells. Examples of these proteins are:

RANTES, monocyte chemotactic and activating factor (MCAF), IL-8, macrophage inflammatory protein (MIP-1α,-β), neutrophil activating protein-2 (NAP-2), IL-3, IL-5, human leukaemia inhibitory factor (LIF), IL-7, IL-I 1, IL-13, GM-CSF, G-CSF and M-CSF.

Particular preference is furthermore given to antineoplastic proteins which, due to their action as enzymes, are able to convert precursors of an antineoplastic active compound into an antineoplastic active compound Examples of these enzymes are:

herpes simplex virus thymidine kinase, varicella zoster virus thymidine kinase, bacterial nitroreductase, bacterial β-glucuronidase, plant β-glucuronidase from Secale cereale, human glucuronidase, human carboxy peptidase, bacterial carboxypeptidase, bacterial β-lactamnase, bacterial cytosine deaminidase, human catalase and/or phosphatase, human alkaline phosphatase, type 5 acid phosphatase, human lysooxidase, human acid D-aminooxidase, human glutathione peroxidase, human eosinophil peroxidase and human thyroid peroxidase.

The abovementioned recombinant constructs can also contain DNA sequences which encode factor VIII or factor IX, or part fragments thereof. These DNA sequences also include other blood clotting factors.

The abovementioned recombinant constructs can also contain DNA sequences which encode a reporter protein. Examples of these reporter proteins are:

Chloramphenicol acetyl transferase (CAT), glow-worm luciferase (LUC), β-galactosidase (β-Gal), secreted alkaline phosphatase (SEAP), human growth hormone (hGH), β-glucuronidase (GUS), green-fluorescing protein (GFP), and all the variants derived therefrom, aquarin and obelin.

Recombinant constructs according to the invention can also contain DNA which encodes the human catalytic telomerase subunit and its variants and fragments in the antisense orientation. Where appropriate, these constructs can also contain other protein subunits of the human telomerase and the telomerase RNA component in the antisense orientation.

The recombinant constructs can, in addition to the DNA which encodes the human catalytic telomerase subunit, and its variants and fragments, also contain other protein subunits of the human telomerase and the telomerase RNA component.

The invention furthermore relates to a vector which contains the abovementioned DNA sequences according to the invention, in particular the 5′-flanking DNA sequences and also one or more of the other DNA sequences mentioned above.

The preferred vector for these constructs is a virus, for example a retrovirus, an adenovirus, an adeno-associated virus, a herpes simplex virus, a vaccina virus, a lentiviral virus, a Sindbis virus and a Semliki forest virus.

Preference is also given to using plasmids as vectors.

The invention furthermore relates to pharmaceutical preparations which comprise recombinant constructs or vectors according to the invention; for example a preparation in a colloidal dispersion system.

Examples of suitable colloidal dispersion systems are liposomes or polylysine ligands.

The preparations of the constructs or vectors according to the invention in colloidal dispersion systems can be supplemented with a ligand which binds to the membrane structures of tumour cells. Such a ligand can, for example, be attached to the construct or the vector or else be a component of the liposome structure.

Suitable ligands are, in particular, polyclonal or monoclonal antibodies, or antibody fragments thereof, which bind, by their variable domains, to the membrane structures of tumour cells, or substances carrying mannose terminally, cytokines or growth factors, or fragments or part sequences thereof, which bind to receptors on tumour cells.

Examples of corresponding membrane structures are receptors for a cytokine or a growth factor, such as IL-1, EGF, PDGF, VEGF, TGF β, insulin or insulin-like growth factor (ILGF), or adhesion molecules, such as SLeX, LFA-1, MAC-1, LECAM-1 or VLA-4, or the mannose-6-phosphate receptor.

The present invention includes pharmaceutical preparations which, in addition to the vector constructs according to the invention, can also comprise non-toxic, inert, pharmaceutically suitable excipients. It is possible to conceive of administering (e.g. intravenously, intraarterially, intramuscularly, subcutaneously, intradermally, anally, vaginally, nasally, transdermally, intraperitoneally, as an aerosol or orally) these preparations at the site of a tumour or administering them systematically.

The vector constructs according to the invention can be employed in gene therapy.

The invention furthermore relates to a recombinant host cell, in particular a recombinant eukaryotic host cell, which harbours the above-described constructs or vectors.

The invention furthermore relates to a process for identifying substances which affect the promoter activity, silencer activity or enhancer activity of the catalytic telomerase subunit, with this process comprising the following steps:
A. adding a candidate substance to a host cell which harbours the regulatory DNA sequence according to the invention, in particular the 5′-flanking regulatory DNA sequence for the gene for the human catalytic telomerase subunit, or a part region thereof which has a regulatory effect, which sequence or part region is functionally linked to a reporter gene, and
B. measuring the effect of the substance on expression of the reporter gene.

The process can be employed for identifying substances which increase the promoter activity, silencer activity or enhancer activity of the catalytic telomerase subunit.

The process can furthermore be employed for identifying substances which inhibit the promoter activity, silencer activity or enhancer activator of the catalytic telomerase subunit.

The invention furthermore relates to a process for identifying factors which bind specifically to fragments of the DNA fragments according to the invention, in particular the 5′-flanking regulation DNA sequence of the catalytic telomerase subunit. This method comprises screening an expression cDNA library using the above-described DNA sequence, or subfragments of widely differing length, as the probe.

The above-described constructs or vectors can also be used for preparing transgenic animals.

The invention furthermore relates to a process for detecting telomerase-associated conditions in a patient, which process comprises the following steps:
A. incubating a construct or vector, which contains the DNA sequence according to the invention, in particular the 5′-flanking regulatory DNA sequence for the gene for the human catalytic telomerase subunit, or a part region thereof having a regulatory effect, and a reporter gene, with body fluids or cell samples,
B. detecting the activity of the reporter gene in order to obtain a diagnostic value; and
C. comparing the diagnostic value with standard values for the reporter gene construct in standardized normal cells or body fluids of the same type as the test sample;

The detection of diagnostic values which are higher or lower than the standard comparative values indicates a telomerase-associated condition, which in turn indicates a pathogenic condition.

### EXPLANATION OF THE FIGURES

FIG. 1: Southern blot analysis using genomic DNA from various species
A: Photograph of an ethidium bromide-stained 0.70% agarose gel containing approximately 4 μg of Eco RI-cut (genomic DNA. Track 1 contains Hind III-cut λ DNA as size markers (23.5, 9.4, 6.7, 4.4, 2.3, 2.0 and 0.6 kb). Tracks 2 to 10 contain human, rhesus monkey. Sprague Dawley rat, BALB/c mouse, dog, bovine, rabbit, chicken and yeast (Saccharomyces cerevisiae) genomic DNA.
B: Autoradiogram, corresponding to FIG. 1 A, of a Southern blot analysis in which radioactively labelled hTC-cDNA probe of about 720 bp in length is used for the hybridization.

FIG. 2: Restriction analysis of the recombinant λ DNA of the phage clone P12, which hybridizes with a probe from the 5′ region of the hTC cDNA.

The figure shows a photograph of an ethidium bromide-stained 0.4% agarose gel. Tracks 1 and 2 contain Eco RI/Hind III-cut λ DNA and a 1 kb ladder from Gibco as size markers. Tracks 3-7 each contain 250 ng of the DNA from the recombinant phage which has been cut with Bam HI (track 3), Eco RI (track 4), Sal I (track 5), Xho I (track 6) and Sac I (track 7). The arrows mark the two λ arms of the vector EMBL3 Sp6/T7.

FIG. 3: Restriction analysis and Southern blot analysis of the recombinant λ DNA of the phage clone which hybridizes with a probe from the 5′ region of the hTC cDNA.
A: The figure shows a photograph of an ethidium bromide-stained 0.8% agarose gel. Tracks 1 and 15 contain a 1 kb ladder from Gibco as size markers. Tracks 2 to 14 each contain 250 ng of cut λ DNA from the recombinant phage clone. The following enzymes were employed: track 2: Sac I, track 3: Xho I, track 4: Xho I, Xba I, track 5: Sac I, Xho I, track 6: Sal I, Xho I, Xba I, track 7: Sac I, Xho I, Xba I, track 8: Sac I, Sal I, Xba I, track 9: Sac I, Sal I, BamH I, track 10: Sac I, Sal I, Xho I, track 11: Not I, track 12: Sma I, track 13: empty. track 14: not digested.
B: Autoradiogram, corresponding to FIG. 3 A, of a Southern blot analysis. A 5′-hTC cDNA fragment of about 420 bp in length was used as the probe for the hybridization.

FIG. 4: Partial DNA sequence of the 5′-flanking region and of the promoter of the gene for the human catalytic telomerase subunit. The ATG start codon in the sequence is printed in bold. The depicted sequence corresponds to SEQ ID. NO 1.

FIG. 5: Use of primer extension analysis to identify the transcription start.

The figure shows an autoradiogram of a denaturing polyacrylamide gel which was selected for depicting a primer extension analysis. An oligonucleotide having the sequence 5′GTTAAGTTGTAGCTTACACTGGTTCTC 3′ was used as the primer. The primer extension reaction was loaded in track 1. Tracks G, A, T and C constitute the sequence reactions using the same primer and the corresponding dideoxynucleotides. The thick arrow marks the main transcription start while the thin arrows point to three subsidiary transcription start points.

FIG. 6: cDNA sequence of the human catalytic telomerase subunit (hTC; cf. our pending application PCT/EP/98/03468). The depicted sequence corresponds to SEQ ID NO 2.

FIG. 7: Structural organization and restriction map of the human hTC gene and its 5′-flanking and 3′-flanking regions.

Exons are shown as consecutively numbered rectangles which are filled-in in black and introns are shown as regions which are not filled in. Untranslated sequence segments in the exons are hatched. Translation starts in exon 1 and ends in exon 16. Restriction enzyme cleavage sites are marked as follows: S, SacI; X, XhoI. The relative arrangement of the five phage clones (P2, P3, P5, P12, P17), and of the product from the genome walking, are shown by thin lines. As the dots indicate, the sequence of intron 16 has only been partly deciphered.

FIG. 8: HTL splice variants.
A: Diagrammatic structure of the hTC mRNA splice variants. The complete hTC mRNA is depicted as a rectangle with a grey background in the upper region of the figure. The 16 exons are depicted in accordance with their size. The translation start (ATG) and the stop codon, and also the telomerase-specific T motif, and the seven RT motifs, are all shown. The hTC variants are subdivided into deletion and insertion variants. The missing exon sequences are marked in the deletions. The insertions are shown by additional white rectangles. The sizes and origins of the inserted sequences are given. Newly formed stop codons are marked. The size of the insertion in variant INS2 is unknown.
B: Exon-intron transitions in the hTC splice variants. Unspliced 5′-flanking and 3′-flanking sequences are shown as white rectangles. The origins of the exon and intron sequences are given. Intron and exon sequences are shown in small letters and large letters, respectively. The donor and acceptor sequences in the splice sites are underlaid as grey rectangles, and their exon and intron origins are also given.

FIG. 9: Identification of the transcription start by means of RT-PCR analysis. The RT-PCR was carried out using a cDNA library prepared from HL 60 cells and genomic DNA as the positive control. A common 3′ primer hybridizes to a region of the exon I sequence. The positions of the different 5′ primers in the coding region or the 5′-flanking region are given. In the negative control, no template DNA was added to the PCR reaction. M: DNA size marker.

FIG. 10: Nucleotide sequence and structural features of the hTC promoter.

The figure depicts 11273 bp of the 5′-flanking hTC gene sequence, beginning with the translation start codon ATG (+1). The putative region of the translation start is underlined. Possible regulatory sequence segments within the 4000 bp upstream of the translation start are ringed. The depicted sequence corresponds to SEQ ID NO 3.

FIG. 11: Activity of the hTC promoter in HEK-293 cells.

The first 5000 bp of the 5′-flanking hTC gene region are shown diagrammatically in the upper part of the figure. The ATG start codon is picked out. CpG-rich islands are marked by grey rectangles. The sizes of the hTC promoter-luciferase construct are shown on the left-hand side of the figure. The promoterless. pGL2 basic construct and the SV40 promoter construct pGL2-Pro were used as controls in each transfection. The relative luciferase activities of the different promoter constructs in HEK cells are shown as continuous bars on the right-hand side of the figure. The standard deviation is indicated. The numerical values represent the average of two independent experiments which were carried out in duplicate.

Tab. 1: Exon-intron transitions in the hTC gene

The table lists the nucleotide sequences at the 3′ and 5′ splice transitions of the hTC gene. The consensus sequences for donor and acceptor sequences (AG and GT) are underlaid with grey rectangles. The table shows the intron sequences (small letters) and exon sequences (large letters) which flank the splice acceptor and donor sites. The sizes of the exons and introns are given in bp.

Tab. 2: Potential binding sites for DNA-binding factors in the nucleotide sequence of intron 2

The search for possible DNA-binding factors (e.g. transcription factors) was carried out using the “find pattern” algorithm from the Genetics Computer Group (Madison, USA) GCG sequence analysis program package. The table lists the abbreviations of the DNA-binding factors which were identified and their location in intron 2.

TAB. 1

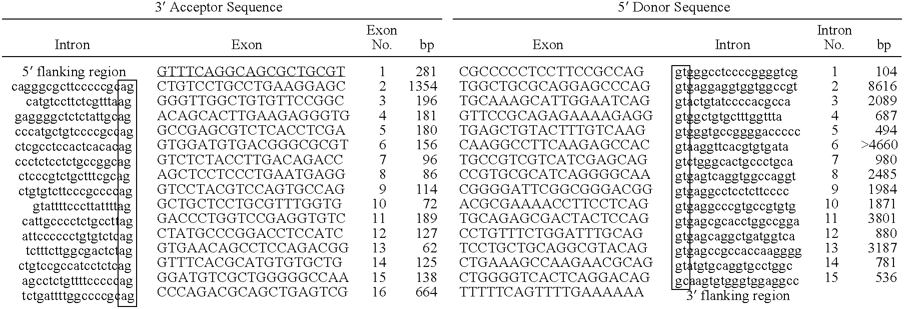

TAB 2

| Factors | Location in intron 2 |
|---|---|
| C/EBP | 2925 |
| CRE.2 | 2749 |
| Sp1 | 2378, 4094, 4526, 4787, 4835, 4995 |
| AP-2 CS3 | 5099 |
| AP-2 CS4 | 2213, 3699, 4667, 5878, 5938, 6059, 6180, 6496 |
| AP-2 CS5 | 5350, 5798, 5880, 5940, 6061, 6182, 6375, 6498 |
| PEA3 | 934, 2505 |
| P53 | 2125 |
| GR uteroglobin | 848, 1487, 2956 |
| PR uteroglobin | 3331 |
| Zeste-white | 1577, 1619, 1703, 1745, 1787, 1829, 1871, 1913, 1955, |
| | 1997, 2039, 2081, 3518, 3709, 4765, 5014, 5055 |
| GRE | 846 |
| MyoD-MCK | 447, 509, 558, 1370, 1595, 1900, 2028, 2099, 4557 |
| right site/rev | |
| MyoD-MCK | 108, 118, 453, 1566, 1608, 1692, 1734, 1818, 1902, |
| left site | 1986, 2372, 2460, 2720, 3491, 5030 |
| Ets-1 CS | 6408 |
| AP1 | 3784, 4406 |
| CREB | 2801 |
| GATA-1 | 839, 1390, 3154 |
| c-Myc | 108, 118, 453, 1566, 1608, 1692, 1734, 1818, 1902, |
| | 1986, 2372, 2460, 2720, 3491, 5030 |
| CACCC site | 991 |
| CCAAT site | 1224 |
| CCAC box | 992 |
| CAAT site | 463, 2395 |
| Rb site | 992, 4663 |
| TATA | 3650 |
| CDEI | 106, 1564, 1606, 1690, 1732, 1816, 1900, 1984 |

### EXAMPLES

The human gene for the catalytic telomerase subunit (ghTC), and the regions of this gene located 5′ and 3′, were cloned, while the start point for transcription was determined, potential binding sites for DNA-binding proteins were identified and active promoter fragments were highlighted. The sequence of the hTC cDNA (FIG. 6) has already been reported in our application PCT/EP/98/03468, which is also pending. Unless otherwise mentioned, all the data refer to the position of the cDNA in this sequence.

### Example 1

A genomic Southern blot analysis was used to determine whether ghTC constitutes a single gene in the human genome or whether there exist several loci for the hTC gene and possibly also ghTC pseudogenes.

In order to do this, a commercially available zoo blot from Clontech was subjected to Southern blot analysis. This blot contains 4 μg of Eco RI-cut genomic DNA from nine different species (human, monkey, rat, mouse, dog, bovine, rabbit, chicken and yeast). With the exception of yeast, chicken and human, the DNA was isolated from kidney tissue. The human genomic DNA was isolated from placenta and the chicken genomic DNA was purified from liver tissue. An hTC cDNA fragment of about 720 bp in length, which was isolated from hTC cDNA, variant Del2 (position 1685 to 2349 plus 2531 to 2590 in FIG. 6 [deletion 2; cf. Example 5 in FIG. 8]), was used as the radioactively labelled probe in the autoradiogram in FIG. 1. The experimental conditions for the blot hybridization and washing steps were taken from Ausubel et al. (1987).

In the case of the human DNA, the probe recognizes two specific DNA fragments. The smaller Eco RI fragment, of from about 1.5 to 1.8 kb in length, probably originates from two Eco RI cleavage sites in an intron in the ghTC DNA. On the basis of this result, it is to be assumed that only one single ghTC gene is present in the human genome.

### Example 2

In order to isolate the 5′ flanking hTC gene sequence, approx. 1.5×10⁶ phages from a human genomic placenta gene library (EMBL 3 SP6/T7 from Clontech, order number HL1067j) were hybridized on nitrocellulose filters (0.45 μm; from Schleicher and Schuell), in accordance with the manufacturer's instructions, with a radioactively labelled 5′-hTC cDNA fragment of about 500 bp in length (position 839 to 1345 in FIG. 6). The nitrocellulose filters were firstly incubated, at 42° C. for two hours, in 2×SSC (0.3 M NaCl; 0.5 M Tris-HCl, pH 8.0) and then in a prehybridization solution (50% formamide; 5×SSPE, pH 7.4; 5×Denhard's solution; 0.25% SDS; 100 μg of herring sperm DNA/ml). For the overnight hybridization, the prehybridization solution was supplemented with 1.5×10⁶ cpm of denatured, radioactively labelled probe/ml of solution. Nonspecifically bound radioactive DNA was removed under stringent conditions, i.e. by means of three five-minute steps of washing with 2×SSC; 0.1% SDS at from 55 to 65° C. The filters were evaluated by autoradiography.

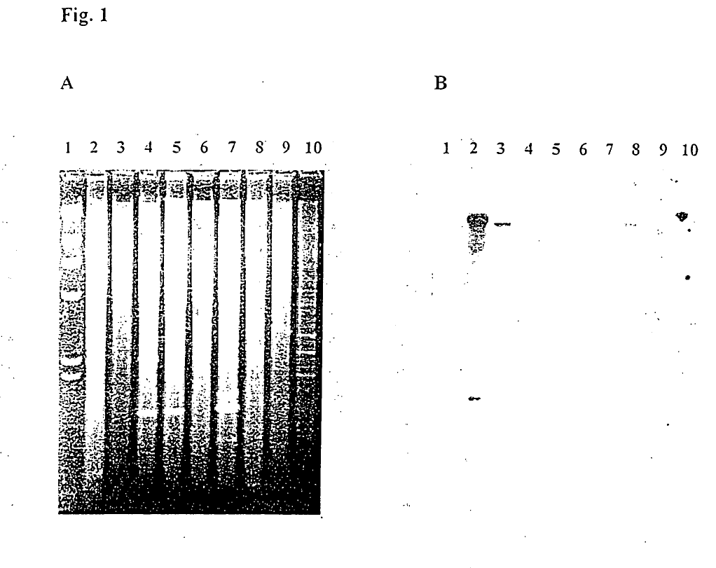

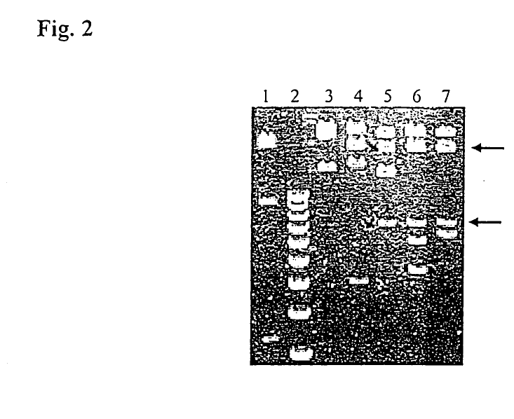

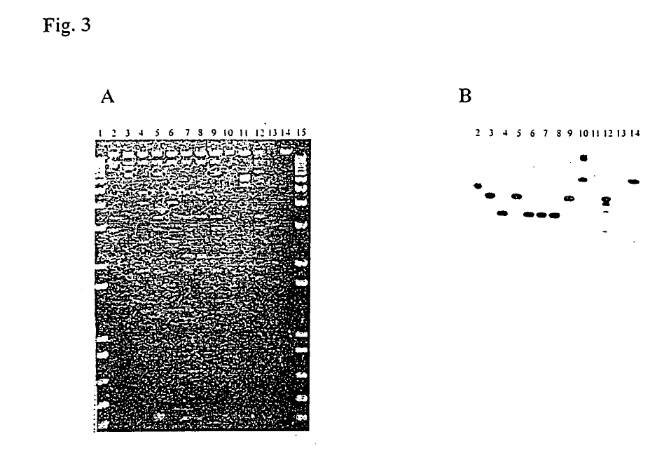

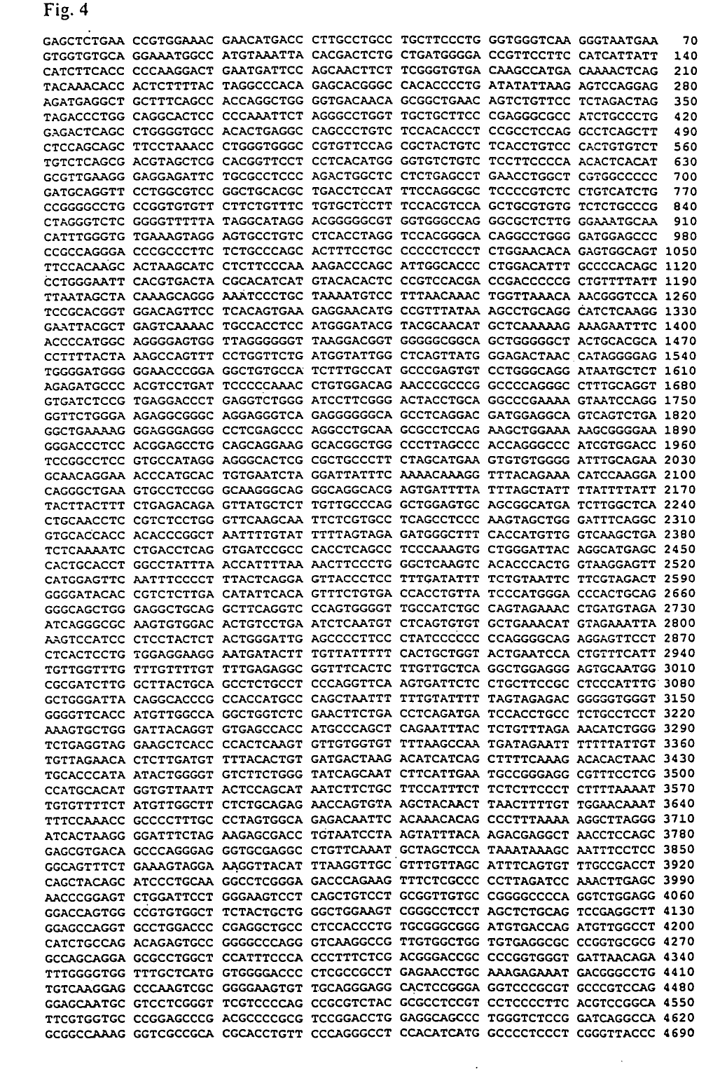

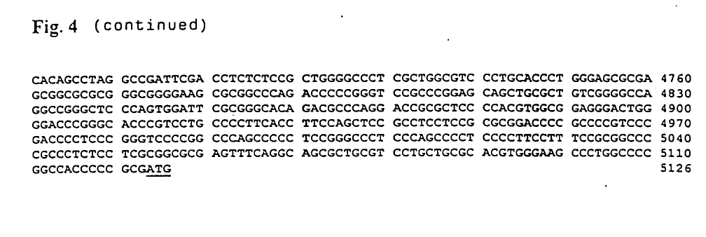

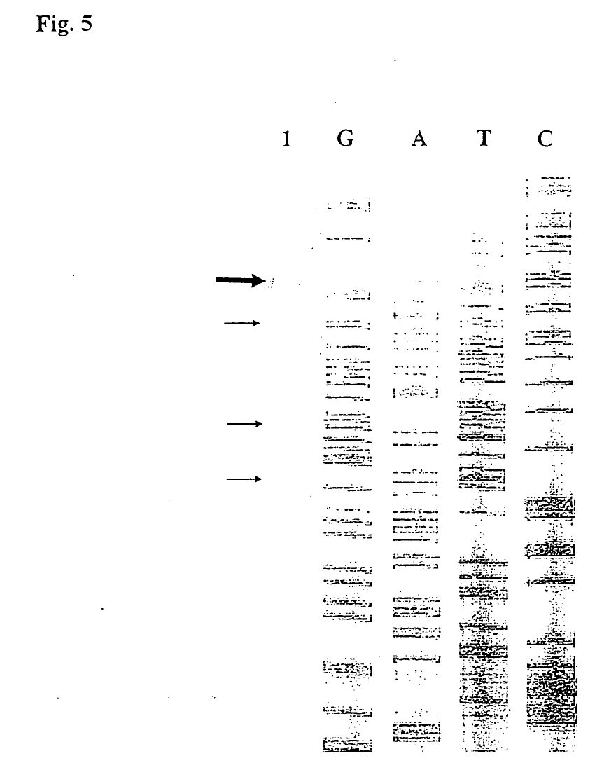

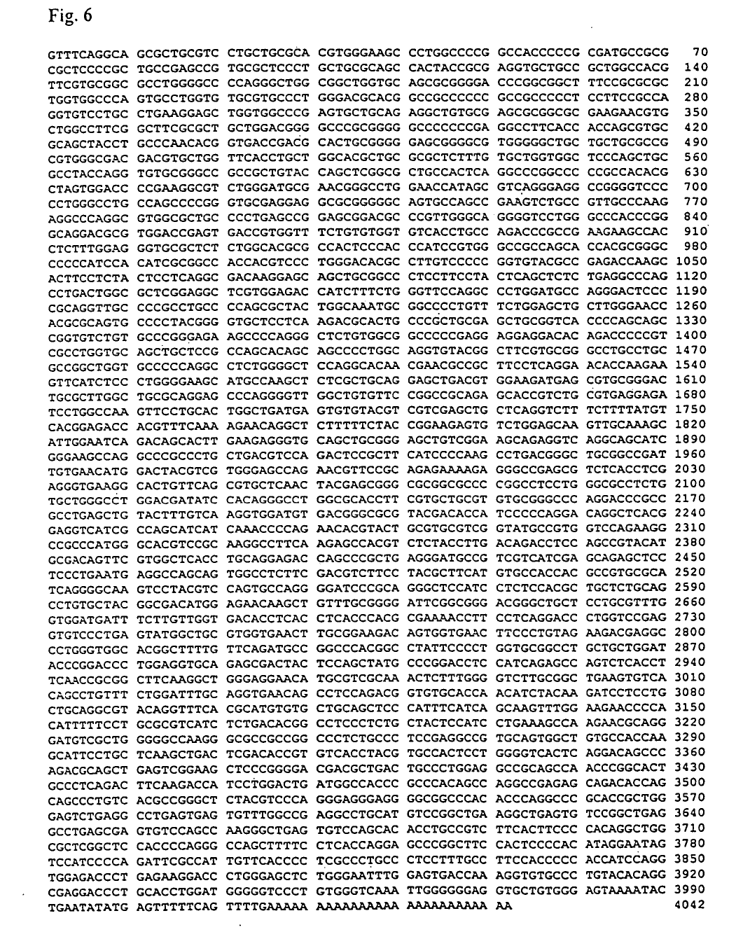

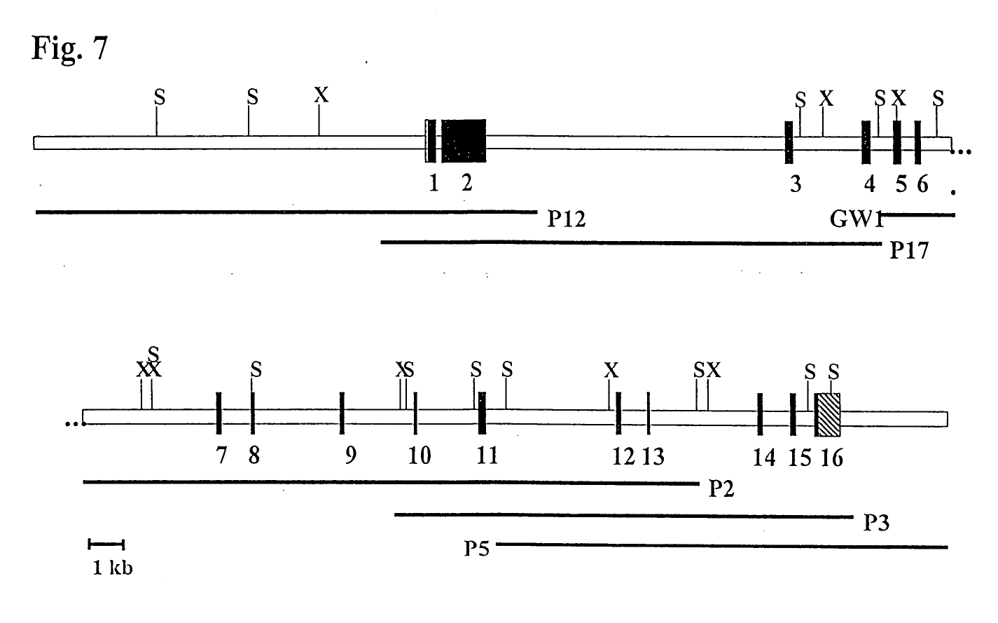

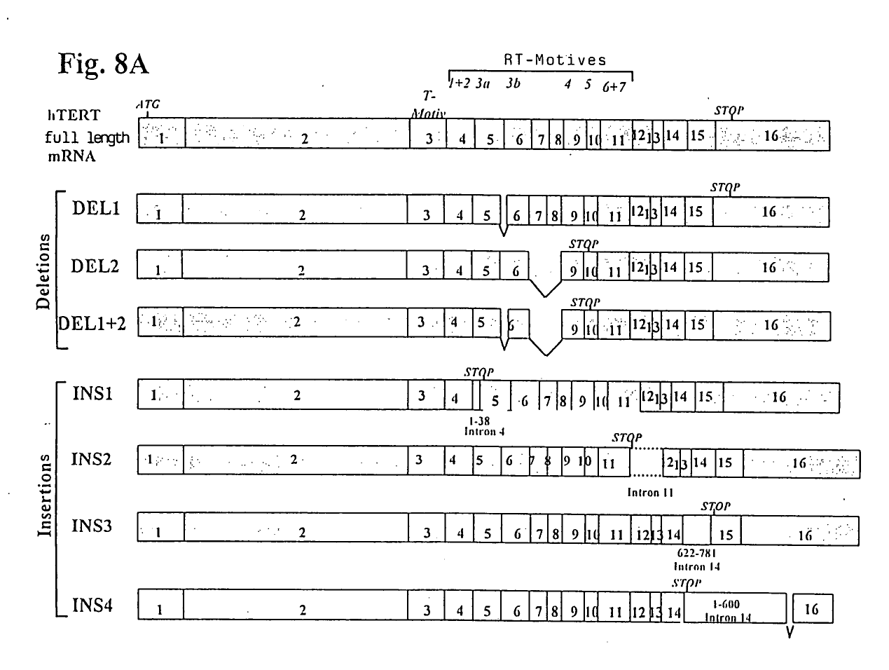

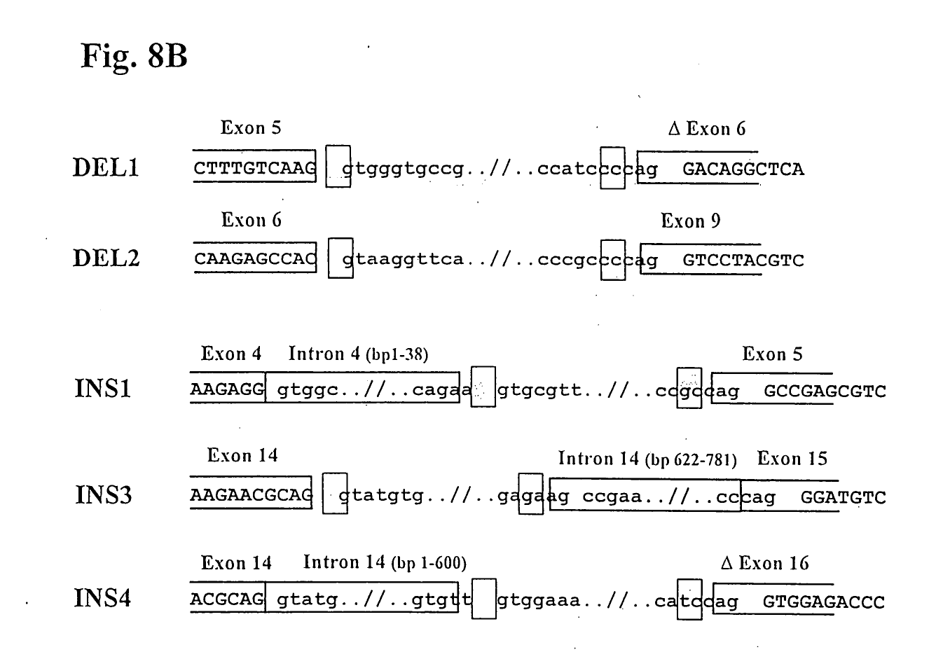

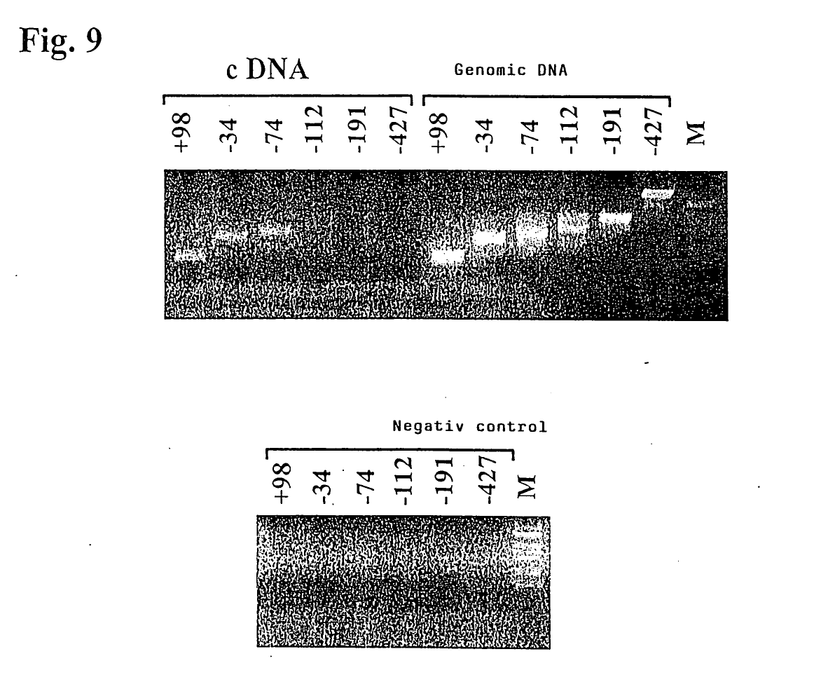

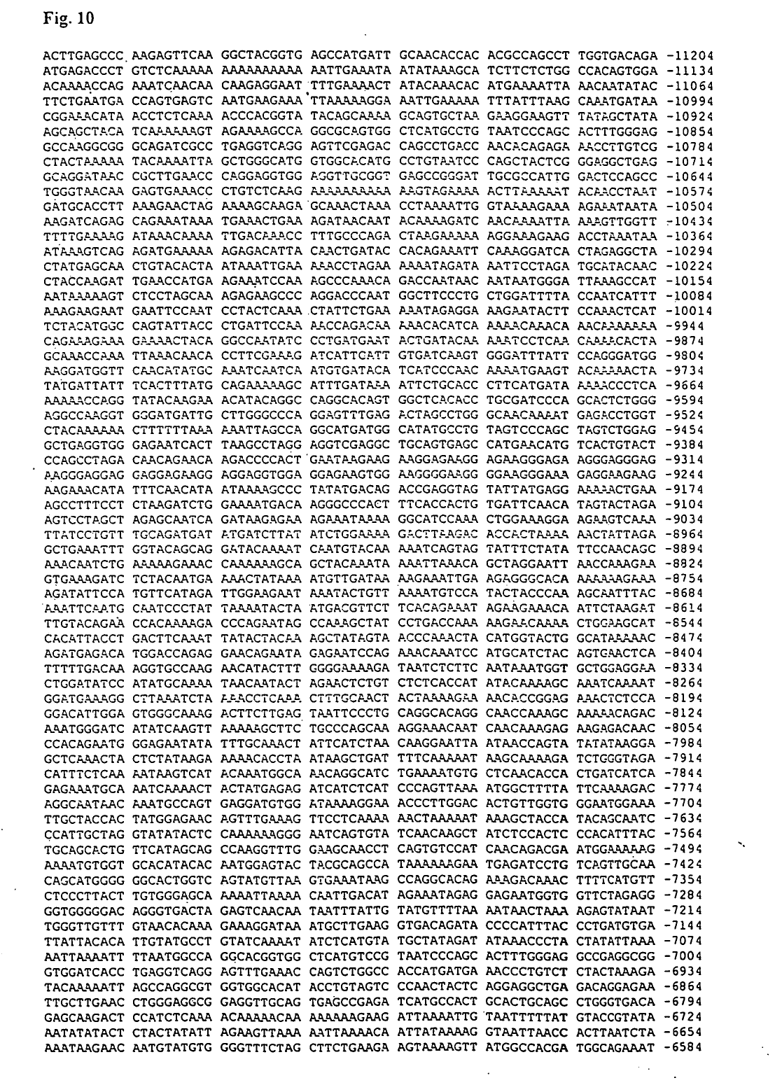

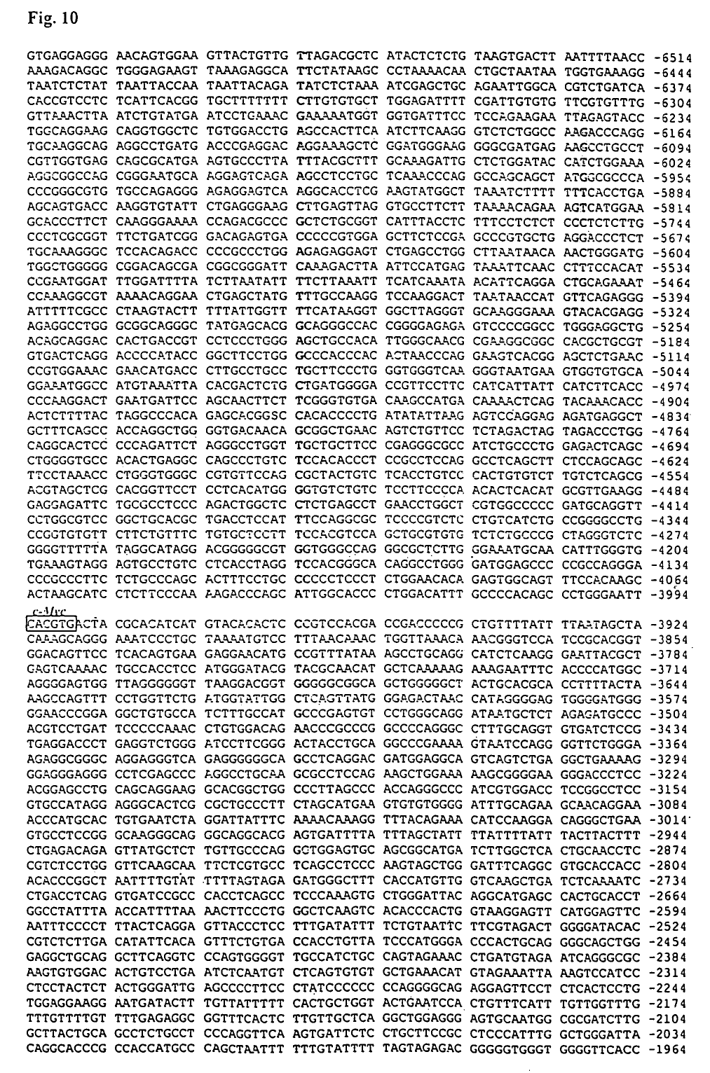

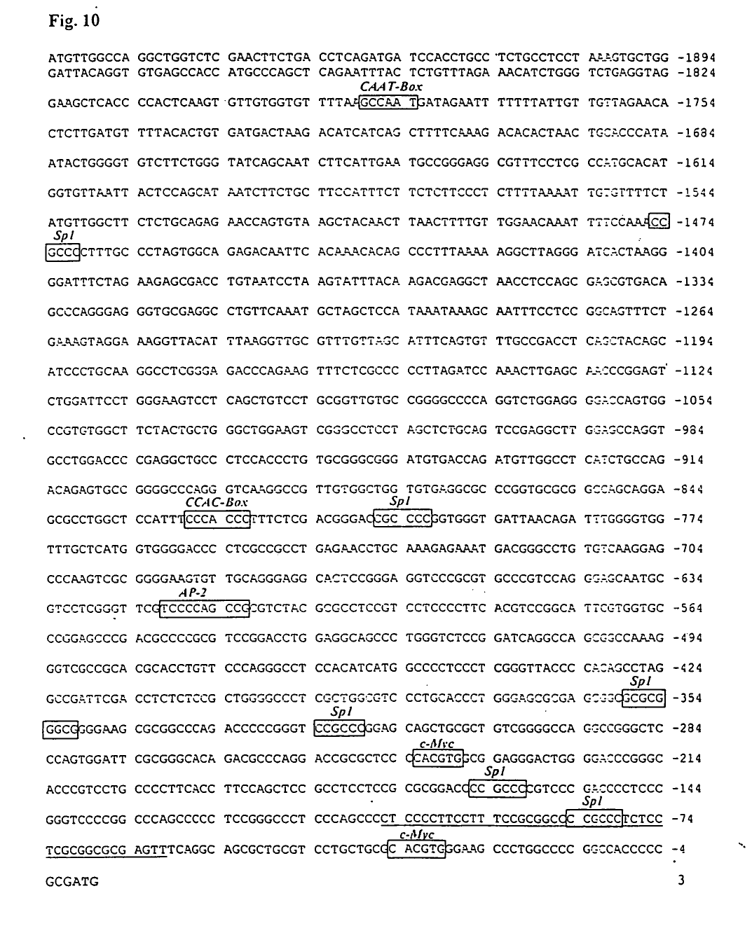

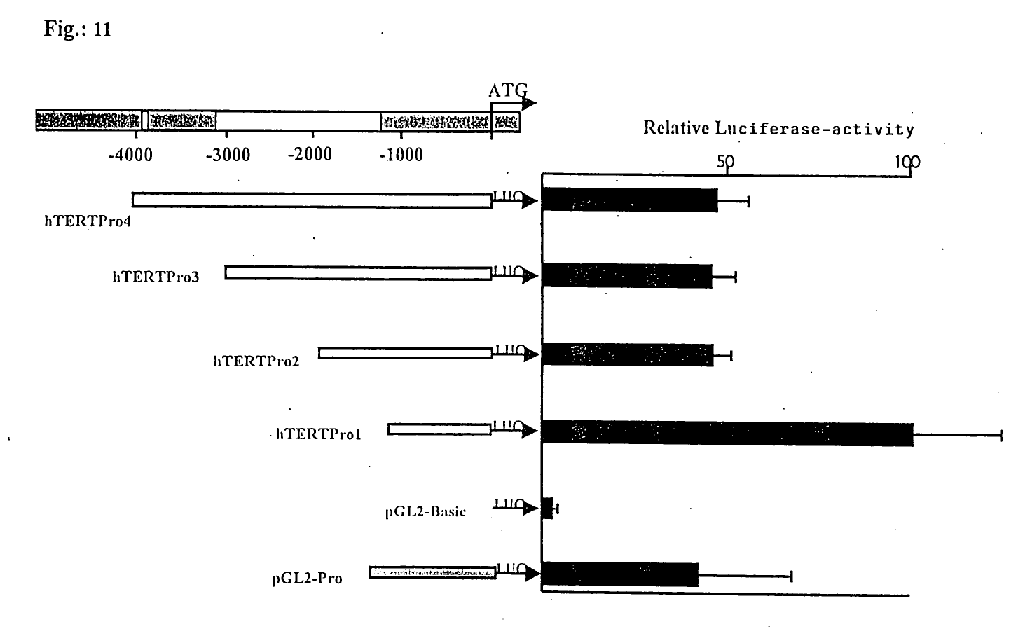

The phage clones which were identified in this primary investigation were purified (Ausubel et al. (1987)). In subsequent analyses, one phage clone, i.e. P12 turned out to be potentially positive. A λ DNA preparation carried out on this phage (Ausubel et al. (1987)), and the subsequent restriction digestion with enzymes which release the genomic insert in fragments, showed that this phage clone contains an insert of approx. 15 kb in the vector (FIG. 2).

In order to isolate the complete hTC gene sequence, in each case from 1 to 1.5×10⁶ phages were screened, in independent experiments, with in each case different radioactively labelled probes, as described above.

The phage clones which were identified in these primary investigations, and which were positive for the corresponding probes, were purified. The phage clone P17 was found to contain an hTC cDNA fragment of about 250 bp in length (position 1787 to 2040 in FIG. 6). The phage clone P2 was identified as containing an hTC cDNA fragment of about 740 bp in length (position 1685 to 2349 plus 2531 to 2607 in FIG. 6 [deletion 2; cf. Example 5]). The phage clones P3 and P5 were found to contain a 3′ hTC cDNA fragment of 420 bp in length (position 3047 to 3470 in FIG. 6). After the λ DNA had been prepared from these phages, and subsequently subjected to restriction digestion with enzymes which release the genomic insert in fragments, the inserts were subcloned into plasmids (Example 4).

### Example 3

In order to investigate whether the 5′ end of the hTC cDNA was also present in the insert in the recombinant phage clone P12, the λ DNA from this clone was hybridized, in a Southern blot analysis, with a radiactively labelled hTC cDNA fragment of about 440 bp in length (position 1 to 440 in FIG. 6) from the extreme 5′ region (FIG. 3).

Since the isolated λ DNA from the positive clone also hybridizes with the extreme 5′ end of the hTC cDNA, this phage probably also contains the 5′ sequence region flanking the ATG start codon.

### Example 4

In order to subclone the entire 15 kb insert in the positive phage clone P12 in the form of subfragments, and subsequently to sequence these fragments, restriction endonucleases which, on the one hand, release the entire insert from EMBL3 Sp6/T7 (cf. Example 2) and, in addition, cut within the insert, were selected for digesting the DNA.

In all, two Xho I subfragments, of about 8.3 and about 6.5 kb in length, respectively, and three Sac I subfragments, of about 8.5, about 3.5 and about 3 kb in length, respectively, were subcloned into the pBluescript KS(+) vector (from Stratagene). The 5123 bp 5′-flanking nucleotide sequence of the ghTC gene region, starting from the ATG start codon, was determined by analysing the sequences of these fragments (FIG. 4; corresponding to SEQ ID NO 1). FIG. 4 depicts the first 5123 bp (starting from the ATG start codon). FIG. 10 depicts the entire cloned 5′ sequence (corresponding to SEQ ID NO 3).

In order to subclone the entire insert, of approx. 14.6 kb in size, in phage clone P17 in the form of subfragments, restriction endonucleases which, on the one hand, release the entire insert from EMLB3 Sp6/T7 and, in addition, cut a few times within the insert, were selected for digesting the DNA. Three XhoI/BamHI fragments, of 7.1 kb, 4.2 kb and 1.5 kb in size, respectively, and one BamHI fragment, of 1.8 kb in size, were subcloned by means of using a combination digestion with the enzymes XhoI and BamHI. Combination restriction digestion with the enzymes XhoI and XbaI resulted in a XhoI/XbaI fragment of 6.5 kb in size, and two XhoI fragments, of 6.5 kb and 1.5 kb in size, respectively, being cloned.

Digestion with the restriction enzyme XhoI was used to subclone the insert, of approx. 17.9 kb in size, in phage clone P2 in the form of subfragments. In all, three XhoI subfragments, of 7.5 kb, 6.4 kb and 1.6 kb in length, respectively, were cloned. Four SacI fragments, of 4.8 kb, 3 kb, 2 kb and 1.8 kb in size, respectively, were additionally subcloned by digesting with the restriction enzyme SacI.

The insert, of approx. 13.5 kb in size, in phage clone P3 was subcloned by digesting with the restriction enzymes SacI and/or XhoI. Six SacI subfragments, of 3.2 kb, 2 kb, 0.9 kb, 0.8 kb, 0.65 kb and 0.5 kb in length, respectively, and two XhoI subfragments, of 6.5 kb and 4.3 kb in length, respectively, were obtained in this connection.

The insert, of approx. 13.2 kb in size, in phage clone P5 was subcloned by digesting with the restriction enzymes SacI and/or XhoI. In all, SacI fragments of 6.5 kb, 3.3 kb, 3.2 kb, 0.8 kb and 0.3 kb in size, and Xhol fragments of 7 kb and 3.2 kb in size, were subcloned.

In order to clone the hTC genomic sequence region located 3′ of phage clone P17 and 5′ of phage clone P2, 3 genomic walkings were carried out using the Clontech GenomeWalker™ kits (catalogue number K1803-1) and various combinations of primers. In a final volume of 50 μl, 10 pmol of dNTP mix were added to 1 μl of human GenomeWalker Library HDL (from Clontech), and a PCR reaction was carried out in 1×Klen Taq PCR reaction buffer and 1×Advantage Klen Taq polymerase mix (from Clontech). 10 pmol of an internal gene-specific primer, and 10 pmol of the adaptor primer AP1 (5′-GTAATACGACTCACTATAGGGC-3′; from Clontech) were added as primers. The PCR was carried out in 3 steps as a touchdown PCR. First of all, denaturation was carried out at 94° C. for 20 sec, and the primers were then annealed, and the DNA chain extended, at 72° C. for 4 min, over 7 cycles. There then followed 37 cycles in which the DNA was denaturated at 94° C. for 20 sec but the subsequent primer extension took place at 67° C. for 4 min. In conclusion, there followed a chain extension at 67° C. for 4 min. After this first PCR, the PCR product was diluted 1:50. One μl of this dilution- was used in a second nested PCR together with 10 pmol of dNTP mix in 1×Klen Taq PCR reaction buffer and 1×Advantage KIen Taq polymerase mix and also 10 pmol of a nested gene-specific primer and 10 pmol of the nested Marathon Adaptor primers AP2 (5′-ACTATAGGGCACGCGTGGT-3′; from Clontech). The PCR conditions corresponded to the parameters which were selected in the first PCR. As the sole exception, only 5 cycles rather than 7 cycles were selected in the first PCR step and only 24 cycles, instead of 37 cycles. were run in the second PCR step. The products of this nested genomic walking PCR were cloned into the TA Cloning Vector pCRII from InVitrogen.

In the first genomic walking, the gene-specific primer C3K2-GSP1 (5′-GACGTGGCTCTTGAAGGCCTTG-3′) and the nested gene-specific primer C3K2-GSP2 (5′-GCCTTCTGGACCACGGCATACC-3′) were used, together with the HDL library 4, and a PCR fragment of 1639 bp in length was obtained. In the second genomic walking, a PCR fragment of 685 bp in length was amplified from the HDL library 4 using the gene-specific primer C3F2 (5′-CGTAGTTGAGCACGCTGAACAGTG-3′) and the nested gene-specific primer C3F (5′-CCTTCACCCTCGAGGTGAGACGCT-3. The third genomic walking mixture, using the gene-specific primer DEL5-GSP1 (5′-GGTGGATGTGACGGGCGCGTACG-3′) and the nested gene-specific primer C5K-GSP1 (5′-GGTATGCCGTGGTCCAGAAGGC-3′), led to a 924 bp PCR fragments being cloned from the HDL library 1. In all, 2100 bp of the genomic hTC region located 3′ of phage clone P17 were identified using this genomic walking method (see FIG. 7).

The subcloned fragments, and the genomic walking products, were sequenced in single-stranded form. The Lasergene Biocomputing Software (DNASTAR Inc. Madison, Wis., USA) was used to identify overlapping regions and form contigs. In all, 2 large contigs were assembled from the sequences collected from phage clones P12, P17, P2, P3 and P5, and also the sequence data from the genomic walking. Contig 1 consists of sequence data from phage clones P12 and P17 and the sequence data from the genomic walking. Contig 2 was put together from the sequences from phage clones P2, P3 and P5. Overlapping phage clone regions are shown diagrammatically in FIG. 7. The sequence data from the 2 contigs are shown below. The ATG start codon in contig 1 is underlined. The TGA stop codon is underlined in contig 2.

| Contig 1: | |
|---|---|
| ACTTGAGCCC AAGAGTTCAA GGCTACGGTG AGCCATGATT GCAACACCAC ACGCCAGCCT TGGTGACAGA | 70 |
| ATGAGACCCT GTCTCAAAAA AAAAAAAAAA AATTGAAATA ATATAAAGCA TCTTCTCTGG CCACAGTGGA | 140 |
| ACAAAACCAG AAATCAACAA CAAGAGGAAT TTTGAAAACT ATACAAACAC ATGAAAATTA AACAATATAC | 210 |
| TTCTGAATGA CCAGTGAGTC AATGAAGAAA TTAAAAAGGA AATTGAAAAA TTTATTTAAG CAAATGATAA | 280 |
| CGGAAACATA ACCTCTCAAA ACCCACGGTA TACAGCAAAA GCAGTGCTAA GAAGGAAGTT TATAGCTATA | 350 |
| AGCAGCTACA TCAAAAAAGT AGAAAAGCCA GGCGCAGTGG CTCATGCCTG TAATCCCAGC ACTTTGGGAG | 420 |
| GCCAAGGCGG GCAGATCGCC TGAGGTCAGG AGTTCGAGAC CAGCCTGACC AACACAGAGA AACCTTCTCG | 490 |
| CTACTAAAAA TACAAAATTA GCTGGGCATG GTGGCACATG CCTGTAATCC CAGCTACTCG GGAGGCTGAG | 560 |
| GCAGGATAAC CGCTTGAACC CAGGAGGTGG AGGTTGCGGT GAGCCGGGAT TGCGCCATTG GACTCCAGCC | 630 |
| TGGGTAACAA GAGTGAAACC CTGTCTCAAG AAAAAAAAAA AAGTAGAAAA ACTTAAAAAT ACAACCTAAT | 700 |
| GATGCACCTT AAAGAACTAG AAAAGCAAGA GCAAACTAAA CCTAAAATTG GTAAAAGAAA AGAAATAATA | 770 |
| AAGATCAGAG CAGAAATAAA TGAAACTGAA AGATAACAAT ACAAAAGATC AACAAAATTA AAAGTTGGTT | 840 |
| TTTTGAAAAG ATAAACAAAA TTGACAAACC TTTGCCCAGA CTAAGAAAAA AGGAAAGAAG ACCTAAATAA | 910 |
| ATAAAGTCAG AGATGAAAAA AGAGACATTA CAACTGATAC CACAGAAATT CAAAGGATCA CTAGAGGCTA | 980 |
| CTATGAGCAA CTGTACACTA ATAAATTGAA AAACCTAGAA AAAATAGATA AATTCCTAGA TGCATACAAC | 1050 |
| CTACCAAGAT TGAACCATGA AGAAATCCAA AGCCCAAACA GACCAATAAC AATAATGGGA TTAAAGCCAT | 1120 |
| AATAAAAAGT CTCCTAGCAA AGAGAAGCCC AGGACCCAAT GGCTTCCCTG CTGGATTTTA CCAATCATTT | 1190 |
| AAAGAAGAAT GAATTCCAAT CCTACTCAAA CTATTCTGAA AAATAGAGGA AAGAATACTT CCAAACTCAT | 1260 |
| TCTACATGGC CAGTATTACC CTGATTCCAA AACCAGACAA AAACACATCA AAAACAAACA AACAAAAAAA | 1330 |
| CAGAAAGAAA GAAAACTACA GGCCAATATC CCTGATGAAT ACTGATACAA AAATCCTCAA CAAAACACTA | 1400 |
| GCAAACCAAA TTAAACAACA CCTTCGAAAG ATCATTCATT GTGATCAAGT GGGATTTATT CCAGGGATGG | 1470 |
| AAGGATGGTT CAACATATGC AAATCAATCA ATGTGATACA TCATCCCAAC AAAATGAAGT ACAAAAACTA | 1540 |
| TATGATTATT TCACTTTATG CAGAAAAAGC ATTTGATAAA ATTCTGCACC CTTCATGATA AAAACCCTCA | 1610 |
| AAAAACCAGG TATACAAGAA ACATACAGGC CAGGCACAGT GGCTCACACC TGCGATCCCA GCACTCTGGG | 1680 |
| AGGCCAAGGT GGGATGATTG CTTGGGCCCA GGAGTTTGAG ACTAGCCTGG GCAACAAAAT GAGACCTGGT | 1750 |
| CTACAAAAAA CTTTTTTAAA AAATTAGCCA GGCATGATGG CATATGCCTG TAGTCCCAGC TAGTCTGGAG | 1820 |
| GCTGAGGTGG GAGAATCACT TAAGCCTAGG AGGTCGAGGC TGCAGTGAGC CATGAACATG TCACTGTACT | 1890 |
| CCAGCCTAGA CAACAGAACA AGACCCCACT GAATAAGAAG AAGGAGAAGG AGAAGGGAGA AGGGAGGGAG | 1960 |
| AAGGGAGGAG GAGGAGAAGG AGGAGGTGGA GGAGAAGTGG AAGGGGAAGG GGAAGGGAAA GAGGAAGAAG | 2030 |
| AAGAAACATA TTTCAACATA ATAAAAGCCC TATATGACAG ACCGAGGTAG TATTATGAGG AAAAACTGAA | 2100 |
| AGCCTTTCCT CTAAGATCTG GAAAATGACA AGGGCCCACT TTCACCACTG TGATTCAACA TAGTACTAGA | 2170 |
| AGTCCTAGCT AGAGCAATCA GATAAGAGAA AGAAATAAAA GGCATCCAAA CTGGAAAGGA AGAAGTCAAA | 2240 |
| TTATCCTGTT TGCAGATGAT ATGATCTTAT ATCTGGAAAA GACTTAAGAC ACCACTAAAA AACTATTAGA | 2310 |
| GCTGAAATTT GGTACAGCAG GATACAAAAT CAATGTACAA AAATCAGTAG TATTTCTATA TTCCAACAGC | 2380 |
| AAACAATCTG AAAAAGAAAC CAAAAAAGCA GCTACAAATA AAATTAAACA GCTAGGAATT AACCAAAGAA | 2450 |
| GTGAAAGATC TCTACAATGA AAACTATAAA ATGTTGATAA AAGAAATTGA AGAGGGCACA AAAAAAGAAA | 2520 |
| AGATATTCCA TGTTCATAGA TTGGAAGAAT AAATACTGTT AAAATGTCCA TACTACCCAA AGCAATTTAC | 2590 |
| AAATTCAATG CAATCCCTAT TAAAATACTA ATGACGTTCT TCACAGAAAT AGAAGAAACA ATTCTAAGAT | 2660 |
| TTGTACAGAA CCACAAAAGA CCCAGAATAG CCAAAGCTAT CCTGACCAAA AAGAACAAAA CTGGAAGCAT | 2730 |
| CACATTACCT GACTTCAAAT TATACTACAA AGCTATAGTA ACCCAAACTA CATGGTACTG GCATAAAAAC | 2800 |
| AGATGAGACA TGGACCAGAG GAACAGAATA GAGAATCCAG AAACAAATCC ATGCATCTAC AGTGAACTCA | 2870 |
| TTTTTGACAA AGGTGCCAAG AACATACTTT GGGGAAAAGA TAATCTCTTC AATAAATGGT GCTGGAGGAA | 2940 |
| CTGGATATCC ATATGCAAAA TAACAATACT AGAACTCTGT CTCTCACCAT ATACAAAAGC AAATCAAAAT | 3010 |
| GGATGAAAGG CTTAAATCTA AAACCTCAAA CTTTGCAACT ACTAAAAGAA AACACCGGAG AAACTCTCCA | 3080 |
| GGACATTGGA GTGGGCAAAG ACTTCTTGAG TAATTCCCTG CAGGCACAGG CAACCAAAGC AAAAACAGAC | 3150 |
| AAATGGGATC ATATCAAGTT AAAAAGCTTC TGCCCAGCAA AGGAAACAAT CAACAAAGAG AAGAGACAAC | 3220 |
| CCACAGAATG GGAGAATATA TTTGCAAACT ATTCATCTAA CAAGGAATTA ATAACCAGTA TATATAAGGA | 3290 |
| GCTCAAACTA CTCTATAAGA AAAACACCTA ATAAGCTGAT TTTCAAAAAT AAGCAAAAGA TCTGGGTAGA | 3360 |
| CATTTCTCAA AATAAGTCAT ACAAATGGCA AACAGGCATC TGAAAATGTG CTCAACACCA CTGATCATCA | 3430 |
| GAGAAATGCA AATCAAAACT ACTATGAGAG ATCATCTCAT CCCAGTTAAA ATGGCTTTTA TTCAAAAGAC | 3500 |
| AGGCAATAAC AAATGCCAGT GAGGATGTGG ATAAAAGGAA ACCCTTGGAC ACTGTTGGTG GGAATGGAAA | 3570 |
| TTGCTACCAC TATGGAGAAC AGTTTGAAAG TTCCTCAAAA AACTAAAAAT AAAGCTACCA TACAGCAATC | 3640 |
| CCATTGCTAG GTATATACTC CAAAAAAGGG AATCAGTGTA TCAACAAGCT ATCTCCACTC CCACATTTAC | 3710 |
| TGCAGCACTG TTCATAGCAG CCAAGGTTTG GAAGCAACCT CAGTGTCCAT CAACAGACGA ATGGAAAAAG | 3780 |
| AAAATGTGGT GCACATACAC AATGGAGTAC TACGCAGCCA TAAAAAAGAA TGAGATCCTG TCAGTTGCAA | 3850 |
| CAGCATGGGG GGCACTGGTC AGTATGTTAA GTGAAATAAG CCAGGCACAG AAAGACAAAC TTTTCATGTT | 3920 |
| CTCCCTTACT TGTGGGAGCA AAAATTAAAA CAATTGACAT AGAAATAGAG GAGAATGGTG GTTCTAGAGG | 3990 |
| GGTGGGGGAC AGGGTGACTA GAGTCAACAA TAATTTATTG TATGTTTTAA AATAACTAAA AGAGTATAAT | 4060 |
| TGGGGTGTTT GTAACACAAA GAAAGGATAA ATGCTTGAAG GTGACAGATA CCCCATTTAC CCTGATGTGA | 4130 |
| TTATTACACA TTGTATGCCT GTATCAAAAT ATCTCATGTA TGCTATAGAT ATAAACCCTA CTATATTAAA | 4200 |
| AATTAAAATT TTAATGGCCA GGCACGGTGG CTCATGTCCG TAATCCCAGC ACTTTGGGAG GCCGAGGCGG | 4270 |
| GTGGATCACC TGAGGTCAGG AGTTTGAAAC CAGTCTGGCC ACCATGATGA AACCCTGTCT CTACTAAAGA | 4340 |
| TACAAAAATT AGCCAGGCGT GGTGGCACAT ACCTGTAGTC CCAACTACTC AGGAGCCTGA GACAGGAGAA | 4410 |
| TTGCTTGAAC CTGGGAGGCG GAGGTTGCAG TGAGCCGAGA TCATGCCACT GCACTGCAGC CTGGGTGACA | 4480 |
| GAGGAAGACT CCATCTCAAA ACAAAAACAA AAAAAAGAAG ATTAAAATTG TAATTTTTAT GTACCGTATA | 4550 |
| AATATATACT CTACTATATT AGAAGTTAAA AATTAAAACA ATTATAAAAG GTAATTAACC ACTTAATCTA | 4620 |
| AAATAAGAAC AATGTATGTG GGGTTTCTAG CTTCTGAAGA AGTAAAAGCT ATGGCCACGA TGGCAGAAAT | 4690 |
| GTGAGGAGGG AACAGTGGAA GTTACTGTTG TTAGACGCTC ATACTCTCTG TAAGTGACTT AATTTCAACC | 4760 |
| AAAGACAGGC TGGGAGAAGT TAAAGAGGCA TTCTATAAGC CCTAAAACAA CTGCTAATAA TGGTGAAAGG | 4830 |
| TAATCTCTAT TAATTACCAA TAATTACAGA TATCTCTAAA ATCGACCTGC AGAATTGGCA CGTCTCATCA | 4900 |
| CACCGTCCTC TCATTCACGG TGCTTTTTTT CTTGTGTGCT TGGAGATTTT CGATTGTGTG TTCGTGTTTG | 4970 |
| GTTAAACTTA ATCTGTATGA ATCCTGAAAC GAAAAATGGT GGTGATTTCC TCCAGAAGAA TTAGAGTACC | 5040 |
| TGGCAGGAAG CAGGTGGCTC TGTGGACCTG AGCCACTTCA ATCTTCAAGG GTCTCTGGCC AAGACCCAGG | 5110 |
| TGCAAGGCAG AGGCCTGATG ACCCGAGGAC AGGAAAGCTC GGATGGGAAG GGGCGATGAG AAGCCTGCCT | 5180 |
| CGTTGGTGAG CAGCGCATGA AGTGCCCTTA TTTACGCTTT GCAAAGATTG CTCTGGATAC CATCTGGAAA | 5250 |
| AGGCGGCCAG CGGGAATGCA AGGAGTCAGA AGCCTCCTGC TCAAACCCAG GCCAGCAGCT ATGGCGCCCA | 5320 |
| CCCGGGCGTG TGCCAGAGGG AGAGGAGTCA AGGCACCTCG AAGTATGGCT TAAATCTTTT TTTCACCTGA | 5390 |
| AGCAGTGACC AAGGTGTATT CTGAGGGAAG CTTGAGTTAG GTGCCTTCTT TAAAACAGAA AGTCATGGAA | 5460 |
| GCACCCTTCT CAAGGGAAAA CCAGACGCCC GCTCTGCGGT CATTTACCTC TTTCCTCTCT CCCTCTCTTG | 5530 |
| CCCTCGCGGT TTCTGATCGG GACAGAGTGA CCCCCGTGGA GCTTCTCCGA GCCCGTGCTG AGGACCCTCT | 5600 |
| TGCAAAGGGC TCCACAGACC CCCGCCCTGG AGAGAGGAGT CTGAGCCTGG CTTAATAACA AACTGGGATC | 5670 |
| TGGCTGGGGG CGGACAGCGA CGGCGGGATT CAAAGACTTA ATTCCATGAG TAAATTCAAC CTTTCCACAT | 5740 |
| CCGAATGGAT TTGGATTTTA TCTTAATATT TTCTTAAATT TCATCAAATA ACATTCAGGA CTGCAGAAAT | 5810 |
| CCAAAGGCGT AAAACAGGAA CTGAGCTATG TTTGCCAAGG TCCAAGGACT TAATAACCAT GTTCAGAGGG | 5880 |
| ATTTTTCGCC CTAAGTACTT TTTATTGGTT TTCATAAGGT GGCTTAGGGT GCAAGGGAAA GTACACGAGG | 5950 |
| AGAGGCCTGG GCGGCAGGGC TATGAGCACG GCAGGGCCAC CGGGGAGAGA GTCCCCGGCC TGGGAGGCTG | 6020 |
| ACAGCAGGAC CACTGACCGT CCTCCCTGGG AGCTGCCACA TTGGGCAACG CGAAGGCGGC CACGCTGCGT | 6090 |
| GTGACTCAGG ACCCCATACC GGCTTCCTGG GCCCACCCAC ACTAACCCAG GAAGTCACGG AGCTCTGAAC | 6160 |
| CCGTGGAAAC GAACATGACC CTTGCCTGCC TGCTTCCCTG GGTGGGTCAA GGGTAATGAA GTGGTGTGCA | 6230 |
| GGAAATGGCC ATGTAAATTA CACGACTCTG CTGATGGGGA CCGTTCCTTC CATCATTATT CATCTTCACC | 6300 |
| CCCAAGGACT GAATGATTCC AGCAACTTCT TCGGGTGTGA CAAGCCATGA CAAAACTCAG TACAAACACC | 6370 |
| ACTCTTTTAC TAGGCCCACA GAGCACGGSC CACACCCCTG ATATATTAAG AGTCCAGGAG AGATGAGGCT | 6440 |
| GCTTTCAGCC ACCAGGCTGG GGTGACAACA GCGGCTGAAC AGTCTGTTCC TCTAGACTAG TAGACCCTGG | 6510 |
| CAGGCACTCC CCCAGATTCT AGGGCCTGGT TGCTGCTTCC CGAGGGCGCC ATCTGCCCTG GAGACTCAGC | 6580 |
| CTGGGGTGCC ACACTGAGGC CAGCCCTGTC TCCACACCCT CCGCCTCCAG GCCTCAGCTT CTCCAGCAGC | 6650 |
| TTCCTAAACC CTGGGTGGGC CGTGTTCCAG CGCTACTGTC TCACCTGTCC CACTGTGTCT TGTCTCAGCG | 6720 |
| ACGTAGCTCG CACGGTTCCT CCTCACATGG GGTGTCTGTC TCCTTCCCCA ACACTCACAT GCGTTGAAGG | 6790 |
| GAGGAGATTC TGCGCCTCCC AGACTGGCTC CTCTGAGCCT GAACCTGGCT CGTGGCCCCC GATGCAGGTT | 6860 |
| CCTGGCGTCC GGCTGCACGC TGACCTCCAT TTCCAGGCGC TCCCCGTCTC CTGTCATCTG CCGGGGCCTG | 6930 |
| CCGGTGTGTT CTTCTGTTTC TGTGCTCCTT TCCACCTCCA GCTGCGTGTG TCTCTGCCCG CTAGGGTCTC | 7000 |
| GGGGTTTTTA TAGGCATAGG ACGGGGGCGT GGTGGGCCAG GGCGCTCTTG GGAAATGCAA CATTTGGGTG | 7070 |
| TGAAAGTAGG AGTGCCTGTC CTCACCTAGG TCCACGGGCA CAGGCCTGGG GATGGAGCCC CCGCCAGGGA | 7140 |
| CCCGCCCTTC TCTGCCCAGC ACTTTCCTGC CCCCCTCCCT CTGGAACACA GAGTGGCAGT TTCCACAAGC | 7210 |
| ACTAAGCATC CTCTTCCCAA AAGACCCAGC ATTGGCACCC CTGGACATTT GCCCCACAGC CCTGGGAATT | 7280 |
| CACGTGACTA CGCACATCAT GTACACACTC CCGTCCACGA CCGACCCCCG CTGTTTTATT TTAATAGCTA | 7350 |
| CAAAGCAGGG AAATCCCTGC TAAAATGTCC TTTAACAAAC TGGTTAAACA AACGGGTCCA TCCGCACGGT | 7420 |
| GGACAGTTCC TCACAGTGAA GAGGAACATG CCGTTTATAA AGCCTGCAGG CATCTCAAGG GAATTACGCT | 7490 |
| GAGTCAAAAC TGCCACCTCC ATGGGATACG TACGCAACAT GCTCAAAAAG AAAGAATTTC ACCCCATGGC | 7560 |
| AGGGGAGTGG TTAGGGGGGT TAAGGACGGT GGGGGCGGCA GCTGGGGGCT ACTGCACGCA CCTTTTACTA | 7630 |
| AAGCCAGTTT CCTGGTTCTG ATGGTATTGG CTCAGTTATG GGAGACTAAC CATAGGGGAG TGGGGATGGG | 7700 |
| GGAACCCGGA GGCTGTGCCA TCTTTGCCAT GCCCGAGTGT CCTGGGCAGG ATAATGCTCT AGAGATGCCC | 7770 |
| ACGTCCTGAT TCCCCCAAAC CTGTGGACAG AACCCGCCCG GCCCCAGGGC CTTTGCAGGT GTGATCTCCG | 7840 |
| TGAGGACCCT GAGGTCTGGG ATCCTTCGGG ACTACCTGCA GGCCCGAAAA GTAATCCAGG GGTTCTGGGA | 7910 |
| AGAGGCGGGC AGGAGGGTCA GAGGGGGGCA GCCTCAGGAC GATGGAGGCA GTCAGTCTGA GGCTGAAAAG | 7980 |
| GGAGGGAGGG CCTCGAGCCC AGGCCTGCAA GCGCCTCCAG AAGCTGGAAA AAGCGGGGAA GGGACCCTCC | 8050 |
| ACGGAGCCTG CAGCAGGAAG GCACGGCTGG CCCTTAGCCC ACCAGGGCCC ATCGTGGACC TCCGGCCTCC | 8120 |
| GTGCCATAGG AGGGCACTCG CGCTGCCCTT CTAGCATGAA GTGTGTGGGG ATTTGCAGAA GCAACAGGAA | 8190 |
| ACCCATGCAC TGTGAATCTA GGATTATTTC AAAACAAAGG TTTACAGAAA CATCCAAGGA CAGGGCTGAA | 8260 |
| GTGCCTCCGG GCAAGGGCAG GGCAGGCACG AGTGATTTTA TTTAGCTATT TTATTTTATT TACTTACTTT | 8330 |
| CTGAGACAGA GTTATGCTCT TGTTGCCCAG GCTGGAGTGC AGCGGCATGA TCTTGGCTCA CTGCAACCTC | 8400 |
| CGTCTCCTGG GTTCAAGCAA TTCTCGTGCC TCAGCCTCCC AAGTAGCTGG GATTTCAGGC GTGCACCACC | 8470 |
| ACACCCGGCT AATTTTGTAT TTTTAGTAGA GATGGGCTTT CACCATGTTG GTCAAGCTGA TCTCAAAATC | 8540 |
| CTGACCTCAG GTGATCCGCC CACCTCAGCC TCCCAAAGTG CTGGGATTAC AGGCATGAGC CACTGCACCT | 8610 |
| GGCCTATTTA ACCATTTTAA AACTTCCCTG GGCTCAAGTC ACACCCACTG GTAAGGAGTT CATGGAGTTC | 8680 |
| AATTTCCCCT TTACTCAGGA GTTACCCTCC TTTGATATTT TCTGTAATTC TTCGTAGACT GGGGATACAC | 8750 |
| CGTCTCTTGA CATATTCACA GTTTCTGTGA CCACCTGTTA TCCCATGGGA CCCACTGCAG GGGCAGCTGG | 8820 |
| GAGGCTGCAG GCTTCAGGTC CCAGTGGGGT TGCCATCTCC CAGTAGAAAC CTGATGTAGA ATCAGGGCGC | 8890 |
| AAGTGTGGAC ACTGTCCTGA ATCTCAATGT CTCAGTGTGT GCTGAAACAT GTAGAAATTA AAGTCCATCC | 8960 |
| CTCCTACTCT ACTGGGATTG AGCCCCTTCC CTATCCCCCC CCAGGGGCAG AGGAGTTCCT CTCACTCCTG | 9030 |
| TGGAGGAAGG AATGATACTT TGTTATTTTT CACTGCTGGT ACTGAATCCA CTGTTTCATT TGTTGGTTTG | 9100 |
| TTTGTTTTGT TTTGAGAGGC GGTTTCACTC TTGTTGCTCA GGCTGGAGGG AGTGCAATGG CGCGATCTTG | 9170 |
| GCTTACTGCA GCCTCTGCCT CCCAGGTTCA AGTGATTCTC CTGCTTCCGC CTCCCATTTG GCTGGGATTA | 9240 |
| CAGGCACCCG CCACCATGCC CAGCTAATTT TTTGTATTTT TAGTAGAGAC GGGGGTGGGT GGGGTTCACC | 9310 |
| ATGTTGGCCA GGCTGGTCTC GAACTTCTGA CCTCAGATGA TCCACCTGCC TCTGCCTCCT AAAGTGCTGG | 9380 |
| GATTACAGGT GTGAGCCACC ATGCCCAGCT CAGAATTTAC TCTGTTTAGA AACATCTGGG TCTGAGGTAG | 9450 |
| GAAGCTCACC CCACTCAAGT GTTGTGGTGT TTTAAGCCAA TGATAGAATT TTTTTATTGT TGTTAGAACA | 9520 |
| CTCTTGATGT TTTACACTGT GATGACTAAG ACATCATCAG CTTTTCAAAG ACACACTAAC TGCACCCATA | 9590 |
| ATACTGGGGT GTCTTCTGGG TATCAGCAAT CTTCATTGAA TGCCGGGAGG CGTTTCCTCG CCATGCACAT | 9660 |
| GGTGTTAATT ACTCCAGCAT AATCTTCTGC TTCCATTTCT TCTCTTCCCT CTTTTAAAAT TGTGTTTTCT | 9730 |
| ATGTTGGCTT CTCTGCAGAG AACCAGTGTA AGCTACAACT TAACTTTTGT TGGAACAAAT TTTCCAAACC | 9800 |
| GCCCCTTTGC CCTAGTGGCA GAGACAATTC ACAAACACAG CCCTTTAAAA AGGCTTAGGG ATCACTAAGG | 9870 |
| GGATTTCTAG AAGAGCGACC TGTAATCCTA AGTATTTACA ACACGAGGCT AACCTCCAGC GAGCGTGACA | 9940 |
| GCCCAGGGAG GGTGCGAGGC CTGTTCAAAT GCTAGCTCCA TAAATAAAGC AATTTCCTCC GGCAGTTTCT | 10010 |
| GAAAGTAGGA AAGGTTACAT TTAAGGTTGC GTTTGTTAGC ATTTCAGTGT TTGCCGACCT CAGCTACAGC | 10080 |
| ATCCCTGCAA GGCCTCGGGA GACCCAGAAG TTTCTCGCCC CCTTAGATCC AAACTTGAGC AACCCGGAGT | 10150 |
| CTGGATTCCT GGGAAGTCCT CAGCTGTCCT GCGGTTGTGC CGGGGCCCCA GGTCTGGAGG GGACCAGTGG | 10220 |
| CCGTGTGGCT TCTACTGCTG GGCTGGAAGT CGGGCCTCCT AGCTCTGCAG TCCGAGGCTT GGAGCCAGGT | 10290 |
| GCCTGGACCC CGAGGCTGCC CTCCACCCTG TGCGGGCGGG ATGTGACCAG ATGTTGGCCT CATCTGCCAC | 10360 |
| ACAGAGTGCC GGGGCCCAGG GTCAAGGCCG TTGTGGCTGG TGTGAGGCGC CCGGTGCGCG GCCAGGAGGA | 10430 |
| GCGCCTGGCT CCATTTCCCA CCCTTTCTCG ACGGGACCGC CCCGGTGGGT GATTAACAGA TTTGGGGTGG | 10500 |
| TTTGCTCATG GTGGGGACCC CTCGCCGCCT GAGAACCTGC AAAGAGAAAT GACGGGCCTG TGTCAAGGAG | 10570 |
| CCCAAGTCGC GGGGAAGTGT TGCAGGGAGG CACTCCGGGA GGTCCCGCGT GCCCGTCCAG CGAGCAATGC | 10640 |
| GTCCTCGGGT TCGTCCCCAG CCGCCTCTAC GCCCCTCCGT CCTCCCCTTC ACGTCCGGCA TTCGTGGTGC | 10710 |
| CCGGAGCCCG ACGCCCCGCG TCCGGACCTG CAGGCAGCCC TGGGTCTCCG GATCAGGCCA GCGGCCAAAG | 10780 |
| GGTCGCCGCA CGCACCTGTT CCCAGGGCCT CCACATCATG GCCCCTCCCT CGGGTTACCC CACAGCCTAG | 10850 |
| GCCGATTCGA CCTCTCTCCC CTGGGGCCCT CGCTGGCGTC CCTGCACCCT GGGAGCGCGA GCGGCGCGCG | 10920 |
| GGCGGGGAAG CGCGGCCCAG ACCCCCGGGT CCGCCCGGAG CAGCTGCGCT GTCGGGGCCA GGCCGGGCTC | 10990 |
| CCAGTGGATT CGCGGGCACA GACGCCCAGG ACCGCGCTCC CCACGTGGCG GAGGGACTGG GGACCCGGGC | 11060 |
| ACCCGTCCTG CCCCTTCACC TTCCAGCTCC GCCTCCTCCG CGCGGACCCC GCCCCGTCCC GACCCCTCCC | 11130 |
| GGGTCCCCGG CCCAGCCCCC TCCGGGCCCT CCCAGCCCCT CCCCTTCCTT TCCGCGGCCC CGCCCTCTCC | 11200 |
| TCGCGGCGCG AGTTTCAGGC AGCGCTGCGT CCTGCTGCGC ACGTGGGAAG CCCTGGCCCC GGCCACCCCC | 11270 |
| GCGATGCCGC GCGCTCCCCG CTGCCGAGCC GTGCGCTCCC TGCTGCGCAG CCACTACCGC GAGGTGCTGC | 11340 |
| CGCTGGCCAC GTTCGTGCGG CGCCTGGGGC CCCACGGCTG GCGGCTGGTG CAGCGCGGGG ACCCGGCGGC | 11410 |
| TTTCCGCGCG CTGGTGGCCC AGTGCCTGGT GTGCGTGCCC TGGGACGCAC GGCCGCCCCC CGCCGCCCCC | 11460 |
| TCCTTCCGCC AGGTGGGCCT CCCCGGGGTC GGCGTCCGGC TGGGGTTGAG GGCGGCCGGG GGGAACCAGC | 11550 |
| GACATGCGGA GAGCAGCGCA GGCGACTCAG GGCGCTTCCC CCGCAGGTGT CCTGCCTGAA GGAGCTGGTG | 11620 |
| GCCCGAGTGC TGCAGAGGCT GTGCGAGCGC GGCGCGAAGA ACGTGCTGGC CTTCGGCTTC GCGCTGCTGG | 11690 |
| ACGGGGCCCG CGGGGGCCCC CCCGAGGCCT TCACCACCAG CGTGCGCAGC TACCTGCCCA ACACGGTGAC | 11760 |
| CGACGCACTG CGGGGGAGCG GGGCGTGGGG GCTGCTGCTG CGCCGCGTGG GCGACGACGT GCTGGTTCAC | 11830 |
| CTGCTGGCAC GCTGCGCGCT CTTTGTGCTG GTGGCTCCCA GCTGCGCCTA CCAGGTGTGC GGGCCGCCGC | 11900 |
| TGTACCAGCT CGGCGCTGCC ACTCAGGCCC GGCCCCCGCC ACACGCTAGT GGACCCCGAA GGCGTCTGGG | 11970 |
| ATGCGAACGG GCCTGGAACC ATAGCGTCAG GGAGGCCGGG GTCCCCCTGG GCCTGCCAGC CCCGGGTGCG | 12040 |
| AGGAGGCGCG GGGGCAGTGC CAGCCGAAGT CTGCCGTTGC CCAAGAGGCC CAGGCGTGGC GCTGCCCCTG | 12110 |
| AGCCGGAGCG GACGCCCGTT GGGCAGGGGT CCTGGGCCCA CCCGGGCAGG ACGCGTGGAC CGAGTGACCG | 12180 |
| TGGTTTCTGT GTGGTGTCAC CTGCCAGACC CGCCGAAGAA GCCACCTCTT TGGAGGGTGC GCTCTCTGGC | 12250 |
| ACGCGCCACT CCCACCCATC CGTGGGCCGC CAGCACCACG CAGGCCCCCC ATCCACATCG CGGCCACCAC | 12320 |
| GTCCCTGGGA CACGCCTTGT CCCCCGGTGT ACGCCGAGAC CAAGCACTTC CTCTACTCCT CAGGCGACAA | 12390 |
| GGAGCAGCTG CGGCCCTCCT TCCTACTCAG CTCTCTGAGG CCCAGCCTGA CTGGCGCTCG GAGGCTCGTG | 12460 |
| GAGACCATCT TTCTGGGTTC CAGGCCCTGG ATGCCAGGGA CTCCCCGCAG GTTGCCCCGC CTGCCCCAGC | 12530 |
| GCTACTGGCA AATGCGGCCC CTGTTTCTGG AGCTGCTTGG GAACCACGCG CAGTGCCCCT ACGGGGTGCT | 12600 |
| CCTCAAGACG CACTGCCCGC TGCGAGCTGC GGTCACCCCA GCAGCCGGTG TCTGTGCCCG GGAGAAGCCC | 12670 |
| CAGGGCTCTG TGGCGGCCCC CGAGGAGGAG GACACAGACC CCCGTCGCCT GGTGCAGCTG CTCCGCCAGC | 12740 |
| ACAGCAGCCC CTGGCAGGTG TACGGCTTCG TGCGGGCCTG CCTGCGCCGG CTGGTGCCCC CAGGCCTCTG | 12810 |
| GGGCTCCAGG CACAACGAAC GCCGCTTCCT CAGGAACACC AAGAAGTTCA TCTCCCTGGG GAAGCATGCC | 12880 |
| AAGCTCTCGC TGCAGGAGCT GACGTGGAAG ATGAGCGTGC GGGACTGCGC TTGGCTGCGC AGGAGCCCAG | 12950 |
| GTGAGGAGGT GGTGGCCGTC GAGGGCCCAG GCCCCAGAGC TGAATGCAGT AGGGGCTCAG AAAAGGGGGC | 13020 |
| AGGCAGAGCC CTGGTCCTCC TGTCTCCATC GTCACGTGGG CACACGTGGC TTTTCGCTCA GGACGTCGAG | 13090 |
| TGGACACGGT GATCTCTGCC TCTGCTCTCC CTCCTGTCCA GTTTGCATAA ACTTACGAGG TTCACCTTCA | 13160 |
| CGTTTTGATG GACACGCGGT TTCCAGGCGC CGAGGCCAGA GCAGTGAACA GAGGAGGCTG GGCGCGGCAG | 13230 |
| TGGAGCCGGG TTGCCGGCAA TGGGGAGAAG TGTCTGGAAG CACAGACGCT CTGGCGAGGG TGCCTGCAGG | 13300 |
| TTACCTATAA TCCTCTTCGC AATTTCAAGG GTGGGAATGA GAGGTGGGGA CGAGAACCCC CTCTTCCTGG | 13370 |
| GGGTGGGAGG TAAGGGTTTT GCAGGTGCAC GTGGTCAGCC AATATGCAGG TTTGTGTTTA AGATTTAATT | 13440 |
| GTGTGTTGAC GGCCAGGTGC GGTGGCTCAC GCCGGTAATC CCAGCACTTT GGGAAGCTGA GGCAGGTGGA | 13510 |
| TCACCTGAGG TCAGGAGTTT GAGACCAGCC TGACCAACAT GGTGAAACCC TATCTGTACT AAAAATACAA | 13580 |
| AAATTAGCTG GGCATGGTGG TGTGTGCCTG TAATCCCAGC TACTTGGGAG GCTGAGGCAG GAGAATCACT | 13650 |
| TGAACCCAGG AGGCGGAGGC TGCAGTGAGC TGAGATTGTG CCATTGTACT CCAGCCTGGG CGACAAGAGT | 13720 |
| GAAACTCTGT CTTTAAAAAA AAAAAGTGTT CGTTGATTGT GCCAGGACAG GGTAGAGGGA GGGAGATAAG | 13790 |
| ACTGTTCTCC AGCACAGATC CTGGTCCCAT CTTTAGGTAT GAAGAGGGCC ACATGGGAGC AGAGGACAGC | 13860 |
| AGATGGCTCC ACCTGCTGAG GAAGGGACAG TGTTTGTGGG TGTTCAGGGG ATGGTGCTGC TGGGCCCTGC | 13930 |
| CGTGTCCCCA CCCTGTTTTT CTGGATTTGA TGTTGAGGAA CCTCCGCTCC AGCCCCCTTT TGGCTCCCAG | 14000 |
| TGCTCCCAGG CCCTACCGTG GCAGCTAGAA GAAGTCCCGA TTTCACCCCC TCCCCACAAA CTCCCAAGAC | 14070 |
| ATGTAAGACT TCCGGCCATG CAGACAAGGA GGGTGACCTT CTTGGGGCTC TTTTTTTTCT TTTTTTCTTT | 14140 |
| TTATGGTGGC AAAAGTCATA TAACATGAGA TTGGCACTCC TAACACCGTT TTCTGTGTAC AGTGCAGAAT | 14210 |
| TGCTAACTCG GCGGTGTTTA CAGCAGGTTG CTTGAAATGC TGCGTCTTGC GTGACTGGAA GTCCCTACCC | 14280 |
| ATCGAACGGC AGCTGCCTCA CACCTGCTGC GGCTCAGGTG GACCACGCCG AGTCAGATAA GCGTCATGCA | 14350 |
| ACCCAGTTTT GCTTTTTGTG CTCCAGCTTC CTTCGTTGAG GAGAGTTTGA GTTCTCTGAT CAGGACTCTG | 14420 |
| CCTGTCATTG CTGTTCTCTG ACTTCAGATG AGGTCACAAT CTGCCCCTGG CTTATGCAGG GAGTGAGGCG | 14490 |
| TGGTCCCCGG GTGTCCCTGT CACGTGCAGG GTGAGTGAGG CGTTGCCCCC AGGTGTCCCT GTCACGTGTA | 14560 |
| GGGTGAGTGA GGCGCGGCCC CCGGGTGTCC CTGTCCCGTG CAGCGTGATT GAGGTGTGGC CCCCGGGTGT | 14630 |
| CCCTGTCACG TGTAGGGTGA GTGAGGCGCC ATCCCCGGGT GTCCCTGTCA CGTGTAGGGT GAGTGAGGCG | 14700 |
| TGGTCCCCGG GTGTCCCTGT CCCGTGCAGG GTGAGTGAGG CACTGTCCCC GGGTGTCCCT GTCACGTGCA | 14770 |
| GGGTGAGTGA GGCGCGGTCC CCGGGTGTCC CTCTCAGGTG TAGGGTGAGT GAGGCGCGGC CCCAGGGTGT | 14840 |
| CCCTGTCACG TGTAGGGTGA GTGAGGCACC GTCCCTGGGT GTCCCTCCCA GGTATAGGGT GAGTGAGGCA | 14910 |
| CTGTCCCCGG GTGTCCCTGT CACGTGCAGG GTGAGTGAGG CGCGGCCCCC GGGTGTCCCT CTCAGGTGCA | 14980 |
| GGGTGAGTGA GGCGCTGTCC CTGGGTGTCC CTGTCTCGTG TAGGGTGAGT GAGGCTCTGT CCCCAGGTGT | 15050 |
| CCTTGGCGTT TGCTCACTTG AGCTTGCTCC TGAATGTTTG CTCTTTCTAT AGCCACAGCT GCGCCGGTTG | 15120 |
| CCCATTGCCT GGGTAGATGG TGCAGGCGCA GTGCTGGTCC CCAAGCCTAT CTTTTCTGAT GCTCGGCTCT | 15190 |
| TCTTGGTCAC CTCTCCGTTC CATTTTGCTA CGGGGACACG GGACTGCAGG CTCTCGCCTC CCGCGTGCCA | 15260 |
| GGCACTGCAG CCACAGCTTC AGGTCCGCTT GCCTCTGTTG GGCCTGGCTT GCTCACCACG TGCCCGCCAC | 15330 |
| ATGCATGCTG CCAATACTCC TCTCCCAGCT TGTCTCATGC CGAGGCTGGA CTCTGGGCTG CCTGTGTCTG | 15400 |
| CTGCCACGTG TTGCTGGAGA CATCCCAGAA AGGGTTCTCT GTGCCCTGAA GGAAAGCAAG TCACCCCAGC | 15470 |
| CCCCTCACTT GTCCTGTTTT CTCCCAAGCT GCCCCTCTGC TTGGCCCCCT TGGGTGGGTG GCAACGCTTG | 15540 |
| TCACCTTATT CTGGGCACCT GCCGCTCATT GCTTAGGCTG GGCTCTGCCT CCAGTCGCCC CCTCACATGG | 15610 |
| ATTGACGTCC AGCCACAGGT TGGAGTGTCT CTGTCTGTCT CCTGCTCTGA GACCCACGTG GAGGGCCGGT | 15680 |
| GTCTCCGCCA GCCTTCGTCA GACTTCCCTC TTGGGTCTTA GTTTTGAATT TCACTGATTT TTAGTTTAGT | 15750 |
| TTTCTATCTC TCCATTGTAT GCTTTTTCTT GGTTTATTCT TTCATTCCTT TTCTAGCTTC TTAGTTTAGT | 15820 |
| CATGCCTTTC CCTCTAAGTG CTGCCTTACC TGCACCCTGT GTTTTGATGT GAAGTAATCT CAACATCAGC | 15890 |
| CACTTTCAAG TGTTCTTAAA ATACTTCAAA GTGTTAATAC TTCTTTTAAG TATTCTTATT CTGTGATTTT | 15960 |
| TTTCTTTGTG CACGCTGTGT TTTGA [symbol] GTGA AATCATTTTG ATATCAGTGA CTTTTAAGTA TTCTTTAGCT | 16030 |
| TATTCTGTGA TTTCTTTGAG CACTGAGTTA TTTGAACACT GTTTATGTTC AAGATATGTA GAGTATCAAG | 16100 |
| ATACGTAGAG TATTTTAAGT TATCATTTTA TTATTGATTT CTAACTCAGT TGTGTAGTGG TCTGTATAAT | 16170 |
| ACCAATTATT TGAAGTTTGC GGAGCCTTGC TTTGTGATCT AGTGTGTGCA TGGTTTCCAG AACTGTCCAT | 16240 |
| TGTAAATTTG ACATCCTGTC AATAGTGGGC ATGCATGTTC ACTATATCCA GCTTATTAAG GTCCAGTGCA | 16310 |
| AAGCTTCTGT CTCCTTCTAG ATGCATGAAA TTCCAAGAAG GAGGCCATAG TCCCTCACCT GGGGGATGGG | 16380 |
| TCTGTTCATT TCTTCTCCTT TGGTAGCATT TATGTGAGGC ATTGTTAGGT GCATGCACGT GGTAGAATTT | 16450 |
| TTATCTTCCT GATGAGTGAA TCTTTTGGAG ACTTCTATGT CTCTAGTAAT CTAGTAATTC TTTTTTTAAA | 16520 |
| TTGCTCTTAG TACTGCCACA CTGGGCTTCT TTTGATTAGT ATTTTCCTGC TGTGTCTGTT TTCTGCCTTT | 16590 |
| AATTTATATA TATATATATA TTTTTTTTTT TTTTGAGACA GAGTCTTGGT CTGTCGCCCA GGGTGAGTGC | 16660 |
| AGTGGTGTGA TCACAGGTCA GTGTAACTTT TACCTTCTGG CCTGAGCCGT CCTCTCACCT CAGCCTCCTG | 16730 |
| AGTAGCTGGA ACTGCAGACA CGCACCGCTA CACCTGGCTA ATTTTTAAAT TTTTTCTGGA GACAGGGTCT | 16800 |
| TGCTGTGTTG CCCAGGCTGG TCTCAAACTC TTGGACTCAA GGGATCCATC TACCTCGGCT TCCCAAAGTG | 16870 |
| CTGAATTACA GGCATGAGCC ACCATGTCTG GCCTAATTTT CAACACTTTT ATATTCTTAT AGTGTGGGTA | 16940 |
| TGTCCTGTTA ACAGCATGTA GGTGAATTTC CAATCCAGTC TGACAGTCGT TGTTTAACTG GATAACCTCA | 17010 |
| TTTATTTTCA TTTTTTTGTC ACTAGAGACC CGCCTGGTGC ACTCTGATTC TCCACTTGCC TGTTGCATGT | 17080 |
| CCTCGTTCCC TTGTTTCTCA CCACCTCTTG GGTTGCCATG TGCGTTTCCT GCCGAGTGTG TGTTGATCCT | 17150 |
| CTCGTTGCCT CCTGGTCACT GGGCATTTGC TTTTATTTCT CTTTGCTTAG TGTTACCCCC TGATCTTTTT | 17220 |
| ATTGTCGTTG TTTGCTTTTG TTTATTGAGA CAGTCTCACT CTGTCACCCA GGCTGGAGTG TAATGGCACA | 17290 |
| ATCTCGGCTC ACTGCAACCT CTGCCTCCTC GGTTCAAGCA GTTCTCATTC CTCAACCTCA TGAGTAGCTG | 17360 |
| GGATTACAGG CGCCCACCAC CACGCCTGGC TAATTTTTGT ATTTTTAGTA GAGATAGGCT TTCACCATGT | 17430 |
| TGGCCAGGCT GGTCTCAAAC TCCTGACCTC AAGTGATCTG CCCGCCTTGG CCTCCCACAG TGCTGGGATT | 17500 |
| ACAGGTGCAA GCCACCGTGC CCGGCATACC TTGATCTTTT AAAATGAAGT CTGAAACATT GGTACCCTTG | 17570 |
| TCCTGAGCAA TAAGACCCTT AGTGTATTTT AGCTCTGGCC ACCCCCCAGC CTGTGTGCTG TTTTCCCTGC | 17640 |
| TGACTTAGTT CTATCTCAGG CATCTTGACA CCCCCACAAG CTAAGCATTA TTAATATTGT TTTCCGTGTT | 17710 |
| GAGTGTTTCT GTAGCTTTGC CCCCGCCCTG CTTTTCCTCC TTTGTTCCCC GTCTGTCTTC TGTCTCAGGC | 17780 |
| CCGCCGTCTG GGGTCCCCTT CCTTGTCCTT TGCGTGGTTC TTCTGTCTTG TTATTGCTGG TAAACCCCAG | 17850 |
| CTTTACCTGT GCTGGCCTCC ATGGCATCTA GCGACGTCCG GGGACCTCTG CTTATGATGC ACAGATGAAG | 17920 |
| ATGTGGAGAC TCACGAGGAG GGCGGTCATC TTGGCCCGTG AGTGTCTGGA GCACCACGTG GCCAGCGTTC | 17990 |
| CTTAGCCAGT GAGTGACAGC AACGTCCGCT CGGCCTGGGT TCAGCCTGGA AAACCCCAGG CATGTCGGGG | 18060 |
| TCTGGTGGCT CCGCGGTGTC GAGTTTGAAA TCGCGCAAAC CTGCGGTGTG GCGCCAGCTC TGACGGTGCT | 18130 |
| GCCTGGCGGG GGAGTGTCTG CTTCCTCCCT TCTGCTTGGG AACCAGGACA AAGGATGAGG CTCCGAGCCG | 18200 |
| TTGTCGCCCA ACAGGAGCAT GACGTGAGCC ATGTGGATAA TTTTAAAATT TCTAGGCTGG GCGCGGTGGC | 18270 |
| TCACGCCTGT AATCCCAGCA CTTTGGGAGG CCAAGGCGGG TGGATCACGA GGTCAGGAGG TCGAGACCAT | 18340 |
| CCTGGCCAAC ATGATGAAAC CCCATCTGTA CTAAAAACAC AAAAATTAGC TGGGCGTGGT GGCGGGTGCC | 18410 |
| TGTAATCCCA GCTACTCGGG AGGCTGAGGC AGGAGAATTG CTTGAACCTG GGAGTTGGAA GTTGCAGTGA | 18480 |
| GCCGACATTG CACCACTGCA CTCCAGCCTG GCAACACAGC GAGACTCTGT CTCAAAAAAA AAAAAAAAAA | 18550 |
| AAAAAAAAAA AATTCTAGTA GCCACATTAA AAAAGTAAAA AAGAAAAGGT GAAATTAATG TAATAATAGA | 18620 |
| TTTTACTGAA GCCCAGCATG TCCACACCTC ATCATTTTAG GGTGTTATTG GTGGGAGCAT CACTCACAGG | 18690 |
| ACATTTGACA TTTTTTGAGC TTTGTCTGCG GGATCCCGTG TGTAGGTCCC GTGCGTGGCC ATCTCGGCCT | 18760 |
| GGACCTGCTG GGCTTCCCAT GGCCATGGCT GTTGTACCAG ATGGTGCAGG TCCGGGATGA GGTCGCCAGG | 18830 |
| CCCTCAGTGA GCTGGATGTG CAGTGTCCGG ATGGTGCACG TCTGGGATGA GGTCGCCAGG CCCTGCTGTG | 18900 |
| AGCTGGATGT GTGGTGTCTG GATGGTGCAG GTCAGGGGTG AGGTCTCCAG GCCCTCGGTG AGCTGGAGGT | 18970 |
| ATGGAGTCCG GATGATGCAG GTCCGGGGTG AGGTCGCCAG GCCCTGCTGT GAGCTGGATG TGTGGTGTCT | 19040 |
| GGATGGTGCA GGTCAGGGGT GAGGTCTCCA GGCCCTCGGT AAGCTGGAGG TATGGAGTCC GGATGATGCA | 19110 |
| GGTCCGGGGT GAGGTCGCCA GGCCCTGCTG TGAGCTGGAT GTGTGGTGTC TGGATGGTGC AGGTCTGGGG | 19180 |
| TGAGGTCACC AGGCCCTGCG GTGAGCTGGG TGTGCGGTGT CTGGATGGTG CAGGTCTGGA GTGAGGTCGC | 19250 |
| CAGACGGTGC CAGACCATGC GGTGAGCTGG ATATGCGGTG TCCGGATGGT GCAGGTCTGG GGTGAGGTTG | 19320 |
| CCAGGCCCTG CTGTGAGTTG GATGTGGGGT GTCCGGATGC TGCAGGTCCG GTGTGAGGTC ACCAGGCCCT | 19390 |
| GCTGTGAGCT GGATGTGTGG TGTCTGGATG GTGCAGGTCT GGGGTGAAGG TCGCCAGGCC CCTGCTTGTG | 19460 |
| AGCTGGATGT GTGGTGTCTG GATGGTGCAG GTCTGGAGTG AGGTCGCCAG GCCCTCGGTG AGCTGGATGT | 19530 |
| GCAGTGTCCA GATGGTGCAG GTCCGGGGTG AGGTCGCCAG ACCCTGCGGT GAGCTGGATG TGCCGTGTCT | 19600 |
| GGATGGTGCA GGTCTGGAGT GAGGTCGCCA GGCCCTCGGT GAGCTGGATG TATGGAGTCC GGATGGTGCC | 19670 |
| GGTCCGGGGT GAGGTCGCCA GACCCTGCTG TGAGCTGGAT GTGCGGTGTC TGGATGGTAC AGGTCTGGAG | 19740 |
| TGAGGTCGCC AGACCCTGCT GTGAGCTGGA TATGCGGTGT CCGGATGGTG CAGGTCAGGG GTGAGGTCTC | 19810 |
| CAGGCCCTCG GTGAGCTGGA GGTATGGAGT CCGGATGATG CAGGTCCGGG GTGAGGTCGC CAGGCCCTGC | 19880 |
| TGTGAACTGG ATGTGCGGCG TCTGGATGGT GCAGGTCTGG GGTGTGGTCG CCAGGCCCTC GGTGAGCTGG | 19950 |
| AGGTATGGAG TCCGGATGAT GCAGGTCCGG GGTGAGGTCG CCAGGCCCTG CTGTGAGCTG GATGTGCGGC | 20020 |
| GTCTGGATGG TGCAGGTCTG GGGTGTGGTC GCCAGGCCCT CGGTGAGCTG GAGGTATGGA GTCCGGATGA | 20090 |
| TGCAGGTCCG GGGTGAGGTT GCCAGGCCCT GCTGTGAGCT GGATGTGCTG TATCCGGATG GTGCAGTCCG | 20160 |
| GGGTGAGGTC GCCAGCCCCT GCTGTGAGCT GGATGTGCTC TATCCGGATG GTGCAGGTCT GGGGTGAGGT | 20230 |
| CACCAGGCCC TGCGGTGAGC TGGTTGTGCG GTGTCCGGTT CCTGCAGGTC CGGGGTGAGT TCGCCAGGCC | 20300 |
| CTCGGTGAGC TGGATGTGCG GTGTCCCCGT GTCCGGATGG TGCAGGTCCA GGGTCAGGTC GCTAGGCCCT | 20370 |
| TGGTGGGCTG GATGTGCCGT GTCCGGATGG TGCAGGTCTG GGGTGAGGTC GCCAGGCCTT TGGTGAGCTG | 20440 |
| GATGTGCGGT GTCTGCATGG TGCAGGTCTG GGGTGAGGTC GCCAGGCCCT TGGTGGGCTG GATGTGTGGT | 20510 |
| GTCCGGATGC TGCAGGTCCG GCGTGAGGTC GCCAGGCCCT GCTGTGAGCT GGATGTGCGG TGTCTGGATG | 20580 |
| GTGCAGGTCC GGGGTGAGGT AGCCAAGGCC TTCGGTGAGC TGGATGTGGG GTGTCCGGAT GGTGCAGGTC | 20650 |
| CGGGGTGAGG TCGCCAGGCC CTGCGGTTAG CTGGATATGC GGTGTCCGGA TGGTGCAGGT CCGGGGTGAG | 20720 |
| GTCACCAGGC CCTGCGGTTA GCTGGATGTG CGGTGTCTGC ATGGTGCAGG TCCGGGGTGA GGTCGCCAGG | 20790 |
| CCCTGCTGTG AGCTGGATGT GCTGTATCCG GATGGTGCAG GTCCGGGGTG AGGTCGCCAG GCCCTGCAGT | 20860 |
| GAGCTGGATG TGCTGTATCC GGATGGTGCA GGTCTGGCGT GAGGTCGCCA GGCCCTGCGG TTAGCTGGAT | 20930 |
| ATGCGGTGTC GGATGGTGCA GGTCCGGGGT GAGGTCACCA GGCCCTGCGG TTAGCTGGAT GTGCGGTGTC | 21000 |
| CGGATGGTGC AGGTCTGGGG TGAGGTCGCC AGGCCCTGCT GTGAGCTGGA TGTGCTGTAT CCGGATGGTG | 21070 |
| CAGGTCCGGG GTGAGGTCGC CAGGCCCTGC GGTGAGCTGG ATGTGCTGTA TCCGGATGGT GCAGGTCTGG | 21140 |
| CGTGAGGTCG CCAGGCCCTG CGGTGAGCTG GATGTGCAGT GTACGGATGG TGCAGGTCCG GGGTGAGGTC | 21210 |
| GCCAGGCCCT GCGGTGGGCT GTATGTGTGT TGTCTGGATG GTGCAGGTCC GGGGTGAGTT CGCCAGGCCC | 21280 |
| TGCGGTGAGC TGGATGTGTG GTGTCTGGAT GCTGCAGGTC CGGGGTGAGT TCGCCAGGCC CTCGGTGAGC | 21350 |
| TGGATATGCG GTGTCCCCGT GTCCGAATGG TGCAGGTCCA GGGTGAGGTC GCCAGGCCCT TGGTGGGCTG | 21420 |
| GATGTGCCGT GTCCGGATGG TGCAGGTCTC GGGTGAGGTC GCCAGGCCCT TGGTGAGCTG GATGTGCGGT | 21490 |
| GTCCGGATCG TGCAGGTCCG GGGTGAGGTC ACCAGGCCCT CGGTGATCTG GATGTGGCAT GTCCTTCTCG | 21560 |
| TTTAAGGGGT TGGCTGTGTT CCGGCCGCAG AGCACCGTCT GCGTGAGGAG ATCCTGGCCA AGTTCCTGCA | 21630 |
| CTGGCTGATG AGTGTGTACG TCGTCGAGCT GCTCAGGTCT TTCTTTTATG TCACGGAGAC CACGTTTCAA | 21700 |
| AAGAACAGGC TCTTTTTCTA CCGGAAGAGT GTCTGGAGCA ACTTGCAAAG CATTGGAATC AGGTACTGTA | 21770 |
| TCCCCACGCC AGGCCTCTGC TTCTCGAAGT CCTGGAACAC CAGCCCGGCC TCAGCATGCG CCTGTCTCCA | 21840 |
| CTTCCCTGTG CTTCCCTGGC TGTGCAGCTC TGGGCTGGGA GCCAGGGGCC CCGTCACAGG CCTGGTCCAA | 21910 |
| GTGGATTCTG TGCAAGGCTC TGACTGCCTG GAGCTCACGT TCTCTTACTT GTAAAATCAG GAGTTTGTGC | 21980 |
| CAAGTGGTCT CTAGGGTTTG TAAAGCAGAA GGGATTTAAA TTAGATGGAA ACACTACCAC TAGCCTCCTT | 22050 |
| GCCTTTCCCT GGGATGTGGG TCTGATTCTC TCTCTCTTTT TTTTTTCTTT TTTGAGATGG AGTCTCACTC | 22120 |
| TGTTGCCCAG GCTGGAGTGC AGTGGCATAA TCTTGGCTCA CTGCAACCTC CACCTCCTGG GTTTAAGCGA | 22190 |
| TTCACCAGCC TCAGCCTCCT AAGTAGCTGG GATTACAGGC ACCTGCCACC ACGCCTCGCT AATTTTTGTA | 22260 |
| CTTTTAGGAG AGACGGGGTT TCACCATGTT CGCCAGGCTG GTCTCGAACT CATGACCTCA GGTGATCCAC | 22330 |
| CCACCTTGGC CTCCCAAAGT GCTGGGTTTA CAGGCTAAGC CACCGTGCCC AGCCCCCGAT TCTCTTTTAA | 22400 |
| TTCATGCTGT TCTGTATGAA TCTTCAATCT ATTGGATTTA GGTCATGAGA CGATAAAATC CCACCCACTT | 22470 |
| GGCGACTCAC TGCAGGGAGC ACCTGTGCAG GGAGCACCTG GGGATAGGAG AGTTCCACCA TGAGCTAACT | 22540 |
| TCTAGGTGGC TGCATTTGAA TGGCTGTGAG ATTTTGTCTG CAATGTTCGG CTGATGAGAG TGTGAGATTG | 22610 |
| TGACAGATTC AAGCTGGATT TGCATCAGTG AGGGACGGGA GCGCTGGTCT GGGAGATGCC AGCCTGGCTG | 22680 |
| AGCCCAGGCC ATGGTATTAG CTTCTCCGTG TCCCGCCCAG GCTGACTGTG GAGGGCTTTA GTCAGAAGAT | 22750 |
| CAGGGCTTCC CCAGCTCCCC TGCACACTCG AGTCCCTGGG GGGCCTTGTG ACACCCCATG CCCCAAATCA | 22820 |
| GCATGTCTGC AGAGGGAGCT GGCAGCAGAC CTCGTCAGAG GTAACACAGC CTCTGGGCTG GGGACCCCGA | 22890 |
| CGTGGTGCTG GGGCCATTTC CTTGCATCTG GGGGAGGGTC AGGGCTTTCC CTGTGGGAAC AAGTTAATAC | 22960 |
| ACAATGCACC TTACTTAGAC TTTACACGTA TTTAATGGTG TGCGACCCAA CATGGTCATT TGACCAGTAT | 23030 |
| TTTGGAAAGA ATTTAATTGG GGTGACCGGA AGGAGCAGAC AGACGTGGTG GTCCCCAAGA TGCTCCTTGT | 23100 |
| CACTACTGGG ACTGTTGTTC TGCCTGGGGG GCCTTGGAGG CCCCTCCTCC CTGGACAGGG TACCGTGCCT | 23170 |
| TTTCTACTCT GCTGGGCCTG CGGCCTGCGG TCAGGGCACC AGCTCCGGAG CACCCGCGGC CCCAGTGTCC | 23240 |
| ACGGAGTGCC AGGCTGTCAG CCACAGATGC CCAGGTCCAG GTGTGGCCGC TCCAGCCCCC GTGCCCCCAT | 23310 |
| GGGTGGTTTT GGGGGAAAAG GCCAAGGGCA GAGGTGTCAG GAGACTGGTG GGCTCATGAG AGCTGATTCT | 23380 |
| GCTCCTTGGC TGAGCTGCCC TGAGCAGCCT CTCCCGCCCT CTCCATCTGA AGGGATGTGG CTCTTTCTAC | 23450 |
| CTGGGGGTCC TGCCTGGGGC CAGCCTTGGG CTACCCCAGT GCCTGTACCA GAGGGACAGG CATCCTGTGT | 23520 |
| GGAGGGGCAT GGGTTCACGT GGCCCCAGAT GCAGCCTGGG ACCAGGCTCC CTGGTGCTGA TGGTGGGACA | 23590 |
| GTCACCCTGG GGGTTGACCG CCGGACTGGG CGTCCCCAGG GTTGACTATA GGACCAGGTG TCCAGGTGCC | 23660 |
| CTGCAAGTAG AGGGGCTCTC AGAGGCGTCT GGCTGGCATG GGTGGACGTG GCCCCGGGCA TGGCCTTCAG | 23730 |
| CGTGTGCTGC CGTGGGTGCC CTGAGCCCTC ACTGAGTCGG TGGGGGCTTG TGGCTTCCCG TGAGCTTCCC | 23800 |
| CCTAGTCTGT TGTCTGGCTG AGCAAGCCTC CTGAGGGGCT CTCTATTGCA GACAGCACTT GAAGAGGGTG | 23870 |
| CAGCTGCGGG AGCTGTCGGA AGCAGAGGTC AGGCAGCATC GGGAAGCCAG GCCCGCCCTG CTGACGTCCA | 23940 |
| GACTCCGCTT CATCCCCAAG CCTGACGGGC TGCGGCCGAT TGTGAACATG GACTACGTCG TGGGAGCCAG | 24010 |
| AACGTTCCGC AGAGAAAAGA GGGTGGCTGT GCTTTGGTTT AACTTCCTTT TTAAACAGAA GTGCGTTTGA | 24080 |
| GCCCCACATT TGGTATCAGC TTAGATGAAG GGCCCGGAGG AGGGGCCACG GGACACAGCC AGGGCCATGG | 24150 |
| CACGGCGCCA ACCCATTTGT GCGCACAGTG AGGTGGCCGA GGTGCCGGTG CCTCCAGAAA AGCAGCGTGG | 24220 |
| GGGTGTAGGG GGAGCTCCTG GGGCAGGGAC AGGCTCTGAG GACCACAAGA AGCAGCCGGG CCAGGGCCTG | 24290 |
| GATGCAGCAC GGCCCGAGGT CCTGGATCCG TGTCCTGCTG TGGTGCGCAG CCTCCGTGCG CTTCCGCTTA | 24360 |
| CGGGGCCCGG GGACCAGGCC ACGACTGCCA GGAGCCCACC GGGCTCTGAG GATCCTGGAC CTTGCCCCAC | 24430 |
| GGCTCCTGCA CCCCACCCCT GTGGCTGCGG TGGCTGCGGT GACCCCGTCA TCTGAGGAGA GTGTGGGGTG | 24500 |
| AGGTGGACAG AGGTGTGGCA TGAGGATCCC GTGTGCAACA CACATGCGGC CAGGAACCCG TTTCAAACAG | 24570 |
| GGTCTGAGGA AGCTGGGAGG GGTTCTAGGT CCCGGGTCTG GGTGGCTGGG GACACTGGGG AGGGGCTGCT | 24640 |
| TCTCCCCTGG GTCCCTATGG TGGGGTGGGC ACTTGGCCGG ATCCACTTTC CTGACTGTCT CCCATGCTGT | 24710 |
| CCCCGCCAGG CCGAGCGTCT CACCTCGAGG GTGAAGGCAC TGTTCAGCGT GCTCAACTAC GAGCGGGCGC | 24780 |
| GGCGCCCCGG CCTCCTGGGC GCCTCTGTGC TGGGCCTGGA CGATATCCAC AGGGCCTGGC GCACCTTCGT | 24850 |
| GCTGCGTGTG CGGGCCCAGG ACCCGCCGCC TGAGCTGTAC TTTGTCAAGG TGGGTGCCGG GGACCCCCGT | 24920 |
| GAGCAGCCCT GCTGGACCTT GGGAGTGGCT GCCTGATTGG CACCTCATGT TGGGTGGAGG AGGTACTCCT | 24990 |
| GGGTGGGCCG CAGGGAGTGC AGGTGACCCT GTCACTGTTG AGGACACACC TGGCACCTAG GGTGGAGGCC | 25060 |
| TTCAGCCTTT CCTGCAGCAC ATGGGGCCGA CTGTGCACCC TGACTGCCCG GGCTCCTATT CCCAAGGAGG | 25130 |
| GTCCCACTGG ATTCCAGTTT CCGTCAGAGA AGGAACCGCA ACGGCTCAGC CACCAGGCCC CGGTGCCTTG | 25200 |
| CACCCCAGTC CTGAGCCAGG GGTCTCCTGT CCTGAGGCTC AGAGAGGGGA CACAGCCCGC CCTGCCCTTG | 25270 |
| GGGTCTGGAG TGGTGGGGGT CAGAGAGAGA GTGGGGGACA CCGCCAGGCC AGGCCCTGAG GGCAGAGGTG | 25340 |
| ATGTCTGAGT TTCTGCGTGG CCACTGTCAG TCTCCTCGCC TCCACTCACA CAGGTGGATG TGACGGGCGC | 25410 |
| GTACGACACC ATCCCCCAGG ACAGGCTCAC GGAGGTCATC GCCAGCATCA TCAAACCCCA GAACACGTAC | 25480 |
| TGCGTGCGTC GGTATGCCGT GGTCCAGAAG GCCGCCCATG GGCACGTCCG CAAGGCCTTC AAGAGCCACG | 25550 |
| TAAGGTTCAC GTGTGATAGT CGTGTCCAGG ATGTGTGTCT CTGGGATATG AATGTGTCTA GAATGCAGTC | 25620 |
| GTGTCTGTGA TGCGTTTCTG TGGTGGAGGT ACTTCCATGA TTTACACATC TGTGATATGC GTGTGTGGCA | 25690 |
| CGTGTGTGTC GTGGTGCATG TATCTGTGGC GTGCATATTT GTGGTGTGTG TGTGTGTGGC ACGTGTGTGT | 25760 |
| CCATGGTGTG TGTGCCTGTG GTGTGCATGT GTGTGTGTCT GTGACACGTG CATGTTCATG CTGTGTGCTG | 25830 |
| CATGTCTGTG ATGTGCCTAT TTGTGGTGTG TGTGTGCATG TGTCCGTGAC ATATGCGTGT CTATGGCATG | 25900 |
| GGTGTGTGTG GCCCCTTGGC CTTACTCCTT CCTCCTCCAG GCATGGTCCG CACCATTGTC CTCACGCTCT | 25970 |
| CGGGTGCTGG TTTGGGGAGC TCCACATTCA GGGTCCTCAC TTCTAGCATG GGTGCCCCTG TCCTGTCACA | 26040 |
| GGGCTGGGCC TTGGAGACTG TAAGCCAGGT TTGAGAGGAG AGTAGGGATG CTGGTGGTAC CTTCCTGGAC | 26110 |
| CCCTGGCACC CCCAGGACCC CAGTCTGGCC TATGCCGGCT CCATGAGATA TAGGAAGGCT GATTCAGGCC | 26180 |
| TCGCTCCCCG GGACACACTC CTCCCAGAGC GGCCGGGGGC CTTGGGGCTC GGCAGGGGTG AAAGGGGCCC | 26250 |
| TGGGCTTGGG TTCCCACCCA GTGGTCATGA GCACGCTGGA GGGGTAAGCC CTCAAAGTCG TGCCAGGCCG | 26320 |
| GGGTGCAGAG GTGAAGAAGT ATCCCTGGAG CTTCGGTCTG GGGAGAGGCA CATGTGGAAA CCCACAAGGA | 26390 |
| CCTCTTTCTC TGACTTCTTG AGCT | 26414 |

| Contig 2: | |
|---|---|
| TGTGGGATTG GTTTTCATGT GTGGGATAGG TGGGGATCTG TGGGATTGGT TTTTATGAGT GGGGTAACAC | 70 |
| AGAGTTCAAG GCGAGCTTTC TTCCTGTAGT GGGTCTGCAG GTGCTCCAAC AGCTTTATTG AGGAGACCAT | 140 |
| ATCTTCCTTT GAACTATGGT CGGGTTTATA GTAAGTCAGG GGTGTGGAGG CCTCCCCTGG GCTCCCTGTT | 210 |
| CTGTTTCTTC CACTCTGGGG TCGTGTGGTG CCTGCTGTGG TGTGTGGCCG GTGGGCAGGG CTTCCAGGCC | 280 |
| TCCTTGTGTT CATTGGCCTG GATGTGGCCC TGGCTACGCT CCGTCCTTGG AATTCCCCTG CGAGTTGGAG | 350 |
| GCTTTCTTTC TTTCTTTTTT TCTTTCTTTT TTTTTTTTTT TGATAACAGA GTCTCGCTCT TTTTTGCCCA | 420 |
| GGCTGGAGTG GTTTGGCGTG ATCTTGGCTC ACTGCAACCT GTGCTTCCTG AGTTCAAGCA ATTCTCTTGC | 490 |
| CTCAGCCTCC CAAGTAGCTG GAATTATAGG CGCCCACCAC CATGCTGACT AATTTTTGTA ATTTTAGTAG | 560 |
| AGACGAGGTT TCTCCATGTT GGCCAGGCTG GTCTCGAACT CCTGACCTCA GGTGATCCTC CCACCTCGGC | 630 |
| CTCCCAAAGT GCTGGGATGA CAGGTGTGAA CCGCCGCGCC CGGCCGAGAC TCGCTTCCTG CAGCTTCCGT | 700 |
| GAGATCTGCA GCGATAGCTG CCTGCAGCCT TGGTGCTGAC AACCTCCGTT TTCCTTCTCC AGGTCTCGCT | 770 |
| AGGGGTCTTT CCATTTCATG ACTCTCTTCA CAGAAGAGTT TCACGTGTGC TGATTTCCCG GCTGTTTCCT | 840 |
| GCGTAATTGG TGTCTGCTGT TTATCGATGG CCTCCTTCCA TTTCCTTTAG GCTTTGTTTA TTGTTGTTTT | 910 |
| TCCGGCTCCT TGAAGGAAAA GTTTCGATTA TGGATGTTTG AACTTTCTTT TCTAAACAAG CATCTGAAGT | 980 |
| TGCCGTTTTC CCTCTAAAGC AGGGATCCCG AGGCCCCTGG CTGTGGAGTG GCACCGGTCT GGGGCCTGTT | 1050 |
| AGGAACCCGG CGCACAGCGG GAGGCTAGGT GGGGTGTGGG GAGCCAGCGT TCCCGCCTGA GCCCCGCCCC | 1120 |
| TCTCAGATCA GCAGTGGCAT GCGGTGCTCA GAGGCGCACA CACCCTACTG AGAACTGTGC GTGAGAGGGG | 1190 |
| TCTAGATTCT GTGCTCCTTA TGGGAATCTA ATGCCTGATG ATCTGAGGTG GAACCGTTTG CTCCCAAAAC | 1260 |
| CATCCCCTTC CCCACTGCTG TCCTGTGGAA AAATCGTCTT CCACGAAACC AGTCCCTGGT ACCACAATGG | 1330 |
| TTGGGGACCC TGTGCTAAAG ACCTGCTTCA GCAGCCTCTC GTCAGTGTTG ATATATTGGC TTTTCTGTGT | 1400 |
| TGAGTCCAGA ATAATTACGG ATTTCTGTGA TGCTTTCCGC CGACCTCAGA CCCATGGGCT ATTTGTGGGC | 1470 |
| GTGTTGCCTG CTCCTGGGTT GGGAAGGGTG CAGGCCCCAT GTACCTTCCT GTTACTGCCT TCCAGGTTGG | 1540 |
| TTCTCAGGGT TGAATCGTAC TCGATGTGGT TTTAGCCCAC GGCCCTGCCG CCAGCTCCTG GGGGCTGGGG | 1610 |
| AACATGCTGA AGCACAGAGT CACCGTGCGC GTCTTTTGAT GCCTCACAAG CTCGAGGCCT CCTGTGTCCG | 1680 |
| TGTTAGTGTG TGTCACGTGC CTGCTCACAT CCTGTCTTGG GGACGCAGGG GCTTAGCAGG TCCCGTAGTA | 1750 |
| AATGACAAGC GTCCTGGGGG AGTCTGCAGA ATAGGAGGTG GGGGTGCCGG TCTCTCTCCC GCGTCTTCAG | 1820 |
| ACTCTTCTCC TGCCTGTGCT GTGGCTGCAC CTGCATCCCT GCAATCCCTC CAGCACTGGG CTGGAGAGGC | 1890 |
| CCGGGAGCTC GAGTGCCACT TGTGCCACGT GACTGTGGAT GGCAGTCGGT CACGGGGGTC TGATGTGTGG | 1960 |
| TGACTGTGGA TGGCGGTTGG TCACAGGGGT CTGATGTGTG GTGACTGTGG ATGGCGGTCG TGGGGTCTGA | 2030 |
| TGTGGTGACT GTGGATGGCG GTCGTGGGGT CTGATGTGTG GTGACTGTGG ATGGCGGTCG TGGGGTCTGA | 2100 |
| TGTGGTGACT GTGGATGGCG GTCGTGGGGT CTGATGTGGT GACTGTGGAT GGCGGTCGTG GGGTCTGATG | 2170 |
| TGGTGACTGT GGATGGCAGT CGTGGGGTCT GATGTGTGGT GACTGTGGAT GGCGGTCGTG GGGTCTGATG | 2240 |
| TGGTGACTGT GGATGGCAGT CGTGGGGTCT GATGTGTGGT GACTGTGGAT GGCGGTCGTG GGGTCTGATG | 2310 |
| TGTGGTGACT GTGGATGGCG GTCGTGGGGT CTGATGTGTG GTGACTGTGG ATGGCGGTCG TGGGGTCTGA | 2380 |
| TGTGTGGTGA CTGTGGATGG CGGTCGTGGG GTCTGATGTG GTGACTGTGG ATGGCGGTCG TGGGGTCTGA | 2450 |
| TGTGTGGTGA CTGTGGATGG TGATCGGTCA CAGGGGTCTG ATGTGTGGTG ACTGTGGATG GCGGTCGTGG | 2520 |
| GGTCTGATGT GTGGTGACTG TGGATGGTGA TCGGTCACAG GGGTCTGATG TGTGGTGACT GTGGATGGCG | 2590 |
| GTCGTGGGGT CTGATGTGTG GTGACTGTGG ATGGCGGTTG GTCCCGGGGG TCTGATGTGT GGTGACTGTG | 2660 |
| GATGGCGATC GGTCACAGGG GTCTGATGTG TGGTGACTGT GGATGGCGGT CGTGGGGTCT GATGTGTGGT | 2730 |
| GACTGTGGAT GGCGGTCGTG GGGTCTGATG TGTGGTGACT GTGGATGGCG GTCGTGGGGT CTGATGTGGT | 2800 |
| GACTGTGGAT GGCGGTCGTG GGGTCTGATG TGGTGACTGT GGATGGCGGT CGTGGGGTCT GATGTGTGGT | 2870 |
| GACTGTGGAT GGCGGTTGGT CCCGGGGGTC TGATGTGTGG TGACTGTGGA TGGCGGTCGT GGGGTCTGAT | 2940 |
| GTGGTGACTG TGGATGGCAG TCGTGGGGTC TGATGTGTGG TGACTGTGGA TGGCGGTCGT GGGGTCTGAT | 3010 |
| GTGTGGTGAC TGTGGATGGC GGTCGTGGGG TCTGATGTGT GGTGACTGTG GATGGCGGTC GTGGGGTCTG | 3080 |
| ATGTGTGGTG ACTGTGGATG GCGGTCGTGG GGTCTGATGT GGTGACTGTG GATGGCGGTC GTGGGGTCTG | 3150 |
| ATGTGTGGTG ACTGTGGATG GTGATCGGTC ACAGGGGTCT GATGTGTGGT GACTGTGGAT GGCGGTCGTG | 3220 |
| GGGTCTGATG TGTGGTGACT GTGGATGGCG GTCGTGGGGT CTGATGTGGT GACTGTGGAT GGCGGTCGTG | 3290 |
| GGGTCTGATG TGTGGTGACT GTGGATGGCG GTCGTAGGGT CTGATGTGTG GTGACTGTGG ATGGCAGTCG | 3360 |
| GTCACAGGGG TCTGATGTGT GGTGACTGTG GATGGCGGTC GTGGGGTCTG ATGTGTGGTG ACTGTGGATG | 3430 |
| GCGGTCGTGG GGTCTGATGT GTGGTGACTG TGGATGGCGG TCGTGGGGTC TGATGTGTGG TGACTGTGGA | 3500 |
| TGGCGGTCGT GGGGTCTGAT GTGGTGACTG TGGATGGTGA TCGGTCACAG GGGTCTGATG TGTGGTAGCT | 3570 |
| GCAGGTGGAG TCCCAGGTGT GTCTGTAGCT ACTTTGCGTC CTCGGCCCCC CGGCCCCCGT TTCCCAAACA | 3640 |
| GAAGCTTCCC AGGCGCTCTC TGGGCTTCAT CCCGCCATCG GGCTTGGCCG CAGGTCCACA CGTCCTGATC | 3710 |
| GGAAGAAACA AGTGCCCAGC TCTGGCCGGG GCAGGCCACA TTTGTGGCTC ATGCCCTCTC CTCTGCCGGC | 3780 |
| AGGTCTCTAC CTTGACAGAC CTCCAGCCGT ACATGCGACA GTTCGTGGCT CACCTGCAGG AGACCAGCCC | 3850 |
| GCTGAGGGAT GCCGTCGTCA TCGAGCAGGT CTGGGCACTG CCCTGCAGGG TTGGGCACGG ACTCCCAGCA | 3920 |
| GTGGGTCCTC CCCTGGGCAA TCACTGGGCT CATGACCGGA CAGACTGTTG GCCCTGGGGG GCAGTGGGGG | 3990 |
| GAATGAGCTG TGATGGGGGC ATGATGAGCT GTGTGCCTTG GCGAAATCTG AGCTGGGCCA TGCCAGGCTG | 4060 |
| CGACAGCTGC TGCATTCAGG CACCTGCTCA CGTTTGACTG CGCGGCCTCT CTCCAGTTCC GCAGTGCCTT | 4130 |
| TGTTCATGAT TTGCTAAATG TCTTCTCTGC CAGTTTTGAT CTTGAGGCCA AAGGAAAGGT GTCCCCCTCC | 4200 |
| TTTAGGAGGG CAGGCCATGT TTGAGCCGTG TCCTGCCCAG CTGGCCCCTC AGTGCTGGGT CTGAGGCCAA | 4270 |
| AGGAAACGTG TCCCCCTTCT TAGGAGGACG GGCCGTGTTT GAGCCACGCC CCGCTGAGCG GGCCTCTCAG | 4340 |
| TGCTGGGTCT GTCCACGTGG CCCTGTGGCC CTTTGCAGAT GTGGTCTGTC CACGTGGCCC TGTGGCTCTT | 4410 |
| TGCAGATGCC TGTTAGCACT TGCTCGGCTC TAGGGGACAG TCGTGTCCAC CGCATGAGGC TCAGAGACCT | 4480 |
| CTGGGCGAAT TTCCTTGGCT CCCAGGGTGG GGGTGGAGGT GGCCTGGGCT GCTGGGACCC AGACCCTGTG | 4550 |
| CCCGGCAGCT GGGCAGCAAC TCCTGGATCA CATATGCCAT CCGGGCCACG GTGGGCTGTG TGGGTGTGAG | 4620 |
| CCCAGCTGGA CCCACAGGTG GCCCAGAGGA GACGTTCTGT GTCACACACT TCGCCTAAGC CCATGTGTGT | 4690 |
| CTGCAGAGAC TCGGCCCGGC CAGCCCACGA TGGCCCTGCA TTCCAGCCCA GCCCCGCACT TCATCACAAA | 4760 |
| CACTGACCCC AAAAGGGACG GAGGGTCTTG GCCACGTGGT CCTGCCTGTC TCAGCACCCA CCGGCTCACT | 4830 |
| CCCATGTGTC TCCCGTCTGC TTTCGCAGAG CTCCTCCCTG AATGAGGCCA GCAGTGGCCT CTTCGACGTC | 4900 |
| TTCCTACGCT TCATGTGCCA CCACGCCGTG CGCATCAGGG GCAAGTGAGT CAGGTGGCCA GGTGCCATTG | 4970 |
| CCCTGCGGGC GGCTGGGCGG GCTGGCAGGG CTTCTGCTCA CCTCTCTCCT GCCCCTTCCC CACTGNCCTT | 5040 |
| CTGCCCGGGG CCACCAGAGT CTCCTTTTCT GGCCCCCGCC CCCTCCGGCT CCTGGGCTGC AGGCTCCCGA | 5110 |
| GGCCCCGGAA ACATGGCTCG GCTTGCGGCA GCCGGAGCGG AGCAGGTGCC ACACGAGGCC TGGAAATGGC | 5180 |
| AAGCGGGGTG TGGAGTTGCT CCTGCGTGGA GGACGAGGGG CGGGGGGTGT GTCTGGGTCA GGTGTGCGCC | 5250 |
| GAGCGTTTGA GCCTGCAGCT TGTCAGCTCC AAGTTACTAC TGACGCTGGA CACCCGGCTC TCACACGCTT | 5320 |
| CTATCTCTCT CTCCCGATAC AAAAGGATTT TATCCGATTC TCATTCCTGT CCCTGTCGTG TGACCCCCGC | 5390 |
| GAGGGCGCGG GCTCTTCTCT CTGTGACTAG ATTTCCCATC TGGAAAGTGC GGGGTTGACC GTGTAGTTTG | 5460 |
| CTCCTCTCGG GGGGCCTGTG GTGGCCATGG CGCAGGCGGC CTGGGAGAGC TGCCGTCACA CAGCCACTGG | 5530 |
| GTGAGCCACA CTCACGGTGG TAGAGCCACA GTGCCTGGTG CCACATCACG TCCTCTGGAT TTTAAGTAAA | 5600 |
| ACCACACACC TCCCGGCAGG CATCTGCCTG CGACCCTGTG TGTGCCTGGG GAGAGTGGTA GCACGGAGGA | 5670 |
| AATTCGTGCA CACTCAACGT CATCAGCAAG GTCATCCGCA GTCAGGTGGA ACGTGGAGGC CTCTCTCTGG | 5740 |
| GATCGTCTCC AGCGGATAAA GGACTGTGCA CAGCTTCGGA AGCTTTTATT TAAAAATATA ACTATTAATT | 5810 |
| ATTGCATTAT AAGTAATCAC TAATGGTATC AGCAATTATA ATATTTATTA AAGTATAATT AGAAATATTA | 5880 |
| AGTAGTACAC ACGTTCTGGA AAAACACAAA TTGCACATGG CAGCAGAGTG AATTTTGGCC GAGGGACACG | 5950 |
| TGTGCACATG TGTGTAAGCC GCCCCCAGGC CCACAGAATT CGCTGACAAA GTCACCTCCC CAGAGAAGCC | 6020 |
| ACCACGGGCC TCCTTCGTGG TCGTGAATTT TATTAACATG GATCAAGTCA CGTACCGTCC ACGTGTGGCA | 6090 |
| GGGCTTTGGG GAATGTGAGG TGATGACTGC GTCCTCATGC CCTGACAGAC AGGAGGTGAC TGTGTCTGTC | 6160 |
| CTGTCCCTAG GACACGGACA GGCCCGAAGC TCTAGTCCCC ATCGTCGTCC AGTTTGGCCT CTGAATAAAA | 6230 |
| ACGTCTTCAA AACCTGTTGC CCCAAAAACT AAGAACAGAG AGAGTTTCCC ATCCCATGTG CTCACAGGGG | 6300 |
| CGTATCTGCT TGCGTTGACT CGCTGGGCTG GCCGGACTCC TAGAGTTGGT GCGTGTGCTT CTGTGCAAAA | 6370 |
| AGTGCAGTCC TCTTGCCCAT CACTGTGATA TCTGCACCAG CAAGGAAAGC CTCTTTTCTT TTCTTTCTTT | 6440 |
| TTTTTTTTTT GAGACGGAAC GTCACTGTTG TCTGCCTGGG CTTGAGTGCA GTGGCGCGAT CTCAACTCAC | 6510 |
| TGCAACCTCC GCCTCCCGGG TTCCAGCATT TCTCCTGCCT CAGCCTCCCG AGCAGCTGAG ATTACAGGCA | 6580 |
| CCCACCCCCT GCGCCTGGCT AATTTTTGTA TTTTTAGTAG AGAGGGGTTT TTGCCATGTT GGCCAGGCTG | 6650 |
| GTCTCCAACT CCTGACCTCA GGTGATCCAC CCACCTCGGC CTCCCAAAGT GCTGGGATTA CAGGTGTGAG | 6720 |
| CCATCACGCC CAGCCGGAAA GCCTCTTTTT AAGGTGACCA CCTATAGCGC TTCCCGAAAA TAACAGGTCT | 6790 |
| TGTTTTTGCA GTAGGCTGCA AGCGTCTCTT AGCAACAGGA GTGGCGTCCT GTGGGCTCTG GGGATGGCTG | 6860 |
| AGGCTCGCGT GGCAGCCATG CCTTCTGTGT GCACCTTTAG GTTCCACGGG GCTATTCTGC TCTCACTGTT | 6930 |
| TGTCTGAAAA CGCACCCTTG GCATCCTTGT TTGGAGAGTT TCTGCTTCTC GTTGGTCATG CTGAAACTAG | 7000 |
| GGGCAAGGTT GTATCCGTTG GCGCGCAGCG GCTACATGTA GGGTCATGAG TCTTTCACCG TGGACAAATT | 7070 |
| CCTTGAAAAA AAAAAAAGGA GTCCGGTTAA GCATTCATTC CGGGTCAAGT GTCTGGTTCT GTGAATAAAC | 7140 |
| TCTAAGATTT AAGAAACCTT AATGAAAGAA AACCTTGATG ATTCAGAGCA AGGATGTGGT CACACCTGTG | 7210 |
| GCTGGATCTG TTTCAGCCGC CCCAGTGCAT GGTGAGAGTG GGGAGCAGGG ATTGTTTGTT CAGAGGTCTC | 7280 |
| ATCTGGTATG TTTCTGAGGT GTTTGCCGGC TGAATGGTAG ACGTGTCGTT TGTGTGTATG AGGTTCTGTG | 7350 |
| TCTGTGTGTG GCTCGGTTTG AGTGTACGCA TGTCCAGCAC ATGCCCTGCC CGTCTCTCAC CTGTGTCTTC | 7420 |
| CCGCCCCAGG TCCTACGTCC AGTGCCAGGG GATCCCGCAG GGCTCCATCC TCTCCACGCT GCTCTGCAGC | 7490 |
| CTGTGCTACG GCGACATGGA GAACAAGCTG TTTGCGGGGA TTCGGCGGGA CGGGTGAGGC CTCCTCTTCC | 7560 |
| CCAGGGGGGC TTGGGTGGGG GTTGATTTGC TTTTGATGCA TTCAGTGTTA ATATTCCTGG TGCTCTGGAG | 7630 |
| ACCATGACTG CTCTGTCTTG AGGAACCAGA CAAGGTTGCA GCCCCTTCTT GGTATGAAGC CGCACGGGAG | 7700 |
| GGGTTGCACA GCCTGAGGAC TGCGGGCTCC ACGCAGGCTC TGTCCAGCGG CCATGTCCAG AGGCCTCAGG | 7770 |
| GCTCAGCAGG CGGGAGGGCC GCTGCCCTGC ATGATGAGCA TGTGAATTCA ACACCGAGGA AGCACACCAG | 7840 |
| CTTCTGTCAC GTCACCCAGG TTCCGTTAGG GTCCTTGGGG AGATGGGGCT GGTGCAGCCT GAGGCCCCAC | 7910 |
| ATCTCCCAGC AGGCCCTCGA CAGGTGGCCT GGACTGGGCG CCTCTTCAGC CCATTGCCCA TCCCACTTGC | 7980 |
| ATGGGGTCTA CACCCAAGGA CGCACACACC TAAATATCGT GCCAACCTAA TGTGGTTCAA CTCAGCTGGC | 8050 |
| TTTTATTGAC AGCAGTTACT TTTTTTTTTT TAATACTTTA AGTTCTAGGG TACATGTGCA CGACGTGCAG | 8120 |
| GTTAGTTACA TATGTATACA TGTGCCATGT TGGTGTGCTG CACCCATTAA CTCATCATTT ACATTAGGTA | 8190 |
| TATCTCCTAA TGCTATCCCT CCCCACTCCC CCCATCCCAT GACAGGCCCT GGTGTGTGAT GTTCCCCACC | 8260 |
| CTGTGTCCAA GTGTTCTCAT TGTTCAGTTC CCACCTGTGA GTGAGAACAT GTGGTGTTTG GTTTTCTTTC | 8330 |
| CTTGCAATAG TTTGCTCAGA GTGATGGTTT CCAGCTTCGT CCATGTCCCT ACAAAGGACA TGAACTCATC | 8400 |
| CTTTTTTATG ACTGCATAGT ATTCCGTGGT GTATATGTGC CACATTTTCT TAATCCAGTC TATCATCGAT | 8470 |
| GGACATTTGG GTTGGTTGCA AGTCTTTGCT ACTGTGAATA GTGCCGCAAT AAACATACGT GTGCATGTGT | 8540 |
| CTTTATAGCA GCATGATTTA TAATCCTTTG GGTATATACC CAGTAATGGG ATGGCTGGGT CAAATGGTAT | 8610 |
| TTCTAGTTCT AGATCCTTGA GGAATCACCA CACTGTCTTC CACAATGGTT GAACTAGTTT ACACTCCCAC | 8680 |
| CAACAGTGTA AAAGTGTTCT GGTGCTGGAG AGGATGTGGA CAGCAGTTAT TTTTTTATGA AAATAGTATC | 8750 |
| ACTGAACAAG CAGACAGTTA GTGAAGGATG CGTCAGGAAG CCTGCAGGCC ACACAGCCAT TTCTCTCGAA | 8820 |
| GACTCCGGGT TTTTCCTGTG CATCTTTTGA AACTCTAGCT CCAATTATAG CATGTACAGT GGATCAAGGT | 8890 |
| TCTTCTTCAT TAAGGTTCAA GTTCTAGATT GAAATAAGTT TATGTAACAG AAACAAAAAT TTCTTGTACA | 8960 |
| CACAACTTGC TCTGGGATTT GGAGGAAAGT GTCCTCGAGC TGGCGGCACA CTGGTCAGCC CTCTGGGACA | 9030 |
| GGATACCTCT GGCCCATGGT CATGGGGCGC TGGGCTTGGG CCTGAGGGTC ACACAGTGCA CCATGCCCAG | 9100 |
| CTTCCTGTGG ATAGGATCTG GGTCTCGGAT CATGCTGAGG ACCACAGCTG CCATGCTGGT AAAGGGCACC | 9170 |
| ACGTCGCTCA GAGGGGGCGA GGTTCCCAGC CCCAGCTTTC TTACCGTCTT CAGTTATTTT TCCCTAAGAG | 9240 |
| TCTGAGAAGT GGGGCCGCGC CTGATGGCCT TCGTTCGTCT TCAGCTGGCA CAGAATTGCA CAAGCTGATG | 9310 |
| GTAAACACTG AGTACTTATA ATGAATGAGG AATTGCTGTA GCAGTTAACT GTAGAGAGCT CGTCTGTTGG | 9380 |
| AAAGAAATTT AAGTTTTTCA TTTAACCGCT TTGGAGAATG TTACTTTATT TATGGCTGTG TAAATTGTTT | 9450 |
| GACATTCAGT CCCTCGTAGA CAGATACTAC GTAAAAAGTG TAAAGTTAAC CTTGCTGTGT ATTTTCCCTT | 9520 |
| ATTTTAGGCT GCTCCTGCGT TTGGTGGATG ATTTCTTGTT GGTGACACCT CACCTCACCC ACGCGAAAAC | 9530 |
| CTTCCTCAGG TGAGGCCCGT GCCGTGTGTC TGTGGGGACC TCCACAGCCT GTGGGCTTTG CAGTTGAGCC | 9650 |
| CCCCGTGTCC TGCCCCTGGC ACCGCAGCGT TGTCTCTGCC AAGTCCTCTC TCTCTGCCGG TGCTGGATCC | 9730 |
| GCAAGAGCAG AGGCGCTTGG CCGTGCACCC AGGCCTGGGG GCGCAGGGGC ACCTTCGGGA GGGAGTGGGT | 9800 |
| ACCGTGCAGG CCCTGGTCCT GCACACACGC ACCCAGGTTA CACACGTGGT GAGTGCAGGC GGTGACCTGG | 9870 |
| CTCCTGCTGC TCTTTGGAAA GTCAAGAGTG GCGGCTCCTG GGGCCCCAGT GAGACCCCCA GGAGCTGTGC | 9940 |
| ACAGGGCCTG CAGGGCCGAG GCGGCAGCCT CCTCCCCAGG GTGCACCTGA GCCTGCGGAG AGCAGGAGCT | 10010 |
| GCTGAGTGAG CTGGCCCACA GCGTTCGCTG CGGTCACGTT CCTGCGTGGG GTTGTTTGGG ATCGGTGGGA | 10080 |
| GAATTTGGAT TTGCTGAGTG CTGCTGTCTT GAACCACGGA GATGGCTAGG AGTGGGTTTC AGAGTTGATT | 10150 |
| TTTGTGAATC AAACTAAAAT CAGGCACAGG GGACCTGGCC TCAGCACAGG GGATTGTCCA ATGTGGTCCC | 10220 |
| CCTCAAGGGC GCCCCACAGA GCCGGTGGGC TTGTTTTAAA GTGCGATTTG ACGAGGGACG AGAAACCTTG | 10290 |
| AAAGCTGTAA AGGGAACCCT CAGAAAATGT GGCCGCCAGG GGTGGTTTCA GGTGCTTTGC TGGGCTGTGT | 10360 |
| TTGTGAAAAC CCATTTGGAC CCGCCCTGCA AGTCCACCCT CCAGGTCCAC CCTCCAGGGC CGCCCTGGGC | 10430 |
| TGGGGGTATG CCTGGCGTTC CTTGTGCCGC AGCCCGGAGC ACAGCAGGCT GTGCACATTT AAATCCACTA | 10500 |
| AGATTCACTC GGGGGGAGCC CAGGTCCCAA GCAATTGAGG GCTCAGGAGT CCTGAGGCTG CTGAGGGGAC | 10570 |
| AGAGCAGACG GGGAACGCTG CTTCTGTGTG GCAACTTCCT GAGGGTGCTG GCCAGGGAGG TGGCTCAGAG | 10640 |
| TGTATGTTGG GGTCCCACCG GGGGCA [symbol] AAC TGTGTCTCTG ATGAGTCGGC AGCCATGTAA CAGGAAGGGG | 10710 |
| TGGCCACAGG GAGCTGGGAA TGCACCAGGG GAGCTGCGCA GCTGGCCGAG GTCCCAGGGC CAGGCCACAG | 10780 |
| GAAGGGCAGG GGGACGCCCG GGGCCACAGC AGAGGCCGCA CGAAGGGAAG GGGATGCCCA CGCCAGAGCA | 10850 |
| GAGGCTACCG GGCACAGGGG GGCTCCCTGA GCTGGGTGAG CGAGGCTCAT GACTCGGCCA GGGAACCTCC | 10920 |
| TTGACGTGAA GCTGACGACT GGTGTTGCCC AGCTCACAGC CCAGCCAGGT CCCGCGCCTG AGCAGGAACT | 10990 |
| CAGAACCCTC CCCTTTGTCT AAAGCACAGC AGATGCCTTC AGGGCATCTA GGAGAAAACA GGCAAAGTCG | 11060 |
| TTGAGAAACG TCTTAAAAGA AGGTGGGATG GTGGCAATTT CTTGTCCAGA TTTTAGTCTG CCCCGGACCA | 11130 |
| CAGATGAGTC TATAACGGGA TTGTGGTGTT GCCATGGGGA CACATGAGAT GGACCATCAC AGAGGCCACT | 11200 |
| GGGGCTGCAC CTCCCATCTG AGTCCTGGCT GTCCCGGGTC CAGGCCAGGT TCTTGCATGC TCACCTACCT | 11270 |
| GTCCTGCCCG GGAGACAGGG AAAGCACCCC GAAGTCTGGA GCAGGGCTGG GTCCAGGCTC CTCAGAGCTC | 11340 |
| CTGCCAGGCC CAGCACCCTG CTCCAAATCA CCACTTCTCT GGGGTTTTCC AAAGCATTTA ACAAGGGTGT | 11410 |
| CAGGTTACCT CCTGGGTGAC GGCCCCGCAT CCTGGGGCTG ACATTGCCCC TCTGCCTTAG GACCCTGGTC | 11480 |
| CGAGGTGTCC CTGAGTATGG CTGCGTGGTG AACTTGCGGA AGACAGTGGT GAACTTCCCT GTAGAAGACG | 11550 |
| AGGCCCTGGG TGGCACGGCT TTTGTTCAGA TGCCGGCCCA CGGCCTATTC CCCTGGTGCG GCCTGCTGCT | 11620 |
| GGATACCCGG ACCCTGGAGG TGCAGAGCGA CTACTCCAGG TGAGCGCACC TGGCCGGAAG TGGAGCCTGT | 11690 |
| GCCCGGCTGG GGCAGGTGCT GCTGCAGGGC CGTTGCGTCC ACCTCTGCTT CCGTGTGGGG CAGGCGACTG | 11760 |
| CCAATCCCAA AGGGTCAGAG GCCACAGGGT GCCCCTCGTC CCATCTGGGG CTGAGCAGAA ATGCATCTTT | 11830 |
| CTGTGGGAGT GAGGGTGCTC ACAACGGGAG CAGTTTTCTG TGCTATTTTG GTAAAAGGAA ATGGTGCACC | 11900 |
| AGACCTGGGT GCACTGAGGT GTCTTCAGAA AGCAGTCTGG ATCCGAACCC AAGACGCCCG GGCCCTGCTG | 11970 |
| GGCGTGAGTC TCTCAAACCC GAACACAGGG GCCCTGCTGG GCATGAGTCC CTCTGAACCC GAGACCCTGG | 12040 |
| GGCCCTGCTG GGCGTGAGTC TCTCCGAACC CAGAGACTTC AGGGCCCTTT TGGGCGTGAG TCTCTCCGCT | 12110 |
| GTGAGCCCCA CACTCCAAGG CTCATCCACA GTCTACAGGA TGCCATGAGT TCATGATCAC GTGTGACCCA | 12180 |
| TCAGGGGACA GGGCCATGGT GTGGGGGGGG TCTCTACAAA ATTCTGGGGT CTTGTTTCCC CAGAGCCCGA | 12250 |
| GAGCTCAAGG CCCCGTCTCA GGCTCAGACA CAAATGAATT GAAGATGGAC ACAGATGCAG AAATCTGTGC | 12320 |
| TGTTTCTTTT ATGAATAAAA AGTATCAACA TTCCAGGCAG GGCAAGGTGG CTCACACCTA TAATCCCAGC | 12390 |
| ACTTTGGGAG GCCGAGGTGG GTGGATCACT TGAGGCCAGG AGTTTGAGGC CAACCTAACC AACATAGTGA | 12460 |
| AATTCCATTT CTACTTAAAA AATACAAAAA TTAGCCTGGC CTGGTGGCAC ACGCCTGTAG TCCCCGCTAT | 12530 |
| GCGGGAGGCT GAGGCAGGAG AATCATTTGA ACCCAGGAGG CAGAGGTTGC AGTGAGCCGA GATCACACCA | 12600 |
| CTGCACTCCA GCCTGGGCAA CAGAGTGAGA CTTCATCTTA AAAAAAAAAA AAAAAGTATC AGCATTCCAA | 12670 |
| AACCATAGTG GACAGGTGTT TTTTTATTCT GTCCTTCGAT AATATTTACT GGTGCTGTGC TAGAGGCCGG | 12740 |
| AACTGGGGGT GCCTTCCTCT GAAAGGCACA CCTTCATGGG AAGAGAAATA AGTGGTGAAT GGTTGTTAAA | 12810 |
| CCAGAGGTTT AAACTGGGGT CCTGTCGTTC TGAGTTAACA GTCCAGATCT GGACTTTGCC TCTTTCCAGA | 12880 |
| ATGCTCCCTG GGGTTTGCTT CATGGGGGAG CAGCAGGTGT GGACACCCTC GTGATGGGGG AGCAGCAGGT | 12950 |
| GCAGACGCCC TCATGATGGG GGAGTGGCAG GTGCAGACAC CCTGGTGCAT GGTGCCCAGC ATGTCCCTGT | 13020 |
| TGCAGCTCCC TCCCCACAAG GATGCCGGTC TCCTGTGCTC CCCACAGTCC CTGCTTCCCT CTCACAGCCT | 13090 |
| TACCTGGTCC TGGCCTCCAC TGGCTTTGTC TGCATGATTT CCACATTTCC TGGGCTCCCA GCACCTCTTC | 13160 |
| GCCTCTCCCA GGCACCTCTG CAGTGCTGGC CATACCAGTC AGCTGTGAAC TGTCCACTGC TTATTTTGCT | 13230 |
| CCCCATGAAA TGTATTTTTT AGGACAGGCA CCCCTGGTTC CAGCCTCTGG CACAGCATCA GTGAATGTTA | 13300 |
| TTGAAGGACA AAGGACAGAC AAACAAATCA GGAAAATGGG TTCTCTCTAA ACACATTGCA AAGCCACAGA | 13370 |
| GGCTAGTGCA GGATGGGTGG GCATCAGGTC ATCAGATGTG GGTCCAATGC CAGAATATTC TGTGCTCCCA | 13440 |
| AAGGCCACTT GGTCAGAGTG TGTGCTTGCA GAGGTGGCTC TAAAAGCTCA GCAGTGGAGG CAGTGGTTCG | 13510 |
| CCATACTCAG GGTGAACTCA CATCCTCTGT GTCTGAAGTA TACAGCAGAG GCTTGAAGGG CATCTGGGAG | 13580 |
| AAGAAAACAG GCAAAATGAT TAAGAAAAGT GAAAAAGGAA AAGTGGTAAG ATGGGAATTT TCTTGTCCAG | 13650 |
| ATTTTAGTCT CCCAAACCAC AGCTCAGATG GTAGAATGTG GTCAGAACTG ATGGACAGAA CAATAGAACA | 13720 |
| AAACGGAAGC CCTATCTCTC AGAAACGTGT GTTAATGTGG TATGTGGCAC AGCTGATGGA AAAGAGAGTG | 13790 |
| TGTGTGTAAT TTTTTTTTCT GAGAAAACTG ACTGGAAGCA AATAAGTTGT GTCTTTACAG CATATACCAG | 13860 |
| AGCAGATTCT AGGTAGAAGA GGAGACACAT GCAAACAACA CCAGCAACAG AAATAAAACA AAAGACTCAA | 13930 |
| AGGGAAGGGA GGTGAACGTT CCCTGGTTTG GTGTTGGGGA AGGACACACA GGGAGGCGGA TGAAACCAGT | 14000 |
| GAGGCAACGG GCATTGCTTT CACTGCAGAG AAACTCAGCT TGCCTGAGCC ACAGTGAAAA TGGCCATTCC | 14070 |
| CTGGAGCGTT TGTGCACGTG ATTTATTTAA GGCGCCCTGT GAGGTCCTGC ACATTCATCC TCTCACTTTG | 14140 |
| TTCTCCTAAC CACCTGAGAG GTAGAGGAGG AAAGGCTCCA GGGGAGCAGC CGCCCTTGGT CACCCAGCTG | 14210 |
| GCAAAGGGCA TGCATGATTG CAGCCTGGCC TCCTGCTCCG GGGCCCTTGC TCTGCCCGAG GACCCCACAC | 14280 |
| AAGTCAGACC CATAGGCTCA GGGTGAGCCG GAGCCCAAGG TCGTGTTGGG GATGGCTGTG AAAGAAGAAA | 14350 |
| TGGACGTCTG ATGCACACTT GGGAAGGTCC TACCAGCAGC GTCAAAGAAA TGCATGTGAA ACTGACAGCG | 14420 |
| AGACCCATCC CTCAAAGAAA CGCACGTGAA ACTGATGGCG AGACCTGTCC CCATCCCTCA TGCTGGCTCC | 14490 |
| TTTTCTGGGC TTGCCAAGAG CCAGCATCAG GTTGAGGCAA GCTGGAAAGA CTTTTCTGGA AAGCAGCTTG | 14560 |
| TTTGCATGGA AGTCCTCACA ATGTCCTGTG TCTTCCCAGT AATTCCACTT CTGAAGTGAC CAGACATTAT | 14630 |
| CACGGGTCTT ATTTACCATT TCCAGTGTTC CAGGCAGGGG GACTTGCCAC AGCAAGTCAC GAACCTGCCC | 14700 |
| AAATACAGGG CTAAGGAGAT ATTATGCATC ACAAAACTTG CTCTGCCATT AAACATTTTT CAAAGAATTT | 14770 |
| TTGAAGAATG TTTAATGGCA CAAAACGTTT ATTTCAATGT AGCAGTGTTC AAAGCTGGAT GTAAAAGAAC | 14840 |
| ACACCCCAGG AGCCTGCCGT GAATGTCATG TGTGTTCATC TTTGGACATG GACATACATG GGCAGTGAGT | 14910 |
| GGTGGTGAGG CCCTGGAGGA CATCGGTGGG ATGCCTCCAT CCTGCCCCTC TGGAGACACC ATGTGTGCCA | 14980 |
| CGTGCACTCA CTGGAGCCCT GTTTAGCTGG TGCCACCTGG CTCTTCCATC CCTGAGATTC AAACACAGTG | 15050 |
| AGATTCCCCA CGCCCAACTC AGTGTTCTCC CACAAAAAAC CTGAGTCACA CCTGTGTTCA CTCGAGGGAC | 15120 |
| GCCCGGGAGC CAGGGCTCCA CAGTTTATTA TGTGTTTTTG GCTGAGTTAT GTGCAGATCT CATCAGGGCA | 15190 |
| GATGATGAGT GCACAAACAC GGCCGTGCGA GGTTTGGATA CACTCAACAT CACTAGCCAG GTCCTGGTGG | 15260 |
| AGTTTGGTCA TGCAGAGTCT GGATGGCATG TAGCATTTGG AGTCCATGGA GTGAGCACCC AGCCCCCTCG | 15330 |
| GGCTGCAGCG CATGCCCCAG GCAGGACAAG GAAGCGGGAG GAAGGCAGGA GGCTCTTTGG AGCAAGCTTT | 15400 |
| GCAGGAGGGG GCTGGGTGTG GGGCAGGCAC CTGTGTCTGA CATTCCCCCC TGTGTCTCAG CTATGCCCGG | 15470 |
| ACCTCCATCA GAGCCAGTCT CACCTTCAAC CGCGGCTTCA AGGCTGGGAG GAACATGCGT CGCAAACTCT | 15540 |
| TTGGGGTCTT GCGGCTGAAG TGTCACAGCC TGTTTCTGGA TTTGCAGGTG AGCAGGCTGA TGGTCAGCAC | 15610 |
| AGAGTTCAGA GTTCAGGAGG TGTGTGCGCA AGTATGTGTG TGTGTGTGTC CGCGCGTGCC TGCAAGGCTG | 15680 |
| ATGGTGACTG GCTGCACGTA AGAGTGCACA TGTACGCATA TACACGTGAG CACATACATG TCTGCATGTG | 15750 |
| TGTACATGAA GGCATGGCAG TGTGTGCACA GGTGTGCAAG GGCACAAGTG TGTGCACATG CGAATGCACA | 15820 |
| CCTGACATGC ATGTGTGTTC GTGCACAGTC GTGTGGGCAT TCACGTGAGG TGCATGCGTG TGGGTGTGCA | 15990 |
| GTGTGAGTAG CATGTGTGCA CATAACATGT ATTGAGGGGT CCTCGTGTTC ACCCCGCTAG GTCCTCAGCA | 15960 |
| CCAGTGCCAC TCCTTACAGG ATGAGACGGG GTCCCAGGCC TTGGTGGGCT GAGGCTCTGA AGCTGCAGCC | 16030 |
| CTGAGGGCAT TGTCCCATCT GGGCATCCGC GTCCACTCCC TCTCCTGTGG GCTTCTGTGT CCACTCCCCC | 16100 |
| TCTCCTGTGG GCATTTACAT CCACTCCACT CCCTCTCTCC TGTGGGCATC CGCGTCCACT CCCCCTCTCT | 16170 |
| GTGGGCATCT GCGTCCACCT CCCCTCTCTG TGGGCATTTG CGTCCACTCC CTCTCCTGGT TCCTTCCTGT | 16240 |
| CTTGGCCGAG CCTCGGGGGC AGGCAGATGA CACAGAGTCT TGACTCGCCC AGGGTGGTTC GCAGCTGCCG | 16310 |
| GGTGAGGGCC AGGCCGGATT TCACTGGGAA GAGGGATAGT TTCTTGTCAA AATGTTCCTC TTTCTTGTTC | 16380 |
| CATCTGAATG GATGATAAAG CAAAAAGTAA AAACTTAAAA TCCCAGAGAG GTTTCTACCG TTTCTCACTC | 16450 |
| TTTCTTGGCG ACTCTAGGTG AACAGCCTCC AGACGGTGTG CACCAACATC TACAAGATCC TCCTGCTCCA | 16520 |
| GGCGTACAGG TGAGCCGCCA CCAAGGGGTG CAGGCCCAGC CTCCAGGGAC CCTCCGCGCT CTGCTCACCT | 16590 |
| CTGACCCGGG GCTTCACCTT GGAACTCCTG GGTTTTAGGG GCAAGGAATG TCTTACGTTT TCAGTGGTGC | 16660 |
| TGCTGCCTGT GCACAGTTCT GTTCGCGTGG CTCTGTGCAA AGCACCTGTT CTCCATCTCT GGGTAGTGGT | 16730 |
| AGGAGCCGGT GTGGCCCCAG GTGTCCCCAC TGTGCCTGTG CACTGGCCGT GGGACGTCAT GGAGGCCATC | 16800 |
| CCAGGGCAGC AGGGGCATGG GGTAAAGAGA TGTTTATGGG GAGTCTTAGC AGAGGAGGCT GGGAAGGTGT | 16870 |
| CTGAACAGTA GATGGGAGAT CAGATGCCCG GAGGATTTGG GGTCTCAGCA AACAGGCCCG AGGTGGGTGC | 16940 |
| AGGTGAGGGT CGCTGGCCCC ACCCCCGGGA AGGTGCAGCA GAGCTGTGGC TCCCCACACA GCCCGGCCAG | 17010 |
| CACCTGTGCT CTGGGCATGG CTGTGCTCCT GGAACGTTCC CTGTCCTGGC TGGTCAGGGG GTGCCCCTGC | 17080 |
| CAAGAATCGA CAACTTTATC ACAGAGGGAA GGGCCAATCT CTGGAGGCCA CAGGGCCAGC TTCTGCCTGG | 17150 |
| AGTCAGGGCA GGTGGTGGCA CAAGCCTCGG GGCTGTACCA AAGGGCAGTC GGGCACCACA GGCCCGGGCC | 17220 |
| TCCACCTCAA CAGGCCTCCC GAGCCACTGG GAGCTGAATG CCAGGAGGCC CAAGCCCTCG CCCCATGAGG | 17290 |
| GCTGAGAAGG AGTGTGAGCA TTTGTGTTAC CCAGGGCCGA GGCTGCGCGA ATTACCGTGC ACACTTGATG | 17360 |
| TGAAATGAGG TCGTCGTCTA TCGTGGAAAC CCAGCAAGGG CTCACGGGAG AGTTTTCCAT TACAAGGTCG | 17430 |
| TACCATGAAA ATGGTTTTTA ACCCGAGTGC TTGCGCCTTC ATGCTCTGGC AGGGAGGGCA GAGCCACAGC | 17500 |
| TGCATGTTAC CGCCTTTGCA CCAGCTCCAG AGGCTTGGGA CCAGGCTGTC TCAGTTCCAG GGTCCGTCCG | 17570 |
| GCTCAGACCG CCCTCCTCTC TGCCTTCTCT CTCTGCCTCA AATCTTCCCT CGTTTGCATC TCCCTGACGC | 17640 |
| GTGCCTGGGC CCTCGTGCAA GCTGCTTGAC TCCTTTCCGG AAACCCTTGG GGTGTGCTGG ATACAGGTGC | 17710 |
| CACTGAGGAC TGGAGGTGTC TGACACTGTG GTTGACCCCA GGGTCCAGCT GGCGTGCTTG GGGCCTCCTT | 17780 |
| GGGCCATGAT GAGGTCAGAG GAGTTTTCCC AGGTGAAAAC TCCTGGGAAA CTCCCAGGGC CATGTGACCT | 17850 |
| GCCACCTGCT CCTCCCATAT TCAGCTCAGT CTTGTCCTCA TTTCCCCACC AGGGTCTCTA GCTCCGAGGA | 17920 |
| GCTCCCGTAG AGGGCCTGGG CTCAGGGCAG GGCGGCTGAG TTTCCCCACC CATGTGGGGA CCCTTGGGTA | 17990 |
| GTCGCTTGAT TGGGTAGCCC TGAGGAGGCC GAGATGCGAT GCGCCACGGG CCGTTTCCAA ACACAGAGTC | 18060 |
| AGGCACGTGG AAGGCCCAGG AATCCCCTTC CCTCGAGGCA GGAGTGGGAG AACGGAGAGC TGGGCCCCGA | 18130 |
| TTTCACGGCA GCCAGGCTGC AGTGGGCGAG GCTGTGGTGG TCCACGTGGC GCTGGGGGCG GGGTCTGATT | 18200 |
| CAAATCCGCT GGGGCTCGGC CTTCCTGGCC CGTGCTGGCC GCGCCTCCAC ACGGGCTTGG GGTGGACGCC | 18270 |
| CCGACCTCTA GCAGGTGGCT ATTTCTCCCT TTGGAAGAGA GCCCCTCACC CATGCTAGGT GTTTCCCTCC | 18340 |
| TGGGTCAGGA GCGTGGCCGT GTGGCAACCC CGGGACCTTA GGCTTATTTA TTTGTTTAAA AACATTCTGG | 18410 |
| GCCTGGCTTC CGTTGTTGCT AAATGGGGAA AAGACATCCC ACCTCAGCAG AGTTACTGAG AGGCTGAAAC | 18480 |
| CGGGGTGCTG GCTTGACTGG TGTGATCTCA GGTCATTCCA GAAGTGGCTC AGGAAGTCAG TGAGACCAGG | 18550 |
| TACATGGGGG GCTCAGGCAG TGGGTGAGAT GAGGTACACG GGGGGCTCAG GCAGTGGGTG AGGCCAGGTA | 18620 |
| CATGGGGGGC TCAGGCACTG GGTGAGATGA GGTACACGGG GGGCTCAGGC AGAGGGTCAG ACCAGGTACA | 18690 |
| CGGGGGCTCT GATCACACGC ACATATGAGC ACATGTGCAC ATGTGCTGTT TCATGGTAGC CAGGTCTGTG | 18760 |
| CACACCTGCC CCAAAGTCCC AGGAAGCTGA GAGGCCAAAG ATGGAGGCTG ACAGGGCTGG CGCGGTGGCT | 18830 |
| CACACCTGTA GTCCCAGCAC TTTGGGAGGC CGAGGCGAGA GGATCCCTTG AGCCCAGGAG TTTAAGACCA | 18900 |
| GCCTGAGCAA CATAGTAGAA CCCCATCTCT ATGAAAAATA AAAACAAAAA TTAGCTGAAC ATGGTGGTGT | 18970 |
| GCGCCTGTAG TTCCAATACT TGGGAGGCTG AAGTGGGAGG ATCACTTGAG CCCAGGAGGT GGAAGCTGCA | 19040 |
| GTGAGCTGAG ATTGCACCAC TGTACTGCAG CCTGGGTGAC AGAGTGAGAG CCCATCTCAA CAACAACAAA | 19110 |
| GAAGACTGAC AAATGCAGTT TCTTGGAAAG AAACATTTAG TAGGAACTTA ACCTACACAC AGAAGCCAAG | 19180 |
| TCGGTGTCTC GGTGTCAGTG AGATGAGATG ATGCGTCCTC ACACCATCAC CCCAGACCCA GGGTTTATGC | 19250 |
| ACCACAGGGG CGGGTGGCTC AGAAGGGATG CGCAGGACGT TGATATACGA TGACATCAAG GTTGTCTGAC | 19320 |
| GAAGGGCAGG ATTCATGATA AGTACCTGCT GGTACACAAG GAACAATGGA TAAACTGGAA ACCTTAGAGG | 19390 |
| CCTTCCCGGA ACAGGGGCTA ATCAGAAGCC AGCATGGGGG GCTGGCATCC AGGATGGAGC TGCTTCAGCC | 19460 |
| TCCACATGCG TGTTCATACA GATGGTGCAC AGAAACGCAC TGTACCTGTG CACACACAGA CACGCAGCTA | 19530 |
| CTCGCACACA CAAGCACACA CACAGACATG CATGCATGCA TCCGTGTGTG TGCACCTGTG CCCATGAGGA | 19600 |
| AACCCATGCA TGTGCATTCA TGCACGCACA CAGGCACCGG TGGGCCCATG CCCACACCCA CGAGCACCGT | 19670 |
| CTGATTAGGA GGCCTTTCCT CTGACGCTGT CCGCCATCCT CTCAGGTTTC ACGCATGTGT GCTGCAGCTC | 19740 |
| CCATTTCATC AGCAAGTTTG GAAGAACCCC ACATTTTTCC TGCGCGTCAT CTCTGACACG GCCTCCCTCT | 19810 |
| GCTACTCCAT CCTGAAAGCC AAGAACGCAG GTATGTGCAG GTGCCTGGCC TCAGTGGCAG CAGTGCCTGC | 19880 |
| CTGCTGGTGT TAGTGTGTCA GGAGACTGAG TGAATCTGGG CTTAGGAAGT TCTTACCCCT TTTCGCATCA | 19950 |
| GGAAGTGGTT TAACCCAACC ACTGTCAGGC TCGTCTGCCC GCCCTCTCGT GGGGTGAGCA GAGCACCTGA | 20020 |
| TGGAAGGGAC AGGACCTGTC TGGGAGCTGC CATCCTTCCC ACCTTGCTCT GCCTGGGGAA GCGCTGGGGG | 20090 |
| GCCTGGTCTC TCCTGTTTGC CCCATGGTGG GATTTGGGCG GCCTGGCCTC TCCTGTTTGC CCTGTGGTGG | 20160 |
| GATTGGGCTG TCTCCCGTCC ATGGCACTTA GGGCCCTTGT GCAAACCCAG GCCAAGGGCT TAGGAGGAGG | 20230 |
| CCAGCCCCAG GCTACCCCAC CCCTCTCAGG AGCAGAGGCC GCGTATCACC ACGACAGAGC CCCGCGCCGT | 20300 |
| CCTCTGCTTC CCAGTCACCG TCCTCTGCCC CTGGACACTT TGTCCAGCAT CAGGGAGGTT TCTGATCCGT | 20370 |
| CTGAAATTCA AGCCATGTCG AACCTGCGGT CCTGAGCTTA ACAGCTTCTA CTTTCTGTTC TTTCTGTGTT | 20440 |
| GTGGAAATTT CACCTGGAGA AGCCGAAGAA AACATTTCTG TCGCGACTCC TGCGGTGCTT GGGTCGGGAC | 20510 |
| AGCCAGAGAT GGAGCCACCC CGCAGACCGT CGGGTGTGGG CAGCTTTCCG GTCTCTCCTG GGAGGGGAGC | 20580 |
| TGGGCTGCGC CTGTGACTCC TCAGCCTCTG TTTTCCCCCA GGGATGTCGC TGGGGGCCAA GGCCGCCCCC | 20650 |
| GGCCCTCTGC CCTCCGAGGC CGTGCAGTGG CTGTGCCACC AAGCATTCCT GCTCAAGCTG ACTCGACACC | 20720 |
| GTGTCACCTA CGTGCCACTC CTGGGGTCAC TCAGGACAGG GAAGTGTGGG TGGACGCCAG TGCGGGCCCC | 20790 |
| ACCTGCCCAG GGGTCATCCT TGAACGCCCT GTGTGGGGCG AGCAGCCTCA GATGCTGCTG AAGTGCAGAC | 20860 |
| GCCCCCGGGC CTGACCCTGG GGGCCTGGAG CCACGCTGGC AGCCCTATGT GATTAAACGC TGGTGTCCCC | 20930 |
| AGGCCACGGA GCCTGGCAGG GTCCCCAACT TCTTGAACCC CTGCTTCCCA TCTCAGGGGC GATGGCTCCC | 21000 |
| CACGCTTGGG AGCCTTCTGA CCCCTGACCT GTGTCCTCTC ACAGCCTCTT CCCTGGCTGC TGCCCTGAGC | 21070 |
| TCCTGGGGTC CTGAGCAAGT TCTCTCCCCG CCCCGCCGCT CCAGCGTCAC TGGGCTGCCT GTCTGCTCGC | 21140 |
| CCCGGTGGAG GGGTGTCTGT CCCTTCACTG AGGTTCCCAC CAGCCAGGGC CACGAGGTGC AGGCCCTGCC | 21210 |
| TGCCCGGCCA CCCACACGTC CTAGGAGGGT TGGAGGATGC CACCTCTGGC CTCTTCTGGA ACGGAGTCTG | 21280 |
| ATTTTGGCCC CGCAGCCCAG ACGCAGCTGA GTCGGAAGCT CCCGGGGACG ACGCTGACTG CCCTGGAGGC | 21350 |
| CGTAGCCAAC CCGGCACTGC CCTCAGACTT CAAGACCATC CTGGACTGAT GGCCACCCGC CCACAGCCAG | 21420 |
| GCCGAGAGCA GACACCAGCA GCCCTGCCAC GCCGGGCTCT ACGTCCCAGG GAGGGAGGGG CGGCCCACAC | 21490 |
| CCAGGCCCGC ACCGCTGGGA GTCTGAGGCC TGAGTGAGTG TTTGGCCGAG GCCTGCATGT CCGGCTGAAG | 21560 |
| GCTGAGTGTC CGGCTGAGGC CTGAG [symbol] GAGT GTCCAGCCAA GGG [symbol] TGAGTG TCCAGCACAC CTGCCGTCTT | 21630 |
| CACTTCCCCA CAGGCTGGCG CTCGGCTCCA CCCCAGGGCC AGCTTTTCCT CACCAGGAGC CCGGCTTCCA | 21700 |
| CTCCCCACAT AGGAATAGTC CATCCCCAGA TTCGCCATTG TTCACCCCTC GCCCTGCCCT CCTTTGCCTT | 21770 |
| CCACCCCCAC CATCCAGGTG GAGACCCTGA GAAGGACCCT GGGAGCTCTG GGAATTTGGA GTGACCAAAG | 21840 |
| GTGTGCCCTG TACACAGGCG AGGACCCTGC ACCTCGATGG CGGTCCCTGT GGGTCAAATT CGGGGGAGGT | 21910 |
| GCTGTGGGAG TAAAATACTG AATATATGAG TTTTTCAGTT TTGAAAAAAA TCTCATGTTT GAATCCTAAT | 21980 |
| GTGCACTGCA TAGACACCAC TGTATGCAAT TACAGAAGCC TGTGAGTGAA CGGGGTGGTG GTCAGTGCGG | 22050 |
| GCCCATGGCC TGGCTGTGCA TTTACGGAAG TCTATGAGTG AATGGGGTTG TGGTCAGTGC GGGCCCATGG | 22120 |
| CCTGGCTGGG CCTGGGAGGT TTCTGATGCT GTGAGGCAGG AGGGGAAGGA GGGTAGGGGA TAGACAGTGG | 22190 |
| GAGCCCCCAC CCTGGAAGAC ATAACAGTAA GTCCAGGCCC GAAGGGCAGC AGGGATGCTG GGGGCCCAGC | 22260 |
| TTGGGCGGCG GGGATGATGG AGGGCCTGGC CAGGGTGGCA GGGATGATGG GGGCCCCAGC TGGGGTGGCA | 22330 |
| GGGGTGATGG GGGGGGCTGG TCTGGGTGGC GGGGAAGATG GGGAAGCCTG GCTGGGCCCC CTCCTCCCCT | 22400 |
| GCCTCCCACC TGCAGCCGTG GATCCGGATG TGCTTCCCTG GTGCACATCC TCTGGGCCAT CAGCTTTCAT | 22470 |
| GGAGGTGGGG GGCAGGGGCA TGACACCATC CTGTATAAAA TCCAGGATTC CTCCTCCTGA ACGCCCCAAC | 22540 |
| TCAGGTTGAA AGTCACATTC CGCCTCTGGC CATTCTCTTA AGAGTAGACC AGGATTCTGA TCTCTGAAGG | 22610 |
| GTGGGTAGGG TGGGGCAGTG GAGGGTGTGG ACACAGGAGG CTTCAGGGTG GGGCTGGTGA TGCTCTCTCA | 22680 |
| TCCTCTTATC ATCTCCCAGT CTCATCTCTC ATCCTCTTAT CATCTCCCAG TCTCATCTGT CTTCCTCTTA | 22750 |
| TCTCCCAGTC TCATCTGTCA TCCTCTTACC ATCTCCCAGT CTCATCTCTT ATCCTCTTAT CTCCTAGTCT | 22820 |
| CATCCAGACT TACCTCCCAG GGCGGGTGCC AGGCTCGCAG TGGAGCTGGA CATACGTCCT TCCTCAGGCA | 22890 |
| GAAGGAACTG GAAGGATTGC AGAGAACAGG AGGGGCGGCT CAGAGGGACG CAGTCTTGGG GTGAAGAAAC | 22960 |
| AGCCCCTCCT CAGAAGTTGG CTTGGGCCAC ACGAAACCGA GGGCCCTGCG TGAGTGGCTC CAGAGCCTTC | 23030 |
| CAGCAGGTCC CTGGTGGGGC CTTATGGTAT GGCCGGGTCC TACTGAGTGC ACCTTGGACA GGGCTTCTGG | 23100 |
| TTTGAGTGCA GCCCGGACGT GCCTGGTGTC GGGGTGGGGG CTTATGGCCA CTGGATATGG CGTCATTTAT | 23170 |
| TGCTGCTGCT TCAGAGAATG TCTGAGTGAC CGAGCCTAAT GTGTATGGTG GGCCCAAGTC CACAGACTGT | 23240 |
| GTCGTAAATG CACTCTGGTG CCTGGAGCCC CCGTATAGGA GCTGTGAGGA AGGAGGGGCT CTTGGCAGCC | 23310 |
| GGCCTGGGGG CGCCTTTGCC CTGCAAACTG GAAGGGAGCG GCCCCGGGCG CCGTGGGCGG ACGACCTCAA | 23380 |
| GTGAGAGGTT GGACAGAACA GGGCGGGGAC TTCCCAGGAG CAGAGGCCGC TGCTCAGGCA CACCTGGGTT | 23450 |
| TGAATCACAG ACCAACAGGT CAGGCCATTG TTCAGCTATC CATCTTCTAC AAAGCTCCAG ATTCCTGTTT | 23520 |
| CTCCGGGTGT TTTTTGTTGA AATTTTACTC AGGATTACTT ATATTTTTTG CTAAAGTATT AGACCCTTAA | 23590 |
| AAAAGGTATT TGCTTTGATA TGGCTTAACT CACTAAGCAC CTACTTTATT TGTCTGTTTT TATTTATTAT | 23660 |
| TATTATTATT ATTAGAGATG GTGTCTACTC TGTCACCCAG GTTGTTAGTG CAGTGGCACA GTCATGGCTC | 23730 |
| GCTGTAGCCG CAAACCCCCA GGCTCAAGTG ATCCTCCGGC CTCAGCTTCC CAGAGTGCTG GGATTACAGG | 23800 |
| TGTGAGCCAC TGCCCTTGCC TGGCACTTTT AAAAACCACT ATGTAAGGTC AGGTCCAGTG GCTTCCACAC | 23870 |
| CTGTCATCCC AGTAGTTTGG GAAGCCGAGG CAGAAGGATT GTCTGAGGCC AGGAGTTTGA GACCAGCATG | 23940 |
| GGTAACATAG GGAGACCCCA TCTCTACAAA AAATGCAAAA AGTTATCCGG GCGTGGGGTC CAGCATCTGT | 24010 |
| AGTCCCAGCT GCTCGGGAGG CTGAGTGGGA GGATCGCTTG AGCCCGGGAG GTCATGGCTG CAGTGAGCTG | 24080 |
| TGATTGTACC ATCGCACTCC AGCCTGGGCA ACAGAGTGAG ACCCTGTCTC AAAAAAAAAA AAAAAAAAAG | 24150 |
| AAGGAGAAGG AGAAGAGAAG AAGAAGGAAG AAGGAAAGAG AAGAAGAAGG AAGAAGGAAG AAAGAAGGAG | 24220 |
| AAGGAGGCCT GCTAGGTGCT AGGTAGACTG TCAAATCTCA GAGCAAAATG AAAATAACAA AGTTTTAAAG | 24290 |
| GGAAAGAAAA ACCCCAGCTC TTTGGACTTC CTTAGGCCTG AACTTCATCT CAAGCAGCTT CCTTCCACAG | 24360 |
| ACAAGCGTGT ATGGAGCGAG TGAGTTCAAA GCAGAAAGGG AGGAGAAGCA GGCAAGGGTG GAGGCTGTGG | 24430 |
| GTGACACCAG CCAGGACCCC TGAAAGGGAG TGGTTGTTTT CCTGCCTCAG CCCCACGCTC CTGCCGGTCC | 24500 |
| TGCACCTGCT GTAACCGTCG ATGTTGGTGC CAGGTGCCCA CCTGGGAAGG ATGCTGTGCA GGGGGCTTGC | 24570 |
| CAAACTTTGG TGGGTTTCAG AAGCCCCAGG CACTTGTGGC AGGCACAATT ACAGCCCCTC CCCAAAGATG | 24640 |
| CCCACGTCCT TCTCCTGGAA CCTGTGAATG TGTCACCCGC AAGGCAGAGG CTGGTGAAGG CTGCAGGTGG | 24710 |
| AATCACGGCT GCCAGTCAGC CGATCTTAAG GTCATCCTGG ATTATCTGGT GGGCCTGATA TGGCCACAAG | 24780 |
| GGTCCCTAGA AGTGAGAGAG GCAGGCAGGG GAGAGTCAGA GAGGGGACGT GAGAAGGACC ACTGGCCACT | 24850 |
| GCTGGCTTTG AGATGGAGGA GGAGGTCCCC AGCCAAGGAA TGGGGGCAGC CGCTCCATGC TGGAAAAGCA | 24920 |
| AGCAATCCTC CCCGGTCCTG AGGGCACACG GCCCTGCCCA CGCCTCGATT TCAGGCCAGT GGGACCTGTT | 24990 |
| TCAGCTTTCC GGCCTCCAGA GCTGTAAGAT GATGCGTTTG TGTTCAGCCA CTAAGCTGCA GTGATTCGTC | 25060 |
| ACAGCAGCAA ATGGAATAGC AGTACAGGGA AATGAATACA GGGACAGTTC TCAGAGTGAC TCTCAGCCCA | 25130 |
| CCCCTGGG | 25138 |

### Example 5

Comparison of the above-described genomic hTC sequence and the sequence of the hTC cDNA (FIG. 6; corresponding to SEQ ID NO 2) made it possible to elucidate the exon-intron structure of the hITC gene. The genomic organization of the hTC gene is illustrated diagrammatically in FIG. 7. The coding region of the hTC gene is composed of 16 exons which var′ in size between 6′ bp and 1354 bp (see Table 1). Exon 1 contains the translation start codon ATG. The translation stop codon TGA and the 3-untranslated region lie on exon 16 (FIG. 8). No possible polyadenylation signal (AATAAA) was found either in exon 16 or in the 3195 bp of the following 3′-flanking region. The exon-intron transitions were determined on the basis of the consensus sequence

| | 5′-Exon Intron 3′-Exon |
|---|---|
| Pre-mRNA | A/C A G \| G T A/G A ... N C A G \| G |
| Frequency (%) | 70 60 80 100 100 95 70 80 100 100 60 |

and listed in Table 1. With the exception of the 5′ splice site between exon 15 and intron 15, all the exon-intron transitions are in accord with the published (Shapiro and Senapathy, 1987) splice consensus sequence. The sizes of the introns are between 104 bp and 8616 bp. Since only part of intron 6 was isolated, it is not possible to determine the precise length of the hTC gene. Based on the part sequence of ˜4660 bp, which was obtained from intron 6, the minimum size of the hTERT gene is 37 kb.

Introns 1-5 and the 5′ region of intron 6, are contained in contig 1:

Intron 1: bp 11493-11596 (SEQ ID NO 4);

Intron 2: bp 12951-21566 (SEQ ID NO 5);

Intron 3: bp 21763-23851 (SEQ ID NO 6);

Intron 4: bp 24033-24719 (SEQ ID NO 7);

Intron 5: bp 24900-25393 (SEQ ID NO 8);

5′ region of intron 6: bp 25550-26414 (SEQ ID NO 9).

The 3′ region of intron 6, and introns 7-15, are located in contig 2 at the following positions:

3′ region of intron 6: bp 1-3782 (SEQ ID NO 10);

Intron 7: bp 3879-4858 (SEQ ID NO 11),

Intron 8: bp 4945-7429 (SEQ ID NO 12);

Intron 9: bp 7544-9527 (SEQ ID NO 13);

Intron 10: bp 9600-11470 (SEQ ID NO 14);

Intron 11: bp 11660- 15460 (SEQ ID NO 15;

Intron 12: bp 15588-16467 (SEQ ID NO 16);

Intron 13: bp 16530-19715 (SEQ ID NO 17);

Intron 14: 19841-20621 (SEQ ID NO 18);

Intron 15: 20760-21295 (SEQ ID NO 19).

The 3′-untranscribed region is also located in contig 2 at position 21960-25138 (SEQ ID NO 20).

The individual sequences of the abovementioned introns are as follows:

| | |
|---|---|
| Intron 1 | |
| GTGGGCCTCCCCGGGGTCGGCGTCCGGCTGGGGTTGAGGGCGGCCGGGGGGAACCAGCGACATGCGGAGAGCAGCGCAGG | (SEQ ID NO 4) |
| CGACTCAGGGCGCTTCCCCCGCAG | |
| Intron 2 | |
| GTGAGGAGGTGGTGGCCGTCGAGGGCCCAGGCCCCAGAGCTGAATGCAGTAGGGGCTCAGAAAAGGGGGCAGGCAGAGCC | (SEQ ID NO 5) |
| CTGGTCCTCCTGTCTCCATCGTCACGTGGGCACACGTGGCTTTTCGCTCAGGACGTCGAGTGGACACGGTGATCTCTGCC | |
| TCTGCTCTCCCTCCTGTCCAGTTTGCATAAACTTACGAGGTTCACCTTCACGTTTTGATGGACACGCGGTTTCCAGGCGC | |
| CGAGGCCAGAGCAGTGAACAGAGGAGGCTGGGCGCGGCAGTGGAGCCGGGTTGCCGGCAATGGGGAGAAGTGTCTGGAAG | |
| CACAGACGCTCTGGCGAGGGTGCCTGCAGGTTACCTATAATCCTCTTCGCAATTTCAAGGGTGGGAATGAGAGGTGGGGA | |
| CGAGAACCCCCTCTTCCTGGGGGTGGGAGGTAAGGGTTTTGCAGGTGCACGTGGTCAGCCAATATGCAGGTTTGTGTTTA | |
| AGATTTAATTGTGTGTTGACGGCCAGGTGCGGTGGCTCACGCCGGTAATCCCAGCACTTTGGGAAGCTGAGGCAGGTGGA | |
| TCACCTGAGGTCAGGAGTTTGAGACCAGCCTGACCAACATGGTGAAACCCTATCTGTACTAAAAATACAAAAATTAGCTG | |
| GGCATGGTGGTGTGTGCCTGTAATCCCAGCTACTTGGGAGGCTGAGGCAGGAGAATCACTTGAACCCAGGAGGCGGAGGC | |
| TGCAGTGAGCTGAGATTGTGCCATTGTACTCCAGCCTGGGCGACAAGAGTGAAACTCTGTCTTTAAAAAAAAAAAGTGTT | |
| CGTTGATTGTGCCAGGACAGGGTAGAGGGAGGGAGATAAGACTGTTCTCCAGCACAGATCCTGGTCCCATCTTTAGGTAT | |
| GAAGAGGGCCACATGGGAGCAGAGGACAGCAGATGGCTCCACCTGCTGAGGAAGGGACAGTGTTTGTGGGTGTTCAGGGG | |
| ATGGTGCTGCTGGGCCCTGCCGTGTCCCCACCCTGTTTTTCTGGATTTGATGTTGAGGAACCTCCGCTCCAGCCCCCTTT | |
| TGGCTCCCAGTGCTCCCAGGCCCTACCGTGGCAGCTAGAAGAAGTCCCGATTTCACCCCCTCCCCACAAACTCCCAAGAC | |
| ATGTAAGACTTCCGGCCATGCAGACAAGGAGGGTGACCTTCTTGGGGCTCTTTTTTTTCTTTTTTTCTTTTTATGGTGGC | |
| AAAAGTCATATAACATGAGATTGGCACTCCTAACACCGTTTTCTGTGTACAGTGCAGAATTGCTAACTCGGCGGTGTTTA | |
| CAGCAGGTTGCTTGAAATGCTGCGTCTTGCGTGACTGGAAGTCCCTACCCATCGAACGGCAGCTGCCTCACACCTGCTGC | |
| GGCTCAGGTGGACCACGCCGAGTCAGATAAGCGTCATGCAACCCAGTTTTGCTTTTTGTGCTCCAGCTTCCTTCGTTGAG | |
| GAGAGTTTGAGTTCTCTGATCAGGACTCTGCCTGTCATTGCTGTTCTCTGACTTCAGATGAGGTCACAATCTGCCCCTGG | |
| CTTATGCAGGGAGTGAGGCGTGGTCCCCGGGTGTCCCTGTCACGTGCAGGGTGAGTGAGGCGTTGCCCCCAGGTGTCCCT | |
| GTCACGTGTAGGGTGAGTGAGGCGCGGCCCCCGGGTGTCCCTGTCCCGTGCAGCGTGATTGAGGTGTGGCCCCCGGGTGT | |
| CCCTGTCACGTGTAGGGTGAGTGAGGCGCCATCCCCGGGTGTCCCTGTCACGTGTAGGGTGAGTGAGGCGTGGTCCCCGG | |
| GTGTCCCTGTCCCGTGCAGGGTGAGTGAGGCACTGTCCCCGGGTGTCCCTGTCACGTGCAGGGTGAGTGAGGCGCGGTCC | |
| CCGGGTGTCCCTCTCAGGTGTAGGGTGAGTGAGGCGCGGCCCCAGGGTGTCCCTGTCACGTGTAGGGTGAGTGAGGCACC | |
| GTCCCTGGGTGTCCCTCCCAGGTATAGGGTGAGTGAGGCACTGTCCCCGGGTGTCCCTGTCACGTGCAGGGTGAGTGAGG | |
| CGCGGCCCCCGGGTGTCCCTCTCAGGTGCAGGGTGAGTGAGGCGCTGTCCCTGGGTGTCCCTGTCTCGTGTAGGGTGAGT | |
| GAGGCTCTGTCCCCAGGTGTCCTTGGCGTTTGCTCACTTGAGCTTGCTCCTGAATGTTTGCTCTTTCTATAGCCACAGCT | |
| GCGCCGGTTGCCCATTGCCTGGGTAGATGGTGCAGGCGCAGTGCTGGTCCCCAAGCCTATCTTTTCTGATGCTCGGCTCT | |
| TCTTGGTCACCTCTCCGTTCCATTTTGCTACGGGGACACGGGACTGCAGGCTCTCGCCTCCCGCGTGCGAGGCACTGCAG | |
| CCACAGCTTCAGGTCCGCTTGCCTCTGTTGGGCCTGGCTTGCTCACCACGTGCCCGCCACATGCATGCTGCCAATACTCC | |
| TCTCCCAGCTTGTCTCATGCCGAGGCTGGACTCTGGGCTGCCTGTGTCTGCTGCCACGTGTTGCTGGAGACATCCCAGAA | |
| AGGGTTCTCTGTGCCCTGAAGGAAAGCAAGTCACCCCAGCCCCCTCACTTGTCCTGTTTTCTCCCAAGCTGCCCCTCTGC | |
| TTGGCCCCCTTGGGTGGGTGGCAACGCTTGTCACCTTATTCTGGGCACCTGCCGCTCATTGCTTAGGCTGGGCTCTGCCT | |
| CCAGTCGCCCCCTCACATGGATTGACGTCCAGCCACAGGTTGGAGTGTCTCTGTCTGTCTCCTGCTCTGAGACCCACGTG | |
| GAGGGCCGGTGTCTCCGCCAGCCTTCGTCAGACTTCCCTCTTGGGTCTTAGTTTTGAATTTCACTGATTTACCTCTGACG | |
| TTTCTATCTCTCCATTGTATGCTTTTTCTTGGTTTATTCTTTCATTCCTTTTCTAGCTTCTTAGTTTAGTCATGCCTTTC | |
| CCTCTAAGTGCTGCCTTACCTGCACCCTGTGTTTTGATGTGAAGTAATCTCAACATCAGCCACTTTCAAGTGTTCTTAAA | |
| ATACTTCAAAGTGTTAATACTTCTTTTAAGTATTCTTATTCTGTGATTTTTTTCTTCGTGCACGCTGTGTTTTGACGTGA | |
| AATCATTTTGATATCAGTGACTTTTAAGTATTCTTTAGCTTATTCTGTGATTTCTTTGAGCAGTGAGTTATTTGAACACT | |
| GTTTATGTTCAAGATATGTAGAGTATCAAGATACGTAGAGTATTCTAAGTTATCATTCTATTATTGATTTCTAACTCAGT | |
| TGTGTAGTGGTCTGTATAATACCAATTATTTGAAGTTTGCGGAGCCTTGCTTTGTGATCTAGTGTGTGCATGGTTTCCAG | |
| AACTGTCCATTGTAAATTTGACATCCTGTCAATAGTGGGCATGCATGTTCACTATATCCAGCTTATTAAGGTCCAGTGCA | |
| AAGCTTCTGTCTCCTTCTAGATGCATGAAATTCCAAGAAGGAGGCCATAGTCCCTCACCTGGGGGATGGGTCTGTTCATT | |
| TCTTCTCGTTTGGTAGCATTTATGTGAGGCATTGTTAGGTGCATGCACGTGGTAGAATTTTTATCTTCCTGATGAGTGAA | |
| TCTTTTGGAGACTTCTATGTCTCTAGTAATCTAGTAATTCTTTTTTTAAATTGCTCTTAGTACTGCCACACTGGGCTTCT | |
| TTTGATTAGTATTTTCCTGCTGTGTCTGTTTTCTGCCTTTAATTTATATATATATATATATTTTTTTTTTTTTTGAGACA | |
| GAGTCTTGGTCTGTCGCCCAGGGTGAGTGCAGTGGTGTGATCACAGGTCAGTGTAACTTTTACCTTCTGGCCTGAGCCGT | |
| CCTCTCACCTCAGCCTCCTGAGTAGCTGGAACTGCAGACACGCACCGCTACACCTGGCTAATTTTTAAATTTTTTCTGGA | |
| GACAGGGTCTTGCTGTGTTGCCCAGGCTGGTCTCAAACTCTTGGACTCAAGGGATCCATCTACCTCGGCTTCCCAAAGTG | |
| CTGAATTACAGGCATGAGCCACCATGTCTGGCCTAATTTTCAACACTTTTATATTCTTATAGTGTGGGTATGTCCTGTTA | |
| ACAGCATGTAGGTGAATTTCCAATCCAGTCTGACAGTCGTTGTTTAACTGGATAACCTGATTTATTTTCATTTTTTTGTC | |
| ACTAGAGACCCGCCTGGTGCACTCTGATTCTCCACTTGCCTGTTGCATGTCCTCGTTCCCTTGTTTCTCACCACCTCTTG | |
| GGTTGCCATGTGCGTTTCCTGCCGAGTGTGTGTTGATCCTCTCGTTGCCTCCTGGTCACTGGGCATTTGCTTTTATTTCT | |
| CTTTGCTTAGTGTTACCCCCTGATCTTTTTATTGTCGTTGTTTGCTTTTGTTTATTGAGACAGTCTCACTCTGTCACCCA | |
| GGCTGGAGTGTAATGGCACAATCTCGGCTCACTGCAACCTCTGCCTCCTCGGTTCAAGCAGTTCTCATTCCTCAACCTCA | |
| TGAGTAGCTGGGATTACAGGCGCCCACCACCACGCCTGGCTAATTTTTGTATTTTTAGTAGAGATAGGCTTTCACCATGT | |
| TGGCCAGGCTGGTCTCAAACTCCTGACCTCAAGTGATCTGCCCGCCTTGGCCTCCCACAGTGCTGGGATTACAGGTGCAA | |
| GCCACCGTGCCCGGCATACCTTGATCTTTTAAAATGAAGTCTGAAACATTGCTACCCTTGTCCTGAGCAATAAGACCCTT | |
| AGTGTATTTTAGCTCTGGCCACCCCCCAGCCTGTGTGCTGTTTTCCCTGCTGACTTAGTTCTATCTCAGGCATCTTGACA | |
| CCCCCACAAGCTAAGCATTATTAATATTGTTTTCCGTGTTGAGTGTTTCTGTAGCTTTGCCCCCGCCCTGCTTTTCCTCC | |
| TTTGTTCCCCGTCTGTCTTCTGTCTCAGGCCCGCCGTCTGGGGTCCCCTTCCTTGTCCTTTGCGTGGTTCTTCTGTCTTG | |
| TTATTGCTGGTAAACCCCAGCTTTACCTGTGCTGGCCTCCATGGCATCTAGCGACGTCCGGGGACCTCTGCTTATGATGC | |
| ACAGATGAAGATGTGGAGACTCACGAGGAGGGCGGTCATCTTGGCCCGTGAGTGTCTGGAGCACCACGTGGCCAGCGTTC | |
| CTTAGCCAGTGAGTGACAGCAACGTCCGCTCGGCCTGGGTTCAGCCTGGAAAACCCCAGGCATGTCGGGGTCTGGTGGCT | |
| CCGCGGTGTCGAGTTTGAAATCGCGCAAACCTGCGGTGTGGCGCCAGCTCTGACGGTGCTGCCTGGCGGGGGAGTGTCTG | |
| CTTCCTCCCTTCTGCTTGGGAACCAGGACAAAGGATGAGGCTCCGAGCCGTTGTCGCCCAACAGGAGCATGACGTGAGCC | |
| ATGTGGATAATTTTAAAATTTCTAGGCTGGGCGCGGTGGCTCACGCCTGTAATCCCAGCACTTTGGGAGGCCAAGGCGGG | |
| TGGATCACGAGGTCAGGAGGTCGAGACCATCCTGGCCAACATGATGAAACCCCATCTGTACTAAAAACACAAAAATTAGC | |
| TGGGCGTGGTGGCGGGTGCCTGTAATCCCAGCTACTCGGGAGGCTGAGGCAGGAGAATTGCTTGAACCTGGGAGTTGGAA | |
| GTTGCAGTGAGCCGACATTGCACCACTGCACTCCAGCCTGGCAACACAGCGAGACTCTGTCTCAAAAAAAAAAAAAAAAA | |
| AAAAAAAAAAAATTCTAGTAGCCACATTAAAAAAGTAAAAAAGAAAAGGTGAAATTAATGTAATAATAGATTTTACTGAA | |
| GCCCAGCATGTCCACACCTCATCATTTTAGGGTGTTATTGGTGGGAGCATCACTCACAGGACATTTGACATTTTTTGAGC | |
| TTTGTCTGCGGGATCCCGTGTGTAGGTCCCGTGCGTGGCCATCTCGGCCTGGACCTGCTGGGCTTCCCATGGCCATGGCT | |
| GTTGTACCAGATGGTGCAGGTCCGGGATGAGGTCGCCAGGCCCTCAGTGAGCTGGATGTGCAGTGTCCGGATGGTGCACG | |
| TCTGGGATGAGGTCGCCAGGCCCTGCTGTGAGCTGGATGTGTGGTGTCTGGATGGTGCAGGTCAGGGGTGAGGTCTCCAG | |
| GCCCTCGGTGAGCTGGAGGTATGGAGTCCGGATGATGCAGGTCCGGGGTGAGGTCGCCAGGCCCTGCTGTGAGCTGGATG | |
| TGTGGTGTCTGGATGGTGCAGGTCAGGGGTGAGGTCTCCAGGCCCTCGGTAAGCTGGAGGTATGGAGTCCGGATGATGCA | |
| GGTCCGGGGTGAGGTCGCCAGGCCCTGCTGTGAGCTGGATGTGTGGTGTCTGGATGGTGCAGGTCTGGGGTGAGGTCACC | |
| GGTCCGGGGTGAGGTCGCCAGGCCCTGCTGTGAGCTGGATGTGTGGTGTCTGGATGGTGCAGGTCTGGGGTGAGGTCACC | |
| AGGCCCTGCGGTGAGCTGGGTGTGCGGTGTCTGGATGGTGCAGGTCTGGAGTGAGGTCGCCAGACGGTGCCAGACCATGC | |
| GGTGAGCTGGATATGCGGTGTCCGGATGGTGCAGGTCTGGGGTGAGGTTGCCAGGCCCTGCTGTGAGTTGGATGTGGGGT | |
| GTCCGGATGCTGCAGGTCCGGTGTGAGGTCACCAGGCCCTGCTGTGAGCTGGATGTGTGGTGTCTGGATGGTGCAGGTCT | |
| GGGGTGAAGGTCGCCAGGCCCCTGCTTGTGAGCTGGATGTGTGGTGTCTGGATGGTGCAGGTCTGGAGTGAGGTCGCCAG | |
| GCCCTCGGTGAGCTGGATGTGCAGTGTCCAGATGGTGCAGGTCCGGGGTGAGGTCGCCAGACCCTGCGGTGAGCTGGATG | |
| TGCGGTGTCTGGATGGTGCAGGTCTGGAGTGAGGTCGCCAGGCCCTCGGTGAGCTGGATGTATGGAGTCCGGATGGTGCC | |
| GGTCCGGGGTGAGGTCGCCAGACCCTGCTGTGAGCTGGATGTGCGGTGTCTGGATGGTACAGGTCTGGAGTGAGGTCGCC | |
| AGACCCTGCTGTGAGCTGGATATGCGGTGTCCGGATGGTGCAGGTCAGGGGTGAGGTCTCCAGGCCCTCGGTGAGCTGGA | |
| GGTATGGAGTCCGGATGATGCAGGTCCGGGGTGAGGTCGCCAGGCCCTGCTGTGAACTGGATGTGCGGCGTCTGGATGGT | |
| GCAGGTCTGGGGTGTGGTCGCCAGGCCCTCGGTGAGCTGGAGGTATGGAGTCCGGATGATGCAGGTCCGGGGTGAGGTCG | |
| CCAGGCCCTGCTGTGAGCTGGATGTGCGGCGTCTGGATGGTGCAGGTCTGGGGTGTGGTCGCCAGGCCCTCGGTGAGCTG | |
| GAGGTATGGAGTCCGGATGATGCAGGTCCGGGGTGAGGTTGCCAGGCCCTGCTGTGAGCTGGATGTGCTGTATCCGGATG | |
| GTGCAGTCCGGGGTGAGGTCGCCAGGCCCTGCTGTGAGCTGGATGTGCTGTATCCGGATGGTGCAGGTCTGGGGTGAGGT | |
| CACCAGGCCCTGCGGTGAGCTGGTTGTGCGGTGTCCGGTTGCTGCAGGTCCGGGGTGAGTTCGCCAGGCCCTCGGTGAGC | |
| TGGATGTGCGGTGTCCCCGTGTCCGGATGGTGCAGGTCCAGGGTGAGGTCGCTAGGCCCTTGGTGGGCTGGATGTGCCGT | |
| GTCCGGATGGTGCAGGTCTGGGGTGAGGTCGCCAGGCCTTTGGTGAGCTGGATGTGCGGTGTCTGCATGGTGCAGGTCTG | |
| GGGTGAGGTCGCCAGGCCCTTGGTGGGCTGGATGTGTGGTGTCCGGATGGTGCAGGTCCGGCGTGAGGTCGCCAGGCCCT | |
| GCTGTGAGCTGGATGTGCGGTGTCTGGATGGTGCAGGTCCGGGGTGAGGTAGCCAAGGCCTTCGGTGAGCTGGATGTGGG | |
| GTGTCCGGATGGTGCAGGTCCGGGGTGAGGTCGCCAGGCCCTGCGGTTAGCTGGATATGCGGTGTCCGGATGGTGCAGGT | |
| CCGGGGTGAGGTCACCAGGCCCTGCGGTTAGCTGGATGTGCGGTGTCTGGATGGTGCAGGTCCGGGGTGAGGTCGCCAGG | |
| CCCTGCTGTGAGCTGGATGTGCTGTATCCGGATGGTGCAGGTCCGGGGTGAGGTCGCCAGGCCCTGCAGTGAGCTGGATG | |
| TGCTGTATCCGGATGGTGCAGGTCTGGCGTGAGGTCGCCAGGCCCTGCGGTTAGCTGGATATGCGGTGTCGGATGGTGCA | |
| GGTCCGGGGTGAGGTCACCAGGCCCTGCGGTTAGCTGGATGTGCGGTGTCCGGATGGTGCAGGTCTGGGGTGAGGTCGCC | |
| AGGCCCTGCTGTGAGCTGGATGTGCTGTATCCGGATGGTGCAGGTCCGGGGTGAGGTCGCCAGGCCCTGCGGTGAGCTGG | |
| ATGTGCTGTATCCGGATGGTGCAGGTCTGGCGTGAGGTCGCCAGGCCCTGCGGTGAGCTGGATGTGCAGTGTACGGATGG | |
| TGCAGGTCCGGGGTGAGGTCGCCAGGCCCTGCGGTGGGCTGTATGTGTGTTGTCTGGATGGTGCAGGTCCGGGGTGAGTT | |
| CGCCAGGCCCTGCGGTGAGCTGGATGTGTGGTGTCTGGATGCTGCAGGTCCGGGGTGAGTTCGCCAGGCCCTCGGTGAGC | |
| TGGATATGCGGTGTCCCCGTGTCCGAATGGTGCAGGTCCAGGGTGAGGTCGCCAGGCCCTTGGTGGGCTGGATGTGCCGT | |
| GTCCGGATGGTGCAGGTCTGGGGTGAGGTCGCCAGGCCCTTGGTGAGCTGGATGTGCGGTGTCCGGATGGTGCAGGTCCG | |
| GGGTGAGGTCACCAGGCCCTCGGTGATCTGGATGTGGCATGTCCTTCTCGTTTAAG | |
| Intron 3 | |
| GTACTGTATCCCCACCCCAGGCCTCTGCTTCTCGAAGTCCTGGAACACCAGCCCGGCCTCAGCATGCGCCTGTCTCCACT | (SEQ ID NO 6) |
| TGCCTGTGCTTCCCTGGCTGTGCAGCTCTGGGCTGGGAGCCAGGGGCCCCGTCACAGGCGTGGTCCAAGTGGATTCTGTG | |
| CAAGGCTCTGACTGCCTGGAGCTCACGTTCTCTTACTTGTAAAATCAGGAGTTTGTGCCAAGTGGTCTCTAGGGTTTGTA | |
| AAGCAGAAGGGATTTAAATTAGATGGAAACACTACCACTAGCCTCCTTGCCTTTCCCTGGGATGTGGGTCTGATTCTCTC | |
| TCTCTTTTTTTTTTCTTTTTTGAGATGGAGTCTCACTCTGTTGCCCAGGCTGGAGTGCAGTGGCATAATCTTGGCTCACT | |
| GCAACCTCCACCTCCTGGGTTTAAGCGATTCACCAGCCTCAGCCTCCTAAGTAGCTGGGATTACAGGCACCTGCCACCAC | |
| GCCTGGCTAATTTTTGTACTTTTAGGAGAGACGGGGTTTCACCATGTTGGCCAGGCTGGTCTCGAACTCATGACCTCAGG | |
| TGATCCACCCACCTTCGCCTCCCAAAGTGCTGGGTTTACAGGCTAAGCCACCGTGCCCAGCCCCCGATTCTCTTTTAATT | |
| CATGCTGTTCTGTATGAATCTTCAATCTATTGGATTTAGGTCATGAGAGGATAAAATCCCACCCACTTGGCGACTCACTG | |
| CAGGGAGCACCTGTGCAGGGAGCACCTGGGGATAGGAGAGTTCCACCATGAGCTAACTTCTAGGTGGCTGCATTTGAATG | |
| GCTGTGAGATTTTGTCTGCAATGTTCGGCTGATGAGAGTGTGAGATTGTGACAGATTCAAGCTGGATTTGCATCAGTGAG | |
| GGACGGGAGCGCTGGTCTGGGAGATGCCAGCCTGGCTGAGCCCAGGCCATGGTATTAGCTTCTCCGTGTCCCGCCCAGGC | |
| TGACTGTGGAGGGCTTTAGTCAGAAGATCAGGGCTTCCCCAGCTCCCCTGCACACTCGAGTCCCTGGGGGGCCTTGTGAC | |
| ACCCCATGCCCCAAATCAGGATGTCTGCAGAGGGAGCTGGCAGCAGACCTCGTCAGAGGTAACACAGCCTCTGGGCTGGG | |
| GACCCCGACGTGGTGCTGGGGCCATTTCCTTGCATCTGGGGGAGGGTCAGGGCTTTCCCTGTGGGAACAAGTTAATACAC | |
| AATGCACCTTACTTAGACTTTACACGTATTTAATGGTGTGCGACCCAACATGGTCATTTGACCAGTATTTTGGAAAGAAT | |
| TTAATTGGGGTGACCGGAAGGAGCAGACAGACGTGGTGGTCCCCAAGATGCTCCTTGTCACTACTGGGACTGTTGTTCTG | |
| CCTGGGGGGCCTTGGAGGCCCCTCCTCCCTGGACAGGGTACCGTGCCTTTTCTACTCTGCTGGGCCTGCGGCCTGCGGTC | |
| AGGGCACCAGCTCCGGAGCACCCGCGGCCCCAGTGTCCACGGAGTGCCAGGCTGTCAGCCACAGATGCCCAGGTCCAGGT | |
| GTGGCCGCTCCAGCCCCCGTGCCCCCATGGGTGGTTTTGGGGGAAAAGGCCAAGGGCAGAGGTGTCAGGAGACTGGTGGG | |
| CTCATGAGAGCTGATTCTGCTCCTTGGCTGAGCTGCCCTGAGCAGCCTCTCCCGCCCTCTCCATCTGAAGGGATGTGGCT | |
| CTTTCTACCTGGGGGTCCTGCCTGGGGCCAGCCTTGGGCTACCCCAGTGGCTGTACCAGAGGGACAGGCATCCTGTGTGG | |
| AGGGGCATGGGTTCACGTGGCCCCAGATGCAGCCTGGGACCAGGCTCCCTGGTGCTGATGGTGGGACAGTCACCCTGGGG | |
| GTTGACCGCCGGACTGGGCGTCCCCAGGGTTGACTATAGGACCAGGTGTCCAGGTGCCCTGCAAGTAGAGGGGCTCTCAG | |
| AGGCGTCTGGCTGGCATGGGTGGACGTGGCCCCGGGCATGGCCTTCAGCGTGTGCTGCCGTGGGTGCCCTGAGCCCTCAC | |
| TGAGTCGGTGGGGGCTTGTGGCTTCCCGTGAGCTTCC [symbol] CCTAGTCTGTTGTCTGGCTGAGCAAGCGTCCTGAGGGGCTCT | |
| CTATTGCAG | |
| Intron 4 | |
| GTGGCTGTGCTTTGGTTTAACTTCCTTTTTAAACAGAAGTGCGTTTGAGCCCCACATTTGGTATCAGCTTAGATGAAGGG | (SEQ ID NO 7) |
| CCCGGAGGAGGGGCCACGGGACACAGCCAGGGCCATGGCACGGCGCCAACCCATTTGTGCGCACAGTGAGGTGGCCGAGG | |
| TGCCGGTGCCTCCAGAAAAGCAGCGTGGGGGTGTAGGGGGAGCTCCTGGGGCAGGGACAGGCTCTGAGGACCACAAGAAG | |
| CAGCCGGGCCAGGGCCTGGATGCAGCACGGCCCGAGGTCCTGGATCCGTGTCCTGCTGTGGTGCGCAGCCTCCGTGCGCT | |
| TCCGCTTACGGGGCCCGGGGACCAGGCCACGACTGCCAGGAGCCCACCGGGCTCTGAGGATCCTGGACCTTGCCCCACGG | |
| CTCCTGCACCCCACCCCTGTGGCTGCGGTGGCTGCGGTGACCCCGTCATCTGAGGAGAGTGTGGGGTGAGGTGGACAGAG | |
| GTGTGGCATGAGGATCCCGTGTGCAACACACATGCGGCCAGGAACCCGTTTCAAACAGGGTCTGAGGAAGCTGGGAGGGG | |
| TTCTAGGTCCCGGGTCTGGGTGGCTGGGGACACTGGGGAGGGGCTGCTTCTCCCCTGGGTCCCTATGGTGGGGTGGGCAC | |
| TTGGCCGGATCCACTTTCCTGACTGTCTCCCATGCTGTCCCCGCCAG | |
| Intron 5 | |
| GTGGGTGCCGGGGACCCCCGTGAGCAGCCCTGCTGGACCTTGGGAGTGGCTGCCTGATTGGCACCTCATGTTGGGTGGAG | (SEQ ID NO 8) |
| GAGGTACTCCTGGGTGGGCCGCAGGGAGTGCAGGTGACCCTGTCACTGTTGAGGACACACCTGGCACCTAGGGTGGAGGC | |
| CTTCAGCCTTTCCTGCAGCACATGGGGCCGACTGTGCACCCTGACTGCCCGGGCTCCTATTCCCAAGGAGGGTCCCACTG | |
| GATTCCAGTTTCCGTCAGAGAAGGAACCGCAACGGCTCAGCCACCAGGCCCCGGTGCCTTGCACCCCAGTCCTGAGCCAG | |
| GGGTCTCCTGTCCTGAGGCTCAGAGAGGGGACACAGCCCGCCCTGCCCTTGGGGTCTGGAGTGGTGGGGGTCAGAGAGAG | |
| AGTGGGGGACACCGCCAGGCCAGGCCCTGAGGGCAGAGGTGATGTCTGAGTTTCTGCGTGGCCACTGTCAGTCTCCTCGC | |
| CTCCACTCACACAG | |
| 5′-region intron 6 | |
| CTAAGGTTCACGTGTGATAGTCGTGTCCAGGATGTGTGTCTCTGGGATATGAATGTGTCTAGAATGCAGTCGTGTCTGTG | (SEQ ID NO 9) |
| ATGCGTTTCTGTCGTGGAGGTACTTCCATGATTTACACATCTGTGATATGCGTGTGTGGCACGTGTGTGTCGTGGTGCAT | |
| GTATCTGTGGCGTGCATATTTGTGGTGTGTGTGTGTGTGGCACGTGTGTGTCCATGGTGTGTGTGCCTGTGGTGTGCATG | |
| TGTGTGTGTCTGTGACACGTGCATGTTCATGCTGTGTGCTGCATGTCTGTGATGTGCCTATTTGTGGTGTGTGTGTGCAT | |
| GTGTCCGTGACATATGCGTGTCTATGGCATGGGTGTGTGTGGCCCCTTGGCCTTACTCCTTCCTCCTCCAGGCATGGTCC | |
| GCACCATTGTCCTCACGCTCTCGGGTGCTGGTTTGGGGAGCTCCACATTCAGGGTCCTCACTTCTAGCATGGGTGCCCCT | |
| GTCCTGTCACAGGGCTGGGCCTTGGAGACTGTAAGCCAGGTTTGAGAGGAGAGTAGGGATGCTGGTGGTACCTTCCTGGA | |
| CCCCTGGCACCCCCAGGACCCCAGTCTGGCCTATGCCGGCTCCATGAGATATAGGAAGGCTGATTCAGGCCTCGCTCCCC | |
| GGGACACACTCCTCCCAGAGCGGCCGGGGGCCTTGGGCCTCGGCAGGGGTGAAAGGGGCCCTGGGCTTGGGTTCCCACCC | |
| AGTGGTCATGAGCACGCTGGAGGGGTAAGCCCTCAAAGTCGTGCCAGGCCGGGGTGCAGAGGTGAAGAAGTATCCCTGGA | |
| GCCTTCGGTCTGGGGAGAGGCACATGTGGAAACCACAAGGACCTCTTTCTCTGACTTCTTGAGCT | |
| 3′-region intron 6 | |
| TGTGGGATTGGTTTTCATGTGTGGGATAGGTGGGGATCTGTGGGATTGGTTTTTATGAGTGGGGTAACACAGAGTTCAAG | (SEQ ID NO 10) |
| GCGAGCTTTCTTCCTGTAGTGGGTCTGCAGGTGCTCCAACAGCTTTATTGAGGAGACCATATCTTCCTTTGAACTATGGT | |
| CGGGTTTATAGTAAGTCAGGGGTGTGGAGGCCTCCCCTGGGCTCCCTGTTCTGTTTCTTCCACTCTGGGGTCGTGTGGTG | |
| CCTGCTGTGGTGTGTGGCCGGTGGGCAGGGCTTCCAGGCCTCCTTGTGTTCATTGGCCTGGATGTGGCCCTGGCTACGCT | |
| CCGTCCTTGGAATTCCCCTGCGAGTTGGAGGCTTTCTTTCTTTCTTTTTTTCTTTCTTTTTTTTTTTTTTTGATAACAGA | |
| GTCTCGCTCTTTTTTGCCCAGGCTGGAGTGGTTTGGCGTGATCTTGGCTCACTGCAACCTGTGCTTCCTGAGTTCAAGCA | |
| ATTCTCTTGCCTCAGCCTCCCAAGTAGCTGGAATTATAGGCGCCCACCACCATGCTGACTAATTTTTGCAATTTTAGTAG | |
| AGACGAGGTTTCTCCATGTTGGCCAGGCTGGTCTCGAACTCCTGACCTCAGGTGATCCTCCCACCTCGGCCTCCCAAAGT | |
| GCTGGGATGACAGGTGTGAACCGCCGCGCCCGGCCGAGACTCGCTTCCTGCAGCTTCCGTGAGATCTGCAGCGATAGCTG | |
| CCTGCAGCCTTGGTGCTGACAACCTCCGTTTTCCTTCTCCAGGTCTCGCTAGGGGTCTTTCCATTTCATGACTCTCTTCA | |
| CAGAAGAGTTTCACGTGTGCTGATTTCCCGGCTGTTTCCTGCGTAATTGGTGTCTGCTGTTTATCGATGGCCTCCTTCCA | |
| TTTCCTTTAGGCTTTGTTTATTGTTGTTTTTCCGGCTCCTTGAAGGAAAAGTTTCGATTATGGATGTTTGAACTTTCTTT | |
| TCTAAACAAGCATCTGAAGTTGCCGTTTTCCCTCTAAAGCAGGGATCCCGAGGCCCCTGGCTGTGGAGTGGCACCGGTCT | |
| GGGGCCTGTTAGGAACCCGGCGCACAGCGGGAGGCTAGGTGGGGTGTGGGGAGCCAGCGTTCCCGCCTGAGCCCCGCCCC | |
| TCTCAGATCAGCAGTGGCATGCGGTGCTCAGAGGCGCACACACCCTACTGAGAACTGTGCGTGAGAGGGGTCTAGATTCT | |
| GTGCTCCTTATGGGAATCTAATGCCTGATGATCTGAGGTGGAACCGTTTGCTCCCAAAACCATCCCCTTCCCCACTGCTG | |
| TCCTGTGGAAAAATCGTCTTCCACGAAACCAGTCCCTGGTACCACAATGGTTGGGGACCCTGTGCTAAAGACCTGCTTCA | |
| GCAGCCTCTCGTCAGTGTTGATATATTGGCTTTTCTGTGTTGAGTCCAGAATAATTACGGATTTCTGTGATGCTTTCCGC | |
| CGACCTCAGACCCATGGGCTATTTGTGGGCGTGTTGCCTGCTCCTGGGTTGGGAAGGGTGCAGGCCCCATGTACCTTCCT | |
| GTTACTGCCTTCCAGGTTGGTTCTCAGGGTTGAATCGTACTCGATGTGGTTTTAGCCCACGGCCCTGCCGCCAGCTCCTG | |
| GGGGCTGGGGAACATGCTGAAGCACAGAGTCACCGTGCGCGTCTTTTGATGCCTCACAAGCTCGAGGCCTCCTGTGTCCG | |
| TGTTAGTGTGTGTCACGTGCCTGCTCACATCCTGTCTTGGGGACGCAGGGGCTTAGCAGGTCCCGTAGTAAATGACAAGC | |
| GTCCTGGGGGAGTCTGCAGAATAGGAGGTGGGGGTGCCGGTCTCTCTCCCGCGTCTTCAGACTCTTCTCCTGCCTGTGCT | |
| GTGGCTGCACCTGCATCCCTGCAATCCCTCCAGCACTGGGCTGGAGAGGCCCGGGAGCTCGAGTGCCACTTGTGCCACGT | |
| GACTGTGGATGGCAGTCGGTCACGGGGGTCTGATGTGTGGTGACTGTGGATGGCGGTTGGTCACAGGGGTCTGATGTGTG | |
| GTGACTGTGGATGGCGGTCGTGGGGTCTGATGTGGTGACTGTGGATGGCGGTCGTGGGGTCTGATGTGTGGTGACTGTGG | |
| ATGGCGGTCGTGGGGTCTGATGTGGTGACTGTGGATGGCGGTCGTGGGGTCTGATGTGGTGACTGTGGATGGCGGTCGTG | |
| GGGTCTGATGTGGTGACTGTGGATGGCAGTCGTGGGGTCTGATGTGTGGTGACTGTGGATGGCGGTCGTGGGGTCTGATG | |
| TGGTGACTGTGGATGGCAGTCGTGGGGTCTGATGTGTGGTGACTGTGGATGGCGGTCGTGGGGTCTGATGTGTGGTGACT | |
| GTGGATGGCGGTCGTGGGGTCTGATGTGTGGTGACTGTGGATGGCGGTCGTGGGGTCTGATGTGTGGTGACTGTGGATGG | |
| CGGTCGTGGGGTCTGATGTGGTGACTGTGGATGGCGGTCGTGGGGTCTGATGTGTGGTGACTGTGGATGGTGATCGGTCA | |
| CAGGGGTCTGATGTGTGGTGACTGTGGATGGCGGTCGTGGGGTCTGATGTGTGGTGACTGTGGATGGTGATCGGTCACAG | |
| GGGTCTGATGTGTGGTGACTGTGGATGGCGGTCGTGGGGTCTGATGTGTGGTGACTGTGGATGGCGGTTGGTCCCGGGGG | |
| TCTGATGTGTGGTGACTGTGGATGGCGATCGGTCACAGGGGTCTGATGTGTGGTGACTGTGGATGGCGGTCGTGGGGTCT | |
| GATGTGTGGTGACTGTGGATGGCGGTCGTGGGGTCTGATGTGTGGTGACTGTGGATGGCGGTCGTGGGGTCTGATGTGGT | |
| GACTGTGGATGGCGGTCGTGGGGTCTGATGTGGTGACTGTGGATGGCGGTCGTGGGGTCTGATGTGTGGTGACTGTGGAT | |
| GGCGGTTGGTCCCGGGGGTCTGATGTGTGGTGACTGTGGATGGCGGTCGTGGGGTCTGATGTGGTGACTGTGGATGGCAG | |
| TCGTGGGGTCTGATGTGTGGTGACTGTGGATGGCGGTCGTGGGGTCTGATGTGTGGTGACTGTGGATGGCGGTCGTGGGG | |
| TCTGATGTGTGGTGACTGTGGATGGCGGTCGTGGGGTCTGATGTGTGGTGACTGTGGATGGCGGTCGTGGGGTCTGATGT | |
| GGTGACTGTGGATGGCGGTCGTGGGGTCTGATGTGTGGTGACTGTGGATGGTGATCGGTCACAGGGGTCTGATGTGTGGT | |
| GACTGTGGATGGCGGTCGTGGGGTCTGATGTGTGGTGACTGTGGATGGCGGTCGTGGGGTCTGATGTGGTGACTGTGGAT | |
| GGCGGTCGTGGGGTCTGATGTGTGGTGACTGTGGATGGCGGTCGTAGGGTCTGATGTGTGGTGACTGTGGATGGCAGTCG | |
| GTCACAGGGGTCTGATGTGTGGTGACTGTGGATGGCGGTCGTGGGGTCTGATGTGTGGTGACTGTGGATGGCGGTCGTGG | |
| GGTCTGATGTGTGGTGACTGTGGATGGCGGTCGTGGGGTCTGATGTGTGGTGACTGTGGATGGCGGTCGTGGGGTCTGAT | |
| GTGGTGACTGTGGATGGTGATCGGTCACAGGGGTCTGATGTGTGGTAGCTGCAGGTGGAGTCCCAGGTGTGTCTGTAGCT | |
| ACTTTGCGTCCTCGGCCCCCCGGCCCCCGTTTCCCAAACAGAAGCTTCCCAGGCGCTCTCTGGGCTTCATCCCGCCATCG | |
| GGCTTGGCCGCAGGTCCACACGTCCTGATCGGAAGAAACAAGTGCCCAGCTCTGGCCGGGGCAGGCCACATTTGTGGCTC | |
| ATGCCCTCTCCTCTGCCGGCAG | |
| Intron 7 | |
| GTCTGGGCACTGCCCTGCAGGGTTGGGCACGGACTCCCAGCAGTGGGTCCTCCCCTGGGCAATCACTGGGCTCATGACCG | (SEQ ID NO 11) |
| GACAGACTGTTGGCCCTGGGGGGCAGTGGGGGGAATGAGCTGTGATGGGGGCATGATGAGCTGTGTGCCTTGGCGAAATC | |
| TGAGCTGGGCCATGCCAGGCTGCGACAGCTGCTGCATTCAGGGACCTGCTCACGTTTGACTGCGCGGCCTCTCTCCAGTT | |
| CCGCAGTGCCTTTGTTCATGATTTGCTAAATGTCTTCTCTGCCAGTTTTGATCTTGAGGCCAAAGGAAAGGTGTCCCCCT | |
| CCTTTAGGAGGGCAGGCCATGTTTGAGCCGTGTCCTGCCCAGCTGGCCCCTCAGTGCTGGGTCTGAGGCCAAAGGAAACG | |
| TGTCCCCCTTCTTAGGAGGACGGGCCGTGTTTGAGCCACGCCCCGCTGAGCGGGCCTCTCAGTGCTGGGTCTGTCCACGT | |
| GGCCCTGTGGCCCTTTGCAGATGTGGTCTGTCCACGTGGCCCTGTGGCTCTTTGCAGATGCCTGTTAGCACTTGCTCGGC | |
| TCTAGGGGACAGTCGTGTCCACCGCATGAGGCTCAGAGACCTCTGGGCGAATTTCCTTGGCTCCCAGGGTGGGGGTGGAG | |
| GTGGCCTGGGCTGCTGGGACCCAGACCCTGTGCCCGGCAGCTGGGCAGCAACTCCTGGATCACATATGCCATCCGGGCCA | |
| CGGTGGGCTGTGTGGGTGTGAGCCCAGCTGGACCCACAGGTGGCCCAGAGGAGACGTTCTGTGTCACACACTCTGCCTAA | |
| GCCCATGTGTGTCTGCAGAGACTCGGCCCGGCCAGCCCACGATGGCCCTGCATTCCAGCCCAGCCCCGCACTTCATCACA | |
| AACACTGACCCCAAAAGGGACGGAGGGTCTTGGCCACGTGGTCCTGCCTGTCTCAGCACCCACCGGCTCACTCCCATGTG | |
| TCTCCCGTCTGCTTTCGCAG | |
| Intron 8 | |
| GTGAGTCAGGTGGCCAGGTGCCATTGCCCTGCGGGTGGCTGGGCGGGCTGGCAGGGCTTCTGCTCACCTCTCTCCTGCCC | (SEQ ID NO 12) |
| CTTCCCCACTGNCCTTCTGCCCGGGGCCACCAGAGTCTCCTTTTCTGGCCCCCGCCCCCTCCGGCTCCTGGGCTGCAGGC | |
| TCCCGAGGCCCCGGAAACATGGCTCGGCTTGCGGCAGCCGGAGCGGAGCAGGTGCCACACGAGGCCTGGAAATGGCAAGC | |
| GGGGTGTGGAGTTGCTCCTGCGTGGAGGACGAGGGGCGGGGGGTGTGTCTGGGTCAGGTGTGCGCCGAGCGTTTGAGCCT | |
| GCAGCTTGTCAGCTCCAAGTTACTACTGACGCTGGACACCCGGCTCTCACACGCTTGTATCTCTCTCTCCCGATACAAAA | |
| GGATTTTATCCGATTCTCATTCCTGTCCCTGTCGTGTGACCCCCGCGAGGGCGCGGGCTCTTCTCTCTGTGACTAGATTT | |
| CCCATCTGGAAAGTGCGGGGTTGACCGTGTAGTTTGCTCCTCTCGGGGGGCCTGTGGTGGCCATGGGGCAGGCGGCCTGG | |
| GAGAGCTGCCGTCACACAGCCACTGGGTGAGCCACACTCACGGTGGTAGAGCCACAGTGCCTGGTGCCACATCACGTCCT | |
| CTGGATTTTAAGTAAAACCACACACCTCCCGGCAGGCATCTGCCTGCGACCCTGTGTGTGCCTGGGGAGAGTGGTAGCAC | |
| GGAGGAAATTCGTGCACACTCAAGGTCATCAGCAAGGTCATCCGCAGTCAGGTGGAACGTGGAGGCCTCTCTCTGGGATC | |
| GTCTCCAGCGGATAAAGGACTGTGCACAGCTTCGGAAGCTTTTATTTAAAAATATAACTATTAATTATTGCATTATAAGT | |
| AATCACTAATGGTATCAGCAATTATAATATTTATTAAAGTATAATTAGAAATATTAAGTAGTACACACGTTCTGGAAAAA | |
| CACAAATTGCACATGGCAGCAGAGTGAATTTTGGCCGAGGGACACGTGTGCACATGTGTGTAAGCGGCCCCCAGGCCCAC | |
| AGAATTCGCTGACAAAGTCACCTCCCCAGAGAAGCCACCACGGGCCTCCTTCGTGGTCGTGAATTTTATTAAGATGGATC | |
| AAGTCACGTACCGTCCACGTGTGGCAGGGCTTTGGGGAATGTGAGGTGATGACTGCGTCCTCATGCCCTGACAGACAGGA | |
| GGTGACTGTGTCTGTCCTGTCCCTAGGACACGGACAGGCCCGAAGCTCTAGTCCCCATCGTGGTCCAGTTTGGCCTCTGA | |
| ATAAAAACGTCTTCAAAACCTGTTGCCCCAAAAACTAAGAACAGAGAGAGTTTCCCATCCCATGTGCTCACAGGGGCGTA | |
| TCTGCTTGCGTTGACTCGCTGGGCTGGCCGGACTCCTAGAGTTGGTGCGTGTGCTTCTGTGCAAAAAGTGCAGTCCTCTT | |
| GCCCATCACTGTGATATCTGCACCAGCAAGGAAAGCCTCTTTTCTTTTCTTTCTTTTTTTTTTTTTGAGACGGAACGTCA | |
| CTGTTGTCTGCCTGGGCTTGAGTGCAGTGGCGCGATCTCAACTCACTGCAACCTCCGCCTCCCGGGTTCCAGCATTTCTC | |
| CTGCCTCAGCCTCCCGAGCAGCTGAGATTACAGGCACCCACCCCCTGCGCCTGGCTAATTTTTGTATTTTTAGTAGAGAG | |
| GGGTTTTTGCCATGTTGGCCAGGCTGGTCTCGAACTCCTGACCTCAGGTGATCCACCCACCTCGGCCTCCCAAAGTGCTG | |
| GGATTACAGGTGTGAGCCATCACGCCCAGCCGGAAAGCCTCTTTTTAAGGTGACCACCTATAGCGCTTCCCGAAAATAAC | |
| AGGTCTTGTTTTTGCAGTAGGCTGCAAGCGTCTCTTAGCAACAGGAGTGGCGTCCTGTGGGCTCTGGGGATGGCTGAGGG | |
| TCGCGTGGCAGCCATGCCTTCTGTGTGCACCTTTAGGTTCCACGGGGCTATTCTGCTCTCACTGTTTGTCTGAAAACGCA | |
| CCCTTGGCATCCTTGTTTGGAGAGTTTCTGCTTCTCGTTGGTCATGCTGAAACTAGGGGCAAGGTTGTATCCGTTGGCGC | |
| GCAGCGGCTACATGTAGGGTCATGAGTCTTTCACCGTGGACAAATTCCTTGAAAAAAAAAAAAGGAGTCCGGTTAAGCAT | |
| TCATTCCGGGTCAAGTGTCTGGTTCTGTGAATAAACTCTAAGATTTAAGAAACCTTAATGAAAGAAAACCTTGATGATTC | |
| AGAGCAAGGATGTGGTCACACCTGTGGCTGGATCTGTTTCAGCCGCCCCAGTGCATGGTGAGAGTGGGGAGCAGGGATTG | |
| TTTGTTCAGAGGTCTCATCTGGTATGTTTCTGAGGTGTTTGCCGGCTGAATGGTAGACGTGTCGTTTGTGTGTATGAGGT | |
| TCTGTGTCTGTGTGTGGCTCGGTTTGAGTGTACGCATGTCCAGCACATGCCCTGCCCGTCTCTCACCTGTGTCTTCCCGC | |
| TCCAG | |
| Intron 9 | |
| CCGAGGCCTCCTCTTCCCCAGGGGGGCTTGGGTGGGGGTTGATTTGCTTTTGATGCATTCAGTGTTAATATTCCTGGTGC | (SEQ ID NO 13) |
| CCTGGAGACCATGACTGCTCTGTCTTGAGGAACCAGACAAGGTTGCAGCCCCTTCTTGGTACGAAGCCGCACGGGAGGGG | |
| TTGCACAGCCTGAGGACTGCGGGCTCCACGCAGGCTCTGTCCAGCGGCCATGTCCAGAGGCCTCAGGGCTCAGCAGGCGG | |
| GAGGGCCGCTGCCCTGCATGATGAGCATGTGAATTCAACACCGAGGAAGCACACCAGCTTCTGTCACGTCACCCAGGTTC | |
| CGTTAGGGTCCTTGGGGAGATGGGGCTGGTGCAGCCTGAGGCCCCACATCTCCCAGCAGGCCCTCGACAGGTGGCCTGGA | |
| CTGGGCGCCTCTTCAGCCCATTGCCCATCCCACTTGCATGGGGTCTACACCCAAGGACGCACACACCTAAATATCGTGCC | |
| AACCTAATGTGGTTCAACTCAGCTGGCTTTTATTGACAGCAGTTACTTTTTTTTTTTTAATACTTTAAGTTCTAGGGTAC | |
| ATGTGCACGACGTGCAGGTTAGTTACATATGTATACATGTGCCATGTTGGTGTGCTGCACCCATTAACTCATCATTTACA | |
| TTAGGTATATCTCCTAATGCTATCCCTCCCCACTCCCCCCATCCCATGACAGGCCCTGGTGTGTGATGTTCCCCACCCTG | |
| TGTCCAAGTGTTCTCATTGTTCAGTTCCCACCTGTGAGTGAGAACATGTGGTGTTTGGTTTTCTTTCCTTGCAATAGTTT | |
| GCTCAGAGTGATGGTTTCCAGCTTCGTCCATGTCCCTACAAAGGACATGAACTCATCCTTTTTTATGACTGCATAGTATT | |
| CCGTGGTGTATATGTGCCACATTTTCTTAATCCAGTCTATCATCGATGGACATTTGGGTTGGTTGCAAGTCTTTGCTACT | |
| GTGAATAGTGCCGCAATAAACATACGTGTGCATGTGTCTTTATAGCAGCATGATTTATAATCCTTTGGGTATATACCCAG | |
| TAATGGGATGGCTGGGTCAAATGGTATTTCTAGTTCTAGATCCTTGAGGAATCACCACACTGTCTTCCACAATGGTTGAA | |
| CTAGTTTACACTCCCACCAACAGTGTAAAAGTGTTCTGGTGCTGGAGAGGATGTGGACAGCAGTTATTTTTTTATGAAAA | |
| TAGTATCACTGAACAAGCAGACAGTTAGTGAAGGATGCGTCAGGAAGCCTGCAGGCCACACAGCCATTTCTCTCGAAGAC | |
| TCCGGGTTTTTCCTGTGCATCTTTTGAAACTCTAGCTCCAATTATAGCATGTACAGTGGATCAAGGTTCTTCTTCATTAA | |
| GGTTCAAGTTCTAGATTGAAATAAGTTTATGTAACAGAAACAAAAATTTCTTGTACACACAACTTGCTCTGGGATTTGGA | |
| GGAAAGTGTCCTCGAGCTGGCGGCACACTGGTCAGCCCTCTGGGACAGGATACCTCTGGCCCATGGTCATGGGGCGCTGG | |
| GCTTGGGCCTGAGGGTCACACAGTGCACCATGCCCAGCTTCCTGTGGATAGGATCTGGGTCTCGGATCATGCTGAGGACC | |
| ACAGCTGCCATGCTGGTAAAGGGCACCACGTGGCTCAGAGGGGGCGAGGTTCCCAGCCCCAGCTTTCTTACCGTCTTCAG | |
| GCTGATGGTAAACACTGAGTACTTATAATGAATGAGGAATTGCTGTAGCAGTTAACTGTAGAGAGCTCGTCTGTTGGAAA | |
| GAAATTTAAGTTTTTCATTTAACCGCTTTGGAGAATGTTACTTTATTTATGGCTGTGTAAATTGTTTGACATTCAGTCCC | |
| TCGTAGACAGATACTACGTAAAAAGTGTAAAGTTAACCTTGCTGTGTATTTTCCCTTATTTTAG | |
| Intron 10 | |
| GTGAGGCCCGTGCCGTGTGTCTGTGGGGACCTCCACAGCCTGTGGGCTTTGCAGTTGAGCCCCCCGTGTCCTGCCCCTGG | (SEQ ID NO 14) |
| CACCGCAGCGTTGTCTCTGCCAAGTCCTCTCTCTCTGCCGGTGCTGGATCCGCAAGAGCAGAGGCGCTTGGCCGTGCACC | |
| CAGGCCTGGGGGCGCAGGGGCACCTTCGGGAGGGAGTGGGTACCGTGCAGGCCCTGGTCCTGCAGAGACGCACCCAGGTT | |
| ACACACGTGGTGAGTGCAGGCGGTGACCTGGCTCCTGCTGCTCTTTGGAAAGTCAAGAGTGGCGGCTCCTGGGGCCCCAG | |
| TGAGACCCCCAGGAGCTGTGCACAGGGCCTGCAGGGCCGAGGCGGCAGCCTCCTCCCCAGGGTGCACCTGAGCCTGCGGA | |
| GAGCAGGAGCTGCTGAGTGAGCTGGCCCACAGCGTTCGCTGCGGTCACGTTCCTGCGTGGGGTTGTTTGGGATCGGTGGG | |
| AGAATTTGGATTTCCTGAGTGCTGCTGTCTTGAACCACGGAGATGGCTAGGAGTGGGTTTCAGAGTTGATTTTTGTGAAT | |
| CAAACTAAAATCAGGCACAGGGGACCTGGCCTCAGCACAGGGGATTGTCCCCCTGTGGTCCCCCTCAAGGGCGCCCACAG | |
| AGCCGGTGGGCTTGTTTTAAAGTGCGATTTGACGAGGGACGAGAAACCTTGAAAGCTGTAAAGGGAACCCTCAGAAAATG | |
| TGGCCGCCAGGGGTGGTTTCAGGTGCTTTGCTGGGCTGTGTTTGTGAAAACCCATTTGGACCCGCCCTCCAAGTCCACCC | |
| TCCAGGTCCACCCTCCAGGGCCGCCCTGGGCTGGGGGTATGCCTGGCGTTCCTTGTGCCGCAGCCCGGAGCACAGCAGGC | |
| TGTGCACATTTAAATCCACTAAGATTCACTCGGGGGGAGCCCAGGTCCCAAGCAACTGAGGGCTCAGGAGTCCTGAGGCT | |
| GCTGAGGGGACAGAGCAGACGGGGAACGCTGCTTCTGTGTGGCAAGTTCCTGAGGGTGCTGGCCAGGGAGGTGGCTCAGA | |
| GTGTATGTTGGGGTCCCACCGGGGGCAGAACTCTGTCTCTGATGAGTCGGCAGCCATGTAACAGGAAGGGGTGGCCACAG | |
| GGAGCTGGGAATGCACCAGGGGAGCTGCGCAGCTGGCCGAGGTCCCAGGGCCAGGCCACAGGAAGGGCAGGGGGACGCCC | |
| GGGGCCACAGCAGAGGCCGCAGGAAGGGAAGGGGATGCCCAGGCCAGAGCAGAGGCTACCGGGCACAGGGGGGCTCCCTG | |
| AGCTGGGTGAGCGAGGCTCATGACTCGGCGAGGGAACCTCCTTGACGTGAAGCTGACGACTGGTGTTGCCCAGCTCACAG | |
| CCCAGCCAGGTCCCGCGCCTGAGCAGGAACTCAGAACCCTCCCCTTTGTCTAAAGCACAGCAGATGCCTTCAGGGCATCT | |
| AGGAGAAAACAGGCAAAGTCGTTGAGAAACGTCTTAAAAGAAGGTGGGATGGTGGCAATTTCTTGTCCAGATCTTAGTCT | |
| GCCCCGGACCACAGATGAGTCTATAACGGGATTGTGGTGTTGCCATGGGGACACATGAGATGGACCATCACACAGGCCAC | |
| TGGGGCTGCACCTCCCATCTGAGTCCTGGCTGTCCCGGGTCCAGGCCAGGTTCTTGCATGCTCACCTACCTC [symbol] GCTGCCC | |
| GGGAGACAGGGAAAGCACCCCGAAGTCTGGAGCAGGGCTGGGTCCAGGCTCCTCAGAGCTCCTGCCAGGCCCAGCACCCT | |
| GCTCCAAATCACCACTTCTCTGGGGTTTTCCAAAGCATTTAACAAGGGTGTCAGGTTACCTCCTGGGTGACGGCCCCGCA | |
| TCCTGGGGCTGACATTGCCCCTCTGCCTTAG | |
| Intron 11 | |
| GTGAGCGCACCTGGCCGGAAGTGGAGCCTGTGCCCGGCTGGGGCAGGTGCTGCTGCAGGGCCGTTGCGTCCACCTCTGCT | (SEQ ID NO 15) |
| TCCGTGTGGGGCAGGCGACTGCCAATCCCAAAGGGTCAGAGGCCACAGGGTGCCCCTCGTCCCATCTGGGGCTGAGCAGA | |
| AATGCATCTTTCTGTGGGAGTGAGGGTGCTCACAACGGGAGCAGTTTTCTGTGCTATTTTGGTAAAAGGAAATGGTGCAC | |
| CAGACCTGGGTGCACTGAGGTGTCTTCAGAAAGCAGTCTGGATCCGAACCCAAGACGCCCGGGCCCTGCTGGGCGTGAGT | |
| CTCTCAAACCCGAACACAGGGGCCCTGCTGGGCATGAGTCCCTCTGAACCCGAGACCCTGGGGCCCTGCTGGGCGTGAGT | |
| CTCTCCGAACCCAGAGACTTCAGGGCCCTTTTGGGCGTGAGTCTCTCCGCTGTGAGCCCCACACTCCAAGGCTCATCCAC | |
| AGTCTACAGGATGCCATGAGTTCATGATCACGTGTGACCCATCAGGGGACAGGGCCATGGTGTGGGGGGGGTCTCTACAA | |
| AATTCTGGGGTCTTGTTTCCCCAGAGCCCGAGAGCTCAAGGCCCCGTCTCAGGCTCAGACACAAATGAATTGAAGATGGA | |
| CACAGATGCAGAAATCTGTGCTGTTTCTTTTATGAATAAAAAGTATCAACATTCCAGGCAGGGCAAGGTGGCTCACACCT | |
| ATAATCCCAGCACTTTGGGAGGCCGAGGTGGGTGGATCACTTGAGGCCAGGAGTTTGAGGCCAACCTAACCAACATAGTG | |
| AAATTCCATTTCTACTTAAAAAATACAAAAATTAGCCTGGCCTGGTGGCACACGCCTGTAGTCCCCGCTATGCGGGAGGC | |
| TGAGGCAGGAGAATCATTTGAACCCAGGAGGCAGAGGTTGCAGTGAGCCGAGATCACACCACTGCACTCCAGCCTGGGCA | |
| ACAGAGTGAGACTTCATCTTAAAAAAAAAAAAAAAAGTATCAGCATTCCAAAACCATAGTGGACAGGTGTTTTTTTATTC | |
| TGTCCTTCGATAATATTTACTGGTGCTGTGCTAGAGGCCGGAACTGGGGGTGCCTTCCTCTGAAAGGCACACCTTCATGG | |
| GAAGAGAAATAAGTGGTGAATGGTTGTTAAACCAGAGGTTTAAACTGGGGTCCTGTCGTTCTGAGTTAACAGTCCAGATC | |
| TGGACTTTGCCTCTTTCCAGAATGCTCCCTGGGGTTTGCTTCATGGGGGAGCAGCAGGTGTGGACACCCTCGTGATGGGG | |
| GAGCAGCAGGTGCAGACGCCCTCATGATGGGGGAGTGGCAGGTGCAGACACCCTTGTGCATGGTGCCCAGCATGTCCCTG | |
| TTGCAGCTCCCTCCCCACAAGGATGCCGGTCTCCTGTGCTCCCCACAGTCCCTGCTTCCCTCTCACAGCCTTACCTGGTC | |
| CTGGCCTCCACTGGCTTTGTCTGCATGATTTCCACATTTCCTGGGCTCCCAGCACCTCTTCGCCTCTCCCAGGCACCTCT | |
| GCAGTCCTGGCCATACCAGTCAGCTGTGAACTGTCCACTGCTTATTTTGCTCCCCATGAAATGTATTTTTTAGGACAGGC | |
| ACCCCTGGTTCCAGCCTCTGGCACAGCATCAGTGAATGTTATTGAAGGACAAAGGACAGACAAACAAATCAGGAAAATGG | |
| GTTCTCTCTAAACACATTGCAAAGCCACAGAGGCTAGTGCAGGATGGGTGGGCATCAGGTCATCAGATGTGGGTCCAATG | |
| CCAGAATATTCTGTGCTCCCAAAGGCCACTTGGTCAGAGTGTGTGCTTGCAGAGGTGGCTCTAAAAGCTCAGCAGTGGAG | |
| GCAGTGGTTCGCCATACTCAGGGTGAACTCACATCCTCTGTGTCTGAAGTATACAGCAGAGGCTTGAAGGGCATCTGGGA | |
| GAAGAAAACAGGCAAAATGATTAAGAAAAGTGAAAAAGGAAAAGTGGTAAGATGGGAATTTTCTTGTCCAGATTTTAGTC | |
| TCCCAAACCACAGCTCAGATGGTAGAATGTGGTCAGAACTGATGGACAGAACAATAGAACAAAACGGAAGCCCTATCTCT | |
| CAGAAACGTGTGTTAATGTGGTATGTGGCACAGCTGATGGAAAAGAGAGTGTGTGTGTAATTTTTTTTTCTGAGAAAACT | |
| GACTGGAAGCAAATAAGTTGTGTCTTTACAGCATATACCAGAGCAGATTCTAGGTAGAAGAGGAGACACATGCAAACAAC | |
| ACCAGCAACAGAAATAAAACAAAAGACTCAAAGGGAAGGGAGGTGAACGTTCCCTGGTTTGGTGTTGGGGAAGGACACAC | |
| AGGGAGGCGGATGAAACCACTGAGGCAACCGGCATTGCTTTCACTGCAGAGAAACTCAGCTTGCCTGAGCCACAGTGAAA | |
| ATGGCCATTCCCTGGAGCGTTTGTGCACGTGATTTATTTAAGGCGCCCTGTGAGGTCCTGCACATTCATCCTCTCACTTT | |
| GTTCTCCTAACCACCTGAGAGGTAGAGGAGGAAAGGCTCCAGGGGAGCAGCCGCCCTTGGTCACCCAGCTGGCAAAGGGC | |
| ATGCATGATTGCAGCCTGGCCTCCTGCTCCGGGGCCCTTGCTCTGCCCGAGGACCCCACACAAGTCAGACCCATAGGCTC | |
| AGGGTGAGCCGGAGCCCAAGGTCGTGTTGGGGATGGCTGTGAAAGAAGAAATGGACGTCTGATGCACACTTGGGAAGGTC | |
| CTACCAGCAGCGTCAAAGAAATGCATGTGAAACTGACAGCGAGACCCATCCCTCAAAGAAACGCACGTGAAACTGATGGC | |
| GAGACCTGTCCCCATCCCTCATGCTGGCTCCTTTTCTGGGCTTGCCAAGAGCCAGCATCAGGTTGAGGCAAGCTGGAAAG | |
| ACTTTTCTGGAAAGCAGCTTGTTTGCATGGAAGTCCTCACAATGTCCTGTGTCTTCCCAGTAATTCCACTTCTGAAGTGA | |
| CCAGACATTATCACGGGTCTTATTTACCATTTCCAGTGTTCCAGGCAGGGGGACTTGCCACAGCAAGTCACGAACCTGCC | |
| CAAATACAGGGCTAAGGAGATATTATGCATCACAAAACTTGCTCTGCCATTAAACATTTTTCAAAGAATTTTTGAAGAAT | |
| GTTTAATGGCACAAAACGTTTATTTCAATGTAGCAGTGTTCAAAGCTGGCTGTAAAAGAACACACCCCAGGAGCCTGCCG | |
| TGAATGTCATGTGTGTTCATCTTTGGACATGGACATACATGGGCAGTGAGTGGTGGTGAGGCCCTGGAGGACATCGGTGG | |
| GATGCCTCCATCCTGCCCCTCTGGAGACACCATGTGTGCCACGTGCACTCACTGGAGCCCTGTTTAGCTGGTGCCACCTG | |
| GCTCTTCCATCCCTGAGATTCAAACACAGTGAGATTCCCCACGCCCAACTCAGTGTTCTCCCACAAAAAACCTGAGTCAC | |
| ACCTGTGTTCACTCGAGGGACGCCCGGGAGCCAGGGCTCCACAGTTTATTATGTGTTTTTGGCTGAGTTATGTGCAGATC | |
| TCATCAGGGCAGATGATGAGTGCACAAACACGGCCGTGCGAGGTTTGGATACACTCAACATCACTAGCCAGGTCCTGGTG | |
| GAGTTTGGTCATGCAGAGTCTGGATGGCATGTAGCATTTGGAGTCCATGGAGTGAGCACCCAGCCCCCTCGGGCTGCAGC | |
| GCATGCCCCAGGCAGGACAAGGAAGCGGGAGGAAGGCAGGAGGCTCTTTGGAGCAAGCTTTGCAGGAGGGGGCTGGGTGT | |
| GGGGCAGGCACCTGTGTCTGACATTCCCCCCTGTGTCTCAG | |
| Intron 12 | |
| GTGAGCAGGCTGATGGTCAGCACAGAGTTCAGAGTTCAGGAGGTGTGTGCGCAAGTATGTGTGTGTGTGTGTGCGCGCGT | (SEQ ID NO 16) |
| GCCTGCAAGGCTGATGGTGACTGGCTGCACGTAAGAGTGCACATGTACGCATATACACGTGAGCACATACATGTGTGCAT | |
| GTGTGTACATGAAGGCATGGCAGTGTGTGCACAGGTGTGCAAGGGCACAAGTGTGTGCACATGCGAATGCACACCTGACA | |
| TGCATGTGTGTTCGTGCACAGTCGTGTGGGCATTCACGTGAGGTGCATGCGTGTGGGTGTGCAGTGTGAGTAGCATGTGT | |
| GCACATAACATGTATTGAGGGGTCCTCGTGTTCACCCCGCTAGGTCCTCAGCACCAGTGCCACTCCTTAGAGGATGAGAC | |
| GGGGTCCCACGCCTTGGTGGGCTGAGGCTCTGAAGCTGCAGCCCTGAGGGCATTGTCCCATCTGGGCATCCGCGTCCACT | |
| CCCTCTCCTGTGGGCTTCTGTGTCCACTCCCCCTCTCCTGTGGGCATTTACATCCACTCCACTCCCTCTCTCCTGTGGGC | |
| ATCCGCGTCCACTCCCCCTCTCTGTGGGCATCTGCGTCCACCTCCCCTCTCTGTGGGCATTTGCGTCCACTCCCTCTCCT | |
| GGTTCCTTCCTGTCTTGGCCGAGCCTCGGGGGCAGGCAGATGACACAGAGTCTTGACTCGCCCAGGGTGGTTCGCAGCTG | |
| CCGGGTGAGGGCCAGGCCGGATTTCACTGGGAAGAGGGATAGTTTCTTGTCAAAATGTTCCTCTTTCTTGTTCCATCTGA | |
| ATGGATGATAAAGCAAAAAGTAAAAACTTAAAATCCCAGAGAGGTTTCTACCGTTTCTCACTCTTTCTTGGCGACTCTAG | |
| Intron 13 | |
| GTGAGCCGCCACCAAGGGGTGCAGGCCCAGCCTCCAGGGACCCTCCGCGCTCTGCTCACCTCTGACCCGGGGCTTCACCT | (SEQ ID NO 17) |
| TGGAACTCCTGGGTTTTAGGGGCAAGGAATGTCTTACGTTTTCAGTGGTGCTGCTGCCTGTGCACAGTTCTGTTCGCGTG | |
| GCTCTGTGCAAAGCACCTGTTCTCCATCTCTGGGTAGTGGTAGGAGCCGGTGTGGCCCCAGGTGTCCCCACTGTGCCTGT | |
| GCACTGGCCGTGGGACGTCATGGAGGCCATCCCAGGGCAGCAGGGGCATGGGGTAAAGAGATGTTTATGGGGAGTCTTAG | |
| CAGAGGAGGCTGGGAAGGTGTCTGAACAGTAGATGGGAGATCAGATGCCCGGAGGATTTGGGGTCTCAGCAAAGAGGGCC | |
| GAGGTGGGTGCAGGTGAGGGTCGCTGGCCCCACCCCCGGGAAGGTGCAGCAGAGCTGTGGCTCCCCACACAGCCCGGCCA | |
| GCACCTGTGCTCTGGGCATGGCTGTGCTCCTGGAACGTTCCCTGTCCTGGCTGGTCAGGGGGTGCCCCTGCCAAGAATCG | |
| ACAACTTTATCACAGAGGGAAGGGCCAATCTGTGGAGGCCACAGGGCCAGCTTCTGCCTGGAGTCAGGGCAGGTGGTGGC | |
| ACAAGCCTCGGGGCTGTACCAAAGGGCAGTCGGGCACCACAGGCCCGGGCCTCCACCTCAACAGGCCTCCCGAGCCACTG | |
| GGAGCTGAATGCCAGGAGGCCGAAGCCCTCGCCCCATGAGGGCTGAGAAGGACTGTGAGCATTTGTGTTACCCAGGGCCG | |
| AGAGCCACAGCTGCATGTTACCGCCTTTGCACCAGCTCCAGAGGCTTGGGACCAGGCTGTCTCAGTTCCAGGGTGCGTCC | |
| GGCTCAGACCGCCCTCCTCTCTGCCTTCTCTCTCTGCCTCAAATCTTCCCTCGTTTGCATCTCCCTGACGCGTGCCTGGG | |
| CCCTCGTGCAAGCTGCTTGACTCCTTTCCGGAAACCCTTGGGGTGTGCTGGATACAGGTGCCACTGAGGACTGGAGGTGT | |
| CTGACACTGTGGTTGACCCCAGGGTCCAGCTGGCGTGCTTGGGGCCTCCTTGGGCCATGATGAGGTCAGAGGAGTTTTCC | |
| CAGGTGAAAACTCCTGGGAAACTCCCAGGGCCATGTGACCTGCCACCTGCTCCTCCCATATTCAGCTCAGTCTTGTCCTC | |
| ATTTCCCCACCAGGGTCTCTAGCTCCGAGGAGCTCCCGTAGAGGGCCTGGGCTCAGGGCAGGGCGGCTGAGTTTCCCCAC | |
| CCATGTGGGGACCCTTGGGTAGTCGCTTGATTGGGTAGCCCTGAGGAGGCCGAGATGCGATGGGCCACGGGCCGTTTCCA | |
| AACACAGAGTCAGGCACGTGGAAGGCCCAGGAATCCCCTTCCCTCGAGGCAGGAGTGGGAGAACGGAGAGCTGGGCCCCG | |
| ATTTCACGGCAGCCAGGCTGCAGTGGGCGAGGCTGTGGTGGTCCACGTGGCGCTGGGGGCGGGGTCTGATTCAAATCCGC | |
| TGGGGCTCGGCCTTCCTGGCCCGTGCTGGCCGCGCCTCCACACGGGCTTGGGGTGGACGCCCCGACCTCTAGCAGGTGGC | |
| TATTTCTCCCTTTGGAAGAGAGCCCCTCACCCATGCTAGGTGTTTCCCTCCTGGGTCAGGAGCGTGGCCGTGTGGCAACC | |
| CCGGGACCTTAGGCTTATTTATTTGTTTAAAAACATTCTGGGCCTGGCTTCCGTTGTTGCTAAATGGGGAAAAGACATCC | |
| CACCTCAGCAGAGTTACTGAGAGGCTGAAACCGGGGTGCTGGCTTGACTGGTGTGATCTCAGGTCATTCCAGAAGTGGCT | |
| CAGGAAGTCAGTGAGACCAGGTACATGGGGGGCTCAGGCAGTGGGTGAGATGAGGTACACGGGGGGCTCAGGCAGTGGGT | |
| GAGGCCAGGTACATGGGGGGCTCAGGCACTGGGTGAGATGAGGTACACGGGGGGCTCAGGCAGAGGGTCAGACCAGGTAC | |
| ACGGGGGCTCTGATCACACGCACATATGAGCACATGTGCACATGTGCTGTTTCATGGTAGCCAGGTCTGTGCACACCTGC | |
| CCCAAAGTCCCAGGAAGCTGAGAGGCCAAAGATGGAGGCTGACAGGGCTGGCGCGGTGGCTCACACCTGTAGTCCCAGCA | |
| CTTTGGGAGGCCGAGGCGAGAGGATCCCTTGAGCCCAGGAGTTTAAGACCAGCCTGAGCAACATAGTAGAACCCCATCTC | |
| TATGAAAAATAAAAACAAAAATTAGCTGAACATGGTGGTGTGCGCCTGTAGTTCCAATACTTGGGAGGCTGAAGTGGGAG | |
| GATCACTTGAGCCCAGGAGGTGGAAGCTGCAGTGAGCTGAGATTGCACCACTGTACTGCAGCCTGGGTGACAGAGTGAGA | |
| GCCCATCTCAACAACAACAAAGAAGACTGACAAATGCAGTTTCTTGGAAAGAAACATTTAGTAGGAACTTAACCTACACA | |
| CAGAAGCCAAGTCGGTGTCTCGGTGTCAGTGAGATGAGATGATGGGTCCTCACACCATCACCCCAGACCCAGGGTTTATG | |
| CACCACAGGGGCGGGTGGCTCAGAAGGGATGCGCAGGACGTTGATATACGATGACATCAAGGTTGTCTGACGAAGGGCAG | |
| GATTCATGATAAGTACCTGCTGGTACACAAGGAACAATGGATAAACTGGAAACCTTAGAGGCCTTCCCGGAACAGGGGCT | |
| AATCAGAAGCCAGCATGGGGGGCTGGCATCCAGGATGGAGCTGCTTCAGCCTCCACATGCGTGTTCATACAGATGGTGCA | |
| CAGAAACGCAGTGTACCTGTGCACACACAGACACGCAGCTACTCGCACACACAAGCACACACACAGACATGCATGCATGC | |
| ATCCGTGTGTGTGCACCTGTGCCCATGAGGAAACCCATGCATGTGCATTCATGCACGCACACAGGCACCGGTGGGCCCAT | |
| GCCCACACCCACGAGCACCGTCTGATTAGGAGGCCTTTCCTCTGACGCTGTCCGCCATCCTCTCAG | |
| Intron 14 | |
| GTATGTGCCGGTGCCTGGCCTCAGTGGCAGCAGTGCCTGCCTGCTGGTGTTAGTGTGTCAGGAGACTGAGTGAATCTGGG | (WEQ ID NO 18) |
| CTTAGGAAGTTCTTACCCCTTTTCGCATCAGGAAGTGGTTTAACCCAACCACTGTCAGGCTCGTCTGCCCGCCCTCTCGT | |
| GGGGTGAGCAGAGCACCTGATGGAAGGGACAGGAGCTGTCTGGGAGCTGCCATCCTTCCCACCTTGCTCTGCCTGGGGAA | |
| GCGCTGGGGGGCCTGGTCTCTCCTGTTTGCCCCATGGTGGGATTTGGGGGGCCTGGCCTCTCCTGTTTGCCCTGTGGTGG | |
| GATTGGGCTGTCTCCCGTCCATGGCACTTAGGGCCCTTGTGCAAACCCAGGCCAAGGGCTTAGGAGGAGGCCAGGCCCAG | |
| GCTACCCCACCCCTCTCAGGAGCAGAGGCCGCGTATCACCACGACAGAGCCCCGCGCCGTCCTCTGCTTCCCAGTCACCG | |
| TCCTCTGCCCCTGGACACTTTGTCCAGCATCAGGGAGGTTTCTGATCCGTCTGAAATTCAAGCCATGTCGAACCTGCGGT | |
| CCTGAGCTTAACAGCTTCTACTTTCTGTTCTTTCTGTGTTGTGGAAATTTCACCTGGAGAAGCCGAAGAAAACATTTCTG | |
| TCGTGACTCCTGCGGTGCTTGGGTCGGGACAGCCAGAGATGGAGCCACCCCGCAGACCGTCGGGTGTGGGCAGCTTTCCG | |
| GTGTCTCCTGGGAGGGGAGCTGGGCTGGGCCTGTGACTCCTCAGCCTCTGTTTTCCCCCAG | |
| Intron 15 | |
| GCAAGTGTGGGTGGAGGCCAGTGCGGGCCCCACCTGCCCAGGGGTCATCCTTGAACGCCCTGTGTGGGGCGAGCAGCCTC | (SEQ ID NO 19) |
| AGATGCTGCTGAAGTGCAGACGCCCCCGGGCCTGACCCTGGGGGCCTGGAGCCACGCTGGCAGCCCTATGTGATTAAACG | |
| CTGGTGTCCCCAGGCCACGGAGCCTGGCAGGGTCCCCAACTTCTTGAACCCCTGCTTCCCATCTCAGGGGCGATGGCTCC | |
| CCACGCTTGGGAGCCTTCTGACCCCTGACCTGTGTCCTCTCACAGCCTCTTCCCTGGCTGCTGCCCTGAGCTCCTGGGGT | |
| CCTGAGCAAGTTCTCTCCCCGCCCCGCCGCTCCAGCGTCACTGGGCTGCCTGTCTGCTCGCCCCGGTGGAGGGGTGTCTG | |
| TCCCTTCACTGAGGTTCCCACCAGCCAGGGCCACGAGGTGCAGGCCCTGCCTGCCCGGCCACCCACACGTCCTAGGAGGG | |
| TTGGAGGATGCCACCTCTCGCCTCTTCTGGAACGGAGTCTGATTTTGGCCCCGCAG | |
| 3′-untranscribed region | |
| ATCTCATGTTTGAATCCTAATGTGCACTGCATAGACACCACTGTATGCAATTACAGAAGCCTGTGAGTGAACGGGGTGGT | (SEQ ID NO 20) |
| GGTCAGTGCGGGCCCATGGCCTGGCTGTGCATTTACGGAAGTCTATGAGTGAATGGGGTTGTGGTCAGTGCGGGCCCATG | |
| GCCTGGCTGGGCCTGGGAGGTTTCTGATGCTGTGAGGCAGGAGGGGAAGGAGGGTAGGGGATAGACAGTGGGAGCCCCCA | |
| CCCTGGAAGACATAACAGTAAGTCCAGGCCCGAAGGGCAGCAGGGATGCTGGGGGCCCAGCTTGGGCGGCGGGGATGATG | |
| GAGGGCCTGGCCAGGGTGGCAGGGATGATGGGGGCCCCAGCTGGGGTGGCAGGGGTGATGGGGGGGGCTGGTCTGGGTGG | |
| CGGGGAAGATGGGGAAGCCTGGCTGGGCCCCCTCCTCCCCTGCCTCCCACCTGCAGCCGTGGATCCGGATGTGCTTCCCT | |
| GGTGCACATCCTCTGGGCCATCAGCTTTCATGGAGGTGGGGGGCAGGGGCATGACACCATCCTGTATAAAATCCAGGATT | |
| CCTCCTCCTGAACGCCCCAACTCAGGTTGAAAGTCACATTCCGCCTCTGGCCATTCTCTTAAGAGTAGACCAGGATTCTG | |
| ATCTCTGAAGGGTGGGTAGGGTGGGGCAGTGGAGGGTGTGGACACAGGAGGCTTCAGGGTGGGGCTGGTGATGCTCTCTC | |
| ATCCTCTTATCATCTCCCAGTCTCATCTCTCATCCTCTTATCATCTCCCAGTCTCATCTGTCTTCCTCTTATCTCCCAGT | |
| CTCATCTGTCATCCTCTTACCATCTCCCAGTCTCATCTCTTATCCTCTTATCTCCTAGTCTCATCCAGACTTACCTCCCA | |
| GGGCGGGTGCCAGGCTCGCAGTGGAGCTGGACATACGTCCTTCCTCAGGCAGAAGGAACTGGAAGGATTGCAGAGAACAG | |
| GAGGGGCGGCTCAGAGGGACGCAGTCTTGGGGTGAAGAAACAGCCCCTCCTCAGAAGTTGCCTTGGGCCACACGAAACCG | |
| AGGGCCCTGCGTGAGTGGCTCCAGAGCCTTCCAGCAGGTCCCTGGTGGGGCCTTATGGTATGGCCGGGTCCTACTGAGTG | |
| CACCTTGGACAGGGCTTCTGGTTTGAGTGCAGCCCGGACGTGCCTGGTGTCGGGGTGGGGGCTTATGGCCACTGGATATG | |
| GCGTCATTTATTGCTGCTGCTTCAGAGAATGTCTGAGTGACCGAGCCTAATGTGTATGGTGGGCCCAAGTCCACAGACTG | |
| TGTCGTAAATGCACTCTGGTGCCTGGAGCCCCCGTATAGGAGCTGTGAGGAAGGAGGGGCTCTTGGCAGCCGGCCTGGGG | |
| GCGCCTTTGCCCTGCAAACTGGAAGGGAGCGGCCCCGGGCGCCGTGGGCGGACGACCTCAAGTGAGAGGTTGGACAGAAC | |
| AGGGCGGGGACTTCCCAGGAGCAGAGGCCGCTGCTCAGGCACACCTGGGTTTGAATCACAGACCAACaGGTCAGGCCATT | |
| GTTCAGCTATCCATCTTCTACAAAGCTCCAGATTCCTGTTTCTCCGGGTGTTTTTTGTTGAAATTTTACTCAGGATTACT | |
| TATATTTTTTGCTAAAGTATTAGACCCTTAAAAAAGGTATTTGCTTTGATATGGCTTAACTCACTAAGCACCTACTTTAT | |
| TTGTCTGTTTTTATTTATTATTATTATTATTATTAGAGATGGTGTCTACTCTGTCACCCAGGTTGTTAGTGCAGTGGCAC | |
| AGTCATGGCTCGCTGTAGCCGCAAACCCCCAGGCTCAAGTGATCCTCCGGCCTCAGCTTCCCAGAGTGCTGGGATTACAG | |
| GTGTGAGCCACTGCCCTTGCCTGGCACTTTTAAAAACCACTATGTAAGGTCAGGTCCAGTGGCTTCCACACCTGTCATCC | |
| CAGTAGTTTGGGAAGCCGAGGCAGAAGGATTGTCTGAGGCCAGGAGTTTGAGACCAGCATGGGTAACATAGGGAGACCCC | |
| ATCTCTACAAAAAATGCAAAAAGTTATCCGGGCGTGGGGTCCAGCATCTGTAGTCCCAGCTGCTCGGGAGGCTGAGTGGG | |
| AGGATCGCTTGAGCCCGGGAGGTCATGGCTGCAGTGAGCTGTGATTGTACCATCGCACTCCAGCCTGGGCAACAGAGTGA | |
| GACCCTGTCTCAAAAAAAAAAAAAAAAAAAGAAGGAGAAGGAGAAGAGAAGAAGAAGGAAGAAGGAAAGAGAAGAAGAAG | |
| GAAGAAGGAAGAAAGAAGGAGAAGGAGGCCTGCTAGGTGCTAGGTAGACTGTCAAATCTCAGAGCAAAATGAAAATAACA | |
| AAGTTTTAAAGGGAAAGAAAAACCCCAGCTCTTTGGACTTCCTTAGGCCTGAACTTCATCTCAAGCAGCTTCCTTCCACA | |
| GACAAGCGTGTATGGAGCGAGTGAGTTCAAAGCAGAAAGGGAGGAGAAGCAGGCAAGGGTGGAGGCTGTGGGTGACACCA | |
| GCCAGGACCCCTGAAAGGGAGTGGTTGTTTTCCTGCCTCAGCCCCACGCTCCTGCCGGTCCTGCACCTGCTGTAACCGTC | |
| GATGTTGGTGCCAGGTGCCCACCTGGGAAGGATGCTGTGCAGGGGGCTTGCCAAACTTTGGTGGGTTTCAGAAGCCCCAG | |
| GCACTTGTGGCAGGCACAATTACAGCCCCTCCCCAAAGATGCCCACGTCCTTCTCCTGGAACCTGTGAATGTGTCACCCG | |
| CAAGGCAGAGGCTGGTGAAGGCTGCAGGTGGAATCACGGCTGCCAGTCAGCCGATCTTAAGGTCATCCTGGATTATCTGG | |
| TGGGCCTGATATGGCCACAAGGGTCCCTAGAAGTGAGAGAGGGAGGCAGGGGAGAGTCAGAGAGGGGACGTGAGAAGGAC | |
| CACTGGCCACTGCTGGCTTTGAGATGGAGGAGGGGGTCCCCAGCCAAGGAATGGGGGCAGCCGCTCCATGCTGGAAAAGC | |
| AAGCAATCCTCCCCGGTCCTGAGGGCACACGGCCCTGCCCACGCCTCGATTTCAGGCCAGTGGGACCTGTTTCAGCTTTC | |
| CGGCCTCCAGAGCTGTAAGATGATGCGTTTGTGTTCAGCCACTAAGCTGCAGTGATTCGTCACAGCAGCAAATGGAATAG | |
| CAGTACAGGGAAATGAATACAGGGACAGTTCTCAGAGTGACTCTCAGCCCACCCCTGGG | |

Characterization of the exons showed, interestingly, that the functionally important hTC protein domains which are described in our Patent Application PCT/EP/98/03469 are arranged on separate exons. The telomerase-characteristic T motif is located on exon 3. The RT (reverse transcriptase) motifs 1-7, which are important for the catalytic function of the telomerase, are located on the following exons: RT motifs 1 and 2 on exon 4, RT motif 4 on exon 9, RT motif 5 on exon 10, and RT motifs 6 and 7 on exon 11. RT motif 3 is shared by exons 5 and 6 (see FIG. 8).

Elucidation of the exon-intron structure of the hTC gene also shows that the four deletions or insertion variants of the hTC CDNA which were described in our Patent Application PCT/EP/98/03469, as well as three additional hTC insertion variants which are described in the literature (Kilian et al., 1997), in all probability represent alternative splicing products. As shown in FIG. 8, the splicing variants can be divided into two groups: deletion variants and insertion variants.

The hTC variants in the deletion group lack specific sequence segments. The 36 bp in-frame deletion in variant DEL1 in all probability results from using an alternative 3′ splice acceptor sequence in exon 6, resulting in a part of RT motif 3 being lost. In variant DEL2, the normal 5′ splice donor and 3′ splice acceptor sequences of introns 6, 7 and 8 are not used. Instead exon 6 is fused directly to exon 9, resulting in a displacement arising in the open reading frame and a stop codon appearing in exon 10. Variant Del3 is a combination of variants 1 and 2.

The insertion variant group is characterized by the insertion of intron sequences which lead to premature cessation of translation. Instead of the 5′ splice donor sequence of intron 5, which is normally used, use is made, in variant INS1, of an alternative, 3′-located splice site, resulting in the insertion of the first 38 bp from intron 4 between exon 4 and exon 5. The insertion, in variant INS2, of a region of the 11 sequence likewise results from using an alternative 5′ splice donor sequence in intron 11. since this variant was only described inadequately in the literature (Kilian et al., 1997), it is not possible to determine the precise alternative 5′ splice donor sequence in this variant. The insertion of intron 14 sequences between exon 14 and exon 15 in variant INS3 comes from using an alternative 3′ splice acceptor sequence, resulting in the 3′ part of intron 14 not being spliced.

The hTC variant INS4 (variante 4), which is described in our Patent Application PCT/EP/98/03469, is characterized by exon 15, and the 5′ part region of exon 16, being replaced by the first 600 bp of intron 14. This variant can be attributed to the use of an alternative internal 5′ splice donor sequence in intron 14 and an alternative 3′ splice acceptor sequence in exon 16, resulting in an altered C terminus.

The in vivo generation of hTC protein variants which are probably non-functional and which could interfere with the function of the complete hTC protein constitutes a possible mechanism, in addition to transcription regulation, for controlling hTC protein function. The function of the hTC splicing variants is not yet known. Although most of these variants presumably encode proteins without reverse transcriptase activity, they could nevertheless play a crucial role as transdominant-negative telomerase regulators by, for example, competing for interaction with important binding partners.

The search for possible transcription factor binding sites was carried out using the ,,find pattern” algorithm from the Genetics Computer Group (Madison, USA) GCG Sequence Analysis program package. This resulted in the identification of a variety of potential binding sites for transcription factors in the nucleotide sequence of intron 2, which binding sites are listed in Tab. 2. In addition, an SpI binding site was found in intron 1 (pos. 43), and a c-Myc binding site was found in the 5′-untranslated region (cDNA position 29-34, cf. FIG. 6).

### Example 6

In order to ascertain the start point(s) of hTC transcription in HL 60 cells, the 5′ end of the hTC mRNA was determined by means of primer extension analysis.

2 μg of polyA⁺ RNA from HL-60 cells were denaturated at 65° C. for 10 min. 1 μl of RNasin (3040 U/ml) and 0.3-1 pmol of radioactively labelled primer (5′GTTAAGTTGTAGCTTACACTGGTTCTC 3′; 2.5-8×10⁵ cpm) were added for primer annealing, and the whole was incubated, at 37° C. for 30 min, in a total volume of 20 μl. After the addition of 10 μl of 5xreverse transcriptase buffer (from Gibco-BRL), 2 μl of 10 mM dNTPs, 2 μl RNasin (see above), 5 μl of 0.1 M DTT (from Gibco-BRL) 2 μl of ThermoScript RT (15 U/μl; from Gibco-BRL) and 9 μl of DEPC-treated water, primer extension took place, at 58° C. for 1 h, in a total volume [lacuna]. The reaction was stopped by adding 4 μl of 0.5 M EDTA, pH 8.0, and the RNA was degraded, at 37° C. for 30 min, after having added 1 μl of RNaseA (10 mg/ml). 2.5 μg of sheared calf thymus DNA and′ 100 μl of TE were then added, and the mixture was extracted once with 150 μl of phenol/chloroform (1:1). The DNA was precipitated, at −70° C. for 45 min, after adding 15 μl of 3 M Na acetate and 450 μl of ethanol, and then centrifuged at 14,000 rpm for 15 min. The precipitate was washed once with 70% ethanol, dried in air and dissolved in 8 μl of sequencing stop solution. After 5 min of denaturation at 80° C., the samples were loaded onto a 6% polyacrylamide gel and fractionated electrophoretically (Ausubel et al., 1987) (FIG. 5).

In this connection, a main transcription start site was identified which is located 1767 bp 5′ of the ATG start codon of the hTC cDNA sequence (nucleotide position 3346 in FIG. 4). In addition to this, the nucleotide sequence around this main transcription start (TTA₋₁TTGT) represents an initiator element (Inr), which, in 6 out of 7 nucleotides, matches the consensus motif (PyPyA₊₁Na/tPyPy) (Smale, 1997) of an initiator element.

It was not possible to identify any unambiguous TATA box in the immediate vicinity of the experimentally identified main transcription start, which means that the hTC promoter has probably to be classified in the family of TATA-less promoters (Smale, 1997). However, a potential TATA box from nucleotide position 1306 to nucleotide position 1311 (FIG. 4) was found by means of bioinformatics analysis. The subsidiary transcription starts which were additionally observed around the main transcription start have also been described in the case of other TATA-less promoters (Geng and Johnson, 1993), for example in the strongly regulated promoters of some cell cycle genes (Wick et al., 1995).

### Example 7

In addition to the start point of the hTC transcript which was described in Example 6 and identified in HL60 cells, a further transcription start region was also identified in HL60 cells. With the aid of RT-PCR analyses, the region of the hTC gene transcription start in HL60 cells was localized to bp -60 to bp -105.

The cDNA for this was synthesized using a First Strand cDNA Synthesis kit (Clontech), in accordance with the manufacturer's instructions, and employing 0.4 μg of HL60 cell polyA RNA (Clontech) and the gene-specific primer GSP13 (5′-CCTCCAAAGAGGTGGCTTCTTCGGC-3′, cDNA position 920-897). In a final volume of 50 μl, 10 pmol dNTP mix were added to 1 μl of cDNA, and a PCR reaction was carried out in 1×PCR reaction buffer F (PCR-Optimizer kit from InVitrogen) and using one unit of platinum Taq DNA polymerase (from Gibco/BRL). 10 pmol of each of the 5′ and 3′ primers defined below were added as primers. The PCR was carried out in 3 steps. A two-minute denaturation at 94° C. was followed by 36 PCR cycles in which the DNA was first of all denatured at 94° C. for 45 sec and, after that, the primers were annealed, and the DNA chain was extended at 68° C. for 5 min. The cycles were concluded by a chain extension at 68° C. for 10 min. In all, six different PCR primers (primer HTRT5B: 5′-CGCAGCCACTACCGCGAGGTGC-3′ cDNA position 105 to 126; primer C5S: 5′-CTGCGTCCTGCTGCGCACGTGGGAAGC-3′, 5′-flanking region -49 to -23; primer PRO-TEST1: 5′-CTCGCGGCGCGAGTTTCAGGCAG-3′, 5′-flanking region -74 to -52; primer PRO-TEST2: 5′-CCAGCCCCTCCCCTTCCTTTCC-3′, 5′-flanking region -112 to -91; primer PRO-TEST4: 5′-CCAGCTCCGCCTCCTCCGCGC-3′, 5′-flanking region -191 to -171; primer RP-3A: 5′-CTAGGCCGATTCGACCTCTCTCC-3′, 5′-flanking region -427 to -405) were combined with the 3′ PCR primer C5Rback (5′-GTCCCAGGGCACGCACACCAG-3′, cDNA position 245 to 225). Genomic DNA was also employed for the PCR, as a control, in addition to the Oligo dT- and GSP13-primed cDNAs. As FIG. 9 shows, a PCR product was only obtained with the primer combinations HTRT5B-C5Rback, C5S-C5Rback and PRO-TEST1-C5Rback, indicating that the start point for hTC transcription lies in the region between bp-60 and bp-105.

### Example 8

Several extremely GC-rich regions, so-called CpG Islands, are located in the isolated 5′-flanking region, of about 11.2 kb in size, of the hTC gene. One CpG Island, having a GC content of >70%, extends from bp -1214 into intron 2. Two further GC-rich regions having a GC content of >60% extend from bp -3872 to bp -3113 and from bp -5363 to bp -3941, respectively. The positions of the CpG Islands are shown graphically in FIG. 11.

The search for possible transcription factor binding sites was carried out using the “Find Pattern” algorithm from the Genetics Computer Group (Madison, USA) GCG Sequence Analysis program package. This resulted in the identification of a variety of potential binding sites in the region up to -900 bp upstream of the translation start codon ATG: five Sp1 binding sites, one c-Myc binding site, and one CCAC box (FIG. 10). In addition, a CCAAT box and a second c-Myc binding site were found at positions -1788 and -3995, respectively, of the 5′-flanking region.

### Example 9

In order to analyse the activity of the hTC promoter, PCR amplification was used to generate four hTC promoter sequence segments of differing length, which segments were cloned into the Promega vector pGL2 5′ in front of the luciferase reporter gene. The 8.5 kb SacI fragment which was subcloned from phage clone P12 was selected as the DNA source for the PCR amplification. In a final volume of 50 μl, 10 pmol of dNTP mix were added to 35 ng of this DNA, and a PCR reaction was carried out in 1×PCR reaction buffer (PCR-Optimizer kit from InVitrogen) and using one unit of platinum Taq DNA polymerase (from Gibco/BRL). In each case 20 pmol of the 5′ and 3′ primers which are defined below were added as primers. The PCR was carried out in three steps. A two-minute denaturation at 94° C. was followed by 30 PCR cycles in which the DNA was first of all denaturated at 94° C. for 45 sec, after which the primers were annealed, and the DNA chain was extended, at 68° C. for 5 min. The cycles were concluded by a chain extension at 68° C. for 10 min. The selected 3′ PCR primer was in each case the primer PK-3A (5′-GCAAGCTTGACGCAGCGCTGCCTGAAACTCG-3′, position 43 to -65), which primer recognizes a sequence region 42 bp upstream of the ATG START codon. A promoter fragment of 4051 bp in size (NPK8) was amplified by combining the PK-3A primers with the 5′ PCR primer PK-5B (5′-CCAGATCTCTGGAACACAGAGTGGCAGTTTCC-3′, position 4093 to -4070). Combining the pair of primers PK-3A and PK-5C (5′-CCAGATCTGCATGAAGTGTGTGGGGATTTGCAG-3′, position -3120 to -3096) led to the amplification of a promoter fragment of 3078 bp in size (NPK15). Use of the primer combination PK-3A and PK-5D (5′-GGAGATCTGATCTTGGCTTACTGCAGCCTCTG-3′, position -2110 to -2087) amplified a promoter fragment of 2068 bp in size (NPK22). Finally, using the primer combination PK-3A and PK-5E (5′-GGAGATCTGTCTGGATTCCTGGGAAGTCCTCA-3′, position - 1125 to -1102) led to the amplification of a promoter fragment of 1083 bp in size (NPK27).

The PK-3A primer contains a HindIII recognition sequence. The different 5′ primers contain a BglII recognition sequence.

The resulting PCR products were purified using the Qiagen QIA quick spin PCR purification kit, in accordance with the manufacturer's instructions, and then digested with the restriction enzymes BglII and HindIII. The pGL2 promoter vector was digested with the same restriction enzymes, and the SV40 promoter contained in this vector was released and removed. The PCR promoter fragments ligated into the vector, which was then transformed into competent DH5α bacteria (from Gibco/BRL). DNA for the promoter activity analyses, which are described below, was isolated from transformed bacterial clones using the Qiagen plasmid kit.

### Example 10

The activity of the hTC promoter was analysed in transient transfections in eukaryotic cells.

All the work with eukaryotic cells was carried out at a sterile workstation. CHO-K1 and HEK 293 cells were obtained from the American Type Culture collection.

CHO-K1 cells were kept in DMEM Nut Mix F-12 cell culture medium (from Gibco-BRL, order number: 21331-020) containing 0.15% streptomycin/penicillin, 2 mM glutamine and 10% FCS (from Gibco-BR1).

HEK 293 cells were cultured in DMOD cell culture medium (from Gibco-BRL, order number: 41965-039) containing 0.15% streptomycin/penicillin, 2 mM glutamine and 10% FCS (from Gibco-BRL).

CHO-K1 and HEK 293 cells were cultured at 37° C. in a water-saturated atmosphere while being gassed with 5% CO₂. When the cell lawn was confluent, the medium was sucked off, after which the cells were washed with PBS (100 mM KH₂PO₄ pH 7.2; 150 mM NaCl) and released by adding a trypsin-EDTA solution (from Gibco-BRL). The trypsin was inactivated by adding medium and the cell count was determined using a Neubauer counting chamber in order to plate out the cells at the desired density.

For the transfection, in each case 2×10⁵ HEK 293 cells were plated out, per well, in a 24-well cell culture plate. The HEK 293 medium was removed after 3 hours. For the transfection, up to 2.5 μg of plasmid DNA, 1 μg of a CMV β-Gal plasmid construct (from Stratagene, order number: 200388), 200 μl of serum-free medium and 10 μl of transfection reagent (DOTAP from Boehringer Mannheim) were incubated at room temperature for 15 minutes and then dropped uniformly onto the HEK 293 cells. 1.5 ml of medium were added after 3 hours. Tbf medium was changed after 20 hours. After a further 24 hours, the cells were harvested for determining the luciferase activity and the β-Gal activity. For this, the cells were lysed, at room temperature for 15 minutes, in the cell culture lysis reagent (25 mM Tris [nH 7.8] containing H₃PO₄; 2 mM CDTA; 2 mM DTT; 10% glycerol; 1% Triton X-100). Twenty μl of this cell lysate were mixed with 100 μl of luciferase assay buffer (20 mM Tricin; 1.07 mM (MgCO₃)₄ Mg(OH)₂.5H₂O; 2.67 mM MgSO₄; 0.1 mM EDTA; 33.3 mM DTT; 270 μM coenzyme A; 470 μM luciferin, 530 μM ATP), and the light generated by the luciferase was measured.

In order to measure the β-galactosidase activity, equal quantities of cell lysate and β-galactosidase assay buffer (100 mM sodium phosphate buffer, pH 7.3; 1 mM MgCl₂; 50 mM β-mercaptoethanol; 0.665 mg of ONPG/ml) were incubated at 37° C. for at least 30 minutes or until a slight yellow coloration appeared. The reaction was stopped by adding 100 μl of 1 M Na₂CO₃, and the absorption was determined at 420 nm.

In order to analyse the hTC promoter, four hTC promoter sequence segments of differing length were cloned 5′ in front of the luciferase reporter gene (cf. Example 9).

The relative luciferase activities of two independent transfections in HEK 293 cells, using the constructs NPK8, NPK15, NPK22 and NPK27, are plotted in FIG. 11. Each experiment was carried out in duplicate. The standard deviation has also been given. The construct NPK 27 exhibits a luciferase activity which is 40 times higher than the basal activity of the promoterless luciferase control construct (pGL2-basic) and from 2 to 3 times higher than that of the SV40 promoter control construct (pGL2PRO). Interestingly, a luciferase activity which was from 2 to 3 times lower than that obtained with the NPK 27 construct was observed in cells which were transfected with longer hTC promoter constructs (NPK8, NPK15, NPK22). Similar results were also observed in CHO cells (data not shown).

### REFERENCES

Allsopp, R. C., Vazire, H., Pattersson, C., Goldstein, S., Younglai, E. V., Futcher, A. B., Greider, C. W. und Harley, C. B. (1992). Telomere length predicts replicative capacity of human fibroblasts. Proc. Natl. Acad. Sci. 89, 10114-10118.
Ausubel, F. M., Brent, R., Kingston, R. E., Moore, D. D., Seidman, J. G., Smith, J. A., Struhl, K. (1987). Current protocols in molecular biology. Greene Publishing Associates and Whiley-lntersciences, New York.
Blasco, M. A., Rizen, M., Greider, C. W. und Hanahan, D. (1996). Differential regulation of telomerase activity and telomerase RNA during multistage tumorigenesis. Nature Genetics 12, 200-204.
Broccoli, D., Young, J. W. und deLange, T. (1995). Telomerase activity in normal and malignant hematopoietic cells. Proc. Natl. Acad. Sci. 92, 9082-9086.

Counter, C. M., Avilion, A. A., LeFeuvre, C. E., Stewart, N. G. Greider, C. W. Harley, C. B. und Bacchetti S. (1992). Telomere shortening associated with chromosome instability is arrested in immortal cells which express telomerase activity. EMBO J. 11, 1921-1929.
Feng, J., Funk, W. D., Wang, S.-S., Weinrich, S. L., Avilion, A. A., Chiu, C.-P., Adams, R. R., Chang, E., Allsopp, R. C., Yu, J., Le, S., West, M. D., Harley, C. B., Andrews, W. H., Greider, C. W. und Villepotioeau, B. (1995). The RNA component of human telomerase. Science 269, 1236-1241.

Geng, Y., and Johnson, L. F. (1993). Lack of an initiator element is responsible for multiple transcriptional initiation sites of the TATA less mouse thymidine synthasse promoter. Mol. Cell. Biol 14:4894.
Goldstein, S. (1990), Replicative senescence: The human fibroblast comes of age Science 249. 1129-1133.
Harley, C. B., Futcher, A. B., Greider, C. W., 1990. Telomeres shorten during ageing of human fibroblasts. Nature 3-45, 458-460.
Hastie, N. D., Dempster, M., Dunlop, M. G., Thompson, A. M., Green, D. K. und Allshire, R. C. (1990). Telomere reduction in human colorectal carcinoma and with ageing. Nature 346. 866-868.
Hiyama, K., Hirai, Y., Kyoizumi, S., Akiyama, M., Hiyama, E., Piatyszek, M. A., Shay, J. W., Ishioka, S. und Yamakido, M. (1995). Activation of telomerase in human lymphocytes and hematopoietic progenitor cells. J. Immunol. 155, 3711-3715.
Kim, N. W., Piatyszek, M. A., Prowse, K. R., Harley, C. B., West, M. D., Ho, P. L. C., Coviello, G. M., Wright, W. E., Weinrich, S. L. und Shay, J. W. (1994). Specific association of human telomerase activity with immortal cells and cancer. Science 266. 2011-2015.
Latchman, D. S. (1991). Eukaryotic transcription factors. Academic Press Limited, London.
Lingner, J., Hughes, T. R., Shevchenko, A., Mann, M., Lundblad, V. und Cech T. R. (1997). Reverse transcriptase motifs in the catalytic subunit of telomerase. Science 276: 561-567.
Lundblad, V. und Szostak, J. W. (1989). A mutant with a defect in telomere elongation leads to senescence in yeast. Cell 57, 633-643.
McClintock, B. (1941). The stability of broken ends of chromosomes in Zea mays. Genetics 26, 234-282.
Meyne, J., Ratliff, R. L. und Moyzis, R. K. (1989). Conservation of the human telomere sequence (TTAGGG)ₙ among vertebrates. Proc. Natl. Acad. Sci. 86, 7049-7053.
Olovnikov, A. M. (1973). A theory of marginotomy J. Theor. Biol. 41, 181-190.
Sandell, L. L. und Zakian, V. A. (1993). Loss of a yeast telomere: Arrest. recovery and chromosome loss. Cell 75 729-739.
Shapiro, M. B., Senapathy, P., 1987. RNA splice junctions of different classes of eukaryotes: sequence statistics and functional implications in gene expression. Nucl. Acids Res. 15. 7155-7174.
Smale, S. T. and Baltimore, D. (1989). The initiator as a transcription control element. Cell 57:103-113.
Smale, S. T. (1997). Transcription initiation from TATA-less promoters within eukaryotic protein-coding genes. Biochimica et Biophysica Acta 1351, 73-88.
Shay, J. W. (1997). Telomerae and Cancer. Ciba Foundation Meeting: Telomeres and Telomerase. London.
Vaziri, H., Dragowskal W., Allsopp, R. C., Thomas, T. E., Harley, C. B. und Landsdorp, P. M. (1994). Evidence for a mitotic clock in human hematopoietic stem cells: Loss of telomeric DNA with age. Proc. Natl. Acad. Sci. 91, 9857-9860.
Wick, M., Haironen, R., Mumberg, D., Burger, C., Olsen, B. R., Budarf, M. L., Apte, S. S. and Miller, R. (1995). Structure of the human TIMP-3 gene and its cell-cycle-regulated promoter. Biochemical Journal 311, 549-554.
Zakian, V. A. (1995). Telomeres: Beginning to understand the end. Science 270. 1601-1607.

1. Regulatory DNA sequences for the gene for the human catalytic telomerase subunit.

2. DNA sequences according to claim 1, characterized in that the sequences are intron sequences in accordance with SEQ ID NO 4, 5, 6, 7, 8, 9, 10, 11, 12, 13, 14, 15, 16, 17, 18, 19 and/or 20 or fragments of these sequences which have a regulatory effect.

3. DNA sequences according to claim 1, characterized in that the sequences are the 5′-flanking regulatory DNA sequence for the gene for the human catalytic telomerase subunit as depicted in FIG. 10 (SEQ ID NO 3), or fragments of this DNA sequence which have a regulatory effect.

4. Recombinant construct which contains a DNA. sequence according to one of claims 1 to 3.

5. Recombinant construct according to claim 4, characterized in that it additionally contains one or more DNA sequences which encode polypeptides or proteins.

6. Vector which contains a recombinant construct according to claim 4 or 5.

7. Use of recombinant constructs or vectors according to one of claims 4 to 6 for preparing medicaments.

8. Recombinant host cells which harbour recombinant constructs or vectors according to one of claims 4 to 6.

9. Process for identifying substances which affect the promoter activity, silencer activity or enhancer activity of the human catalytic telomerase subunit, comprising the following steps:
A. adding a candidate substance to a host cell which harbours DNA sequences according to one of claims 1 to 3, which sequences are functionally linked to a reporter gene, and
B. measuring the effect of the substance on expression of the reporter gene.

10. Process for identifying factors which bind specifically to the DNA according to one of claims 1 to 3, or to fragments thereof, characterized in that an expression cDNA library is screened using a DNA sequence according to one of claims 1 to 3, or subfragments of widely differing length, as the probe.

11. Transgenic animals which harbour recombinant constructs or vectors according to claims 4 to 6.

12. Process for detecting telomerase-associated conditions in a patient, comprising the following steps:
A. incubating a recombinant construct or vector according to claims 4 to 6, which additionally contains a reporter gene, with body fluids or cell samples,
B. detecting the activity of the reporter gene in order to obtain a diagnostic value, and
C. comparing the diagnostic value with standard values for the reporter gene construct in standardized normal cells or body fluids of the same type as the test sample.


SEQUENCE LISTING (44 sequences)

SEQ ID NO: 1 - DNA, 5126 bp, Homo sapiens
gagctctgaaccgtggaaacgaacatgacccttgcctgcctgcttccctgggtgggtcaagggtaatgaagtggtgtgcaggaaatggccatgtaaattacacgactctgctgatggggaccgttccttccatcattattcatcttcacccccaaggactgaatgattccagcaacttcttcgggtgtgacaagccatgacaaaactcagtacaaacaccactcttttactaggcccacagagcacgggccacacccctgatatattaagagtccaggagagatgaggctgctttcagccaccaggctggggtgacaacagcggctgaacagtctgttcctctagactagtagaccctggcaggcactcccccaaattctagggcctggttgctgcttcccgagggcgccatctgccctggagactcagcctggggtgccacactgaggccagccctgtctccacaccctccgcctccaggcctcagcttctccagcagcttcctaaaccctgggtgggccgtgttccagcgctactgtctcacctgtcccactgtgtcttgtctcagcgacgtagctcgcacggttcctcctcacatggggtgtctgtctccttccccaacactcacatgcgttgaagggaggagattctgcgcctcccagactggctcctctgagcctgaacctggctcgtggcccccgatgcaggttcctggcgtccggctgcacgctgacctccatttccaggcgctccccgtctcctgtcatctgccggggcctgccggtgtgttcttctgtttctgtgctcctttccacgtccagctgcgtgtgtctctgcccgctagggtctcggggtttttataggcataggacgggggcgtggtgggccagggcgctcttgggaaatgcaacatttgggtgtgaaagtaggagtgcctgtcctcacctaggtccacgggcacaggcctggggatggagcccccgccagggacccgcccttctctgcccagcactttcctgcccccctccctctggaacacagagtggcagtttccacaagcactaagcatcctcttcccaaaagacccagcattggcacccctggacatttgccccacagccctgggaattcacgtgactacgcacatcatgtacacactcccgtccacgaccgacccccgctgttttattttaatagctacaaagcagggaaatccctgctaaaatgtcctttaacaaactggttaaacaaacgggtccatccgcacggtggacagttcctcacagtgaagaggaacatgccgtttataaagcctgcaggcatctcaagggaattacgctgagtcaaaactgccacctccatgggatacgtacgcaacatgctcaaaaagaaagaatttcaccccatggcaggggagtggttaggggggttaaggacggtgggggcggcagctgggggctactgcacgcaccttttactaaagccagtttcctggttctgatggtattggctcagttatgggagactaaccataggggagtggggatgggggaacccggaggctgtgccatctttgccatgcccgagtgtcctgggcaggataatgctctagagatgcccacgtcctgattcccccaaacctgtggacagaacccgcccggccccagggcctttgcaggtgtgatctccgtgaggaccctgaggtctgggatccttcgggactacctgcaggcccgaaaagtaatccaggggttctgggaagaggcgggcaggagggtcagaggggggcagcctcaggacgatggaggcagtcagtctgaggctgaaaagggagggagggcctcgagcccaggcctgcaagcgcctccagaagctggaaaaagcggggaagggaccctccacggagcctgcagcaggaaggcacggctggcccttagcccaccagggcccatcgtggacctccggcctccgtgccataggagggcactcgcgctgcccttctagcatgaagtgtgtggggatttgcagaagcaacaggaaacccatgcactgtgaatctaggattatttcaaaacaaaggtttacagaaacatccaaggacagggctgaagtgcctccgggcaagggcagggcaggcacgagtgattttatttagctattttattttatttacttactttctgagacagagttatgctcttgttgcccaggctggagtgcagcggcatgatcttggctcactgcaacctccgtctcctgggttcaagcaattctcgtgcctcagcctcccaagtagctgggatttcaggcgtgcaccaccacacccggctaattttgtatttttagtagagatgggctttcaccatgttggtcaagctgatctcaaaatcctgacctcaggtgatccgcccacctcagcctcccaaagtgctgggattacaggcatgagccactgcacctggcctatttaaccattttaaaacttccctgggctcaagtcacacccactggtaaggagttcatggagttcaatttcccctttactcaggagttaccctcctttgatattttctgtaattcttcgtagactggggatacaccgtctcttgacatattcacagtttctgtgaccacctgttatcccatgggacccactgcaggggcagctgggaggctgcaggcttcaggtcccagtggggttgccatctgccagtagaaacctgatgtagaatcagggcgcaagtgtggacactgtcctgaatctcaatgtctcagtgtgtgctgaaacatgtagaaattaaagtccatccctcctactctactgggattgagccccttccctatccccccccaggggcagaggagttcctctcactcctgtggaggaaggaatgatactttgttatttttcactgctggtactgaatccactgtttcatttgttggtttgtttgttttgttttgagaggcggtttcactcttgttgctcaggctggagggagtgcaatggcgcgatcttggcttactgcagcctctgcctcccaggttcaagtgattctcctgcttccgcctcccatttggctgggattacaggcacccgccaccatgcccagctaattttttgtatttttagtagagacgggggtgggtggggttcaccatgttggccaggctggtctcgaacttctgacctcagatgatccacctgcctctgcctcctaaagtgctgggattacaggtgtgagccaccatgcccagctcagaatttactctgtttagaaacatctgggtctgaggtaggaagctcaccccactcaagtgttgtggtgttttaagccaatgatagaatttttttattgttgttagaacactcttgatgttttacactgtgatgactaagacatcatcagcttttcaaagacacactaactgcacccataatactggggtgtcttctgggtatcagcaatcttcattgaatgccgggaggcgtttcctcgccatgcacatggtgttaattactccagcataatcttctgcttccatttcttctcttccctcttttaaaattgtgttttctatgttggcttctctgcagagaaccagtgtaagctacaacttaacttttgttggaacaaattttccaaaccgcccctttgccctagtggcagagacaattcacaaacacagccctttaaaaaggcttagggatcactaaggggatttctagaagagcgacctgtaatcctaagtatttacaagacgaggctaacctccagcgagcgtgacagcccagggagggtgcgaggcctgttcaaatgctagctccataaataaagcaatttcctccggcagtttctgaaagtaggaaaggttacatttaaggttgcgtttgttagcatttcagtgtttgccgacctcagctacagcatccctgcaaggcctcgggagacccagaagtttctcgcccccttagatccaaacttgagcaacccggagtctggattcctgggaagtcctcagctgtcctgcggttgtgccggggccccaggtctggaggggaccagtggccgtgtggcttctactgctgggctggaagtcgggcctcctagctctgcagtccgaggcttggagccaggtgcctggaccccgaggctgccctccaccctgtgcgggcgggatgtgaccagatgttggcctcatctgccagacagagtgccggggcccagggtcaaggccgttgtggctggtgtgaggcgcccggtgcgcggccagcaggagcgcctggctccatttcccaccctttctcgacgggaccgccccggtgggtgattaacagatttggggtggtttgctcatggtggggacccctcgccgcctgagaacctgcaaagagaaatgacgggcctgtgtcaaggagcccaagtcgcggggaagtgttgcagggaggcactccgggaggtcccgcgtgcccgtccagggagcaatgcgtcctcgggttcgtccccagccgcgtctacgcgcctccgtcctccccttcacgtccggcattcgtggtgcccggagcccgacgccccgcgtccggacctggaggcagccctgggtctccggatcaggccagcggccaaagggtcgccgcacgcacctgttcccagggcctccacatcatggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgatg

SEQ ID NO: 2 - DNA, 4042 bp, Homo sapiens
gtttcaggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaaaaaaaaaaaaaaaaaaaaaa

SEQ ID NO: 3 - DNA, 11276 bp, Homo sapiens
acttgagcccaagagttcaaggctacggtgagccatgattgcaacaccacacgccagccttggtgacagaatgagaccctgtctcaaaaaaaaaaaaaaaaattgaaataatataaagcatcttctctggccacagtggaacaaaaccagaaatcaacaacaagaggaattttgaaaactatacaaacacatgaaaattaaacaatatacttctgaatgaccagtgagtcaatgaagaaattaaaaaggaaattgaaaaatttatttaagcaaatgataacggaaacataacctctcaaaacccacggtatacagcaaaagcagtgctaagaaggaagtttatagctataagcagctacatcaaaaaagtagaaaagccaggcgcagtggctcatgcctgtaatcccagcactttgggaggccaaggcgggcagatcgcctgaggtcaggagttcgagaccagcctgaccaacacagagaaaccttgtcgctactaaaaatacaaaattagctgggcatggtggcacatgcctgtaatcccagctactcgggaggctgaggcaggataaccgcttgaacccaggaggtggaggttgcggtgagccgggattgcgccattggactccagcctgggtaacaagagtgaaaccctgtctcaagaaaaaaaaaaaagtagaaaaacttaaaaatacaacctaatgatgcaccttaaagaactagaaaagcaagagcaaactaaacctaaaattggtaaaagaaaagaaataataaagatcagagcagaaataaatgaaactgaaagataacaatacaaaagatcaacaaaattaaaagttggttttttgaaaagataaacaaaattgacaaacctttgcccagactaagaaaaaaggaaagaagacctaaataaataaagtcagagatgaaaaaagagacattacaactgataccacagaaattcaaaggatcactagaggctactatgagcaactgtacactaataaattgaaaaacctagaaaaaatagataaattcctagatgcatacaacctaccaagattgaaccatgaagaaatccaaagcccaaacagaccaataacaataatgggattaaagccataataaaaagtctcctagcaaagagaagcccaggacccaatggcttccctgctggattttaccaatcatttaaagaagaatgaattccaatcctactcaaactattctgaaaaatagaggaaagaatacttccaaactcattctacatggccagtattaccctgattccaaaaccagacaaaaacacatcaaaaacaaacaaacaaaaaaacagaaagaaagaaaactacaggccaatatccctgatgaatactgatacaaaaatcctcaacaaaacactagcaaaccaaattaaacaacaccttcgaaagatcattcattgtgatcaagtgggatttattccagggatggaaggatggttcaacatatgcaaatcaatcaatgtgatacatcatcccaacaaaatgaagtacaaaaactatatgattatttcactttatgcagaaaaagcatttgataaaattctgcacccttcatgataaaaaccctcaaaaaaccaggtatacaagaaacatacaggccaggcacagtggctcacacctgcgatcccagcactctgggaggccaaggtgggatgattgcttgggcccaggagtttgagactagcctgggcaacaaaatgagacctggtctacaaaaaacttttttaaaaaattagccaggcatgatggcatatgcctgtagtcccagctagtctggaggctgaggtgggagaatcacttaagcctaggaggtcgaggctgcagtgagccatgaacatgtcactgtactccagcctagacaacagaacaagaccccactgaataagaagaaggagaaggagaagggagaagggagggagaagggaggaggaggagaaggaggaggtggaggagaagtggaaggggaaggggaagggaaagaggaagaagaagaaacatatttcaacataataaaagccctatatgacagaccgaggtagtattatgaggaaaaactgaaagcctttcctctaagatctggaaaatgacaagggcccactttcaccactgtgattcaacatagtactagaagtcctagctagagcaatcagataagagaaagaaataaaaggcatccaaactggaaaggaagaagtcaaattatcctgtttgcagatgatatgatcttatatctggaaaagacttaagacaccactaaaaaactattagagctgaaatttggtacagcaggatacaaaatcaatgtacaaaaatcagtagtatttctatattccaacagcaaacaatctgaaaaagaaaccaaaaaagcagctacaaataaaattaaacagctaggaattaaccaaagaagtgaaagatctctacaatgaaaactataaaatgttgataaaagaaattgaagagggcacaaaaaaagaaaagatattccatgttcatagattggaagaataaatactgttaaaatgtccatactacccaaagcaatttacaaattcaatgcaatccctattaaaatactaatgacgttcttcacagaaatagaagaaacaattctaagatttgtacagaaccacaaaagacccagaatagccaaagctatcctgaccaaaaagaacaaaactggaagcatcacattacctgacttcaaattatactacaaagctatagtaacccaaactacatggtactggcataaaaacagatgagacatggaccagaggaacagaatagagaatccagaaacaaatccatgcatctacagtgaactcatttttgacaaaggtgccaagaacatactttggggaaaagataatctcttcaataaatggtgctggaggaactggatatccatatgcaaaataacaatactagaactctgtctctcaccatatacaaaagcaaatcaaaatggatgaaaggcttaaatctaaaacctcaaactttgcaactactaaaagaaaacaccggagaaactctccaggacattggagtgggcaaagacttcttgagtaattccctgcaggcacaggcaaccaaagcaaaaacagacaaatgggatcatatcaagttaaaaagcttctgcccagcaaaggaaacaatcaacaaagagaagagacaacccacagaatgggagaatatatttgcaaactattcatctaacaaggaattaataaccagtatatataaggagctcaaactactctataagaaaaacacctaataagctgattttcaaaaataagcaaaagatctgggtagacatttctcaaaataagtcatacaaatggcaaacaggcatctgaaaatgtgctcaacaccactgatcatcagagaaatgcaaatcaaaactactatgagagatcatctcatcccagttaaaatggcttttattcaaaagacaggcaataacaaatgccagtgaggatgtggataaaaggaaacccttggacactgttggtgggaatggaaattgctaccactatggagaacagtttgaaagttcctcaaaaaactaaaaataaagctaccatacagcaatcccattgctaggtatatactccaaaaaagggaatcagtgtatcaacaagctatctccactcccacatttactgcagcactgttcatagcagccaaggtttggaagcaacctcagtgtccatcaacagacgaatggaaaaagaaaatgtggtgcacatacacaatggagtactacgcagccataaaaaagaatgagatcctgtcagttgcaacagcatggggggcactggtcagtatgttaagtgaaataagccaggcacagaaagacaaacttttcatgttctcccttacttgtgggagcaaaaattaaaacaattgacatagaaatagaggagaatggtggttctagaggggtgggggacagggtgactagagtcaacaataatttattgtatgttttaaaataactaaaagagtataattgggttgtttgtaacacaaagaaaggataaatgcttgaaggtgacagataccccatttaccctgatgtgattattacacattgtatgcctgtatcaaaatatctcatgtatgctatagatataaaccctactatattaaaaattaaaattttaatggccaggcacggtggctcatgtccgtaatcccagcactttgggaggccgaggcgggtggatcacctgaggtcaggagtttgaaaccagtctggccaccatgatgaaaccctgtctctactaaagatacaaaaattagccaggcgtggtggcacatacctgtagtcccaactactcaggaggctgagacaggagaattgcttgaacctgggaggcggaggttgcagtgagccgagatcatgccactgcactgcagcctgggtgacagagcaagactccatctcaaaacaaaaacaaaaaaaagaagattaaaattgtaatttttatgtaccgtataaatatatactctactatattagaagttaaaaattaaaacaattataaaaggtaattaaccacttaatctaaaataagaacaatgtatgtggggtttctagcttctgaagaagtaaaagttatggccacgatggcagaaatgtgaggagggaacagtggaagttactgttgttagacgctcatactctctgtaagtgacttaattttaaccaaagacaggctgggagaagttaaagaggcattctataagccctaaaacaactgctaataatggtgaaaggtaatctctattaattaccaataattacagatatctctaaaatcgagctgcagaattggcacgtctgatcacaccgtcctctcattcacggtgctttttttcttgtgtgcttggagattttcgattgtgtgttcgtgtttggttaaacttaatctgtatgaatcctgaaacgaaaaatggtggtgatttcctccagaagaattagagtacctggcaggaagcaggtggctctgtggacctgagccacttcaatcttcaagggtctctggccaagacccaggtgcaaggcagaggcctgatgacccgaggacaggaaagctcggatgggaaggggcgatgagaagcctgcctcgttggtgagcagcgcatgaagtgcccttatttacgctttgcaaagattgctctggataccatctggaaaaggcggccagcgggaatgcaaggagtcagaagcctcctgctcaaacccaggccagcagctatggcgcccacccgggcgtgtgccagagggagaggagtcaaggcacctcgaagtatggcttaaatctttttttcacctgaagcagtgaccaaggtgtattctgagggaagcttgagttaggtgccttctttaaaacagaaagtcatggaagcacccttctcaagggaaaaccagacgcccgctctgcggtcatttacctctttcctctctccctctcttgccctcgcggtttctgatcgggacagagtgacccccgtggagcttctccgagcccgtgctgaggaccctcttgcaaagggctccacagacccccgccctggagagaggagtctgagcctggcttaataacaaactgggatgtggctgggggcggacagcgacggcgggattcaaagacttaattccatgagtaaattcaacctttccacatccgaatggatttggattttatcttaatattttcttaaatttcatcaaataacattcaggactgcagaaatccaaaggcgtaaaacaggaactgagctatgtttgccaaggtccaaggacttaataaccatgttcagagggatttttcgccctaagtactttttattggttttcataaggtggcttagggtgcaagggaaagtacacgaggagaggcctgggcggcagggctatgagcacggcagggccaccggggagagagtccccggcctgggaggctgacagcaggaccactgaccgtcctccctgggagctgccacattgggcaacgcgaaggcggccacgctgcgtgtgactcaggaccccataccggcttcctgggcccacccacactaacccaggaagtcacggagctctgaacccgtggaaacgaacatgacccttgcctgcctgcttccctgggtgggtcaagggtaatgaagtggtgtgcaggaaatggccatgtaaattacacgactctgctgatggggaccgttccttccatcattattcatcttcacccccaaggactgaatgattccagcaacttcttcgggtgtgacaagccatgacaaaactcagtacaaacaccactcttttactaggcccacagagcacggsccacacccctgatatattaagagtccaggagagatgaggctgctttcagccaccaggctggggtgacaacagcggctgaacagtctgttcctctagactagtagaccctggcaggcactcccccagattctagggcctggttgctgcttcccgagggcgccatctgccctggagactcagcctggggtgccacactgaggccagccctgtctccacaccctccgcctccaggcctcagcttctccagcagcttcctaaaccctgggtgggccgtgttccagcgctactgtctcacctgtcccactgtgtcttgtctcagcgacgtagctcgcacggttcctcctcacatggggtgtctgtctccttccccaacactcacatgcgttgaagggaggagattctgcgcctcccagactggctcctctgagcctgaacctggctcgtggcccccgatgcaggttcctggcgtccggctgcacgctgacctccatttccaggcgctccccgtctcctgtcatctgccggggcctgccggtgtgttcttctgtttctgtgctcctttccacgtccagctgcgtgtgtctctgcccgctagggtctcggggtttttataggcataggacgggggcgtggtgggccagggcgctcttgggaaatgcaacatttgggtgtgaaagtaggagtgcctgtcctcacctaggtccacgggcacaggcctggggatggagcccccgccagggacccgcccttctctgcccagcactttcctgcccccctccctctggaacacagagtggcagtttccacaagcactaagcatcctcttcccaaaagacccagcattggcacccctggacatttgccccacagccctgggaattcacgtgactacgcacatcatgtacacactcccgtccacgaccgacccccgctgttttattttaatagctacaaagcagggaaatccctgctaaaatgtcctttaacaaactggttaaacaaacgggtccatccgcacggtggacagttcctcacagtgaagaggaacatgccgtttataaagcctgcaggcatctcaagggaattacgctgagtcaaaactgccacctccatgggatacgtacgcaacatgctcaaaaagaaagaatttcaccccatggcaggggagtggttaggggggttaaggacggtgggggcggcagctgggggctactgcacgcaccttttactaaagccagtttcctggttctgatggtattggctcagttatgggagactaaccataggggagtggggatgggggaacccggaggctgtgccatctttgccatgcccgagtgtcctgggcaggataatgctctagagatgcccacgtcctgattcccccaaacctgtggacagaacccgcccggccccagggcctttgcaggtgtgatctccgtgaggaccctgaggtctgggatccttcgggactacctgcaggcccgaaaagtaatccaggggttctgggaagaggcgggcaggagggtcagaggggggcagcctcaggacgatggaggcagtcagtctgaggctgaaaagggagggagggcctcgagcccaggcctgcaagcgcctccagaagctggaaaaagcggggaagggaccctccacggagcctgcagcaggaaggcacggctggcccttagcccaccagggcccatcgtggacctccggcctccgtgccataggagggcactcgcgctgcccttctagcatgaagtgtgtggggatttgcagaagcaacaggaaacccatgcactgtgaatctaggattatttcaaaacaaaggtttacagaaacatccaaggacagggctgaagtgcctccgggcaagggcagggcaggcacgagtgattttatttagctattttattttatttacttactttctgagacagagttatgctcttgttgcccaggctggagtgcagcggcatgatcttggctcactgcaacctccgtctcctgggttcaagcaattctcgtgcctcagcctcccaagtagctgggatttcaggcgtgcaccaccacacccggctaattttgtatttttagtagagatgggctttcaccatgttggtcaagctgatctcaaaatcctgacctcaggtgatccgcccacctcagcctcccaaagtgctgggattacaggcatgagccactgcacctggcctatttaaccattttaaaacttccctgggctcaagtcacacccactggtaaggagttcatggagttcaatttcccctttactcaggagttaccctcctttgatattttctgtaattcttcgtagactggggatacaccgtctcttgacatattcacagtttctgtgaccacctgttatcccatgggacccactgcaggggcagctgggaggctgcaggcttcaggtcccagtggggttgccatctgccagtagaaacctgatgtagaatcagggcgcaagtgtggacactgtcctgaatctcaatgtctcagtgtgtgctgaaacatgtagaaattaaagtccatccctcctactctactgggattgagccccttccctatccccccccaggggcagaggagttcctctcactcctgtggaggaaggaatgatactttgttatttttcactgctggtactgaatccactgtttcatttgttggtttgtttgttttgttttgagaggcggtttcactcttgttgctcaggctggagggagtgcaatggcgcgatcttggcttactgcagcctctgcctcccaggttcaagtgattctcctgcttccgcctcccatttggctgggattacaggcacccgccaccatgcccagctaattttttgtatttttagtagagacgggggtgggtggggttcaccatgttggccaggctggtctcgaacttctgacctcagatgatccacctgcctctgcctcctaaagtgctgggattacaggtgtgagccaccatgcccagctcagaatttactctgtttagaaacatctgggtctgaggtaggaagctcaccccactcaagtgttgtggtgttttaagccaatgatagaatttttttattgttgttagaacactcttgatgttttacactgtgatgactaagacatcatcagcttttcaaagacacactaactgcacccataatactggggtgtcttctgggtatcagcaatcttcattgaatgccgggaggcgtttcctcgccatgcacatggtgttaattactccagcataatcttctgcttccatttcttctcttccctcttttaaaattgtgttttctatgttggcttctctgcagagaaccagtgtaagctacaacttaacttttgttggaacaaattttccaaaccgcccctttgccctagtggcagagacaattcacaaacacagccctttaaaaaggcttagggatcactaaggggatttctagaagagcgacctgtaatcctaagtatttacaagacgaggctaacctccagcgagcgtgacagcccagggagggtgcgaggcctgttcaaatgctagctccataaataaagcaatttcctccggcagtttctgaaagtaggaaaggttacatttaaggttgcgtttgttagcatttcagtgtttgccgacctcagctacagcatccctgcaaggcctcgggagacccagaagtttctcgcccccttagatccaaacttgagcaacccggagtctggattcctgggaagtcctcagctgtcctgcggttgtgccggggccccaggtctggaggggaccagtggccgtgtggcttctactgctgggctggaagtcgggcctcctagctctgcagtccgaggcttggagccaggtgcctggaccccgaggctgccctccaccctgtgcgggcgggatgtgaccagatgttggcctcatctgccagacagagtgccggggcccagggtcaaggccgttgtggctggtgtgaggcgcccggtgcgcggccagcaggagcgcctggctccatttcccaccctttctcgacgggaccgccccggtgggtgattaacagatttggggtggtttgctcatggtggggacccctcgccgcctgagaacctgcaaagagaaatgacgggcctgtgtcaaggagcccaagtcgcggggaagtgttgcagggaggcactccgggaggtcccgcgtgcccgtccagggagcaatgcgtcctcgggttcgtccccagccgcgtctacgcgcctccgtcctccccttcacgtccggcattcgtggtgcccggagcccgacgccccgcgtccggacctggaggcagccctgggtctccggatcaggccagcggccaaagggtcgccgcacgcacctgttcccagggcctccacatcatggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgatg

SEQ ID NO: 4 - DNA, 104 bp, Homo sapiens
gtgggcctccccggggtcggcgtccggctggggttgagggcggccggggggaaccagcgacatgcggagagcagcgcaggcgactcagggcgcttcccccgcag

SEQ ID NO: 5 - DNA, 8616 bp, Homo sapiens
gtgaggaggtggtggccgtcgagggcccaggccccagagctgaatgcagtaggggctcagaaaagggggcaggcagagccctggtcctcctgtctccatcgtcacgtgggcacacgtggcttttcgctcaggacgtcgagtggacacggtgatctctgcctctgctctccctcctgtccagtttgcataaacttacgaggttcaccttcacgttttgatggacacgcggtttccaggcgccgaggccagagcagtgaacagaggaggctgggcgcggcagtggagccgggttgccggcaatggggagaagtgtctggaagcacagacgctctggcgagggtgcctgcaggttacctataatcctcttcgcaatttcaagggtgggaatgagaggtggggacgagaaccccctcttcctgggggtgggaggtaagggttttgcaggtgcacgtggtcagccaatatgcaggtttgtgtttaagatttaattgtgtgttgacggccaggtgcggtggctcacgccggtaatcccagcactttgggaagctgaggcaggtggatcacctgaggtcaggagtttgagaccagcctgaccaacatggtgaaaccctatctgtactaaaaatacaaaaattagctgggcatggtggtgtgtgcctgtaatcccagctacttgggaggctgaggcaggagaatcacttgaacccaggaggcggaggctgcagtgagctgagattgtgccattgtactccagcctgggcgacaagagtgaaactctgtctttaaaaaaaaaaagtgttcgttgattgtgccaggacagggtagagggagggagataagactgttctccagcacagatcctggtcccatctttaggtatgaagagggccacatgggagcagaggacagcagatggctccacctgctgaggaagggacagtgtttgtgggtgttcaggggatggtgctgctgggccctgccgtgtccccaccctgtttttctggatttgatgttgaggaacctccgctccagcccccttttggctcccagtgctcccaggccctaccgtggcagctagaagaagtcccgatttcaccccctccccacaaactcccaagacatgtaagacttccggccatgcagacaaggagggtgaccttcttggggctcttttttttctttttttctttttatggtggcaaaagtcatataacatgagattggcactcctaacaccgttttctgtgtacagtgcagaattgctaactcggcggtgtttacagcaggttgcttgaaatgctgcgtcttgcgtgactggaagtccctacccatcgaacggcagctgcctcacacctgctgcggctcaggtggaccacgccgagtcagataagcgtcatgcaacccagttttgctttttgtgctccagcttccttcgttgaggagagtttgagttctctgatcaggactctgcctgtcattgctgttctctgacttcagatgaggtcacaatctgcccctggcttatgcagggagtgaggcgtggtccccgggtgtccctgtcacgtgcagggtgagtgaggcgttgcccccaggtgtccctgtcacgtgtagggtgagtgaggcgcggcccccgggtgtccctgtcccgtgcagcgtgattgaggtgtggcccccgggtgtccctgtcacgtgtagggtgagtgaggcgccatccccgggtgtccctgtcacgtgtagggtgagtgaggcgtggtccccgggtgtccctgtcccgtgcagggtgagtgaggcactgtccccgggtgtccctgtcacgtgcagggtgagtgaggcgcggtccccgggtgtccctctcaggtgtagggtgagtgaggcgcggccccagggtgtccctgtcacgtgtagggtgagtgaggcaccgtccctgggtgtccctcccaggtatagggtgagtgaggcactgtccccgggtgtccctgtcacgtgcagggtgagtgaggcgcggcccccgggtgtccctctcaggtgcagggtgagtgaggcgctgtccctgggtgtccctgtctcgtgtagggtgagtgaggctctgtccccaggtgtccttggcgtttgctcacttgagcttgctcctgaatgtttgctctttctatagccacagctgcgccggttgcccattgcctgggtagatggtgcaggcgcagtgctggtccccaagcctatcttttctgatgctcggctcttcttggtcacctctccgttccattttgctacggggacacgggactgcaggctctcgcctcccgcgtgccaggcactgcagccacagcttcaggtccgcttgcctctgttgggcctggcttgctcaccacgtgcccgccacatgcatgctgccaatactcctctcccagcttgtctcatgccgaggctggactctgggctgcctgtgtctgctgccacgtgttgctggagacatcccagaaagggttctctgtgccctgaaggaaagcaagtcaccccagccccctcacttgtcctgttttctcccaagctgcccctctgcttggcccccttgggtgggtggcaacgcttgtcaccttattctgggcacctgccgctcattgcttaggctgggctctgcctccagtcgccccctcacatggattgacgtccagccacaggttggagtgtctctgtctgtctcctgctctgagacccacgtggagggccggtgtctccgccagccttcgtcagacttccctcttgggtcttagttttgaatttcactgatttacctctgacgtttctatctctccattgtatgctttttcttggtttattctttcattccttttctagcttcttagtttagtcatgcctttccctctaagtgctgccttacctgcaccctgtgttttgatgtgaagtaatctcaacatcagccactttcaagtgttcttaaaatacttcaaagtgttaatacttcttttaagtattcttattctgtgatttttttctttgtgcacgctgtgttttgacgtgaaatcattttgatatcagtgacttttaagtattctttagcttattctgtgatttctttgagcagtgagttatttgaacactgtttatgttcaagatatgtagagtatcaagatacgtagagtattttaagttatcattttattattgatttctaactcagttgtgtagtggtctgtataataccaattatttgaagtttgcggagccttgctttgtgatctagtgtgtgcatggtttccagaactgtccattgtaaatttgacatcctgtcaatagtgggcatgcatgttcactatatccagcttattaaggtccagtgcaaagcttctgtctccttctagatgcatgaaattccaagaaggaggccatagtccctcacctgggggatgggtctgttcatttcttctcgtttggtagcatttatgtgaggcattgttaggtgcatgcacgtggtagaatttttatcttcctgatgagtgaatcttttggagacttctatgtctctagtaatctagtaattctttttttaaattgctcttagtactgccacactgggcttcttttgattagtattttcctgctgtgtctgttttctgcctttaatttatatatatatatatattttttttttttttgagacagagtcttggtctgtcgcccagggtgagtgcagtggtgtgatcacaggtcagtgtaacttttaccttctggcctgagccgtcctctcacctcagcctcctgagtagctggaactgcagacacgcaccgctacacctggctaatttttaaattttttctggagacagggtcttgctgtgttgcccaggctggtctcaaactcttggactcaagggatccatctacctcggcttcccaaagtgctgaattacaggcatgagccaccatgtctggcctaattttcaacacttttatattcttatagtgtgggtatgtcctgttaacagcatgtaggtgaatttccaatccagtctgacagtcgttgtttaactggataacctgatttattttcatttttttgtcactagagacccgcctggtgcactctgattctccacttgcctgttgcatgtcctcgttcccttgtttctcaccacctcttgggttgccatgtgcgtttcctgccgagtgtgtgttgatcctctcgttgcctcctggtcactgggcatttgcttttatttctctttgcttagtgttaccccctgatctttttattgtcgttgtttgcttttgtttattgagacagtctcactctgtcacccaggctggagtgtaatggcacaatctcggctcactgcaacctctgcctcctcggttcaagcagttctcattcctcaacctcatgagtagctgggattacaggcgcccaccaccacgcctggctaatttttgtatttttagtagagataggctttcaccatgttggccaggctggtctcaaactcctgacctcaagtgatctgcccgccttggcctcccacagtgctgggattacaggtgcaagccaccgtgcccggcataccttgatcttttaaaatgaagtctgaaacattgctacccttgtcctgagcaataagacccttagtgtattttagctctggccaccccccagcctgtgtgctgttttccctgctgacttagttctatctcaggcatcttgacacccccacaagctaagcattattaatattgttttccgtgttgagtgtttctgtagctttgcccccgccctgcttttcctcctttgttccccgtctgtcttctgtctcaggcccgccgtctggggtccccttccttgtcctttgcgtggttcttctgtcttgttattgctggtaaaccccagctttacctgtgctggcctccatggcatctagcgacgtccggggacctctgcttatgatgcacagatgaagatgtggagactcacgaggagggcggtcatcttggcccgtgagtgtctggagcaccacgtggccagcgttccttagccagtgagtgacagcaacgtccgctcggcctgggttcagcctggaaaaccccaggcatgtcggggtctggtggctccgcggtgtcgagtttgaaatcgcgcaaacctgcggtgtggcgccagctctgacggtgctgcctggcgggggagtgtctgcttcctcccttctgcttgggaaccaggacaaaggatgaggctccgagccgttgtcgcccaacaggagcatgacgtgagccatgtggataattttaaaatttctaggctgggcgcggtggctcacgcctgtaatcccagcactttgggaggccaaggcgggtggatcacgaggtcaggaggtcgagaccatcctggccaacatgatgaaaccccatctgtactaaaaacacaaaaattagctgggcgtggtggcgggtgcctgtaatcccagctactcgggaggctgaggcaggagaattgcttgaacctgggagttggaagttgcagtgagccgacattgcaccactgcactccagcctggcaacacagcgagactctgtctcaaaaaaaaaaaaaaaaaaaaaaaaaaaaattctagtagccacattaaaaaagtaaaaaagaaaaggtgaaattaatgtaataatagattttactgaagcccagcatgtccacacctcatcattttagggtgttattggtgggagcatcactcacaggacatttgacattttttgagctttgtctgcgggatcccgtgtgtaggtcccgtgcgtggccatctcggcctggacctgctgggcttcccatggccatggctgttgtaccagatggtgcaggtccgggatgaggtcgccaggccctcagtgagctggatgtgcagtgtccggatggtgcacgtctgggatgaggtcgccaggccctgctgtgagctggatgtgtggtgtctggatggtgcaggtcaggggtgaggtctccaggccctcggtgagctggaggtatggagtccggatgatgcaggtccggggtgaggtcgccaggccctgctgtgagctggatgtgtggtgtctggatggtgcaggtcaggggtgaggtctccaggccctcggtaagctggaggtatggagtccggatgatgcaggtccggggtgaggtcgccaggccctgctgtgagctggatgtgtggtgtctggatggtgcaggtctggggtgaggtcaccaggccctgcggtgagctgggtgtgcggtgtctggatggtgcaggtctggagtgaggtcgccagacggtgccagaccatgcggtgagctggatatgcggtgtccggatggtgcaggtctggggtgaggttgccaggccctgctgtgagttggatgtggggtgtccggatgctgcaggtccggtgtgaggtcaccaggccctgctgtgagctggatgtgtggtgtctggatggtgcaggtctggggtgaaggtcgccaggcccctgcttgtgagctggatgtgtggtgtctggatggtgcaggtctggagtgaggtcgccaggccctcggtgagctggatgtgcagtgtccagatggtgcaggtccggggtgaggtcgccagaccctgcggtgagctggatgtgcggtgtctggatggtgcaggtctggagtgaggtcgccaggccctcggtgagctggatgtatggagtccggatggtgccggtccggggtgaggtcgccagaccctgctgtgagctggatgtgcggtgtctggatggtacaggtctggagtgaggtcgccagaccctgctgtgagctggatatgcggtgtccggatggtgcaggtcaggggtgaggtctccaggccctcggtgagctggaggtatggagtccggatgatgcaggtccggggtgaggtcgccaggccctgctgtgaactggatgtgcggcgtctggatggtgcaggtctggggtgtggtcgccaggccctcggtgagctggaggtatggagtccggatgatgcaggtccggggtgaggtcgccaggccctgctgtgagctggatgtgcggcgtctggatggtgcaggtctggggtgtggtcgccaggccctcggtgagctggaggtatggagtccggatgatgcaggtccggggtgaggttgccaggccctgctgtgagctggatgtgctgtatccggatggtgcagtccggggtgaggtcgccaggccctgctgtgagctggatgtgctgtatccggatggtgcaggtctggggtgaggtcaccaggccctgcggtgagctggttgtgcggtgtccggttgctgcaggtccggggtgagttcgccaggccctcggtgagctggatgtgcggtgtccccgtgtccggatggtgcaggtccagggtgaggtcgctaggcccttggtgggctggatgtgccgtgtccggatggtgcaggtctggggtgaggtcgccaggcctttggtgagctggatgtgcggtgtctgcatggtgcaggtctggggtgaggtcgccaggcccttggtgggctggatgtgtggtgtccggatggtgcaggtccggcgtgaggtcgccaggccctgctgtgagctggatgtgcggtgtctggatggtgcaggtccggggtgaggtagccaaggccttcggtgagctggatgtggggtgtccggatggtgcaggtccggggtgaggtcgccaggccctgcggttagctggatatgcggtgtccggatggtgcaggtccggggtgaggtcaccaggccctgcggttagctggatgtgcggtgtctggatggtgcaggtccggggtgaggtcgccaggccctgctgtgagctggatgtgctgtatccggatggtgcaggtccggggtgaggtcgccaggccctgcagtgagctggatgtgctgtatccggatggtgcaggtctggcgtgaggtcgccaggccctgcggttagctggatatgcggtgtcggatggtgcaggtccggggtgaggtcaccaggccctgcggttagctggatgtgcggtgtccggatggtgcaggtctggggtgaggtcgccaggccctgctgtgagctggatgtgctgtatccggatggtgcaggtccggggtgaggtcgccaggccctgcggtgagctggatgtgctgtatccggatggtgcaggtctggcgtgaggtcgccaggccctgcggtgagctggatgtgcagtgtacggatggtgcaggtccggggtgaggtcgccaggccctgcggtgggctgtatgtgtgttgtctggatggtgcaggtccggggtgagttcgccaggccctgcggtgagctggatgtgtggtgtctggatgctgcaggtccggggtgagttcgccaggccctcggtgagctggatatgcggtgtccccgtgtccgaatggtgcaggtccagggtgaggtcgccaggcccttggtgggctggatgtgccgtgtccggatggtgcaggtctggggtgaggtcgccaggcccttggtgagctggatgtgcggtgtccggatggtgcaggtccggggtgaggtcaccaggccctcggtgatctggatgtggcatgtccttctcgtttaag

SEQ ID NO: 6 - DNA, 2089 bp, Homo sapiens
gtactgtatccccacgccaggcctctgcttctcgaagtcctggaacaccagcccggcctcagcatgcgcctgtctccacttgcctgtgcttccctggctgtgcagctctgggctgggagccaggggccccgtcacaggcctggtccaagtggattctgtgcaaggctctgactgcctggagctcacgttctcttacttgtaaaatcaggagtttgtgccaagtggtctctagggtttgtaaagcagaagggatttaaattagatggaaacactaccactagcctccttgcctttccctgggatgtgggtctgattctctctctcttttttttttcttttttgagatggagtctcactctgttgcccaggctggagtgcagtggcataatcttggctcactgcaacctccacctcctgggtttaagcgattcaccagcctcagcctcctaagtagctgggattacaggcacctgccaccacgcctggctaatttttgtacttttaggagagacggggtttcaccatgttggccaggctggtctcgaactcatgacctcaggtgatccacccaccttggcctcccaaagtgctgggtttacaggctaagccaccgtgcccagcccccgattctcttttaattcatgctgttctgtatgaatcttcaatctattggatttaggtcatgagaggataaaatcccacccacttggcgactcactgcagggagcacctgtgcagggagcacctggggataggagagttccaccatgagctaacttctaggtggctgcatttgaatggctgtgagattttgtctgcaatgttcggctgatgagagtgtgagattgtgacagattcaagctggatttgcatcagtgagggacgggagcgctggtctgggagatgccagcctggctgagcccaggccatggtattagcttctccgtgtcccgcccaggctgactgtggagggctttagtcagaagatcagggcttccccagctcccctgcacactcgagtccctggggggccttgtgacaccccatgccccaaatcaggatgtctgcagagggagctggcagcagacctcgtcagaggtaacacagcctctgggctggggaccccgacgtggtgctggggccatttccttgcatctgggggagggtcagggctttccctgtgggaacaagttaatacacaatgcaccttacttagactttacacgtatttaatggtgtgcgacccaacatggtcatttgaccagtattttggaaagaatttaattggggtgaccggaaggagcagacagacgtggtggtccccaagatgctccttgtcactactgggactgttgttctgcctggggggccttggaggcccctcctccctggacagggtaccgtgccttttctactctgctgggcctgcggcctgcggtcagggcaccagctccggagcacccgcggccccagtgtccacggagtgccaggctgtcagccacagatgcccaggtccaggtgtggccgctccagcccccgtgcccccatgggtggttttgggggaaaaggccaagggcagaggtgtcaggagactggtgggctcatgagagctgattctgctccttggctgagctgccctgagcagcctctcccgccctctccatctgaagggatgtggctctttctacctgggggtcctgcctggggccagccttgggctaccccagtggctgtaccagagggacaggcatcctgtgtggaggggcatgggttcacgtggccccagatgcagcctgggaccaggctccctggtgctgatggtgggacagtcaccctgggggttgaccgccggactgggcgtccccagggttgactataggaccaggtgtccaggtgccctgcaagtagaggggctctcagaggcgtctggctggcatgggtggacgtggccccgggcatggccttcagcgtgtgctgccgtgggtgccctgagccctcactgagtcggtgggggcttgtggcttcccgtgagcttccccctagtctgttgtctggctgagcaagcctcctgaggggctctctattgcag

SEQ ID NO: 7 - DNA, 687 bp, Homo sapiens
gtggctgtgctttggtttaacttcctttttaaacagaagtgcgtttgagccccacatttggtatcagcttagatgaagggcccggaggaggggccacgggacacagccagggccatggcacggcgccaacccatttgtgcgcacagtgaggtggccgaggtgccggtgcctccagaaaagcagcgtgggggtgtagggggagctcctggggcagggacaggctctgaggaccacaagaagcagccgggccagggcctggatgcagcacggcccgaggtcctggatccgtgtcctgctgtggtgcgcagcctccgtgcgcttccgcttacggggcccggggaccaggccacgactgccaggagcccaccgggctctgaggatcctggaccttgccccacggctcctgcaccccacccctgtggctgcggtggctgcggtgaccccgtcatctgaggagagtgtggggtgaggtggacagaggtgtggcatgaggatcccgtgtgcaacacacatgcggccaggaacccgtttcaaacagggtctgaggaagctgggaggggttctaggtcccgggtctgggtggctggggacactggggaggggctgcttctcccctgggtccctatggtggggtgggcacttggccggatccactttcctgactgtctcccatgctgtccccgccag

SEQ ID NO: 8 - DNA, 494 bp, Homo sapiens
gtgggtgccggggacccccgtgagcagccctgctggaccttgggagtggctgcctgattggcacctcatgttgggtggaggaggtactcctgggtgggccgcagggagtgcaggtgaccctgtcactgttgaggacacacctggcacctagggtggaggccttcagcctttcctgcagcacatggggccgactgtgcaccctgactgcccgggctcctattcccaaggagggtcccactggattccagtttccgtcagagaaggaaccgcaacggctcagccaccaggccccggtgccttgcaccccagtcctgagccaggggtctcctgtcctgaggctcagagaggggacacagcccgccctgcccttggggtctggagtggtgggggtcagagagagagtgggggacaccgccaggccaggccctgagggcagaggtgatgtctgagtttctgcgtggccactgtcagtctcctcgcctccactcacacag

SEQ ID NO: 9 - DNA, 865 bp, Homo sapiens
gtaaggttcacgtgtgatagtcgtgtccaggatgtgtgtctctgggatatgaatgtgtctagaatgcagtcgtgtctgtgatgcgtttctgtggtggaggtacttccatgatttacacatctgtgatatgcgtgtgtggcacgtgtgtgtcgtggtgcatgtatctgtggcgtgcatatttgtggtgtgtgtgtgtgtggcacgtgtgtgtccatggtgtgtgtgcctgtggtgtgcatgtgtgtgtgtctgtgacacgtgcatgttcatgctgtgtgctgcatgtctgtgatgtgcctatttgtggtgtgtgtgtgcatgtgtccgtgacatatgcgtgtctatggcatgggtgtgtgtggccccttggccttactccttcctcctccaggcatggtccgcaccattgtcctcacgctctcgggtgctggtttggggagctccacattcagggtcctcacttctagcatgggtgcccctgtcctgtcacagggctgggccttggagactgtaagccaggtttgagaggagagtagggatgctggtggtaccttcctggacccctggcacccccaggaccccagtctggcctatgccggctccatgagatataggaaggctgattcaggcctcgctccccgggacacactcctcccagagcggccgggggccttggggctcggcaggggtgaaaggggccctgggcttgggttcccacccagtggtcatgagcacgctggaggggtaagccctcaaagtcgtgccaggccggggtgcagaggtgaagaagtatccctggagcttcggtctggggagaggcacatgtggaaacccacaaggacctctttctctgacttcttgagct

SEQ ID NO: 10 - DNA, 3782 bp, Homo sapiens
tgtgggattggttttcatgtgtgggataggtggggatctgtgggattggtttttatgagtggggtaacacagagttcaaggcgagctttcttcctgtagtgggtctgcaggtgctccaacagctttattgaggagaccatatcttcctttgaactatggtcgggtttatagtaagtcaggggtgtggaggcctcccctgggctccctgttctgtttcttccactctggggtcgtgtggtgcctgctgtggtgtgtggccggtgggcagggcttccaggcctccttgtgttcattggcctggatgtggccctggctacgctccgtccttggaattcccctgcgagttggaggctttctttctttctttttttctttctttttttttttttttgataacagagtctcgctcttttttgcccaggctggagtggtttggcgtgatcttggctcactgcaacctgtgcttcctgagttcaagcaattctcttgcctcagcctcccaagtagctggaattataggcgcccaccaccatgctgactaatttttgtaattttagtagagacgaggtttctccatgttggccaggctggtctcgaactcctgacctcaggtgatcctcccacctcggcctcccaaagtgctgggatgacaggtgtgaaccgccgcgcccggccgagactcgcttcctgcagcttccgtgagatctgcagcgatagctgcctgcagccttggtgctgacaacctccgttttccttctccaggtctcgctaggggtctttccatttcatgactctcttcacagaagagtttcacgtgtgctgatttcccggctgtttcctgcgtaattggtgtctgctgtttatcgatggcctccttccatttcctttaggctttgtttattgttgtttttccggctccttgaaggaaaagtttcgattatggatgtttgaactttcttttctaaacaagcatctgaagttgccgttttccctctaaagcagggatcccgaggcccctggctgtggagtggcaccggtctggggcctgttaggaacccggcgcacagcgggaggctaggtggggtgtggggagccagcgttcccgcctgagccccgcccctctcagatcagcagtggcatgcggtgctcagaggcgcacacaccctactgagaactgtgcgtgagaggggtctagattctgtgctccttatgggaatctaatgcctgatgatctgaggtggaaccgtttgctcccaaaaccatccccttccccactgctgtcctgtggaaaaatcgtcttccacgaaaccagtccctggtaccacaatggttggggaccctgtgctaaagacctgcttcagcagcctctcgtcagtgttgatatattggcttttctgtgttgagtccagaataattacggatttctgtgatgctttccgccgacctcagacccatgggctatttgtgggcgtgttgcctgctcctgggttgggaagggtgcaggccccatgtaccttcctgttactgccttccaggttggttctcagggttgaatcgtactcgatgtggttttagcccacggccctgccgccagctcctgggggctggggaacatgctgaagcacagagtcaccgtgcgcgtcttttgatgcctcacaagctcgaggcctcctgtgtccgtgttagtgtgtgtcacgtgcctgctcacatcctgtcttggggacgcaggggcttagcaggtcccgtagtaaatgacaagcgtcctgggggagtctgcagaataggaggtgggggtgccggtctctctcccgcgtcttcagactcttctcctgcctgtgctgtggctgcacctgcatccctgcaatccctccagcactgggctggagaggcccgggagctcgagtgccacttgtgccacgtgactgtggatggcagtcggtcacgggggtctgatgtgtggtgactgtggatggcggttggtcacaggggtctgatgtgtggtgactgtggatggcggtcgtggggtctgatgtggtgactgtggatggcggtcgtggggtctgatgtgtggtgactgtggatggcggtcgtggggtctgatgtggtgactgtggatggcggtcgtggggtctgatgtggtgactgtggatggcggtcgtggggtctgatgtggtgactgtggatggcagtcgtggggtctgatgtgtggtgactgtggatggcggtcgtggggtctgatgtggtgactgtggatggcagtcgtggggtctgatgtgtggtgactgtggatggcggtcgtggggtctgatgtgtggtgactgtggatggcggtcgtggggtctgatgtgtggtgactgtggatggcggtcgtggggtctgatgtgtggtgactgtggatggcggtcgtggggtctgatgtggtgactgtggatggcggtcgtggggtctgatgtgtggtgactgtggatggtgatcggtcacaggggtctgatgtgtggtgactgtggatggcggtcgtggggtctgatgtgtggtgactgtggatggtgatcggtcacaggggtctgatgtgtggtgactgtggatggcggtcgtggggtctgatgtgtggtgactgtggatggcggttggtcccgggggtctgatgtgtggtgactgtggatggcgatcggtcacaggggtctgatgtgtggtgactgtggatggcggtcgtggggtctgatgtgtggtgactgtggatggcggtcgtggggtctgatgtgtggtgactgtggatggcggtcgtggggtctgatgtggtgactgtggatggcggtcgtggggtctgatgtggtgactgtggatggcggtcgtggggtctgatgtgtggtgactgtggatggcggttggtcccgggggtctgatgtgtggtgactgtggatggcggtcgtggggtctgatgtggtgactgtggatggcagtcgtggggtctgatgtgtggtgactgtggatggcggtcgtggggtctgatgtgtggtgactgtggatggcggtcgtggggtctgatgtgtggtgactgtggatggcggtcgtggggtctgatgtgtggtgactgtggatggcggtcgtggggtctgatgtggtgactgtggatggcggtcgtggggtctgatgtgtggtgactgtggatggtgatcggtcacaggggtctgatgtgtggtgactgtggatggcggtcgtggggtctgatgtgtggtgactgtggatggcggtcgtggggtctgatgtggtgactgtggatggcggtcgtggggtctgatgtgtggtgactgtggatggcggtcgtagggtctgatgtgtggtgactgtggatggcagtcggtcacaggggtctgatgtgtggtgactgtggatggcggtcgtggggtctgatgtgtggtgactgtggatggcggtcgtggggtctgatgtgtggtgactgtggatggcggtcgtggggtctgatgtgtggtgactgtggatggcggtcgtggggtctgatgtggtgactgtggatggtgatcggtcacaggggtctgatgtgtggtagctgcaggtggagtcccaggtgtgtctgtagctactttgcgtcctcggccccccggcccccgtttcccaaacagaagcttcccaggcgctctctgggcttcatcccgccatcgggcttggccgcaggtccacacgtcctgatcggaagaaacaagtgcccagctctggccggggcaggccacatttgtggctcatgccctctcctctgccggcag

SEQ ID NO: 11 - DNA, 980 bp, Homo sapiens
gtctgggcactgccctgcagggttgggcacggactcccagcagtgggtcctcccctgggcaatcactgggctcatgaccggacagactgttggccctggggggcagtggggggaatgagctgtgatgggggcatgatgagctgtgtgccttggcgaaatctgagctgggccatgccaggctgcgacagctgctgcattcaggcacctgctcacgtttgactgcgcggcctctctccagttccgcagtgcctttgttcatgatttgctaaatgtcttctctgccagttttgatcttgaggccaaaggaaaggtgtccccctcctttaggagggcaggccatgtttgagccgtgtcctgcccagctggcccctcagtgctgggtctgaggccaaaggaaacgtgtcccccttcttaggaggacgggccgtgtttgagccacgccccgctgagcgggcctctcagtgctgggtctgtccacgtggccctgtggccctttgcagatgtggtctgtccacgtggccctgtggctctttgcagatgcctgttagcacttgctcggctctaggggacagtcgtgtccaccgcatgaggctcagagacctctgggcgaatttccttggctcccagggtgggggtggaggtggcctgggctgctgggacccagaccctgtgcccggcagctgggcagcaactcctggatcacatatgccatccgggccacggtgggctgtgtgggtgtgagcccagctggacccacaggtggcccagaggagacgttctgtgtcacacactctgcctaagcccatgtgtgtctgcagagactcggcccggccagcccacgatggccctgcattccagcccagccccgcacttcatcacaaacactgaccccaaaagggacggagggtcttggccacgtggtcctgcctgtctcagcacccaccggctcactcccatgtgtctcccgtctgctttcgcag

SEQ ID NO: 12 - DNA, 2485 bp, Homo sapiens
  misc_feature (1)..(2485): n is a, c, g, or t.
gtgagtcaggtggccaggtgccattgccctgcgggtggctgggcgggctggcagggcttctgctcacctctctcctgccccttccccactgnccttctgcccggggccaccagagtctccttttctggcccccgccccctccggctcctgggctgcaggctcccgaggccccggaaacatggctcggcttgcggcagccggagcggagcaggtgccacacgaggcctggaaatggcaagcggggtgtggagttgctcctgcgtggaggacgaggggcggggggtgtgtctgggtcaggtgtgcgccgagcgtttgagcctgcagcttgtcagctccaagttactactgacgctggacacccggctctcacacgcttgtatctctctctcccgatacaaaaggattttatccgattctcattcctgtccctgtcgtgtgacccccgcgagggcgcgggctcttctctctgtgactagatttcccatctggaaagtgcggggttgaccgtgtagtttgctcctctcggggggcctgtggtggccatggggcaggcggcctgggagagctgccgtcacacagccactgggtgagccacactcacggtggtagagccacagtgcctggtgccacatcacgtcctctggattttaagtaaaaccacacacctcccggcaggcatctgcctgcgaccctgtgtgtgcctggggagagtggtagcacggaggaaattcgtgcacactcaaggtcatcagcaaggtcatccgcagtcaggtggaacgtggaggcctctctctgggatcgtctccagcggataaaggactgtgcacagcttcggaagcttttatttaaaaatataactattaattattgcattataagtaatcactaatggtatcagcaattataatatttattaaagtataattagaaatattaagtagtacacacgttctggaaaaacacaaattgcacatggcagcagagtgaattttggccgagggacacgtgtgcacatgtgtgtaagcggcccccaggcccacagaattcgctgacaaagtcacctccccagagaagccaccacgggcctccttcgtggtcgtgaattttattaagatggatcaagtcacgtaccgtccacgtgtggcagggctttggggaatgtgaggtgatgactgcgtcctcatgccctgacagacaggaggtgactgtgtctgtcctgtccctaggacacggacaggcccgaagctctagtccccatcgtggtccagtttggcctctgaataaaaacgtcttcaaaacctgttgccccaaaaactaagaacagagagagtttcccatcccatgtgctcacaggggcgtatctgcttgcgttgactcgctgggctggccggactcctagagttggtgcgtgtgcttctgtgcaaaaagtgcagtcctcttgcccatcactgtgatatctgcaccagcaaggaaagcctcttttcttttctttctttttttttttttgagacggaacgtcactgttgtctgcctgggcttgagtgcagtggcgcgatctcaactcactgcaacctccgcctcccgggttccagcatttctcctgcctcagcctcccgagcagctgagattacaggcacccaccccctgcgcctggctaatttttgtatttttagtagagaggggtttttgccatgttggccaggctggtctcgaactcctgacctcaggtgatccacccacctcggcctcccaaagtgctgggattacaggtgtgagccatcacgcccagccggaaagcctctttttaaggtgaccacctatagcgcttcccgaaaataacaggtcttgtttttgcagtaggctgcaagcgtctcttagcaacaggagtggcgtcctgtgggctctggggatggctgagggtcgcgtggcagccatgccttctgtgtgcacctttaggttccacggggctattctgctctcactgtttgtctgaaaacgcacccttggcatccttgtttggagagtttctgcttctcgttggtcatgctgaaactaggggcaaggttgtatccgttggcgcgcagcggctacatgtagggtcatgagtctttcaccgtggacaaattccttgaaaaaaaaaaaaggagtccggttaagcattcattccgggtcaagtgtctggttctgtgaataaactctaagatttaagaaaccttaatgaaagaaaaccttgatgattcagagcaaggatgtggtcacacctgtggctggatctgtttcagccgccccagtgcatggtgagagtggggagcagggattgtttgttcagaggtctcatctggtatgtttctgaggtgtttgccggctgaatggtagacgtgtcgtttgtgtgtatgaggttctgtgtctgtgtgtggctcggtttgagtgtacgcatgtccagcacatgccctgcccgtctctcacctgtgtcttcccgccccag

SEQ ID NO: 13 - DNA, 1984 bp, Homo sapiens
gtgaggcctcctcttccccaggggggcttgggtgggggttgatttgcttttgatgcattcagtgttaatattcctggtgctctggagaccatgactgctctgtcttgaggaaccagacaaggttgcagccccttcttggtatgaagccgcacgggaggggttgcacagcctgaggactgcgggctccacgcaggctctgtccagcggccatgtccagaggcctcagggctcagcaggcgggagggccgctgccctgcatgatgagcatgtgaattcaacaccgaggaagcacaccagcttctgtcacgtcacccaggttccgttagggtccttggggagatggggctggtgcagcctgaggccccacatctcccagcaggccctcgacaggtggcctggactgggcgcctcttcagcccattgcccatcccacttgcatggggtctacacccaaggacgcacacacctaaatatcgtgccaacctaatgtggttcaactcagctggcttttattgacagcagttacttttttttttttaatactttaagttctagggtacatgtgcacgacgtgcaggttagttacatatgtatacatgtgccatgttggtgtgctgcacccattaactcatcatttacattaggtatatctcctaatgctatccctccccactccccccatcccatgacaggccctggtgtgtgatgttccccaccctgtgtccaagtgttctcattgttcagttcccacctgtgagtgagaacatgtggtgtttggttttctttccttgcaatagtttgctcagagtgatggtttccagcttcgtccatgtccctacaaaggacatgaactcatccttttttatgactgcatagtattccgtggtgtatatgtgccacattttcttaatccagtctatcatcgatggacatttgggttggttgcaagtctttgctactgtgaatagtgccgcaataaacatacgtgtgcatgtgtctttatagcagcatgatttataatcctttgggtatatacccagtaatgggatggctgggtcaaatggtatttctagttctagatccttgaggaatcaccacactgtcttccacaatggttgaactagtttacactcccaccaacagtgtaaaagtgttctggtgctggagaggatgtggacagcagttatttttttatgaaaatagtatcactgaacaagcagacagttagtgaaggatgcgtcaggaagcctgcaggccacacagccatttctctcgaagactccgggtttttcctgtgcatcttttgaaactctagctccaattatagcatgtacagtggatcaaggttcttcttcattaaggttcaagttctagattgaaataagtttatgtaacagaaacaaaaatttcttgtacacacaacttgctctgggatttggaggaaagtgtcctcgagctggcggcacactggtcagccctctgggacaggatacctctggcccatggtcatggggcgctgggcttgggcctgagggtcacacagtgcaccatgcccagcttcctgtggataggatctgggtctcggatcatgctgaggaccacagctgccatgctggtaaagggcaccacgtggctcagagggggcgaggttcccagccccagctttcttaccgtcttcagttatttttccctaagagtctgagaagtggggccgcgcctgatggccttcgttcgtcttcagctggcacagaattgcacaagctgatggtaaacactgagtacttataatgaatgaggaattgctgtagcagttaactgtagagagctcgtctgttggaaagaaatttaagtttttcatttaaccgctttggagaatgttactttatttatggctgtgtaaattgtttgacattcagtccctcgtagacagatactacgtaaaaagtgtaaagttaaccttgctgtgtattttcccttattttag

SEQ ID NO: 14 - DNA, 1871 bp, Homo sapiens
gtgaggcccgtgccgtgtgtctgtggggacctccacagcctgtgggctttgcagttgagccccccgtgtcctgcccctggcaccgcagcgttgtctctgccaagtcctctctctctgccggtgctggatccgcaagagcagaggcgcttggccgtgcacccaggcctgggggcgcaggggcaccttcgggagggagtgggtaccgtgcaggccctggtcctgcagagacgcacccaggttacacacgtggtgagtgcaggcggtgacctggctcctgctgctctttggaaagtcaagagtggcggctcctggggccccagtgagacccccaggagctgtgcacagggcctgcagggccgaggcggcagcctcctccccagggtgcacctgagcctgcggagagcaggagctgctgagtgagctggcccacagcgttcgctgcggtcacgttcctgcgtggggttgtttgggatcggtgggagaatttggatttgctgagtgctgctgtcttgaaccacggagatggctaggagtgggtttcagagttgatttttgtgaatcaaactaaaatcaggcacaggggacctggcctcagcacaggggattgtccaatgtggtccccctcaagggcgccccacagagccggtgggcttgttttaaagtgcgatttgacgagggacgagaaaccttgaaagctgtaaagggaaccctcagaaaatgtggccgccaggggtggtttcaggtgctttgctgggctgtgtttgtgaaaacccatttggacccgccctccaagtccaccctccaggtccaccctccagggccgccctgggctgggggtatgcctggcgttccttgtgccgcagcccggagcacagcaggctgtgcacatttaaatccactaagattcactcggggggagcccaggtcccaagcaactgagggctcaggagtcctgaggctgctgaggggacagagcagacggggaacgctgcttctgtgtggcaagttcctgagggtgctggccagggaggtggctcagagtgtatgttggggtcccaccgggggcagaactctgtctctgatgagtcggcagccatgtaacaggaaggggtggccacagggagctgggaatgcaccaggggagctgcgcagctggccgaggtcccagggccaggccacaggaagggcagggggacgcccggggccacagcagaggccgcaggaagggaaggggatgcccaggccagagcagaggctaccgggcacaggggggctccctgagctgggtgagcgaggctcatgactcggcgagggaacctccttgacgtgaagctgacgactggtgttgcccagctcacagcccagccaggtcccgcgcctgagcaggaactcagaaccctcccctttgtctaaagcacagcagatgccttcagggcatctaggagaaaacaggcaaagtcgttgagaaacgtcttaaaagaaggtgggatggtggcaatttcttgtccagattttagtctgccccggaccacagatgagtctataacgggattgtggtgttgccatggggacacatgagatggaccatcacagaggccactggggctgcacctcccatctgagtcctggctgtcccgggtccaggccaggttcttgcatgctcacctacctgtcctgcccgggagacagggaaagcaccccgaagtctggagcagggctgggtccaggctcctcagagctcctgccaggcccagcaccctgctccaaatcaccacttctctggggttttccaaagcatttaacaagggtgtcaggttacctcctgggtgacggccccgcatcctggggctgacattgcccctctgccttag

SEQ ID NO: 15 - DNA, 3801 bp, Homo sapiens
gtgagcgcacctggccggaagtggagcctgtgcccggctggggcaggtgctgctgcagggccgttgcgtccacctctgcttccgtgtggggcaggcgactgccaatcccaaagggtcagaggccacagggtgcccctcgtcccatctggggctgagcagaaatgcatctttctgtgggagtgagggtgctcacaacgggagcagttttctgtgctattttggtaaaaggaaatggtgcaccagacctgggtgcactgaggtgtcttcagaaagcagtctggatccgaacccaagacgcccgggccctgctgggcgtgagtctctcaaacccgaacacaggggccctgctgggcatgagtccctctgaacccgagaccctggggccctgctgggcgtgagtctctccgaacccagagacttcagggcccttttgggcgtgagtctctccgctgtgagccccacactccaaggctcatccacagtctacaggatgccatgagttcatgatcacgtgtgacccatcaggggacagggccatggtgtggggggggtctctacaaaattctggggtcttgtttccccagagcccgagagctcaaggccccgtctcaggctcagacacaaatgaattgaagatggacacagatgcagaaatctgtgctgtttcttttatgaataaaaagtatcaacattccaggcagggcaaggtggctcacacctataatcccagcactttgggaggccgaggtgggtggatcacttgaggccaggagtttgaggccaacctaaccaacatagtgaaattccatttctacttaaaaaatacaaaaattagcctggcctggtggcacacgcctgtagtccccgctatgcgggaggctgaggcaggagaatcatttgaacccaggaggcagaggttgcagtgagccgagatcacaccactgcactccagcctgggcaacagagtgagacttcatcttaaaaaaaaaaaaaaaagtatcagcattccaaaaccatagtggacaggtgtttttttattctgtccttcgataatatttactggtgctgtgctagaggccggaactgggggtgccttcctctgaaaggcacaccttcatgggaagagaaataagtggtgaatggttgttaaaccagaggtttaaactggggtcctgtcgttctgagttaacagtccagatctggactttgcctctttccagaatgctccctggggtttgcttcatgggggagcagcaggtgtggacaccctcgtgatgggggagcagcaggtgcagacgccctcatgatgggggagtggcaggtgcagacacccttgtgcatggtgcccagcatgtccctgttgcagctccctccccacaaggatgccggtctcctgtgctccccacagtccctgcttccctctcacagccttacctggtcctggcctccactggctttgtctgcatgatttccacatttcctgggctcccagcacctcttcgcctctcccaggcacctctgcagtgctggccataccagtcagctgtgaactgtccactgcttattttgctccccatgaaatgtattttttaggacaggcacccctggttccagcctctggcacagcatcagtgaatgttattgaaggacaaaggacagacaaacaaatcaggaaaatgggttctctctaaacacattgcaaagccacagaggctagtgcaggatgggtgggcatcaggtcatcagatgtgggtccaatgccagaatattctgtgctcccaaaggccacttggtcagagtgtgtgcttgcagaggtggctctaaaagctcagcagtggaggcagtggttcgccatactcagggtgaactcacatcctctgtgtctgaagtatacagcagaggcttgaagggcatctgggagaagaaaacaggcaaaatgattaagaaaagtgaaaaaggaaaagtggtaagatgggaattttcttgtccagattttagtctcccaaaccacagctcagatggtagaatgtggtcagaactgatggacagaacaatagaacaaaacggaagccctatctctcagaaacgtgtgttaatgtggtatgtggcacagctgatggaaaagagagtgtgtgtgtaatttttttttctgagaaaactgactggaagcaaataagttgtgtctttacagcatataccagagcagattctaggtagaagaggagacacatgcaaacaacaccagcaacagaaataaaacaaaagactcaaagggaagggaggtgaacgttccctggtttggtgttggggaaggacacacagggaggcggatgaaaccagtgaggcaacgggcattgctttcactgcagagaaactcagcttgcctgagccacagtgaaaatggccattccctggagcgtttgtgcacgtgatttatttaaggcgccctgtgaggtcctgcacattcatcctctcactttgttctcctaaccacctgagaggtagaggaggaaaggctccaggggagcagccgcccttggtcacccagctggcaaagggcatgcatgattgcagcctggcctcctgctccggggcccttgctctgcccgaggaccccacacaagtcagacccataggctcagggtgagccggagcccaaggtcgtgttggggatggctgtgaaagaagaaatggacgtctgatgcacacttgggaaggtcctaccagcagcgtcaaagaaatgcatgtgaaactgacagcgagacccatccctcaaagaaacgcacgtgaaactgatggcgagacctgtccccatccctcatgctggctccttttctgggcttgccaagagccagcatcaggttgaggcaagctggaaagacttttctggaaagcagcttgtttgcatggaagtcctcacaatgtcctgtgtcttcccagtaattccacttctgaagtgaccagacattatcacgggtcttatttaccatttccagtgttccaggcagggggacttgccacagcaagtcacgaacctgcccaaatacagggctaaggagatattatgcatcacaaaacttgctctgccattaaacatttttcaaagaatttttgaagaatgtttaatggcacaaaacgtttatttcaatgtagcagtgttcaaagctggatgtaaaagaacacaccccaggagcctgccgtgaatgtcatgtgtgttcatctttggacatggacatacatgggcagtgagtggtggtgaggccctggaggacatcggtgggatgcctccatcctgcccctctggagacaccatgtgtgccacgtgcactcactggagccctgtttagctggtgccacctggctcttccatccctgagattcaaacacagtgagattccccacgcccaactcagtgttctcccacaaaaaacctgagtcacacctgtgttcactcgagggacgcccgggagccagggctccacagtttattatgtgtttttggctgagttatgtgcagatctcatcagggcagatgatgagtgcacaaacacggccgtgcgaggtttggatacactcaacatcactagccaggtcctggtggagtttggtcatgcagagtctggatggcatgtagcatttggagtccatggagtgagcacccagccccctcgggctgcagcgcatgccccaggcaggacaaggaagcgggaggaaggcaggaggctctttggagcaagctttgcaggagggggctgggtgtggggcaggcacctgtgtctgacattcccccctgtgtctcag

SEQ ID NO: 16 - DNA, 880 bp, Homo sapiens
gtgagcaggctgatggtcagcacagagttcagagttcaggaggtgtgtgcgcaagtatgtgtgtgtgtgtgtgcgcgcgtgcctgcaaggctgatggtgactggctgcacgtaagagtgcacatgtacgcatatacacgtgagcacatacatgtgtgcatgtgtgtacatgaaggcatggcagtgtgtgcacaggtgtgcaagggcacaagtgtgtgcacatgcgaatgcacacctgacatgcatgtgtgttcgtgcacagtcgtgtgggcattcacgtgaggtgcatgcgtgtgggtgtgcagtgtgagtagcatgtgtgcacataacatgtattgaggggtcctcgtgttcaccccgctaggtcctcagcaccagtgccactccttacaggatgagacggggtcccaggccttggtgggctgaggctctgaagctgcagccctgagggcattgtcccatctgggcatccgcgtccactccctctcctgtgggcttctgtgtccactccccctctcctgtgggcatttacatccactccactccctctctcctgtgggcatccgcgtccactccccctctctgtgggcatctgcgtccacctcccctctctgtgggcatttgcgtccactccctctcctggttccttcctgtcttggccgagcctcgggggcaggcagatgacacagagtcttgactcgcccagggtggttcgcagctgccgggtgagggccaggccggatttcactgggaagagggatagtttcttgtcaaaatgttcctctttcttgttccatctgaatggatgataaagcaaaaagtaaaaacttaaaatcccagagaggtttctaccgtttctcactctttcttggcgactctag

SEQ ID NO: 17 - DNA, 3186 bp, Homo sapiens
gtgagccgccaccaaggggtgcaggcccagcctccagggaccctccgcgctctgctcacctctgacccggggcttcaccttggaactcctgggttttaggggcaaggaatgtcttacgttttcagtggtgctgctgcctgtgcacagttctgttcgcgtggctctgtgcaaagcacctgttctccatctctgggtagtggtaggagccggtgtggccccaggtgtccccactgtgcctgtgcactggccgtgggacgtcatggaggccatcccagggcagcaggggcatggggtaaagagatgtttatggggagtcttagcagaggaggctgggaaggtgtctgaacagtagatgggagatcagatgcccggaggatttggggtctcagcaaagagggccgaggtgggtgcaggtgagggtcgctggccccacccccgggaaggtgcagcagagctgtggctccccacacagcccggccagcacctgtgctctgggcatggctgtgctcctggaacgttccctgtcctggctggtcagggggtgcccctgccaagaatcgacaactttatcacagagggaagggccaatctgtggaggccacagggccagcttctgcctggagtcagggcaggtggtggcacaagcctcggggctgtaccaaagggcagtcgggcaccacaggcccgggcctccacctcaacaggcctcccgagccactgggagctgaatgccaggaggccgaagccctcgccccatgagggctgagaaggagtgtgagcatttgtgttacccagggccgaggctgcgcgaattaccgtgcacacttgatgtgaaatgaggtcgtcgtctatcgtggaaacccagcaagggctcacgggagagttttccattacaaggtcgtaccatgaaaatggtttttaacccgagtgcttgcgccttcatgctctggcagggagggcagagccacagctgcatgttaccgcctttgcaccagctccagaggcttgggaccaggctgtctcagttccagggtgcgtccggctcagaccgccctcctctctgccttctctctctgcctcaaatcttccctcgtttgcatctccctgacgcgtgcctgggccctcgtgcaagctgcttgactcctttccggaaacccttggggtgtgctggatacaggtgccactgaggactggaggtgtctgacactgtggttgaccccagggtccagctggcgtgcttggggcctccttgggccatgatgaggtcagaggagttttcccaggtgaaaactcctgggaaactcccagggccatgtgacctgccacctgctcctcccatattcagctcagtcttgtcctcatttccccaccagggtctctagctccgaggagctcccgtagagggcctgggctcagggcagggcggctgagtttccccacccatgtggggacccttgggtagtcgcttgattgggtagccctgaggaggccgagatgcgatgggccacgggccgtttccaaacacagagtcaggcacgtggaaggcccaggaatccccttccctcgaggcaggagtgggagaacggagagctgggccccgatttcacggcagccaggctgcagtgggcgaggctgtggtggtccacgtggcgctgggggcggggtctgattcaaatccgctggggctcggccttcctggcccgtgctggccgcgcctccacacgggcttggggtggacgccccgacctctagcaggtggctatttctccctttggaagagagcccctcacccatgctaggtgtttccctcctgggtcaggagcgtggccgtgtggcaaccccgggaccttaggcttatttatttgtttaaaaacattctgggcctggcttccgttgttgctaaatggggaaaagacatcccacctcagcagagttactgagaggctgaaaccggggtgctggcttgactggtgtgatctcaggtcattccagaagtggctcaggaagtcagtgagaccaggtacatggggggctcaggcagtgggtgagatgaggtacacggggggctcaggcagtgggtgaggccaggtacatggggggctcaggcactgggtgagatgaggtacacggggggctcaggcagagggtcagaccaggtacacgggggctctgatcacacgcacatatgagcacatgtgcacatgtgctgtttcatggtagccaggtctgtgcacacctgccccaaagtcccaggaagctgagaggccaaagatggaggctgacagggctggcgcggtggctcacacctgtagtcccagcactttgggaggccgaggcgagaggatcccttgagcccaggagtttaagaccagcctgagcaacatagtagaaccccatctctatgaaaaataaaaacaaaaattagctgaacatggtggtgtgcgcctgtagttccaatacttgggaggctgaagtgggaggatcacttgagcccaggaggtggaagctgcagtgagctgagattgcaccactgtactgcagcctgggtgacagagtgagagcccatctcaacaacaacaaagaagactgacaaatgcagtttcttggaaagaaacatttagtaggaacttaacctacacacagaagccaagtcggtgtctcggtgtcagtgagatgagatgatgggtcctcacaccatcaccccagacccagggtttatgcaccacaggggcgggtggctcagaagggatgcgcaggacgttgatatacgatgacatcaaggttgtctgacgaagggcaggattcatgataagtacctgctggtacacaaggaacaatggataaactggaaaccttagaggccttcccggaacaggggctaatcagaagccagcatggggggctggcatccaggatggagctgcttcagcctccacatgcgtgttcatacagatggtgcacagaaacgcagtgtacctgtgcacacacagacacgcagctactcgcacacacaagcacacacacagacatgcatgcatgcatccgtgtgtgtgcacctgtgcccatgaggaaacccatgcatgtgcattcatgcacgcacacaggcaccggtgggcccatgcccacacccacgagcaccgtctgattaggaggcctttcctctgacgctgtccgccatcctctcag

SEQ ID NO: 18 - DNA, 781 bp, Homo sapiens
gtatgtgcaggtgcctggcctcagtggcagcagtgcctgcctgctggtgttagtgtgtcaggagactgagtgaatctgggcttaggaagttcttaccccttttcgcatcaggaagtggtttaacccaaccactgtcaggctcgtctgcccgccctctcgtggggtgagcagagcacctgatggaagggacaggagctgtctgggagctgccatccttcccaccttgctctgcctggggaagcgctggggggcctggtctctcctgtttgccccatggtgggatttggggggcctggcctctcctgtttgccctgtggtgggattgggctgtctcccgtccatggcacttagggcccttgtgcaaacccaggccaagggcttaggaggaggccaggcccaggctaccccacccctctcaggagcagaggccgcgtatcaccacgacagagccccgcgccgtcctctgcttcccagtcaccgtcctctgcccctggacactttgtccagcatcagggaggtttctgatccgtctgaaattcaagccatgtcgaacctgcggtcctgagcttaacagcttctactttctgttctttctgtgttgtggaaatttcacctggagaagccgaagaaaacatttctgtcgtgactcctgcggtgcttgggtcgggacagccagagatggagccaccccgcagaccgtcgggtgtgggcagctttccggtgtctcctgggaggggagctgggctgggcctgtgactcctcagcctctgttttcccccag

SEQ ID NO: 19 - DNA, 536 bp, Homo sapiens
gcaagtgtgggtggaggccagtgcgggccccacctgcccaggggtcatccttgaacgccctgtgtggggcgagcagcctcagatgctgctgaagtgcagacgcccccgggcctgaccctgggggcctggagccacgctggcagccctatgtgattaaacgctggtgtccccaggccacggagcctggcagggtccccaacttcttgaacccctgcttcccatctcaggggcgatggctccccacgcttgggagccttctgacccctgacctgtgtcctctcacagcctcttccctggctgctgccctgagctcctggggtcctgagcaagttctctccccgccccgccgctccagcgtcactgggctgcctgtctgctcgccccggtggaggggtgtctgtcccttcactgaggttcccaccagccagggccacgaggtgcaggccctgcctgcccggccacccacacgtcctaggagggttggaggatgccacctctggcctcttctggaacggagtctgattttggccccgcag

SEQ ID NO: 20 - DNA, 3179 bp, Homo sapiens
atctcatgtttgaatcctaatgtgcactgcatagacaccactgtatgcaattacagaagcctgtgagtgaacggggtggtggtcagtgcgggcccatggcctggctgtgcatttacggaagtctatgagtgaatggggttgtggtcagtgcgggcccatggcctggctgggcctgggaggtttctgatgctgtgaggcaggaggggaaggagggtaggggatagacagtgggagcccccaccctggaagacataacagtaagtccaggcccgaagggcagcagggatgctgggggcccagcttgggcggcggggatgatggagggcctggccagggtggcagggatgatgggggccccagctggggtggcaggggtgatggggggggctggtctgggtggcggggaagatggggaagcctggctgggccccctcctcccctgcctcccacctgcagccgtggatccggatgtgcttccctggtgcacatcctctgggccatcagctttcatggaggtggggggcaggggcatgacaccatcctgtataaaatccaggattcctcctcctgaacgccccaactcaggttgaaagtcacattccgcctctggccattctcttaagagtagaccaggattctgatctctgaagggtgggtagggtggggcagtggagggtgtggacacaggaggcttcagggtggggctggtgatgctctctcatcctcttatcatctcccagtctcatctctcatcctcttatcatctcccagtctcatctgtcttcctcttatctcccagtctcatctgtcatcctcttaccatctcccagtctcatctcttatcctcttatctcctagtctcatccagacttacctcccagggcgggtgccaggctcgcagtggagctggacatacgtccttcctcaggcagaaggaactggaaggattgcagagaacaggaggggcggctcagagggacgcagtcttggggtgaagaaacagcccctcctcagaagttggcttgggccacacgaaaccgagggccctgcgtgagtggctccagagccttccagcaggtccctggtggggccttatggtatggccgggtcctactgagtgcaccttggacagggcttctggtttgagtgcagcccggacgtgcctggtgtcggggtgggggcttatggccactggatatggcgtcatttattgctgctgcttcagagaatgtctgagtgaccgagcctaatgtgtatggtgggcccaagtccacagactgtgtcgtaaatgcactctggtgcctggagcccccgtataggagctgtgaggaaggaggggctcttggcagccggcctgggggcgcctttgccctgcaaactggaagggagcggccccgggcgccgtgggcggacgacctcaagtgagaggttggacagaacagggcggggacttcccaggagcagaggccgctgctcaggcacacctgggtttgaatcacagaccaacaggtcaggccattgttcagctatccatcttctacaaagctccagattcctgtttctccgggtgttttttgttgaaattttactcaggattacttatattttttgctaaagtattagacccttaaaaaaggtatttgctttgatatggcttaactcactaagcacctactttatttgtctgtttttatttattattattattattattagagatggtgtctactctgtcacccaggttgttagtgcagtggcacagtcatggctcgctgtagccgcaaacccccaggctcaagtgatcctccggcctcagcttcccagagtgctgggattacaggtgtgagccactgcccttgcctggcacttttaaaaaccactatgtaaggtcaggtccagtggcttccacacctgtcatcccagtagtttgggaagccgaggcagaaggattgtctgaggccaggagtttgagaccagcatgggtaacatagggagaccccatctctacaaaaaatgcaaaaagttatccgggcgtggggtccagcatctgtagtcccagctgctcgggaggctgagtgggaggatcgcttgagcccgggaggtcatggctgcagtgagctgtgattgtaccatcgcactccagcctgggcaacagagtgagaccctgtctcaaaaaaaaaaaaaaaaaaagaaggagaaggagaagagaagaagaaggaagaaggaaagagaagaagaaggaagaaggaagaaagaaggagaaggaggcctgctaggtgctaggtagactgtcaaatctcagagcaaaatgaaaataacaaagttttaaagggaaagaaaaaccccagctctttggacttccttaggcctgaacttcatctcaagcagcttccttccacagacaagcgtgtatggagcgagtgagttcaaagcagaaagggaggagaagcaggcaagggtggaggctgtgggtgacaccagccaggacccctgaaagggagtggttgttttcctgcctcagccccacgctcctgccggtcctgcacctgctgtaaccgtcgatgttggtgccaggtgcccacctgggaaggatgctgtgcagggggcttgccaaactttggtgggtttcagaagccccaggcacttgtggcaggcacaattacagcccctccccaaagatgcccacgtccttctcctggaacctgtgaatgtgtcacccgcaaggcagaggctggtgaaggctgcaggtggaatcacggctgccagtcagccgatcttaaggtcatcctggattatctggtgggcctgatatggccacaagggtccctagaagtgagagagggaggcaggggagagtcagagaggggacgtgagaaggaccactggccactgctggctttgagatggaggagggggtccccagccaaggaatgggggcagccgctccatgctggaaaagcaagcaatcctccccggtcctgagggcacacggccctgcccacgcctcgatttcaggccagtgggacctgtttcagctttccggcctccagagctgtaagatgatgcgtttgtgttcagccactaagctgcagtgattcgtcacagcagcaaatggaatagcagtacagggaaatgaatacagggacagttctcagagtgactctcagcccacccctggg

SEQ ID NO: 21 - DNA, 27 bp, Homo sapiens
gttaagttgtagcttacactggttctc

SEQ ID NO: 22 - DNA, 22 bp, Adapter primer
gtaatacgactcactatagggc

SEQ ID NO: 23 - DNA, 19 bp, Adapter primer
actatagggcacgcgtggt

SEQ ID NO: 24 - DNA, 22 bp, Primer
gacgtggctcttgaaggccttg

SEQ ID NO: 25 - DNA, 22 bp, Primer
gccttctggaccacggcatacc

SEQ ID NO: 26 - DNA, 24 bp, Primer
cgtagttgagcacgctgaacagtg

SEQ ID NO: 27 - DNA, 24 bp, Primer
ccttcaccctcgaggtgagacgct

SEQ ID NO: 28 - DNA, 23 bp, Primer
ggtggatgtgacgggcgcgtacg

SEQ ID NO: 29 - DNA, 22 bp, Primer
ggtatgccgtggtccagaaggc

SEQ ID NO: 30 - DNA, 25 bp, Primer
cctccaaagaggtggcttcttcggc

SEQ ID NO: 31 - DNA, 22 bp, Primer
cgcagccactaccgcgaggtgc

SEQ ID NO: 32 - DNA, 27 bp, Primer
ctgcgtcctgctgcgcacgtgggaagc

SEQ ID NO: 33 - DNA, 23 bp, Primer
ctcgcggcgcgagtttcaggcag

SEQ ID NO: 34 - DNA, 22 bp, Primer
ccagcccctccccttcctttcc

SEQ ID NO: 35 - DNA, 21 bp, Primer
ccagctccgcctcctccgcgc

SEQ ID NO: 36 - DNA, 23 bp, Primer
ctaggccgattcgacctctctcc

SEQ ID NO: 37 - DNA, 21 bp, Primer
gtcccagggcacgcacaccag

SEQ ID NO: 38 - DNA, 31 bp, Primer
gcaagcttgacgcagcgctgcctgaaactcg

SEQ ID NO: 39 - DNA, 32 bp, Primer
ccagatctctggaacacagagtggcagtttcc

SEQ ID NO: 40 - DNA, 33 bp, Primer
ccagatctgcatgaagtgtgtggggatttgcag

SEQ ID NO: 41 - DNA, 32 bp, Primer
ggagatctgatcttggcttactgcagcctctg

SEQ ID NO: 42 - DNA, 32 bp, Primer
ggagatctgtctggattcctgggaagtcctca

SEQ ID NO: 43 - DNA, 26414 bp, Homo sapiens
acttgagcccaagagttcaaggctacggtgagccatgattgcaacaccacacgccagccttggtgacagaatgagaccctgtctcaaaaaaaaaaaaaaaaattgaaataatataaagcatcttctctggccacagtggaacaaaaccagaaatcaacaacaagaggaattttgaaaactatacaaacacatgaaaattaaacaatatacttctgaatgaccagtgagtcaatgaagaaattaaaaaggaaattgaaaaatttatttaagcaaatgataacggaaacataacctctcaaaacccacggtatacagcaaaagcagtgctaagaaggaagtttatagctataagcagctacatcaaaaaagtagaaaagccaggcgcagtggctcatgcctgtaatcccagcactttgggaggccaaggcgggcagatcgcctgaggtcaggagttcgagaccagcctgaccaacacagagaaaccttgtcgctactaaaaatacaaaattagctgggcatggtggcacatgcctgtaatcccagctactcgggaggctgaggcaggataaccgcttgaacccaggaggtggaggttgcggtgagccgggattgcgccattggactccagcctgggtaacaagagtgaaaccctgtctcaagaaaaaaaaaaaagtagaaaaacttaaaaatacaacctaatgatgcaccttaaagaactagaaaagcaagagcaaactaaacctaaaattggtaaaagaaaagaaataataaagatcagagcagaaataaatgaaactgaaagataacaatacaaaagatcaacaaaattaaaagttggttttttgaaaagataaacaaaattgacaaacctttgcccagactaagaaaaaaggaaagaagacctaaataaataaagtcagagatgaaaaaagagacattacaactgataccacagaaattcaaaggatcactagaggctactatgagcaactgtacactaataaattgaaaaacctagaaaaaatagataaattcctagatgcatacaacctaccaagattgaaccatgaagaaatccaaagcccaaacagaccaataacaataatgggattaaagccataataaaaagtctcctagcaaagagaagcccaggacccaatggcttccctgctggattttaccaatcatttaaagaagaatgaattccaatcctactcaaactattctgaaaaatagaggaaagaatacttccaaactcattctacatggccagtattaccctgattccaaaaccagacaaaaacacatcaaaaacaaacaaacaaaaaaacagaaagaaagaaaactacaggccaatatccctgatgaatactgatacaaaaatcctcaacaaaacactagcaaaccaaattaaacaacaccttcgaaagatcattcattgtgatcaagtgggatttattccagggatggaaggatggttcaacatatgcaaatcaatcaatgtgatacatcatcccaacaaaatgaagtacaaaaactatatgattatttcactttatgcagaaaaagcatttgataaaattctgcacccttcatgataaaaaccctcaaaaaaccaggtatacaagaaacatacaggccaggcacagtggctcacacctgcgatcccagcactctgggaggccaaggtgggatgattgcttgggcccaggagtttgagactagcctgggcaacaaaatgagacctggtctacaaaaaacttttttaaaaaattagccaggcatgatggcatatgcctgtagtcccagctagtctggaggctgaggtgggagaatcacttaagcctaggaggtcgaggctgcagtgagccatgaacatgtcactgtactccagcctagacaacagaacaagaccccactgaataagaagaaggagaaggagaagggagaagggagggagaagggaggaggaggagaaggaggaggtggaggagaagtggaaggggaaggggaagggaaagaggaagaagaagaaacatatttcaacataataaaagccctatatgacagaccgaggtagtattatgaggaaaaactgaaagcctttcctctaagatctggaaaatgacaagggcccactttcaccactgtgattcaacatagtactagaagtcctagctagagcaatcagataagagaaagaaataaaaggcatccaaactggaaaggaagaagtcaaattatcctgtttgcagatgatatgatcttatatctggaaaagacttaagacaccactaaaaaactattagagctgaaatttggtacagcaggatacaaaatcaatgtacaaaaatcagtagtatttctatattccaacagcaaacaatctgaaaaagaaaccaaaaaagcagctacaaataaaattaaacagctaggaattaaccaaagaagtgaaagatctctacaatgaaaactataaaatgttgataaaagaaattgaagagggcacaaaaaaagaaaagatattccatgttcatagattggaagaataaatactgttaaaatgtccatactacccaaagcaatttacaaattcaatgcaatccctattaaaatactaatgacgttcttcacagaaatagaagaaacaattctaagatttgtacagaaccacaaaagacccagaatagccaaagctatcctgaccaaaaagaacaaaactggaagcatcacattacctgacttcaaattatactacaaagctatagtaacccaaactacatggtactggcataaaaacagatgagacatggaccagaggaacagaatagagaatccagaaacaaatccatgcatctacagtgaactcatttttgacaaaggtgccaagaacatactttggggaaaagataatctcttcaataaatggtgctggaggaactggatatccatatgcaaaataacaatactagaactctgtctctcaccatatacaaaagcaaatcaaaatggatgaaaggcttaaatctaaaacctcaaactttgcaactactaaaagaaaacaccggagaaactctccaggacattggagtgggcaaagacttcttgagtaattccctgcaggcacaggcaaccaaagcaaaaacagacaaatgggatcatatcaagttaaaaagcttctgcccagcaaaggaaacaatcaacaaagagaagagacaacccacagaatgggagaatatatttgcaaactattcatctaacaaggaattaataaccagtatatataaggagctcaaactactctataagaaaaacacctaataagctgattttcaaaaataagcaaaagatctgggtagacatttctcaaaataagtcatacaaatggcaaacaggcatctgaaaatgtgctcaacaccactgatcatcagagaaatgcaaatcaaaactactatgagagatcatctcatcccagttaaaatggcttttattcaaaagacaggcaataacaaatgccagtgaggatgtggataaaaggaaacccttggacactgttggtgggaatggaaattgctaccactatggagaacagtttgaaagttcctcaaaaaactaaaaataaagctaccatacagcaatcccattgctaggtatatactccaaaaaagggaatcagtgtatcaacaagctatctccactcccacatttactgcagcactgttcatagcagccaaggtttggaagcaacctcagtgtccatcaacagacgaatggaaaaagaaaatgtggtgcacatacacaatggagtactacgcagccataaaaaagaatgagatcctgtcagttgcaacagcatggggggcactggtcagtatgttaagtgaaataagccaggcacagaaagacaaacttttcatgttctcccttacttgtgggagcaaaaattaaaacaattgacatagaaatagaggagaatggtggttctagaggggtgggggacagggtgactagagtcaacaataatttattgtatgttttaaaataactaaaagagtataattgggttgtttgtaacacaaagaaaggataaatgcttgaaggtgacagataccccatttaccctgatgtgattattacacattgtatgcctgtatcaaaatatctcatgtatgctatagatataaaccctactatattaaaaattaaaattttaatggccaggcacggtggctcatgtccgtaatcccagcactttgggaggccgaggcgggtggatcacctgaggtcaggagtttgaaaccagtctggccaccatgatgaaaccctgtctctactaaagatacaaaaattagccaggcgtggtggcacatacctgtagtcccaactactcaggaggctgagacaggagaattgcttgaacctgggaggcggaggttgcagtgagccgagatcatgccactgcactgcagcctgggtgacagagcaagactccatctcaaaacaaaaacaaaaaaaagaagattaaaattgtaatttttatgtaccgtataaatatatactctactatattagaagttaaaaattaaaacaattataaaaggtaattaaccacttaatctaaaataagaacaatgtatgtggggtttctagcttctgaagaagtaaaagttatggccacgatggcagaaatgtgaggagggaacagtggaagttactgttgttagacgctcatactctctgtaagtgacttaattttaaccaaagacaggctgggagaagttaaagaggcattctataagccctaaaacaactgctaataatggtgaaaggtaatctctattaattaccaataattacagatatctctaaaatcgagctgcagaattggcacgtctgatcacaccgtcctctcattcacggtgctttttttcttgtgtgcttggagattttcgattgtgtgttcgtgtttggttaaacttaatctgtatgaatcctgaaacgaaaaatggtggtgatttcctccagaagaattagagtacctggcaggaagcaggtggctctgtggacctgagccacttcaatcttcaagggtctctggccaagacccaggtgcaaggcagaggcctgatgacccgaggacaggaaagctcggatgggaaggggcgatgagaagcctgcctcgttggtgagcagcgcatgaagtgcccttatttacgctttgcaaagattgctctggataccatctggaaaaggcggccagcgggaatgcaaggagtcagaagcctcctgctcaaacccaggccagcagctatggcgcccacccgggcgtgtgccagagggagaggagtcaaggcacctcgaagtatggcttaaatctttttttcacctgaagcagtgaccaaggtgtattctgagggaagcttgagttaggtgccttctttaaaacagaaagtcatggaagcacccttctcaagggaaaaccagacgcccgctctgcggtcatttacctctttcctctctccctctcttgccctcgcggtttctgatcgggacagagtgacccccgtggagcttctccgagcccgtgctgaggaccctcttgcaaagggctccacagacccccgccctggagagaggagtctgagcctggcttaataacaaactgggatgtggctgggggcggacagcgacggcgggattcaaagacttaattccatgagtaaattcaacctttccacatccgaatggatttggattttatcttaatattttcttaaatttcatcaaataacattcaggactgcagaaatccaaaggcgtaaaacaggaactgagctatgtttgccaaggtccaaggacttaataaccatgttcagagggatttttcgccctaagtactttttattggttttcataaggtggcttagggtgcaagggaaagtacacgaggagaggcctgggcggcagggctatgagcacggcagggccaccggggagagagtccccggcctgggaggctgacagcaggaccactgaccgtcctccctgggagctgccacattgggcaacgcgaaggcggccacgctgcgtgtgactcaggaccccataccggcttcctgggcccacccacactaacccaggaagtcacggagctctgaacccgtggaaacgaacatgacccttgcctgcctgcttccctgggtgggtcaagggtaatgaagtggtgtgcaggaaatggccatgtaaattacacgactctgctgatggggaccgttccttccatcattattcatcttcacccccaaggactgaatgattccagcaacttcttcgggtgtgacaagccatgacaaaactcagtacaaacaccactcttttactaggcccacagagcacggsccacacccctgatatattaagagtccaggagagatgaggctgctttcagccaccaggctggggtgacaacagcggctgaacagtctgttcctctagactagtagaccctggcaggcactcccccagattctagggcctggttgctgcttcccgagggcgccatctgccctggagactcagcctggggtgccacactgaggccagccctgtctccacaccctccgcctccaggcctcagcttctccagcagcttcctaaaccctgggtgggccgtgttccagcgctactgtctcacctgtcccactgtgtcttgtctcagcgacgtagctcgcacggttcctcctcacatggggtgtctgtctccttccccaacactcacatgcgttgaagggaggagattctgcgcctcccagactggctcctctgagcctgaacctggctcgtggcccccgatgcaggttcctggcgtccggctgcacgctgacctccatttccaggcgctccccgtctcctgtcatctgccggggcctgccggtgtgttcttctgtttctgtgctcctttccacgtccagctgcgtgtgtctctgcccgctagggtctcggggtttttataggcataggacgggggcgtggtgggccagggcgctcttgggaaatgcaacatttgggtgtgaaagtaggagtgcctgtcctcacctaggtccacgggcacaggcctggggatggagcccccgccagggacccgcccttctctgcccagcactttcctgcccccctccctctggaacacagagtggcagtttccacaagcactaagcatcctcttcccaaaagacccagcattggcacccctggacatttgccccacagccctgggaattcacgtgactacgcacatcatgtacacactcccgtccacgaccgacccccgctgttttattttaatagctacaaagcagggaaatccctgctaaaatgtcctttaacaaactggttaaacaaacgggtccatccgcacggtggacagttcctcacagtgaagaggaacatgccgtttataaagcctgcaggcatctcaagggaattacgctgagtcaaaactgccacctccatgggatacgtacgcaacatgctcaaaaagaaagaatttcaccccatggcaggggagtggttaggggggttaaggacggtgggggcggcagctgggggctactgcacgcaccttttactaaagccagtttcctggttctgatggtattggctcagttatgggagactaaccataggggagtggggatgggggaacccggaggctgtgccatctttgccatgcccgagtgtcctgggcaggataatgctctagagatgcccacgtcctgattcccccaaacctgtggacagaacccgcccggccccagggcctttgcaggtgtgatctccgtgaggaccctgaggtctgggatccttcgggactacctgcaggcccgaaaagtaatccaggggttctgggaagaggcgggcaggagggtcagaggggggcagcctcaggacgatggaggcagtcagtctgaggctgaaaagggagggagggcctcgagcccaggcctgcaagcgcctccagaagctggaaaaagcggggaagggaccctccacggagcctgcagcaggaaggcacggctggcccttagcccaccagggcccatcgtggacctccggcctccgtgccataggagggcactcgcgctgcccttctagcatgaagtgtgtggggatttgcagaagcaacaggaaacccatgcactgtgaatctaggattatttcaaaacaaaggtttacagaaacatccaaggacagggctgaagtgcctccgggcaagggcagggcaggcacgagtgattttatttagctattttattttatttacttactttctgagacagagttatgctcttgttgcccaggctggagtgcagcggcatgatcttggctcactgcaacctccgtctcctgggttcaagcaattctcgtgcctcagcctcccaagtagctgggatttcaggcgtgcaccaccacacccggctaattttgtatttttagtagagatgggctttcaccatgttggtcaagctgatctcaaaatcctgacctcaggtgatccgcccacctcagcctcccaaagtgctgggattacaggcatgagccactgcacctggcctatttaaccattttaaaacttccctgggctcaagtcacacccactggtaaggagttcatggagttcaatttcccctttactcaggagttaccctcctttgatattttctgtaattcttcgtagactggggatacaccgtctcttgacatattcacagtttctgtgaccacctgttatcccatgggacccactgcaggggcagctgggaggctgcaggcttcaggtcccagtggggttgccatctgccagtagaaacctgatgtagaatcagggcgcaagtgtggacactgtcctgaatctcaatgtctcagtgtgtgctgaaacatgtagaaattaaagtccatccctcctactctactgggattgagccccttccctatccccccccaggggcagaggagttcctctcactcctgtggaggaaggaatgatactttgttatttttcactgctggtactgaatccactgtttcatttgttggtttgtttgttttgttttgagaggcggtttcactcttgttgctcaggctggagggagtgcaatggcgcgatcttggcttactgcagcctctgcctcccaggttcaagtgattctcctgcttccgcctcccatttggctgggattacaggcacccgccaccatgcccagctaattttttgtatttttagtagagacgggggtgggtggggttcaccatgttggccaggctggtctcgaacttctgacctcagatgatccacctgcctctgcctcctaaagtgctgggattacaggtgtgagccaccatgcccagctcagaatttactctgtttagaaacatctgggtctgaggtaggaagctcaccccactcaagtgttgtggtgttttaagccaatgatagaatttttttattgttgttagaacactcttgatgttttacactgtgatgactaagacatcatcagcttttcaaagacacactaactgcacccataatactggggtgtcttctgggtatcagcaatcttcattgaatgccgggaggcgtttcctcgccatgcacatggtgttaattactccagcataatcttctgcttccatttcttctcttccctcttttaaaattgtgttttctatgttggcttctctgcagagaaccagtgtaagctacaacttaacttttgttggaacaaattttccaaaccgcccctttgccctagtggcagagacaattcacaaacacagccctttaaaaaggcttagggatcactaaggggatttctagaagagcgacctgtaatcctaagtatttacaagacgaggctaacctccagcgagcgtgacagcccagggagggtgcgaggcctgttcaaatgctagctccataaataaagcaatttcctccggcagtttctgaaagtaggaaaggttacatttaaggttgcgtttgttagcatttcagtgtttgccgacctcagctacagcatccctgcaaggcctcgggagacccagaagtttctcgcccccttagatccaaacttgagcaacccggagtctggattcctgggaagtcctcagctgtcctgcggttgtgccggggccccaggtctggaggggaccagtggccgtgtggcttctactgctgggctggaagtcgggcctcctagctctgcagtccgaggcttggagccaggtgcctggaccccgaggctgccctccaccctgtgcgggcgggatgtgaccagatgttggcctcatctgccagacagagtgccggggcccagggtcaaggccgttgtggctggtgtgaggcgcccggtgcgcggccagcaggagcgcctggctccatttcccaccctttctcgacgggaccgccccggtgggtgattaacagatttggggtggtttgctcatggtggggacccctcgccgcctgagaacctgcaaagagaaatgacgggcctgtgtcaaggagcccaagtcgcggggaagtgttgcagggaggcactccgggaggtcccgcgtgcccgtccagggagcaatgcgtcctcgggttcgtccccagccgcgtctacgcgcctccgtcctccccttcacgtccggcattcgtggtgcccggagcccgacgccccgcgtccggacctggaggcagccctgggtctccggatcaggccagcggccaaagggtcgccgcacgcacctgttcccagggcctccacatcatggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgggcctccccggggtcggcgtccggctggggttgagggcggccggggggaaccagcgacatgcggagagcagcgcaggcgactcagggcgcttcccccgcaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcaggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggtgaggaggtggtggccgtcgagggcccaggccccagagctgaatgcagtaggggctcagaaaagggggcaggcagagccctggtcctcctgtctccatcgtcacgtgggcacacgtggcttttcgctcaggacgtcgagtggacacggtgatctctgcctctgctctccctcctgtccagtttgcataaacttacgaggttcaccttcacgttttgatggacacgcggtttccaggcgccgaggccagagcagtgaacagaggaggctgggcgcggcagtggagccgggttgccggcaatggggagaagtgtctggaagcacagacgctctggcgagggtgcctgcaggttacctataatcctcttcgcaatttcaagggtgggaatgagaggtggggacgagaaccccctcttcctgggggtgggaggtaagggttttgcaggtgcacgtggtcagccaatatgcaggtttgtgtttaagatttaattgtgtgttgacggccaggtgcggtggctcacgccggtaatcccagcactttgggaagctgaggcaggtggatcacctgaggtcaggagtttgagaccagcctgaccaacatggtgaaaccctatctgtactaaaaatacaaaaattagctgggcatggtggtgtgtgcctgtaatcccagctacttgggaggctgaggcaggagaatcacttgaacccaggaggcggaggctgcagtgagctgagattgtgccattgtactccagcctgggcgacaagagtgaaactctgtctttaaaaaaaaaaagtgttcgttgattgtgccaggacagggtagagggagggagataagactgttctccagcacagatcctggtcccatctttaggtatgaagagggccacatgggagcagaggacagcagatggctccacctgctgaggaagggacagtgtttgtgggtgttcaggggatggtgctgctgggccctgccgtgtccccaccctgtttttctggatttgatgttgaggaacctccgctccagcccccttttggctcccagtgctcccaggccctaccgtggcagctagaagaagtcccgatttcaccccctccccacaaactcccaagacatgtaagacttccggccatgcagacaaggagggtgaccttcttggggctcttttttttctttttttctttttatggtggcaaaagtcatataacatgagattggcactcctaacaccgttttctgtgtacagtgcagaattgctaactcggcggtgtttacagcaggttgcttgaaatgctgcgtcttgcgtgactggaagtccctacccatcgaacggcagctgcctcacacctgctgcggctcaggtggaccacgccgagtcagataagcgtcatgcaacccagttttgctttttgtgctccagcttccttcgttgaggagagtttgagttctctgatcaggactctgcctgtcattgctgttctctgacttcagatgaggtcacaatctgcccctggcttatgcagggagtgaggcgtggtccccgggtgtccctgtcacgtgcagggtgagtgaggcgttgcccccaggtgtccctgtcacgtgtagggtgagtgaggcgcggcccccgggtgtccctgtcccgtgcagcgtgattgaggtgtggcccccgggtgtccctgtcacgtgtagggtgagtgaggcgccatccccgggtgtccctgtcacgtgtagggtgagtgaggcgtggtccccgggtgtccctgtcccgtgcagggtgagtgaggcactgtccccgggtgtccctgtcacgtgcagggtgagtgaggcgcggtccccgggtgtccctctcaggtgtagggtgagtgaggcgcggccccagggtgtccctgtcacgtgtagggtgagtgaggcaccgtccctgggtgtccctcccaggtatagggtgagtgaggcactgtccccgggtgtccctgtcacgtgcagggtgagtgaggcgcggcccccgggtgtccctctcaggtgcagggtgagtgaggcgctgtccctgggtgtccctgtctcgtgtagggtgagtgaggctctgtccccaggtgtccttggcgtttgctcacttgagcttgctcctgaatgtttgctctttctatagccacagctgcgccggttgcccattgcctgggtagatggtgcaggcgcagtgctggtccccaagcctatcttttctgatgctcggctcttcttggtcacctctccgttccattttgctacggggacacgggactgcaggctctcgcctcccgcgtgccaggcactgcagccacagcttcaggtccgcttgcctctgttgggcctggcttgctcaccacgtgcccgccacatgcatgctgccaatactcctctcccagcttgtctcatgccgaggctggactctgggctgcctgtgtctgctgccacgtgttgctggagacatcccagaaagggttctctgtgccctgaaggaaagcaagtcaccccagccccctcacttgtcctgttttctcccaagctgcccctctgcttggcccccttgggtgggtggcaacgcttgtcaccttattctgggcacctgccgctcattgcttaggctgggctctgcctccagtcgccccctcacatggattgacgtccagccacaggttggagtgtctctgtctgtctcctgctctgagacccacgtggagggccggtgtctccgccagccttcgtcagacttccctcttgggtcttagttttgaatttcactgatttacctctgacgtttctatctctccattgtatgctttttcttggtttattctttcattccttttctagcttcttagtttagtcatgcctttccctctaagtgctgccttacctgcaccctgtgttttgatgtgaagtaatctcaacatcagccactttcaagtgttcttaaaatacttcaaagtgttaatacttcttttaagtattcttattctgtgatttttttctttgtgcacgctgtgttttgacgtgaaatcattttgatatcagtgacttttaagtattctttagcttattctgtgatttctttgagcagtgagttatttgaacactgtttatgttcaagatatgtagagtatcaagatacgtagagtattttaagttatcattttattattgatttctaactcagttgtgtagtggtctgtataataccaattatttgaagtttgcggagccttgctttgtgatctagtgtgtgcatggtttccagaactgtccattgtaaatttgacatcctgtcaatagtgggcatgcatgttcactatatccagcttattaaggtccagtgcaaagcttctgtctccttctagatgcatgaaattccaagaaggaggccatagtccctcacctgggggatgggtctgttcatttcttctcgtttggtagcatttatgtgaggcattgttaggtgcatgcacgtggtagaatttttatcttcctgatgagtgaatcttttggagacttctatgtctctagtaatctagtaattctttttttaaattgctcttagtactgccacactgggcttcttttgattagtattttcctgctgtgtctgttttctgcctttaatttatatatatatatatattttttttttttttgagacagagtcttggtctgtcgcccagggtgagtgcagtggtgtgatcacaggtcagtgtaacttttaccttctggcctgagccgtcctctcacctcagcctcctgagtagctggaactgcagacacgcaccgctacacctggctaatttttaaattttttctggagacagggtcttgctgtgttgcccaggctggtctcaaactcttggactcaagggatccatctacctcggcttcccaaagtgctgaattacaggcatgagccaccatgtctggcctaattttcaacacttttatattcttatagtgtgggtatgtcctgttaacagcatgtaggtgaatttccaatccagtctgacagtcgttgtttaactggataacctgatttattttcatttttttgtcactagagacccgcctggtgcactctgattctccacttgcctgttgcatgtcctcgttcccttgtttctcaccacctcttgggttgccatgtgcgtttcctgccgagtgtgtgttgatcctctcgttgcctcctggtcactgggcatttgcttttatttctctttgcttagtgttaccccctgatctttttattgtcgttgtttgcttttgtttattgagacagtctcactctgtcacccaggctggagtgtaatggcacaatctcggctcactgcaacctctgcctcctcggttcaagcagttctcattcctcaacctcatgagtagctgggattacaggcgcccaccaccacgcctggctaatttttgtatttttagtagagataggctttcaccatgttggccaggctggtctcaaactcctgacctcaagtgatctgcccgccttggcctcccacagtgctgggattacaggtgcaagccaccgtgcccggcataccttgatcttttaaaatgaagtctgaaacattgctacccttgtcctgagcaataagacccttagtgtattttagctctggccaccccccagcctgtgtgctgttttccctgctgacttagttctatctcaggcatcttgacacccccacaagctaagcattattaatattgttttccgtgttgagtgtttctgtagctttgcccccgccctgcttttcctcctttgttccccgtctgtcttctgtctcaggcccgccgtctggggtccccttccttgtcctttgcgtggttcttctgtcttgttattgctggtaaaccccagctttacctgtgctggcctccatggcatctagcgacgtccggggacctctgcttatgatgcacagatgaagatgtggagactcacgaggagggcggtcatcttggcccgtgagtgtctggagcaccacgtggccagcgttccttagccagtgagtgacagcaacgtccgctcggcctgggttcagcctggaaaaccccaggcatgtcggggtctggtggctccgcggtgtcgagtttgaaatcgcgcaaacctgcggtgtggcgccagctctgacggtgctgcctggcgggggagtgtctgcttcctcccttctgcttgggaaccaggacaaaggatgaggctccgagccgttgtcgcccaacaggagcatgacgtgagccatgtggataattttaaaatttctaggctgggcgcggtggctcacgcctgtaatcccagcactttgggaggccaaggcgggtggatcacgaggtcaggaggtcgagaccatcctggccaacatgatgaaaccccatctgtactaaaaacacaaaaattagctgggcgtggtggcgggtgcctgtaatcccagctactcgggaggctgaggcaggagaattgcttgaacctgggagttggaagttgcagtgagccgacattgcaccactgcactccagcctggcaacacagcgagactctgtctcaaaaaaaaaaaaaaaaaaaaaaaaaaaaattctagtagccacattaaaaaagtaaaaaagaaaaggtgaaattaatgtaataatagattttactgaagcccagcatgtccacacctcatcattttagggtgttattggtgggagcatcactcacaggacatttgacattttttgagctttgtctgcgggatcccgtgtgtaggtcccgtgcgtggccatctcggcctggacctgctgggcttcccatggccatggctgttgtaccagatggtgcaggtccgggatgaggtcgccaggccctcagtgagctggatgtgcagtgtccggatggtgcacgtctgggatgaggtcgccaggccctgctgtgagctggatgtgtggtgtctggatggtgcaggtcaggggtgaggtctccaggccctcggtgagctggaggtatggagtccggatgatgcaggtccggggtgaggtcgccaggccctgctgtgagctggatgtgtggtgtctggatggtgcaggtcaggggtgaggtctccaggccctcggtaagctggaggtatggagtccggatgatgcaggtccggggtgaggtcgccaggccctgctgtgagctggatgtgtggtgtctggatggtgcaggtctggggtgaggtcaccaggccctgcggtgagctgggtgtgcggtgtctggatggtgcaggtctggagtgaggtcgccagacggtgccagaccatgcggtgagctggatatgcggtgtccggatggtgcaggtctggggtgaggttgccaggccctgctgtgagttggatgtggggtgtccggatgctgcaggtccggtgtgaggtcaccaggccctgctgtgagctggatgtgtggtgtctggatggtgcaggtctggggtgaaggtcgccaggcccctgcttgtgagctggatgtgtggtgtctggatggtgcaggtctggagtgaggtcgccaggccctcggtgagctggatgtgcagtgtccagatggtgcaggtccggggtgaggtcgccagaccctgcggtgagctggatgtgcggtgtctggatggtgcaggtctggagtgaggtcgccaggccctcggtgagctggatgtatggagtccggatggtgccggtccggggtgaggtcgccagaccctgctgtgagctggatgtgcggtgtctggatggtacaggtctggagtgaggtcgccagaccctgctgtgagctggatatgcggtgtccggatggtgcaggtcaggggtgaggtctccaggccctcggtgagctggaggtatggagtccggatgatgcaggtccggggtgaggtcgccaggccctgctgtgaactggatgtgcggcgtctggatggtgcaggtctggggtgtggtcgccaggccctcggtgagctggaggtatggagtccggatgatgcaggtccggggtgaggtcgccaggccctgctgtgagctggatgtgcggcgtctggatggtgcaggtctggggtgtggtcgccaggccctcggtgagctggaggtatggagtccggatgatgcaggtccggggtgaggttgccaggccctgctgtgagctggatgtgctgtatccggatggtgcagtccggggtgaggtcgccaggccctgctgtgagctggatgtgctgtatccggatggtgcaggtctggggtgaggtcaccaggccctgcggtgagctggttgtgcggtgtccggttgctgcaggtccggggtgagttcgccaggccctcggtgagctggatgtgcggtgtccccgtgtccggatggtgcaggtccagggtgaggtcgctaggcccttggtgggctggatgtgccgtgtccggatggtgcaggtctggggtgaggtcgccaggcctttggtgagctggatgtgcggtgtctgcatggtgcaggtctggggtgaggtcgccaggcccttggtgggctggatgtgtggtgtccggatggtgcaggtccggcgtgaggtcgccaggccctgctgtgagctggatgtgcggtgtctggatggtgcaggtccggggtgaggtagccaaggccttcggtgagctggatgtggggtgtccggatggtgcaggtccggggtgaggtcgccaggccctgcggttagctggatatgcggtgtccggatggtgcaggtccggggtgaggtcaccaggccctgcggttagctggatgtgcggtgtctggatggtgcaggtccggggtgaggtcgccaggccctgctgtgagctggatgtgctgtatccggatggtgcaggtccggggtgaggtcgccaggccctgcagtgagctggatgtgctgtatccggatggtgcaggtctggcgtgaggtcgccaggccctgcggttagctggatatgcggtgtcggatggtgcaggtccggggtgaggtcaccaggccctgcggttagctggatgtgcggtgtccggatggtgcaggtctggggtgaggtcgccaggccctgctgtgagctggatgtgctgtatccggatggtgcaggtccggggtgaggtcgccaggccctgcggtgagctggatgtgctgtatccggatggtgcaggtctggcgtgaggtcgccaggccctgcggtgagctggatgtgcagtgtacggatggtgcaggtccggggtgaggtcgccaggccctgcggtgggctgtatgtgtgttgtctggatggtgcaggtccggggtgagttcgccaggccctgcggtgagctggatgtgtggtgtctggatgctgcaggtccggggtgagttcgccaggccctcggtgagctggatatgcggtgtccccgtgtccgaatggtgcaggtccagggtgaggtcgccaggcccttggtgggctggatgtgccgtgtccggatggtgcaggtctggggtgaggtcgccaggcccttggtgagctggatgtgcggtgtccggatggtgcaggtccggggtgaggtcaccaggccctcggtgatctggatgtggcatgtccttctcgtttaaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcaggtactgtatccccacgccaggcctctgcttctcgaagtcctggaacaccagcccggcctcagcatgcgcctgtctccacttgcctgtgcttccctggctgtgcagctctgggctgggagccaggggccccgtcacaggcctggtccaagtggattctgtgcaaggctctgactgcctggagctcacgttctcttacttgtaaaatcaggagtttgtgccaagtggtctctagggtttgtaaagcagaagggatttaaattagatggaaacactaccactagcctccttgcctttccctgggatgtgggtctgattctctctctcttttttttttcttttttgagatggagtctcactctgttgcccaggctggagtgcagtggcataatcttggctcactgcaacctccacctcctgggtttaagcgattcaccagcctcagcctcctaagtagctgggattacaggcacctgccaccacgcctggctaatttttgtacttttaggagagacggggtttcaccatgttggccaggctggtctcgaactcatgacctcaggtgatccacccaccttggcctcccaaagtgctgggtttacaggctaagccaccgtgcccagcccccgattctcttttaattcatgctgttctgtatgaatcttcaatctattggatttaggtcatgagaggataaaatcccacccacttggcgactcactgcagggagcacctgtgcagggagcacctggggataggagagttccaccatgagctaacttctaggtggctgcatttgaatggctgtgagattttgtctgcaatgttcggctgatgagagtgtgagattgtgacagattcaagctggatttgcatcagtgagggacgggagcgctggtctgggagatgccagcctggctgagcccaggccatggtattagcttctccgtgtcccgcccaggctgactgtggagggctttagtcagaagatcagggcttccccagctcccctgcacactcgagtccctggggggccttgtgacaccccatgccccaaatcaggatgtctgcagagggagctggcagcagacctcgtcagaggtaacacagcctctgggctggggaccccgacgtggtgctggggccatttccttgcatctgggggagggtcagggctttccctgtgggaacaagttaatacacaatgcaccttacttagactttacacgtatttaatggtgtgcgacccaacatggtcatttgaccagtattttggaaagaatttaattggggtgaccggaaggagcagacagacgtggtggtccccaagatgctccttgtcactactgggactgttgttctgcctggggggccttggaggcccctcctccctggacagggtaccgtgccttttctactctgctgggcctgcggcctgcggtcagggcaccagctccggagcacccgcggccccagtgtccacggagtgccaggctgtcagccacagatgcccaggtccaggtgtggccgctccagcccccgtgcccccatgggtggttttgggggaaaaggccaagggcagaggtgtcaggagactggtgggctcatgagagctgattctgctccttggctgagctgccctgagcagcctctcccgccctctccatctgaagggatgtggctctttctacctgggggtcctgcctggggccagccttgggctaccccagtggctgtaccagagggacaggcatcctgtgtggaggggcatgggttcacgtggccccagatgcagcctgggaccaggctccctggtgctgatggtgggacagtcaccctgggggttgaccgccggactgggcgtccccagggttgactataggaccaggtgtccaggtgccctgcaagtagaggggctctcagaggcgtctggctggcatgggtggacgtggccccgggcatggccttcagcgtgtgctgccgtgggtgccctgagccctcactgagtcggtgggggcttgtggcttcccgtgagcttccccctagtctgttgtctggctgagcaagcctcctgaggggctctctattgcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggtggctgtgctttggtttaacttcctttttaaacagaagtgcgtttgagccccacatttggtatcagcttagatgaagggcccggaggaggggccacgggacacagccagggccatggcacggcgccaacccatttgtgcgcacagtgaggtggccgaggtgccggtgcctccagaaaagcagcgtgggggtgtagggggagctcctggggcagggacaggctctgaggaccacaagaagcagccgggccagggcctggatgcagcacggcccgaggtcctggatccgtgtcctgctgtggtgcgcagcctccgtgcgcttccgcttacggggcccggggaccaggccacgactgccaggagcccaccgggctctgaggatcctggaccttgccccacggctcctgcaccccacccctgtggctgcggtggctgcggtgaccccgtcatctgaggagagtgtggggtgaggtggacagaggtgtggcatgaggatcccgtgtgcaacacacatgcggccaggaacccgtttcaaacagggtctgaggaagctgggaggggttctaggtcccgggtctgggtggctggggacactggggaggggctgcttctcccctgggtccctatggtggggtgggcacttggccggatccactttcctgactgtctcccatgctgtccccgccaggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtgggtgccggggacccccgtgagcagccctgctggaccttgggagtggctgcctgattggcacctcatgttgggtggaggaggtactcctgggtgggccgcagggagtgcaggtgaccctgtcactgttgaggacacacctggcacctagggtggaggccttcagcctttcctgcagcacatggggccgactgtgcaccctgactgcccgggctcctattcccaaggagggtcccactggattccagtttccgtcagagaaggaaccgcaacggctcagccaccaggccccggtgccttgcaccccagtcctgagccaggggtctcctgtcctgaggctcagagaggggacacagcccgccctgcccttggggtctggagtggtgggggtcagagagagagtgggggacaccgccaggccaggccctgagggcagaggtgatgtctgagtttctgcgtggccactgtcagtctcctcgcctccactcacacaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtaaggttcacgtgtgatagtcgtgtccaggatgtgtgtctctgggatatgaatgtgtctagaatgcagtcgtgtctgtgatgcgtttctgtggtggaggtacttccatgatttacacatctgtgatatgcgtgtgtggcacgtgtgtgtcgtggtgcatgtatctgtggcgtgcatatttgtggtgtgtgtgtgtgtggcacgtgtgtgtccatggtgtgtgtgcctgtggtgtgcatgtgtgtgtgtctgtgacacgtgcatgttcatgctgtgtgctgcatgtctgtgatgtgcctatttgtggtgtgtgtgtgcatgtgtccgtgacatatgcgtgtctatggcatgggtgtgtgtggccccttggccttactccttcctcctccaggcatggtccgcaccattgtcctcacgctctcgggtgctggtttggggagctccacattcagggtcctcacttctagcatgggtgcccctgtcctgtcacagggctgggccttggagactgtaagccaggtttgagaggagagtagggatgctggtggtaccttcctggacccctggcacccccaggaccccagtctggcctatgccggctccatgagatataggaaggctgattcaggcctcgctccccgggacacactcctcccagagcggccgggggccttggggctcggcaggggtgaaaggggccctgggcttgggttcccacccagtggtcatgagcacgctggaggggtaagccctcaaagtcgtgccaggccggggtgcagaggtgaagaagtatccctggagcttcggtctggggagaggcacatgtggaaacccacaaggacctctttctctgacttcttgagct

SEQ ID NO: 44 - DNA, 25138 bp, Homo sapiens
  misc_feature (1)..(25138): n is a, c, g, or t.
tgtgggattggttttcatgtgtgggataggtggggatctgtgggattggtttttatgagtggggtaacacagagttcaaggcgagctttcttcctgtagtgggtctgcaggtgctccaacagctttattgaggagaccatatcttcctttgaactatggtcgggtttatagtaagtcaggggtgtggaggcctcccctgggctccctgttctgtttcttccactctggggtcgtgtggtgcctgctgtggtgtgtggccggtgggcagggcttccaggcctccttgtgttcattggcctggatgtggccctggctacgctccgtccttggaattcccctgcgagttggaggctttctttctttctttttttctttctttttttttttttttgataacagagtctcgctcttttttgcccaggctggagtggtttggcgtgatcttggctcactgcaacctgtgcttcctgagttcaagcaattctcttgcctcagcctcccaagtagctggaattataggcgcccaccaccatgctgactaatttttgtaattttagtagagacgaggtttctccatgttggccaggctggtctcgaactcctgacctcaggtgatcctcccacctcggcctcccaaagtgctgggatgacaggtgtgaaccgccgcgcccggccgagactcgcttcctgcagcttccgtgagatctgcagcgatagctgcctgcagccttggtgctgacaacctccgttttccttctccaggtctcgctaggggtctttccatttcatgactctcttcacagaagagtttcacgtgtgctgatttcccggctgtttcctgcgtaattggtgtctgctgtttatcgatggcctccttccatttcctttaggctttgtttattgttgtttttccggctccttgaaggaaaagtttcgattatggatgtttgaactttcttttctaaacaagcatctgaagttgccgttttccctctaaagcagggatcccgaggcccctggctgtggagtggcaccggtctggggcctgttaggaacccggcgcacagcgggaggctaggtggggtgtggggagccagcgttcccgcctgagccccgcccctctcagatcagcagtggcatgcggtgctcagaggcgcacacaccctactgagaactgtgcgtgagaggggtctagattctgtgctccttatgggaatctaatgcctgatgatctgaggtggaaccgtttgctcccaaaaccatccccttccccactgctgtcctgtggaaaaatcgtcttccacgaaaccagtccctggtaccacaatggttggggaccctgtgctaaagacctgcttcagcagcctctcgtcagtgttgatatattggcttttctgtgttgagtccagaataattacggatttctgtgatgctttccgccgacctcagacccatgggctatttgtgggcgtgttgcctgctcctgggttgggaagggtgcaggccccatgtaccttcctgttactgccttccaggttggttctcagggttgaatcgtactcgatgtggttttagcccacggccctgccgccagctcctgggggctggggaacatgctgaagcacagagtcaccgtgcgcgtcttttgatgcctcacaagctcgaggcctcctgtgtccgtgttagtgtgtgtcacgtgcctgctcacatcctgtcttggggacgcaggggcttagcaggtcccgtagtaaatgacaagcgtcctgggggagtctgcagaataggaggtgggggtgccggtctctctcccgcgtcttcagactcttctcctgcctgtgctgtggctgcacctgcatccctgcaatccctccagcactgggctggagaggcccgggagctcgagtgccacttgtgccacgtgactgtggatggcagtcggtcacgggggtctgatgtgtggtgactgtggatggcggttggtcacaggggtctgatgtgtggtgactgtggatggcggtcgtggggtctgatgtggtgactgtggatggcggtcgtggggtctgatgtgtggtgactgtggatggcggtcgtggggtctgatgtggtgactgtggatggcggtcgtggggtctgatgtggtgactgtggatggcggtcgtggggtctgatgtggtgactgtggatggcagtcgtggggtctgatgtgtggtgactgtggatggcggtcgtggggtctgatgtggtgactgtggatggcagtcgtggggtctgatgtgtggtgactgtggatggcggtcgtggggtctgatgtgtggtgactgtggatggcggtcgtggggtctgatgtgtggtgactgtggatggcggtcgtggggtctgatgtgtggtgactgtggatggcggtcgtggggtctgatgtggtgactgtggatggcggtcgtggggtctgatgtgtggtgactgtggatggtgatcggtcacaggggtctgatgtgtggtgactgtggatggcggtcgtggggtctgatgtgtggtgactgtggatggtgatcggtcacaggggtctgatgtgtggtgactgtggatggcggtcgtggggtctgatgtgtggtgactgtggatggcggttggtcccgggggtctgatgtgtggtgactgtggatggcgatcggtcacaggggtctgatgtgtggtgactgtggatggcggtcgtggggtctgatgtgtggtgactgtggatggcggtcgtggggtctgatgtgtggtgactgtggatggcggtcgtggggtctgatgtggtgactgtggatggcggtcgtggggtctgatgtggtgactgtggatggcggtcgtggggtctgatgtgtggtgactgtggatggcggttggtcccgggggtctgatgtgtggtgactgtggatggcggtcgtggggtctgatgtggtgactgtggatggcagtcgtggggtctgatgtgtggtgactgtggatggcggtcgtggggtctgatgtgtggtgactgtggatggcggtcgtggggtctgatgtgtggtgactgtggatggcggtcgtggggtctgatgtgtggtgactgtggatggcggtcgtggggtctgatgtggtgactgtggatggcggtcgtggggtctgatgtgtggtgactgtggatggtgatcggtcacaggggtctgatgtgtggtgactgtggatggcggtcgtggggtctgatgtgtggtgactgtggatggcggtcgtggggtctgatgtggtgactgtggatggcggtcgtggggtctgatgtgtggtgactgtggatggcggtcgtagggtctgatgtgtggtgactgtggatggcagtcggtcacaggggtctgatgtgtggtgactgtggatggcggtcgtggggtctgatgtgtggtgactgtggatggcggtcgtggggtctgatgtgtggtgactgtggatggcggtcgtggggtctgatgtgtggtgactgtggatggcggtcgtggggtctgatgtggtgactgtggatggtgatcggtcacaggggtctgatgtgtggtagctgcaggtggagtcccaggtgtgtctgtagctactttgcgtcctcggccccccggcccccgtttcccaaacagaagcttcccaggcgctctctgggcttcatcccgccatcgggcttggccgcaggtccacacgtcctgatcggaagaaacaagtgcccagctctggccggggcaggccacatttgtggctcatgccctctcctctgccggcaggtctctaccttgacagacctccagccgtacatgcgacagttcgtggctcacctgcaggagaccagcccgctgagggatgccgtcgtcatcgagcaggtctgggcactgccctgcagggttgggcacggactcccagcagtgggtcctcccctgggcaatcactgggctcatgaccggacagactgttggccctggggggcagtggggggaatgagctgtgatgggggcatgatgagctgtgtgccttggcgaaatctgagctgggccatgccaggctgcgacagctgctgcattcaggcacctgctcacgtttgactgcgcggcctctctccagttccgcagtgcctttgttcatgatttgctaaatgtcttctctgccagttttgatcttgaggccaaaggaaaggtgtccccctcctttaggagggcaggccatgtttgagccgtgtcctgcccagctggcccctcagtgctgggtctgaggccaaaggaaacgtgtcccccttcttaggaggacgggccgtgtttgagccacgccccgctgagcgggcctctcagtgctgggtctgtccacgtggccctgtggccctttgcagatgtggtctgtccacgtggccctgtggctctttgcagatgcctgttagcacttgctcggctctaggggacagtcgtgtccaccgcatgaggctcagagacctctgggcgaatttccttggctcccagggtgggggtggaggtggcctgggctgctgggacccagaccctgtgcccggcagctgggcagcaactcctggatcacatatgccatccgggccacggtgggctgtgtgggtgtgagcccagctggacccacaggtggcccagaggagacgttctgtgtcacacactctgcctaagcccatgtgtgtctgcagagactcggcccggccagcccacgatggccctgcattccagcccagccccgcacttcatcacaaacactgaccccaaaagggacggagggtcttggccacgtggtcctgcctgtctcagcacccaccggctcactcccatgtgtctcccgtctgctttcgcagagctcctccctgaatgaggccagcagtggcctcttcgacgtcttcctacgcttcatgtgccaccacgccgtgcgcatcaggggcaagtgagtcaggtggccaggtgccattgccctgcgggtggctgggcgggctggcagggcttctgctcacctctctcctgccccttccccactgnccttctgcccggggccaccagagtctccttttctggcccccgccccctccggctcctgggctgcaggctcccgaggccccggaaacatggctcggcttgcggcagccggagcggagcaggtgccacacgaggcctggaaatggcaagcggggtgtggagttgctcctgcgtggaggacgaggggcggggggtgtgtctgggtcaggtgtgcgccgagcgtttgagcctgcagcttgtcagctccaagttactactgacgctggacacccggctctcacacgcttgtatctctctctcccgatacaaaaggattttatccgattctcattcctgtccctgtcgtgtgacccccgcgagggcgcgggctcttctctctgtgactagatttcccatctggaaagtgcggggttgaccgtgtagtttgctcctctcggggggcctgtggtggccatggggcaggcggcctgggagagctgccgtcacacagccactgggtgagccacactcacggtggtagagccacagtgcctggtgccacatcacgtcctctggattttaagtaaaaccacacacctcccggcaggcatctgcctgcgaccctgtgtgtgcctggggagagtggtagcacggaggaaattcgtgcacactcaaggtcatcagcaaggtcatccgcagtcaggtggaacgtggaggcctctctctgggatcgtctccagcggataaaggactgtgcacagcttcggaagcttttatttaaaaatataactattaattattgcattataagtaatcactaatggtatcagcaattataatatttattaaagtataattagaaatattaagtagtacacacgttctggaaaaacacaaattgcacatggcagcagagtgaattttggccgagggacacgtgtgcacatgtgtgtaagcggcccccaggcccacagaattcgctgacaaagtcacctccccagagaagccaccacgggcctccttcgtggtcgtgaattttattaagatggatcaagtcacgtaccgtccacgtgtggcagggctttggggaatgtgaggtgatgactgcgtcctcatgccctgacagacaggaggtgactgtgtctgtcctgtccctaggacacggacaggcccgaagctctagtccccatcgtggtccagtttggcctctgaataaaaacgtcttcaaaacctgttgccccaaaaactaagaacagagagagtttcccatcccatgtgctcacaggggcgtatctgcttgcgttgactcgctgggctggccggactcctagagttggtgcgtgtgcttctgtgcaaaaagtgcagtcctcttgcccatcactgtgatatctgcaccagcaaggaaagcctcttttcttttctttctttttttttttttgagacggaacgtcactgttgtctgcctgggcttgagtgcagtggcgcgatctcaactcactgcaacctccgcctcccgggttccagcatttctcctgcctcagcctcccgagcagctgagattacaggcacccaccccctgcgcctggctaatttttgtatttttagtagagaggggtttttgccatgttggccaggctggtctcgaactcctgacctcaggtgatccacccacctcggcctcccaaagtgctgggattacaggtgtgagccatcacgcccagccggaaagcctctttttaaggtgaccacctatagcgcttcccgaaaataacaggtcttgtttttgcagtaggctgcaagcgtctcttagcaacaggagtggcgtcctgtgggctctggggatggctgagggtcgcgtggcagccatgccttctgtgtgcacctttaggttccacggggctattctgctctcactgtttgtctgaaaacgcacccttggcatccttgtttggagagtttctgcttctcgttggtcatgctgaaactaggggcaaggttgtatccgttggcgcgcagcggctacatgtagggtcatgagtctttcaccgtggacaaattccttgaaaaaaaaaaaaggagtccggttaagcattcattccgggtcaagtgtctggttctgtgaataaactctaagatttaagaaaccttaatgaaagaaaaccttgatgattcagagcaaggatgtggtcacacctgtggctggatctgtttcagccgccccagtgcatggtgagagtggggagcagggattgtttgttcagaggtctcatctggtatgtttctgaggtgtttgccggctgaatggtagacgtgtcgtttgtgtgtatgaggttctgtgtctgtgtgtggctcggtttgagtgtacgcatgtccagcacatgccctgcccgtctctcacctgtgtcttcccgccccaggtcctacgtccagtgccaggggatcccgcagggctccatcctctccacgctgctctgcagcctgtgctacggcgacatggagaacaagctgtttgcggggattcggcgggacgggtgaggcctcctcttccccaggggggcttgggtgggggttgatttgcttttgatgcattcagtgttaatattcctggtgctctggagaccatgactgctctgtcttgaggaaccagacaaggttgcagccccttcttggtatgaagccgcacgggaggggttgcacagcctgaggactgcgggctccacgcaggctctgtccagcggccatgtccagaggcctcagggctcagcaggcgggagggccgctgccctgcatgatgagcatgtgaattcaacaccgaggaagcacaccagcttctgtcacgtcacccaggttccgttagggtccttggggagatggggctggtgcagcctgaggccccacatctcccagcaggccctcgacaggtggcctggactgggcgcctcttcagcccattgcccatcccacttgcatggggtctacacccaaggacgcacacacctaaatatcgtgccaacctaatgtggttcaactcagctggcttttattgacagcagttacttttttttttttaatactttaagttctagggtacatgtgcacgacgtgcaggttagttacatatgtatacatgtgccatgttggtgtgctgcacccattaactcatcatttacattaggtatatctcctaatgctatccctccccactccccccatcccatgacaggccctggtgtgtgatgttccccaccctgtgtccaagtgttctcattgttcagttcccacctgtgagtgagaacatgtggtgtttggttttctttccttgcaatagtttgctcagagtgatggtttccagcttcgtccatgtccctacaaaggacatgaactcatccttttttatgactgcatagtattccgtggtgtatatgtgccacattttcttaatccagtctatcatcgatggacatttgggttggttgcaagtctttgctactgtgaatagtgccgcaataaacatacgtgtgcatgtgtctttatagcagcatgatttataatcctttgggtatatacccagtaatgggatggctgggtcaaatggtatttctagttctagatccttgaggaatcaccacactgtcttccacaatggttgaactagtttacactcccaccaacagtgtaaaagtgttctggtgctggagaggatgtggacagcagttatttttttatgaaaatagtatcactgaacaagcagacagttagtgaaggatgcgtcaggaagcctgcaggccacacagccatttctctcgaagactccgggtttttcctgtgcatcttttgaaactctagctccaattatagcatgtacagtggatcaaggttcttcttcattaaggttcaagttctagattgaaataagtttatgtaacagaaacaaaaatttcttgtacacacaacttgctctgggatttggaggaaagtgtcctcgagctggcggcacactggtcagccctctgggacaggatacctctggcccatggtcatggggcgctgggcttgggcctgagggtcacacagtgcaccatgcccagcttcctgtggataggatctgggtctcggatcatgctgaggaccacagctgccatgctggtaaagggcaccacgtggctcagagggggcgaggttcccagccccagctttcttaccgtcttcagttatttttccctaagagtctgagaagtggggccgcgcctgatggccttcgttcgtcttcagctggcacagaattgcacaagctgatggtaaacactgagtacttataatgaatgaggaattgctgtagcagttaactgtagagagctcgtctgttggaaagaaatttaagtttttcatttaaccgctttggagaatgttactttatttatggctgtgtaaattgtttgacattcagtccctcgtagacagatactacgtaaaaagtgtaaagttaaccttgctgtgtattttcccttattttaggctgctcctgcgtttggtggatgatttcttgttggtgacacctcacctcacccacgcgaaaaccttcctcaggtgaggcccgtgccgtgtgtctgtggggacctccacagcctgtgggctttgcagttgagccccccgtgtcctgcccctggcaccgcagcgttgtctctgccaagtcctctctctctgccggtgctggatccgcaagagcagaggcgcttggccgtgcacccaggcctgggggcgcaggggcaccttcgggagggagtgggtaccgtgcaggccctggtcctgcagagacgcacccaggttacacacgtggtgagtgcaggcggtgacctggctcctgctgctctttggaaagtcaagagtggcggctcctggggccccagtgagacccccaggagctgtgcacagggcctgcagggccgaggcggcagcctcctccccagggtgcacctgagcctgcggagagcaggagctgctgagtgagctggcccacagcgttcgctgcggtcacgttcctgcgtggggttgtttgggatcggtgggagaatttggatttgctgagtgctgctgtcttgaaccacggagatggctaggagtgggtttcagagttgatttttgtgaatcaaactaaaatcaggcacaggggacctggcctcagcacaggggattgtccaatgtggtccccctcaagggcgccccacagagccggtgggcttgttttaaagtgcgatttgacgagggacgagaaaccttgaaagctgtaaagggaaccctcagaaaatgtggccgccaggggtggtttcaggtgctttgctgggctgtgtttgtgaaaacccatttggacccgccctccaagtccaccctccaggtccaccctccagggccgccctgggctgggggtatgcctggcgttccttgtgccgcagcccggagcacagcaggctgtgcacatttaaatccactaagattcactcggggggagcccaggtcccaagcaactgagggctcaggagtcctgaggctgctgaggggacagagcagacggggaacgctgcttctgtgtggcaagttcctgagggtgctggccagggaggtggctcagagtgtatgttggggtcccaccgggggcagaactctgtctctgatgagtcggcagccatgtaacaggaaggggtggccacagggagctgggaatgcaccaggggagctgcgcagctggccgaggtcccagggccaggccacaggaagggcagggggacgcccggggccacagcagaggccgcaggaagggaaggggatgcccaggccagagcagaggctaccgggcacaggggggctccctgagctgggtgagcgaggctcatgactcggcgagggaacctccttgacgtgaagctgacgactggtgttgcccagctcacagcccagccaggtcccgcgcctgagcaggaactcagaaccctcccctttgtctaaagcacagcagatgccttcagggcatctaggagaaaacaggcaaagtcgttgagaaacgtcttaaaagaaggtgggatggtggcaatttcttgtccagattttagtctgccccggaccacagatgagtctataacgggattgtggtgttgccatggggacacatgagatggaccatcacagaggccactggggctgcacctcccatctgagtcctggctgtcccgggtccaggccaggttcttgcatgctcacctacctgtcctgcccgggagacagggaaagcaccccgaagtctggagcagggctgggtccaggctcctcagagctcctgccaggcccagcaccctgctccaaatcaccacttctctggggttttccaaagcatttaacaagggtgtcaggttacctcctgggtgacggccccgcatcctggggctgacattgcccctctgccttaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccaggtgagcgcacctggccggaagtggagcctgtgcccggctggggcaggtgctgctgcagggccgttgcgtccacctctgcttccgtgtggggcaggcgactgccaatcccaaagggtcagaggccacagggtgcccctcgtcccatctggggctgagcagaaatgcatctttctgtgggagtgagggtgctcacaacgggagcagttttctgtgctattttggtaaaaggaaatggtgcaccagacctgggtgcactgaggtgtcttcagaaagcagtctggatccgaacccaagacgcccgggccctgctgggcgtgagtctctcaaacccgaacacaggggccctgctgggcatgagtccctctgaacccgagaccctggggccctgctgggcgtgagtctctccgaacccagagacttcagggcccttttgggcgtgagtctctccgctgtgagccccacactccaaggctcatccacagtctacaggatgccatgagttcatgatcacgtgtgacccatcaggggacagggccatggtgtggggggggtctctacaaaattctggggtcttgtttccccagagcccgagagctcaaggccccgtctcaggctcagacacaaatgaattgaagatggacacagatgcagaaatctgtgctgtttcttttatgaataaaaagtatcaacattccaggcagggcaaggtggctcacacctataatcccagcactttgggaggccgaggtgggtggatcacttgaggccaggagtttgaggccaacctaaccaacatagtgaaattccatttctacttaaaaaatacaaaaattagcctggcctggtggcacacgcctgtagtccccgctatgcgggaggctgaggcaggagaatcatttgaacccaggaggcagaggttgcagtgagccgagatcacaccactgcactccagcctgggcaacagagtgagacttcatcttaaaaaaaaaaaaaaaagtatcagcattccaaaaccatagtggacaggtgtttttttattctgtccttcgataatatttactggtgctgtgctagaggccggaactgggggtgccttcctctgaaaggcacaccttcatgggaagagaaataagtggtgaatggttgttaaaccagaggtttaaactggggtcctgtcgttctgagttaacagtccagatctggactttgcctctttccagaatgctccctggggtttgcttcatgggggagcagcaggtgtggacaccctcgtgatgggggagcagcaggtgcagacgccctcatgatgggggagtggcaggtgcagacacccttgtgcatggtgcccagcatgtccctgttgcagctccctccccacaaggatgccggtctcctgtgctccccacagtccctgcttccctctcacagccttacctggtcctggcctccactggctttgtctgcatgatttccacatttcctgggctcccagcacctcttcgcctctcccaggcacctctgcagtgctggccataccagtcagctgtgaactgtccactgcttattttgctccccatgaaatgtattttttaggacaggcacccctggttccagcctctggcacagcatcagtgaatgttattgaaggacaaaggacagacaaacaaatcaggaaaatgggttctctctaaacacattgcaaagccacagaggctagtgcaggatgggtgggcatcaggtcatcagatgtgggtccaatgccagaatattctgtgctcccaaaggccacttggtcagagtgtgtgcttgcagaggtggctctaaaagctcagcagtggaggcagtggttcgccatactcagggtgaactcacatcctctgtgtctgaagtatacagcagaggcttgaagggcatctgggagaagaaaacaggcaaaatgattaagaaaagtgaaaaaggaaaagtggtaagatgggaattttcttgtccagattttagtctcccaaaccacagctcagatggtagaatgtggtcagaactgatggacagaacaatagaacaaaacggaagccctatctctcagaaacgtgtgttaatgtggtatgtggcacagctgatggaaaagagagtgtgtgtgtaatttttttttctgagaaaactgactggaagcaaataagttgtgtctttacagcatataccagagcagattctaggtagaagaggagacacatgcaaacaacaccagcaacagaaataaaacaaaagactcaaagggaagggaggtgaacgttccctggtttggtgttggggaaggacacacagggaggcggatgaaaccagtgaggcaacgggcattgctttcactgcagagaaactcagcttgcctgagccacagtgaaaatggccattccctggagcgtttgtgcacgtgatttatttaaggcgccctgtgaggtcctgcacattcatcctctcactttgttctcctaaccacctgagaggtagaggaggaaaggctccaggggagcagccgcccttggtcacccagctggcaaagggcatgcatgattgcagcctggcctcctgctccggggcccttgctctgcccgaggaccccacacaagtcagacccataggctcagggtgagccggagcccaaggtcgtgttggggatggctgtgaaagaagaaatggacgtctgatgcacacttgggaaggtcctaccagcagcgtcaaagaaatgcatgtgaaactgacagcgagacccatccctcaaagaaacgcacgtgaaactgatggcgagacctgtccccatccctcatgctggctccttttctgggcttgccaagagccagcatcaggttgaggcaagctggaaagacttttctggaaagcagcttgtttgcatggaagtcctcacaatgtcctgtgtcttcccagtaattccacttctgaagtgaccagacattatcacgggtcttatttaccatttccagtgttccaggcagggggacttgccacagcaagtcacgaacctgcccaaatacagggctaaggagatattatgcatcacaaaacttgctctgccattaaacatttttcaaagaatttttgaagaatgtttaatggcacaaaacgtttatttcaatgtagcagtgttcaaagctggatgtaaaagaacacaccccaggagcctgccgtgaatgtcatgtgtgttcatctttggacatggacatacatgggcagtgagtggtggtgaggccctggaggacatcggtgggatgcctccatcctgcccctctggagacaccatgtgtgccacgtgcactcactggagccctgtttagctggtgccacctggctcttccatccctgagattcaaacacagtgagattccccacgcccaactcagtgttctcccacaaaaaacctgagtcacacctgtgttcactcgagggacgcccgggagccagggctccacagtttattatgtgtttttggctgagttatgtgcagatctcatcagggcagatgatgagtgcacaaacacggccgtgcgaggtttggatacactcaacatcactagccaggtcctggtggagtttggtcatgcagagtctggatggcatgtagcatttggagtccatggagtgagcacccagccccctcgggctgcagcgcatgccccaggcaggacaaggaagcgggaggaaggcaggaggctctttggagcaagctttgcaggagggggctgggtgtggggcaggcacctgtgtctgacattcccccctgtgtctcagctatgcccggacctccatcagagccagtctcaccttcaaccgcggcttcaaggctgggaggaacatgcgtcgcaaactctttggggtcttgcggctgaagtgtcacagcctgtttctggatttgcaggtgagcaggctgatggtcagcacagagttcagagttcaggaggtgtgtgcgcaagtatgtgtgtgtgtgtgtgcgcgcgtgcctgcaaggctgatggtgactggctgcacgtaagagtgcacatgtacgcatatacacgtgagcacatacatgtgtgcatgtgtgtacatgaaggcatggcagtgtgtgcacaggtgtgcaagggcacaagtgtgtgcacatgcgaatgcacacctgacatgcatgtgtgttcgtgcacagtcgtgtgggcattcacgtgaggtgcatgcgtgtgggtgtgcagtgtgagtagcatgtgtgcacataacatgtattgaggggtcctcgtgttcaccccgctaggtcctcagcaccagtgccactccttacaggatgagacggggtcccaggccttggtgggctgaggctctgaagctgcagccctgagggcattgtcccatctgggcatccgcgtccactccctctcctgtgggcttctgtgtccactccccctctcctgtgggcatttacatccactccactccctctctcctgtgggcatccgcgtccactccccctctctgtgggcatctgcgtccacctcccctctctgtgggcatttgcgtccactccctctcctggttccttcctgtcttggccgagcctcgggggcaggcagatgacacagagtcttgactcgcccagggtggttcgcagctgccgggtgagggccaggccggatttcactgggaagagggatagtttcttgtcaaaatgttcctctttcttgttccatctgaatggatgataaagcaaaaagtaaaaacttaaaatcccagagaggtttctaccgtttctcactctttcttggcgactctaggtgaacagcctccagacggtgtgcaccaacatctacaagatcctcctgctgcaggcgtacaggtgagccgccaccaaggggtgcaggcccagcctccagggaccctccgcgctctgctcacctctgacccggggcttcaccttggaactcctgggttttaggggcaaggaatgtcttacgttttcagtggtgctgctgcctgtgcacagttctgttcgcgtggctctgtgcaaagcacctgttctccatctctgggtagtggtaggagccggtgtggccccaggtgtccccactgtgcctgtgcactggccgtgggacgtcatggaggccatcccagggcagcaggggcatggggtaaagagatgtttatggggagtcttagcagaggaggctgggaaggtgtctgaacagtagatgggagatcagatgcccggaggatttggggtctcagcaaagagggccgaggtgggtgcaggtgagggtcgctggccccacccccgggaaggtgcagcagagctgtggctccccacacagcccggccagcacctgtgctctgggcatggctgtgctcctggaacgttccctgtcctggctggtcagggggtgcccctgccaagaatcgacaactttatcacagagggaagggccaatctgtggaggccacagggccagcttctgcctggagtcagggcaggtggtggcacaagcctcggggctgtaccaaagggcagtcgggcaccacaggcccgggcctccacctcaacaggcctcccgagccactgggagctgaatgccaggaggccgaagccctcgccccatgagggctgagaaggagtgtgagcatttgtgttacccagggccgaggctgcgcgaattaccgtgcacacttgatgtgaaatgaggtcgtcgtctatcgtggaaacccagcaagggctcacgggagagttttccattacaaggtcgtaccatgaaaatggtttttaacccgagtgcttgcgccttcatgctctggcagggagggcagagccacagctgcatgttaccgcctttgcaccagctccagaggcttgggaccaggctgtctcagttccagggtgcgtccggctcagaccgccctcctctctgccttctctctctgcctcaaatcttccctcgtttgcatctccctgacgcgtgcctgggccctcgtgcaagctgcttgactcctttccggaaacccttggggtgtgctggatacaggtgccactgaggactggaggtgtctgacactgtggttgaccccagggtccagctggcgtgcttggggcctccttgggccatgatgaggtcagaggagttttcccaggtgaaaactcctgggaaactcccagggccatgtgacctgccacctgctcctcccatattcagctcagtcttgtcctcatttccccaccagggtctctagctccgaggagctcccgtagagggcctgggctcagggcagggcggctgagtttccccacccatgtggggacccttgggtagtcgcttgattgggtagccctgaggaggccgagatgcgatgggccacgggccgtttccaaacacagagtcaggcacgtggaaggcccaggaatccccttccctcgaggcaggagtgggagaacggagagctgggccccgatttcacggcagccaggctgcagtgggcgaggctgtggtggtccacgtggcgctgggggcggggtctgattcaaatccgctggggctcggccttcctggcccgtgctggccgcgcctccacacgggcttggggtggacgccccgacctctagcaggtggctatttctccctttggaagagagcccctcacccatgctaggtgtttccctcctgggtcaggagcgtggccgtgtggcaaccccgggaccttaggcttatttatttgtttaaaaacattctgggcctggcttccgttgttgctaaatggggaaaagacatcccacctcagcagagttactgagaggctgaaaccggggtgctggcttgactggtgtgatctcaggtcattccagaagtggctcaggaagtcagtgagaccaggtacatggggggctcaggcagtgggtgagatgaggtacacggggggctcaggcagtgggtgaggccaggtacatggggggctcaggcactgggtgagatgaggtacacggggggctcaggcagagggtcagaccaggtacacgggggctctgatcacacgcacatatgagcacatgtgcacatgtgctgtttcatggtagccaggtctgtgcacacctgccccaaagtcccaggaagctgagaggccaaagatggaggctgacagggctggcgcggtggctcacacctgtagtcccagcactttgggaggccgaggcgagaggatcccttgagcccaggagtttaagaccagcctgagcaacatagtagaaccccatctctatgaaaaataaaaacaaaaattagctgaacatggtggtgtgcgcctgtagttccaatacttgggaggctgaagtgggaggatcacttgagcccaggaggtggaagctgcagtgagctgagattgcaccactgtactgcagcctgggtgacagagtgagagcccatctcaacaacaacaaagaagactgacaaatgcagtttcttggaaagaaacatttagtaggaacttaacctacacacagaagccaagtcggtgtctcggtgtcagtgagatgagatgatgggtcctcacaccatcaccccagacccagggtttatgcaccacaggggcgggtggctcagaagggatgcgcaggacgttgatatacgatgacatcaaggttgtctgacgaagggcaggattcatgataagtacctgctggtacacaaggaacaatggataaactggaaaccttagaggccttcccggaacaggggctaatcagaagccagcatggggggctggcatccaggatggagctgcttcagcctccacatgcgtgttcatacagatggtgcacagaaacgcagtgtacctgtgcacacacagacacgcagctactcgcacacacaagcacacacacagacatgcatgcatgcatccgtgtgtgtgcacctgtgcccatgaggaaacccatgcatgtgcattcatgcacgcacacaggcaccggtgggcccatgcccacacccacgagcaccgtctgattaggaggcctttcctctgacgctgtccgccatcctctcaggtttcacgcatgtgtgctgcagctcccatttcatcagcaagtttggaagaaccccacatttttcctgcgcgtcatctctgacacggcctccctctgctactccatcctgaaagccaagaacgcaggtatgtgcaggtgcctggcctcagtggcagcagtgcctgcctgctggtgttagtgtgtcaggagactgagtgaatctgggcttaggaagttcttaccccttttcgcatcaggaagtggtttaacccaaccactgtcaggctcgtctgcccgccctctcgtggggtgagcagagcacctgatggaagggacaggagctgtctgggagctgccatccttcccaccttgctctgcctggggaagcgctggggggcctggtctctcctgtttgccccatggtgggatttggggggcctggcctctcctgtttgccctgtggtgggattgggctgtctcccgtccatggcacttagggcccttgtgcaaacccaggccaagggcttaggaggaggccaggcccaggctaccccacccctctcaggagcagaggccgcgtatcaccacgacagagccccgcgccgtcctctgcttcccagtcaccgtcctctgcccctggacactttgtccagcatcagggaggtttctgatccgtctgaaattcaagccatgtcgaacctgcggtcctgagcttaacagcttctactttctgttctttctgtgttgtggaaatttcacctggagaagccgaagaaaacatttctgtcgtgactcctgcggtgcttgggtcgggacagccagagatggagccaccccgcagaccgtcgggtgtgggcagctttccggtgtctcctgggaggggagctgggctgggcctgtgactcctcagcctctgttttcccccagggatgtcgctgggggccaagggcgccgccggccctctgccctccgaggccgtgcagtggctgtgccaccaagcattcctgctcaagctgactcgacaccgtgtcacctacgtgccactcctggggtcactcaggacaggcaagtgtgggtggaggccagtgcgggccccacctgcccaggggtcatccttgaacgccctgtgtggggcgagcagcctcagatgctgctgaagtgcagacgcccccgggcctgaccctgggggcctggagccacgctggcagccctatgtgattaaacgctggtgtccccaggccacggagcctggcagggtccccaacttcttgaacccctgcttcccatctcaggggcgatggctccccacgcttgggagccttctgacccctgacctgtgtcctctcacagcctcttccctggctgctgccctgagctcctggggtcctgagcaagttctctccccgccccgccgctccagcgtcactgggctgcctgtctgctcgccccggtggaggggtgtctgtcccttcactgaggttcccaccagccagggccacgaggtgcaggccctgcctgcccggccacccacacgtcctaggagggttggaggatgccacctctggcctcttctggaacggagtctgattttggccccgcagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaatctcatgtttgaatcctaatgtgcactgcatagacaccactgtatgcaattacagaagcctgtgagtgaacggggtggtggtcagtgcgggcccatggcctggctgtgcatttacggaagtctatgagtgaatggggttgtggtcagtgcgggcccatggcctggctgggcctgggaggtttctgatgctgtgaggcaggaggggaaggagggtaggggatagacagtgggagcccccaccctggaagacataacagtaagtccaggcccgaagggcagcagggatgctgggggcccagcttgggcggcggggatgatggagggcctggccagggtggcagggatgatgggggccccagctggggtggcaggggtgatggggggggctggtctgggtggcggggaagatggggaagcctggctgggccccctcctcccctgcctcccacctgcagccgtggatccggatgtgcttccctggtgcacatcctctgggccatcagctttcatggaggtggggggcaggggcatgacaccatcctgtataaaatccaggattcctcctcctgaacgccccaactcaggttgaaagtcacattccgcctctggccattctcttaagagtagaccaggattctgatctctgaagggtgggtagggtggggcagtggagggtgtggacacaggaggcttcagggtggggctggtgatgctctctcatcctcttatcatctcccagtctcatctctcatcctcttatcatctcccagtctcatctgtcttcctcttatctcccagtctcatctgtcatcctcttaccatctcccagtctcatctcttatcctcttatctcctagtctcatccagacttacctcccagggcgggtgccaggctcgcagtggagctggacatacgtccttcctcaggcagaaggaactggaaggattgcagagaacaggaggggcggctcagagggacgcagtcttggggtgaagaaacagcccctcctcagaagttggcttgggccacacgaaaccgagggccctgcgtgagtggctccagagccttccagcaggtccctggtggggccttatggtatggccgggtcctactgagtgcaccttggacagggcttctggtttgagtgcagcccggacgtgcctggtgtcggggtgggggcttatggccactggatatggcgtcatttattgctgctgcttcagagaatgtctgagtgaccgagcctaatgtgtatggtgggcccaagtccacagactgtgtcgtaaatgcactctggtgcctggagcccccgtataggagctgtgaggaaggaggggctcttggcagccggcctgggggcgcctttgccctgcaaactggaagggagcggccccgggcgccgtgggcggacgacctcaagtgagaggttggacagaacagggcggggacttcccaggagcagaggccgctgctcaggcacacctgggtttgaatcacagaccaacaggtcaggccattgttcagctatccatcttctacaaagctccagattcctgtttctccgggtgttttttgttgaaattttactcaggattacttatattttttgctaaagtattagacccttaaaaaaggtatttgctttgatatggcttaactcactaagcacctactttatttgtctgtttttatttattattattattattattagagatggtgtctactctgtcacccaggttgttagtgcagtggcacagtcatggctcgctgtagccgcaaacccccaggctcaagtgatcctccggcctcagcttcccagagtgctgggattacaggtgtgagccactgcccttgcctggcacttttaaaaaccactatgtaaggtcaggtccagtggcttccacacctgtcatcccagtagtttgggaagccgaggcagaaggattgtctgaggccaggagtttgagaccagcatgggtaacatagggagaccccatctctacaaaaaatgcaaaaagttatccgggcgtggggtccagcatctgtagtcccagctgctcgggaggctgagtgggaggatcgcttgagcccgggaggtcatggctgcagtgagctgtgattgtaccatcgcactccagcctgggcaacagagtgagaccctgtctcaaaaaaaaaaaaaaaaaaagaaggagaaggagaagagaagaagaaggaagaaggaaagagaagaagaaggaagaaggaagaaagaaggagaaggaggcctgctaggtgctaggtagactgtcaaatctcagagcaaaatgaaaataacaaagttttaaagggaaagaaaaaccccagctctttggacttccttaggcctgaacttcatctcaagcagcttccttccacagacaagcgtgtatggagcgagtgagttcaaagcagaaagggaggagaagcaggcaagggtggaggctgtgggtgacaccagccaggacccctgaaagggagtggttgttttcctgcctcagccccacgctcctgccggtcctgcacctgctgtaaccgtcgatgttggtgccaggtgcccacctgggaaggatgctgtgcagggggcttgccaaactttggtgggtttcagaagccccaggcacttgtggcaggcacaattacagcccctccccaaagatgcccacgtccttctcctggaacctgtgaatgtgtcacccgcaaggcagaggctggtgaaggctgcaggtggaatcacggctgccagtcagccgatcttaaggtcatcctggattatctggtgggcctgatatggccacaagggtccctagaagtgagagagggaggcaggggagagtcagagaggggacgtgagaaggaccactggccactgctggctttgagatggaggagggggtccccagccaaggaatgggggcagccgctccatgctggaaaagcaagcaatcctccccggtcctgagggcacacggccctgcccacgcctcgatttcaggccagtgggacctgtttcagctttccggcctccagagctgtaagatgatgcgtttgtgttcagccactaagctgcagtgattcgtcacagcagcaaatggaatagcagtacagggaaatgaatacagggacagttctcagagtgactctcagcccacccctggg